(19) United States
(12) Patent Application Publication
(10) Pub. No.: US 2025/0101436 A1
(43) Pub. Date: Mar. 27, 2025

(54) APTAMERS AND SMALL MOLECULE LIGANDS

(71) Applicant: MeiraGTx UK II Limited, London (GB)

(72) Inventors: Xuecui Guo, Oyster Bay, NY (US); Kevin G. Liu, West Windsor, NJ (US); Ji-In Kim, Princeton, NJ (US); Alexandria Forbes, New York, NY (US)

(21) Appl. No.: 18/719,538

(22) PCT Filed: Dec. 15, 2022
(86) PCT No.: PCT/IB2022/000762

§ 371 Date: Jun. 13, 2024

Related U.S. Application Data
(60) Provisional application No. 63/361,400, filed on Dec. 15, 2021.

(51) Int. Cl. C12N 15/115; C07D 241/42; C07D 401/12; C07D 401/14; C07D 471/04; C07D 487/04; C07D 487/08; C07D 487/10; C12N 15/85

(52) U.S. Cl. CPC C12N 15/115; C07D 241/42; C07D 401/12; C07D 401/14; C07D 471/04; C07D 487/04; C07D 487/08; C07D 487/10; C12N 15/85; C12N 2310/16; C12N 2310/531; C12N 2800/107; C12N 2840/002

(57) ABSTRACT

The present disclosure provides aptamers that bind to certain small molecules. Also contemplated are riboswitches and polynucleotide cassettes for regulating the expression of a target gene, wherein the polynucleotide cassettes comprise the aptamers disclosed herein. Further provided are small molecules that bind to the aptamers disclosed herein and are modulators of target gene expression where the target gene contains a riboswitch comprising an aptamer described herein.

### FIELD

The present disclosure relates to oligonucleotide aptamers that bind to certain small molecules and methods of generating aptamers that bind to the small molecules. Also contemplated are riboswitches and polynucleotide cassettes for regulating the expression of a target gene, wherein the polynucleotide cassettes comprise the aptamers disclosed herein. Further provided are small molecules that are modulators of target gene expression where the target gene contains a riboswitch comprising an aptamer described herein.

### BACKGROUND

Aptamers are oligonucleotides that bind to a target ligand with high affinity and specificity. These nucleic acid sequences have proven to be of high therapeutic and diagnostic value with recent FDA approval of the first aptamer drug and additional ones in the clinical pipelines. Their high degree of specificity and versatility have established RNA aptamers as one of the pivotal tools of the emerging RNA nanotechnology field in the fight against human diseases including cancer, viral infections and other diseases.

In addition, aptamers may be utilized as part of a riboswitch that has certain effects in the presence or absence of an aptamer ligand. For example, riboswitches may be used to regulate gene expression in response to the presence or absence of the aptamer ligand.

However, aptamers/ligands derived from prokaryotic sources or generated using in vitro selection methods often fail to demonstrate the functionality required for the expression of therapeutic targets genes in eukaryotic systems. For example, the ligand for the aptamer may be a cellular molecule that would not be appropriate for use in systems for regulating a therapeutic gene product, for example, because presence of the ligand would interfere in the regulation of target gene expression, or because the ligand is not otherwise appropriate for administration to cell or tissue. As such, new aptamer sequences, small molecule ligands, and aptamer/ligand combinations able to regulate gene expression in response to the presence or absence of the small molecule ligand are needed.

### SUMMARY

Provided herein are aptamer sequences that bind to small molecules of Formula I to XXII, including those listed in Table A, and analogs or derivatives thereof. Also contemplated are riboswitches and polynucleotide cassettes for regulating the expression of a target gene, wherein the polynucleotide cassettes comprise the aptamers disclosed herein. Further provided are methods of using said aptamers, riboswitches, and/or polynucleotide cassettes for the regulation of target genes, including therapeutic genes. Also provided herein are small molecules that are modulators of target gene expression where the target gene contains a riboswitch comprising an aptamer described herein.

In one aspect, the disclosure provides an aptamer comprising the aptamer encoding sequence disclosed herein. In embodiments, the aptamer encoding sequence comprises:
CTGGGGAGTCCTTCATGCGGGGCTGAGAGGATGGAAGX₁X₂AX₃X₄X₅X₆CCAT CGACCCX₇X₈X₉X₁₀X₁₁X₁₂CCTX₁₃X₁₄X₁₅CCGGX₁₆X₁₇X₁₈X₁₉X₂₀X₂₁CCGGX₂₂X₂₃C AGGGAG (SEQ ID NO:2); wherein:
X₁ is C or T;
X₂ is any nucleotide;
X₃ is any nucleotide;
X₄ is G or T;
X₅ is A, G, or T;
X₆ is A or G;
X₇ is A or T;
X₈ is A, C, or T;
X₉ is A, C, or T;
X₁₀ is any nucleotide;
X₁₁ is any nucleotide or no nucleotide;
X₁₂ is A;
X₁₃ is A, C, or G;
X₁₄ is any nucleotide;
X₁₅ is C, G, or T;
X₁₆ is G or T;
X₁₇ is A or T;
X₁₈ is any nucleotide;
X₁₉ is A or G;
X₂₀ is A, G, T;
X₂₁ is C, G, T;
X₂₂ is T; and
X₂₃ is A, G, or T (taken together SEQ ID NO:681).

In embodiments, the aptamer encoding sequence comprises:
CTGGGGAGTCCTTCATGCGGGGCTGAGAGGATGGAAGX₁X₂AX₃X₄X₅X₆CCAT CGACCCX₇X₈X₉X₁₀X₁₁X₁₂CCTX₁₃X₁₄X₁₅CCGGX₁₆X₁₇X₁₈X₁₉X₂₀X₂₁CCGGX₂₂X₂₃C AGGGAG (SEQ ID NO:2); wherein:
X₁ is C or T;
X₂ is any nucleotide;
X₃ is any nucleotide;
X₄ is G or T;
X₅ is A, G, or T;
X₆ is A or G;
X₇ is A;
X₈ is A, C, or T;
X₉ is A, C, or T;
X₁₀ is any nucleotide;
X₁₁ is any nucleotide or no nucleotide;
X₁₂ is A;
X₁₃ is A, C, or G;
X₁₄ is any nucleotide;
X₁₅ is C, G, or T;
X₁₆ is G or T;
X₁₇ is A or T;
X₁₈ is any nucleotide;
X₁₉ is A or G;
X₂₀ is A, G, T;
X₂₁ is C, G, T;
X₂₂ is T; and
X₂₃ is A, G, or T (taken together SEQ ID NO:682).

In embodiments, the aptamer encoding sequence comprises:
CTGGGGAGTCCTTCATGCGGGGCTGAGAGGATGGAAGCAATCGACCATCGA CCCX₇X₈X₉X₁₀X₁₁X₁₂CCTGATCCGGATCATGCCGGCGCAGGGAG (SEQ ID NO:4); wherein:
X₇ is A, G, or T;
X₈ is any nucleotide;
X₉ is any nucleotide;
X₁₀ is any nucleotide;
X₁₁ is any nucleotide or no nucleotide;
X₁₂ is A, C, or T (taken together SEQ ID NO:683).

In embodiments, X₇—X₁₂ are not simultaneously A, T, T, G, C, and A, respectively.

In embodiments, the aptamer encoding sequence comprises: CTGGGGAGTCCTTCATGCGGGGCTGAGAGGATGGAAGCAATCGACCATCGA CCCX₇X₈X₉X₁₀X₁₁X₁₂CCTGATCCGGATCATGCCGGCGCAGGGAG (SEQ ID NO:4); wherein:
X₇ is A or T;
X₈ is A, C, or T;
X₉ is A, C, or T;
X₁₀ is any nucleotide;
X₁₁ is any nucleotide or no nucleotide; and
X₁₂ is A (taken together SEQ ID NO:684).

In embodiments, X₇X₁₂ are not simultaneously A, T, T, G, C, and A, respectively.

In embodiments, the aptamer encoding sequence comprises: CTGGGGAGTCCTTCATGCGGGGCTGAGAGGATGGAAGCAATCGACCATCGA CCCX₇X₈X₉X₁₀X₁₁X₁₂CCTGATCCGGATCATGCCGGCGCAGGGAG (SEQ ID NO:4); wherein:
X₇ is A;
X₈ is A, C, or T;
X₉ is A, C, or T;
X₁₀ is any nucleotide;
X₁₁ is any nucleotide or no nucleotide; and
X₁₂ is A (taken together SEQ ID NO:685).

In embodiments, X₇—X₁₂ are not simultaneously A, T, T, G, C, and A, respectively.

In embodiments, the aptamer encoding sequence comprises:

(SEQ ID NO: 3)
CTGGGGAGTCCTTCATGCGGGGCTGAGAGGATGGAAGX₁X₂AX₃X₄X₅X₆C
CATCGACCCATTGCACCTGATCCGGATCATGCCGGCGCAGGGAG;

wherein:
X₁ is C, G, or T;
X₂ is any nucleotide;
X₃ is any nucleotide;
X₄ is any nucleotide;
X₅ is any nucleotide; and
X₆ is any nucleotide (taken together SEQ ID NO:686).

In embodiments, X₁—X₆ are not simultaneously C, A, T, C, G, and A, respectively.

In embodiments, the aptamer encoding sequence comprises:

(SEQ ID NO: 3)
CTGGGGAGTCCTTCATGCGGGGCTGAGAGGATGGAAGX₁X₂AX₃X₄X₅X₆C
CATCGACCCATTGCACCTGATCCGGATCATGCCGGCGCAGGGAG;

wherein:
X₁ is C or T;
X₂ is any nucleotide;
X₃ is any nucleotide;
X₄ is any nucleotide;
X₅ is A, G, or T; and
X₆ is any nucleotide (taken together SEQ ID NO:687).

In embodiments, X₁—X₆ are not simultaneously C, A, T, C, G, and A, respectively.

In embodiments, the aptamer encoding sequence comprises:

(SEQ ID NO: 3)
CTGGGGAGTCCTTCATGCGGGGCTGAGAGGATGGAAGX₁X₂AX₃X₄X₅X₆C
CATCGACCCATTGCACCTGATCCGGATCATGCCGGCGCAGGGAG;

wherein:
X₁ is C or T;
X₂ is any nucleotide;
X₃ is any nucleotide;
X₄ is G or T;
X₅ is A, G, or T; and
X₆ is A or G (taken together SEQ ID NO:688).

In embodiments, the aptamer encoding sequence comprises:
CTGGGGAGTCCTTCATGCGGGGCTGAGAGGATGGAAGCAATCGACCATCGA CCCATTGCACCTX₁₃X₁₄X₁₅CCGGATCATGCCGGX₂₂X₂₃CAGGGAG (SEQ ID NO:5); wherein:
X₁₃, X₁₄, X₁₅, X₂₂, and X₂₃ is any nucleotide.

In embodiments, X₁₃, X₁₄, X₁₅, X₂₂, and X₂₃ are not simultaneously G, A, T, C, and G, respectively.

In embodiments, the aptamer encoding sequence comprises:
CTGGGGAGTCCTTCATGCGGGGCTGAGAGGATGGAAGCAATCGACCATCGA CCCATTGCACCTX₁₃X₁₄X₁₅CCGGATCATGCCGGX₂₂X₂₃CAGGGAG (SEQ ID NO:5); wherein:
X₁₃ is A, C, or G;
X₁₄ is any nucleotide;
X₁₅ is C, G, or T;
X₂₂ is T; and
X₂₃ is A, G, or T (taken together SEQ ID NO:689).

In embodiments, the aptamer encoding sequence comprises:
CTGGGGAGTCCTTCATGCGGGGCTGAGAGGATGGAAGCAATCGACCATCGA CCCATTGCACCTGATCCGGX₁₆X₁₇X₁₈X₁₉X₂₀X₂₁CCGGCGCAGGGAG (SEQ ID NO:6); wherein:
X₁₆ is any nucleotide;
X₁₇ is any nucleotide;
X₁₈ is any nucleotide;
X₁₉ is any nucleotide;
X₂₀ is any nucleotide; and
X₂₁ is C, G, T (taken together SEQ ID NO:690).

In embodiments, X₁₆—X₂₁, are not simultaneously A, T, C, A, T, and G, respectively.

In embodiments, the aptamer encoding sequence comprises: CTGGGGAGTCCTTCATGCGGGGCTGAGAGGATGGAAGCAATCGACCATCGA CCCATTGCACCTGATCCGGX₁₆X₁₇X₁₈X₁₉X₂₀X₂₁CCGGCGCAGGGAG (SEQ ID NO:6); wherein:
X₁₆ is G or T;
X₁₇ is A or T;
X₁₈ is any nucleotide;
X₁₉ is A or G;
X₂₀ is A, G, T; and
X₂₁ is C, G, T (taken together SEQ ID NO:691).

In embodiments, the aptamer encoding sequence comprises a sequence that is at least 95% identical, or at least 99% identical to a sequence selected from the group consisting of SEQ ID NOs: 1 and 7-558. In embodiments, the aptamer encoding sequence comprises a sequence that is selected from the group consisting of SEQ ID NOs: 1 and 7-558.

In embodiments, the aptamer encoding sequence comprises a sequence that is at least 95% identical, or at least 99% identical to a sequence selected from the group consisting of SEQ ID NOs: 7-17, 89-96, 174-349, and 358-583. In embodiments, the aptamer encoding sequence comprises a sequence that is selected from the group consisting of SEQ ID NOs: 7-17, 89-96, 174-349, and 358-583.

In embodiments, the aptamer encoding sequence comprises a sequence that is at least 95% identical, or at least 99% identical to a sequence selected from the group consisting of SEQ ID NOs: 7-11, 89-94, 174-349, and 358-447. In embodiments, the aptamer encoding sequence comprises a sequence that is selected from the group consisting of SEQ ID NOs: 7-11, 89-94, 174-349, and 358-447.

In embodiments, the aptamer encoding sequence comprises a sequence that is at least 95% identical, or at least 99% identical to a sequence selected from the group consisting of SEQ ID NOs: 174, 358, 363, and 378. In embodiments, the aptamer encoding sequence comprises a sequence that is selected from the group consisting of SEQ ID NOs: 174, 358, 363, and 378.

In embodiments, the aptamer sequence disclosed herein, further comprises additional sequence at the 5′ and 3′ ends that is complementary and capable of forming part of the aptamer P1 stem. In embodiments, this P1 stem of the aptamer is, comprises, or overlaps with the effector region of the riboswitches disclosed herein. In embodiments, the aptamer P1 stem comprises a 5′ splice site sequence of a 3′ intron and sequence complementary thereto. For example, the P1 stem may comprise A G G G T G A G T; A A A G T A A G C; G C A G T A A G T; G A G G T G T G G; A/C A G G T A/G A G T; N A G G T A/G A G T; N A G G T A A G T; A/C A/T G G T A N G T; or N A G/A G T A A G T (where N can be A, G, C, or T).

In embodiments, the aptamers disclosed herein bind to one or more of the small molecules of Formula I to XXII, including those listed in Table A.

In one aspect, the disclosure provides the RNA aptamer encoded by the aptamer encoding sequences disclosed herein.

In one aspect, the disclosure provides nucleic acid sequence encoding a recombinant riboswitch for the regulation of target gene expression in response to a small molecule, wherein the riboswitch comprises an aptamer disclosed herein.

In another aspect, the disclosure provides a polynucleotide cassette for regulating the expression of a target gene, wherein the polynucleotide cassette comprises a sequence encoding an aptamer that binds to a small molecule, wherein the aptamer encoding sequence comprises an aptamer encoding sequence disclosed herein.

In embodiments, the polynucleotide cassette comprises sequence encoding:
(a) a riboswitch; and
(b) an alternatively-spliced exon, flanked by a 5′ intron and a 3′ intron, wherein the riboswitch comprises (i) an effector region comprising a stem forming sequence that includes the 5′ splice site sequence of the 3′ intron (and sequence complementary to the 5′ splice site sequence of the 3′ intron), and (ii) the aptamer comprises an aptamer sequence disclosed herein; and wherein the alternatively-spliced exon comprises a stop codon that is in-frame with the target gene when the alternatively-spliced exon is spliced into the target gene mRNA.

In embodiments, the effector stem is, or comprises, a P1 stem of the aptamers disclosed herein. In other words, the effector stem comprises a first sequence that is linked to the 5′ end of the aptamers disclosed herein and a second sequence that is linked to the 3′ end of the aptamers disclosed herein.

In embodiments, the polynucleotide cassette is located in the protein coding sequence of the target gene. In embodiments, the polynucleotide cassette is located in an untranslated region of the target gene or in an intron of the target gene.

In embodiments, the small molecule has the structure according to Formula I.

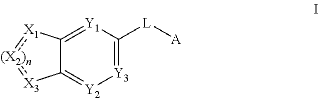

wherein
   X₁, X₂, and X₃ are, in each instance, independently selected from CR₁, CHR₁, N, NH, O and S, wherein adjacent X₁, X₂, and X₃ are not simultaneously selected to be O or S;
   the dashed lines represent optional double bonds;
   Y₁, Y₂, and Y₃ are, in each instance, independently selected from CR₂ and N;
   n is 1 or 2, wherein when n is 1, only one of the dashed lines is a double bond;
L-A is

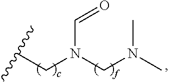

or
L is selected from

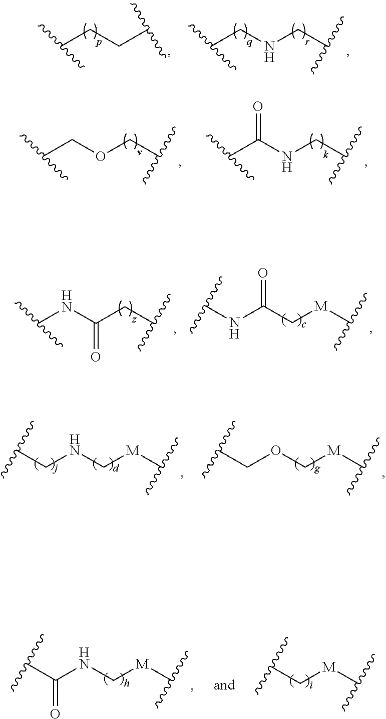

wherein k, p, q, r, and v are independently selected from integers 0, 1, 2, 3, 4, 5, 6, 7, 8, 9, and 10, z is selected from integers 1, 2, 3, 4, and 5;
c, d, e, f, g, h and i are independently selected from integers 1, 2, 3, 4, 5, 6, 7, 8, 9, and 10; j is selected from integers 0, 1, 2, 3, 4, 5, 6, 7, 8, 9, and 10;
   M is selected from —NH—, —O—, —NHC(═O)—, —C(═O)NH—, —S—, and —C(═O)—; and
   A is selected from

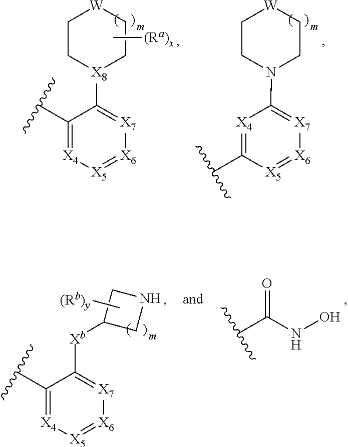

wherein X₄, X₅, X₆, and X₇, are independently selected from CR₃ and N;
X₈ is N or CH;
X^{b} is selected from O, NH, and NCH₃;
   wherein each of R₁, R₂, and R₃ are independently selected from —H, —Cl, —Br, —I, —F, —CF₃, —CH₂F, —CHF₂, —OH, —CN, —NO₂, —NH₂, —NH(C₁-C₆ alkyl), —N(C₁-C₆ alkyl)₂, —COOH, —COO(C₁-C₆ alkyl), —CO(C₁-C₆ alkyl), —O(C₁-C₆ alkyl), —OCO(C₁-C₆ alkyl), —NCO(C₁-C₆ alkyl), —CONH(C₁-C₆ alkyl), and substituted or unsubstituted C₁-C₆ alkyl;
   additionally or alternatively, two R₃ on adjacent ring positions may be taken together to form a 5- or 6-membered aromatic ring having from 0 to 2 heteroatoms selected from O, S, N and NH;
   m is 1 or 2;
each R^{a} is independently selected from C₁ to C₃ alkyl, —OCH₃, —CF₃, —CH₂F, —CHF₂, halo, hydroxyl and amino; or additionally or alternatively, two R^{a} attached to the same carbon atom form an oxo group, or two R^{a} attached to different carbon atoms form a 4- to 6-membered carbocyclic ring or a 4- to 6-membered heterocyclic ring having 1 or 2 heteroatoms selected from O and NH;
x is 0, 1, 2 or 3;
each R^{b} is independently selected from C₁ to C₃ alkyl, —OCH₃, —CF₃, —CH₂F, —CHF₂, halo, hydroxyl and amino; or additionally or alternatively, two R^{b} attached to the same carbon atom form an oxo group; or two R^{b} attached to different carbon atoms form a 4- to 6-membered carbocyclic ring or a 4- to 6-membered heterocyclic ring having 1 or 2 heteroatoms selected from O and NH;
y is 0, 1, 2 or 3; and
W is 0 or NR₄, wherein R₄ is selected from selected from —H, —CO(C₁-C₆ alkyl), substituted or unsubstituted C₁-C₆ alkyl, substituted or unsubstituted aryl, substituted or unsubstituted heteroaryl, substituted or unsubstituted cycloalkyl, —CO(aryl), —CO(heteroaryl), and —CO(cycloalkyl);
provided that at least two of X₁, X₂, X₃, X₄, X₅, X₆, and X₇ are N; or a pharmaceutically acceptable salt thereof.

In embodiments, the small molecule has a structure according to Formula II-XXII, including, e.g., a structure provided in Table A.

In one aspect the disclosure provides a vector comprising a polynucleotide cassette, an aptamer encoding sequence/aptamer sequence, or riboswitch disclosed herein. In embodiments, the vector is a viral vector or a non-viral vector. In embodiments, the viral vector is an adenoviral vector, an adeno-associated virus vector, and a lentiviral vector.

In one aspect, the disclosure provides a cell comprising a vector, a polynucleotide cassette, an aptamer encoding sequence/aptamer sequence, or riboswitch disclosed herein.

The disclosure also provides methods for modulating the expression of a target gene using a polynucleotide cassette, an aptamer encoding sequence/aptamer sequence, or riboswitch disclosed herein, by provided to a cell or tissue a small molecule of Formula I-XXII, including, e.g., a small molecule provided in Table A.

### BRIEF DESCRIPTION OF THE FIGURES

FIGS. 1a-1d. TPP aptamer homologous sequence regulates gene expression in response to TPP and fursultiamine. FIG. 1a: schematics of the synthetic riboswitch cassette containing intron-alternative exon-aptamer-intron. FIG. 1b: In the presence of aptamer ligand, aptamer ligand binding facilitates for the formation of hairpin stem that sequesters the accessibility of the 5′ splice site (5′ ss) of the alternative exon, resulting in the exclusion of the stop codon containing alternative exon and target gene expression. Riboswitch 12C6-1 regulates luciferase gene expression in response to TPP (FIG. 1c) and fursultiamine (FIG. 1d) treatment. HEK 293 cells were transfected with luciferase construct containing 12C6-1 riboswitch. The transfected cells were treated with TPP or fursultiamine at the indicated concentrations. The fold induction was calculated as the quotient of the luciferase activity obtained from cells treated with compounds divided by the luciferase activity obtained from cells without compound treatment.

FIGS. 2a-2d. Comp. 004 activates TPP aptamer riboswitches in regulating luciferase expression in HEK 293 cells. Cells were transfected with the indicated riboswitch constructs and treated with various concentration of Comp. 004. Luciferase activity was measured 20 hours post compound treatment, and fold induction was calculated as the quotient of the luciferase activity obtained from cells treated with compounds divided by the luciferase activity obtained from cells without treatment. Riboswitch containing E. Coli thiM or Alishewanella tabrizica thiC aptamer (FIG. 2a, 2b) or 12C6-1 aptamer (FIG. 2c, 2d) regulates luciferase expression in response to Comp. 004 treatment.

FIGS. 3a-3b. FIG. 3a: The predicated secondary structure of the 12C6-1 aptamer sequence with the flanking C and the U1 binding sequence at the 5′ end and the flanking G and the complementary sequence of U1 binding site at the 3′ end, respectively. FIG. 3b: 12C6-1 parent sequence and the template sequence for each aptamer library with N denoting a random base.

FIGS. 4a-4e. Riboswitches containing aptamers derived from 12C-1 regulate luciferase expression in HEK 293 cells in response to treatment with Comp. 004 (FIGS. 4a, 4b, and 4c) and analogues (FIGS. 4d and 4e). FIG. 4d: Compounds analogous to Comp. 004 (Comps. 003, 005, 008, 009, and 011) bind 12C6-1 and activate derivative riboswitches in regulating Luciferase expression. The fold induction was calculated as the quotient of the luciferase activity obtained from cells treated with compounds divided by the luciferase activity obtained from cells without treatment. FIG. 4e: Additional analogues (Comps. 012, 013, 014, 015, 016, 018, and 019) regulate expression in a dose-dependent fashion.

FIGS. 5a-5d. Riboswitch-regulated expression of mouse Epo gene and human growth hormone gene in mammalian cells. Cells transfected with indicated constructs containing riboswitch cassette N4-1C11 or N5-12G6 were treated with or without indicated concentrations of Comp. 004, and the secreted mEpo or hGH was detected and quantified ELISA. FIG. 5a: Riboswitch 12G6 and 1C11 regulate mEpo expression in AML12 cells in response to Comp. 004 treatment. FIG. 5b: the fold induction of mEpo by Comp. 004. The fold induction was calculated as the quotient of the mEpo level obtained from cells treated with Comp. 004 divided by mEpo level obtained from cells without compound treatment. FIG. 5c: Riboswitch regulates mEpo expression in C1C12 cells in response to Comp. 004 treatment. FIG. 5d: Riboswitch regulates mEpo expression in HEK 293 cells in response to Comp. 004 treatment.

FIGS. 6a-6d. Riboswitch-regulated luciferase expression in the muscle and liver in mice. FIGS. 6a-6b: Balb/c mice (n=5) were injected intravenously (I.V.) with AAV8.Luci.Con1 (non-regulatable) or AAV8.Luci.12G6 (regulatable) and administered orally with the indicated doses of Comp. 004. The luciferase activity was measured at the indicated time points post compound oral dosing. FIG. 6a: Bioluminescence image of a representative mouse from each AAV-injected group before and after Comp. 004 treatment. FIG. 6b: The luciferase luminescence signal of whole-body imaging from the mice (n=5) in each AAV-injected group before and after Comp. 004 treatment. FIG. 6c: Balb/c mice were injected intravenously (I.V.) with the indicated amounts of AAV8.Luci.Con1 (non-regulatable) or AAV8.Luci.12G6 (regulatable) and administered orally with the indicated doses of Comp. 004. FIG. 6d: shows luciferase expression in liver from AAV8.Luci.Con1 and AAV8.Luci.12G6 following dose of 30 mg/kg Comp. 004.

FIGS. 7a-7c. Riboswitch-regulated luciferase expression in the muscle in mice. FIGS. 7a-7b: Balb/c mice were injected intramuscularly (I.M.) with AAV8.Luci.Con1 (non-regulatable) or AAV8.Luci.12G6 (regulatable) and administered orally with the indicated doses of Comp. 004. The luciferase activity was measured at the indicated time points post compound oral dosing. FIG. 7a: Bioluminescence image of a representative mouse from each AAV-injected group before and after Comp. 004 treatment. FIG. 7b: the luciferase luminescence signal of whole-body imaging from the mice (n=5) in each AAV-injected group before and after Comp. 004 treatment. FIG. 7c: Balb/c mice were injected intramuscularly with the indicated amounts of AAV8.Luci.Con1 (non-regulatable) or AAV8.Luci.12G6 (regulatable) and administered orally with the indicated doses of Comp. 004.

FIG. 8. Riboswitch-regulated mouse Epo expression in the muscle in vivo. Mice were injected with AAV8.mEpo-12G6 vectors at the indicated amounts and treated with indicated doses of Comp. 004 via oral administration. Serum mouse Epo expression was measured using mouse Epo specific ELISA.

FIGS. 9a-9b. Expression of Erythropoietin (Epo) restores hemocrit in chronic kidney disease (CKD) associated anemia in a dose response to oral small molecule. The effect of riboswitch-regulated expression of Epo on hematocrit was evaluated in a mouse model of chronic kidney disease (CKD)-associated anemia. After 20 doses of compound 004 by oral administration, the hematocrit of anemic mice was increased, with the biggest increase in the 100 mg/kg dose group. However, the hematocrits of anemic mice injected with AAV8.mEpo.12G6 but were not treated with compound 004 did not increase, remaining the same hematocrit as that from anemic mice without delivered AAV8.mEpo.12G6 (FIG. 9a). When mice treated with higher compound dose at 300 mg/kg for 15 days and 10 doses, the hematocrit was restored to normal level in the mouse group injected with lower AAV dose (1×10¹⁰ vg per mouse). In contrast, the hematocrits of mice injected with relatively higher AAV dose (2.5×10¹⁰ vg per mouse) exceeded the normal hematocrit level. (FIG. 9b). These results indicate that Epo was induced from the delivered AAV vector after riboswitch inducer treatment and the induced Epo stimulated erythropoiesis leading to hematocrit increase in anemic animal.

FIGS. 10a-10c. Controlled secretion of parathyroid hormone (PTH) increases serum calcium. Riboswitch 12G6 regulated hPTH expression in dose dependent manner (FIG. 10a). When this regulated hPTH was delivered into mice via AAV vector, Compound 004 treatment induced dose-dependent production of hPTH (FIG. 10b) in mice and accordingly inducing the increase in the serum calcium concentration (FIG. 10c).

### DETAILED DESCRIPTION

Provided herein are aptamer sequences that bind to, or otherwise respond to the presence of, small molecules of Formula I-XXII. In some embodiments, the aptamer sequences provided herein are useful for the regulation of the expression of a target gene in response to the presence or absence of the small molecule ligand. Also contemplated are recombinant riboswitches comprising the aptamer sequences disclosed herein, as well as recombinant polynucleotide cassettes for regulating the expression of a target gene, wherein the polynucleotide cassettes comprise sequences encoding the riboswitches disclosed herein. Also provided herein are methods of using the aptamers, riboswitches, and/or polynucleotide cassettes for the regulation of target genes, including therapeutic genes, and for the treatment of subjects in need thereof.

### Aptamers

Aptamers are single-stranded nucleic acid molecules that non-covalently bind to specific ligands with high affinity and specificity by folding into three-dimensional structures. Aptamer ligands include ions, small molecules, proteins, viruses, and cells.

Aptamer ligands can be, for example, an organic compound, amino acid, steroid, carbohydrate, or nucleotide. Non-limiting examples of small molecule aptamer ligands include antibiotics, therapeutics, dyes, cofactors, metabolites, molecular markers, neurotransmitters, pollutants, toxins, food adulterants, carcinogens, drugs of abuse. As such, aptamers are useful for the detection of small molecules. Application of small-molecule detection by aptamers include environmental monitoring, food safety, medicine (including diagnostics), microbiology, analytical chemistry, forensic science, agriculture, and basic biology research.

The term “aptamer” as used herein refers to an RNA polynucleotide (or DNA sequence encoding the RNA polynucleotide) that specifically binds to a class of ligands. The term “ligand” refers to a molecule that is specifically bound by an aptamer. Aptamers have binding regions that are capable of forming complexes with an intended target molecule (i.e., the ligand). An aptamer will typically be between about 15 and about 200 nucleotides in length. More commonly, an aptamer will be between about 30 and about 100 nucleotides in length, for example, 70 to 90 nucleotides in length. Aptamers typically comprise multiple paired (P) regions in which the aptamer forms a stem and unpaired regions where the aptamer forms a joining (J) region or a loop (L) region. The paired regions can be numbered sequentially starting at the 5′ end (P1) and numbering each stem sequentially (P2, P3, etc.). The loops (L1, L2, etc.) are numbered based on the adjacent paired region and the joining regions are numbered according to the paired regions that they link.

In one aspect, the disclosure provides an aptamer that binds to a small molecule (e.g., one or more of the small molecules disclosed herein), wherein the aptamer encoding sequence comprises: CTGGGGAGTCCTTCATGCGGGGCTGAGAGGATGGAAGX₁X₂AX₃X₄X₅X₆CCAT CGACCCX₇X₈X₉X₁₀X₁₁X₁₂CCTX₁₃X₁₄X₁₅CCGGX₁₆X₁₇X₁₈X₁₉X₂₀X₂₁CCGGX₂₂X₂₃C AGGGAG (SEQ ID NO:2); wherein:
X₁ is C G, or T;
X₂-X₅ is any nucleotide;
X₆ is any nucleotideX₇ is A, G, or T;
X₈—X₁₀ is any nucleotide;
X₁₁ is any nucleotide or no nucleotide;
X₁₂ is A, C, or T;
X₁₃—X₂₀;
X₂₁ is C, G, T; and
X₂₂, and X₂₃ is any nucleotide;
In embodiments, X₁—X₆ are not simultaneously C, A, T, C, G, and A, respectively;
X₇—X₁₂ are not simultaneously A, T, T, G, C, and A, respectively; X₁₃, X₁₄, X₁₅, X₂₂, and X₂₃ are not simultaneously G, A, T, C, and G, respectively; and/or X₁₆—X₂₁, are not simultaneously A, T, C, A, T, and G, respectively. In embodiments, one or more of the above limitations applies to the aptamer when the 5′ and 3′ end of the aptamer sequence disclosed herein is not C and G, respectively.

In one aspect, the disclosure provides an aptamer that binds to a small molecule, wherein the aptamer encoding sequence comprises:
CTGGGGAGTCCTTCATGCGGGGCTGAGAGGATGGAAGX₁X₂AX₃X₄X₅X₆CCAT CGACCCX₇X₈X₉X₁₀X₁₁X₁₂CCTX₁₃X₁₄X₁₅CCGGX₁₆X₁₇X₁₈X₁₉X₂₀X₂₁CCGGX₂₂X₂₃C AGGGAG (SEQ ID NO:2); wherein:
X₁ is C or T;
X₂ is any nucleotide;
X₃ is any nucleotide;
X₄ is G or T;
X₅ is A, G, or T;
X₆ is A or G;
X₇ is A or T;
X₈ is A, C, or T;
X₉ is A, C, or T;
X₁₀ is any nucleotide;
X₁₁ is any nucleotide or no nucleotide;
X₁₂ is A;
X₁₃ is A, C, or G;
X₁₄ is any nucleotide;
X₁₅ is C, G, or T;
X₁₆ is G or T;
X₁₇ is A or T;
X₁₈ is any nucleotide;
X₁₉ is A or G;
X₂₀ is A, G, T;
X₂₁ is C, G, T;
X₂₂ is T; and
X₂₃ is A, G, or T (taken together SEQ ID NO:681—that is SEQ ID NO: 681 has the recited limitations for X₁ to X₂₃ recited in this paragraph).

In embodiments, the aptamer encoding sequence comprises:
CTGGGGAGTCCTTCATGCGGGGCTGAGAGGATGGAAGX₁X₂AX₃X₄X₅X₆CCAT CGACCCX₇X₈X₉X₁₀X₁₁X₂CCTX₁₃X₁₄X₁₅CCGGX₁₆X₁₇X₁₈X₁₉X₂₀X₂₁CCGGX₂₂X₂₃C AGGGAG (SEQ ID NO:2); wherein:
X₁ is C or T;
X₂ is any nucleotide;
X₃ is any nucleotide;
X₄ is G or T;
X₅ is A, G, or T;
X₆ is A or G;
X₇ is A;
X₈ is A, C, or T;
X₉ is A, C, or T;
X₁₀ is any nucleotide;
X₁₁ is any nucleotide or no nucleotide;
X₁₂ is A;
X₁₃ is A, C, or G;
X₁₄ is any nucleotide;
X₁₅ is C, G, or T;
X₁₆ is G or T;
X₁₇ is A or T;
X₁₈ is any nucleotide;
X₁₉ is A or G;
X₂₀ is A, G, T;
X₂₁ is C, G, T X₂₂ is T; and
X₂₃ is A, G, or T (taken together SEQ ID NO:682).

In embodiments, the aptamer encoding sequence comprises:
CTGGGGAGTCCTTCATGCGGGGCTGAGAGGATGGAAGCAATCGACCATCGA CCCX₇X₈X₉X₁₀X₁₁X₁₂CCTGATCCGGATCATGCCGGCGCAGGGAG (SEQ ID NO:4); wherein:
X₇ is A, G, or T;
X₈ is any nucleotide;
X₉ is any nucleotide;
X₁₀ is any nucleotide;
X₁₁ is any nucleotide or no nucleotide;
X₁₂ is A, C, or T (taken together SEQ ID NO:683).

In embodiments, X₇—X₁₂ are not simultaneously A, T, T, G, C, and A, respectively.

In embodiments, the aptamer encoding sequence comprises:
CTGGGGAGTCCTTCATGCGGGGCTGAGAGGATGGAAGCAATCGACCATCGA CCCX₇X₈X₉X₁₀X₁₁X₁₂CCTGATCCGGATCATGCCGGCGCAGGGAG (SEQ ID NO:4); wherein:
X₇ is A or T;
X₈ is A, C, or T;
X₉ is A, C, or T;
X₁₀ is any nucleotide;
X₁₁ is any nucleotide or no nucleotide; and
X₁₂ is A (taken together SEQ ID NO:684).

In embodiments, X₇—X₁₂ are not simultaneously A, T, T, G, C, and A, respectively.

In embodiments, the aptamer encoding sequence comprises:
CTGGGGAGTCCTTCATGCGGGGCTGAGAGGATGGAAGCAATCGACCATCGA CCCX₇X₈X₉X₁₀X₁₁X₁₂CCTGATCCGGATCATGCCGGCGCAGGGAG (SEQ ID NO:4); wherein:
X₇ is A;
X₈ is A, C, or T;
X₉ is A, C, or T;
X₁₀ is any nucleotide;
X₁₁ is any nucleotide or no nucleotide; and
X₁₂ is A (taken together SEQ ID NO:685).

In embodiments, X₇—X₁₂ are not simultaneously A, T, T, G, C, and A, respectively.

In embodiments, the aptamer encoding sequence comprises:

(SEQ ID NO: 3)
CTGGGGAGTCCTTCATGCGGGGCTGAGAGGATGGAAGX₁X₂AX₃X₄X₅X₆C
CATCGACCCATTGCACCTGATCCGGATCATGCCGGCGCAGGGAG;

wherein:
X₁ is C, G, or T;
X₂ is any nucleotide;
X₃ is any nucleotide;
X₄ is any nucleotide;
X₅ is any nucleotide; and
X₆ is any nucleotide (taken together SEQ ID NO:686).

In embodiments, X₁—X₆ are not simultaneously C, A, T, C, G, and A, respectively.

In embodiments, the aptamer encoding sequence comprises:

(SEQ ID NO: 3)
CTGGGGAGTCCTTCATGCGGGGCTGAGAGGATGGAAGX₁X₂AX₃X₄X₅X₆C
CATCGACCCATTGCACCTGATCCGGATCATGCCGGCGCAGGGAG;

wherein:
X₁ is C or T;
X₂ is any nucleotide;
X₃ is any nucleotide;
X₄ is any nucleotide;
X₅ is A, G, or T; and
X₆ is any nucleotide (taken together SEQ ID NO:687)

In embodiments, X₁—X₆ are not simultaneously C, A, T, C, G, and A, respectively.

In embodiments, the aptamer encoding sequence comprises:

(SEQ ID NO: 3)
CTGGGGAGTCCTTCATGCGGGGCTGAGAGGATGGAAGX₁X₂AX₃X₄X₅X₆C
CATCGACCCATTGCACCTGATCCGGATCATGCCGGCGCAGGGAG;

wherein:
X₁ is C or T;
X₂ is any nucleotide;
X₃ is any nucleotide;
X₄ is G or T;
X₅ is A, G, or T; and
X₆ is A or G (taken together SEQ ID NO:688).

In embodiments, the aptamer encoding sequence comprises:
CTGGGGAGTCCTTCATGCGGGGCTGAGAGGATGGAAGCAATCGACCATCGA CCCATTGCACCTX₁₃X₁₄X₁₅CCGGATCATGCCGGX₂₂X₂₃CAGGGAG (SEQ ID NO:5); wherein:
X₁₃, X₁₄, X₁₅, X₂₂, and X₂₃ is any nucleotide.

In embodiments, X₁₃, X₁₄, X₁₅, X₂₂, and X₂₃ are not simultaneously G, A, T, C, and G, respectively.

In embodiments, the aptamer encoding sequence comprises:
CTGGGGAGTCCTTCATGCGGGGCTGAGAGGATGGAAGCAATCGACCATCGA CCCATTGCACCTX₁₃X₁₄X₁₅CCGGATCATGCCGGX₂₂X₂₃CAGGGAG (SEQ ID NO:5); wherein:
X₁₃ is A, C, or G;
X₁₄ is any nucleotide;
X₁₅ is C, G, or T;
X₂₂ is T; and
X₂₃ is A, G, or T (taken together SEQ ID NO:689).

In embodiments, the aptamer encoding sequence comprises:
CTGGGGAGTCCTTCATGCGGGGCTGAGAGGATGGAAGCAATCGACCATCGA CCCATTGCACCTGATCCGGX₁₆X₁₇X₁₈X₁₉X₂₀X₂₁CCGGCGCAGGGAG (SEQ ID NO:6); wherein:
X₁₆ is any nucleotide;
X₁₇ is any nucleotide;
X₁₈ is any nucleotide;
X₁₉ is any nucleotide;
X₂₀ is any nucleotide; and
X₂₁ is C, G, T (taken together SEQ ID NO:690).

In embodiments, X₁₆—X₂₁, are not simultaneously A, T, C, A, T, and G, respectively.

In embodiments, the aptamer encoding sequence comprises:
CTGGGGAGTCCTTCATGCGGGGCTGAGAGGATGGAAGCAATCGACCATCGA CCCATTGCACCTGATCCGGX₁₆X₁₇X₁₈X₁₉X₂₀X₂₁CCGGCGCAGGGAG (SEQ ID NO:6); wherein:
X₁₆ is G or T;
X₁₇ is A or T;
X₁₈ is any nucleotide;
X₁₉ is A or G;
X₂₀ is A, G, T; and
X₂₁ is C, G, T (taken together SEQ ID NO:691).

In embodiments, the aptamer encoding sequence comprises a sequence that is at least 95% identical, or at least 99% identical to a sequence selected from the group consisting of SEQ ID NOs: 1 and 7-558. In embodiments, the aptamer encoding sequence comprises a sequence that is selected from the group consisting of SEQ ID NOs: 1 and 7-558.

In embodiments, the aptamer encoding sequence comprises a sequence that is at least 95% identical, or at least 99% identical to a sequence selected from the group consisting of SEQ ID NOs: 7-17, 89-96, 174-349, and 358-583. In embodiments, the aptamer encoding sequence comprises a sequence that is selected from the group consisting of SEQ ID NOs: 7-17, 89-96, 174-349, and 358-583.

In embodiments, the aptamer encoding sequence comprises a sequence that is at least 95% identical, or at least 99% identical to a sequence selected from the group consisting of SEQ ID NOs: 7-11, 89-94, 174-349, and 358-447. In embodiments, the aptamer encoding sequence comprises a sequence that is selected from the group consisting of SEQ ID NOs: 7-11, 89-94, 174-349, and 358-447.

In embodiments, the aptamer encoding sequence comprises a sequence that is at least 95% identical, or at least 99% identical to a sequence selected from the group consisting of SEQ ID NOs: 174, 358, 363, and 378. In embodiments, the aptamer encoding sequence comprises a sequence that is selected from the group consisting of SEQ ID NOs: 174, 358, 363, and 378.

In embodiments, the first and the last nucleotide of the aptamer encoding sequence can be any nucleotide or no nucleotide. In embodiments, the first two and the last two nucleotides of the aptamer encoding sequence can be any nucleotide or no nucleotide. In these embodiments, additional sequence that is 5′ and 3′ of the aptamer encoding sequence may be present and form part of the stem forming sequence of the riboswitch.

In one aspect, the disclosure provides the aptamer encoded by the aptamer encoding sequences disclosed herein.

The ordinarily-skilled artisan would understand that the aptamers described herein may be ribonucleic acid (RNA) molecules. In embodiments, the aptamers described herein are part of a longer RNA polynucleotide, including, for example, hnRNA, mRNA, siRNA, or miRNA.

### Aptamer Ligands

In embodiments, an aptamer disclosed herein binds to, or otherwise responds to the presence or addition of, a small molecule (ligand) disclosed herein, including small molecules having the structure according to Formula I to XXII, including the small molecules in Table A.

In embodiments, the small molecule has the structure according to Formula I:

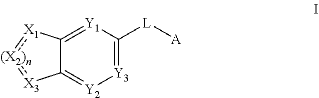

wherein
   X₁, X₂, and X₃ are, in each instance, independently selected from CR₁, CHR₁, N, NH, O and S, wherein adjacent X₁, X₂, and X₃ are not simultaneously selected to be O or S; the dashed lines represent optional double bonds;
   Y₁, Y₂, and Y₃ are, in each instance, independently selected from CR₂ and N;
   n is 1 or 2, wherein when n is 1, only one of the dashed lines is a double bond;
L-A is

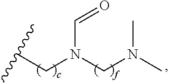

or
L is selected from

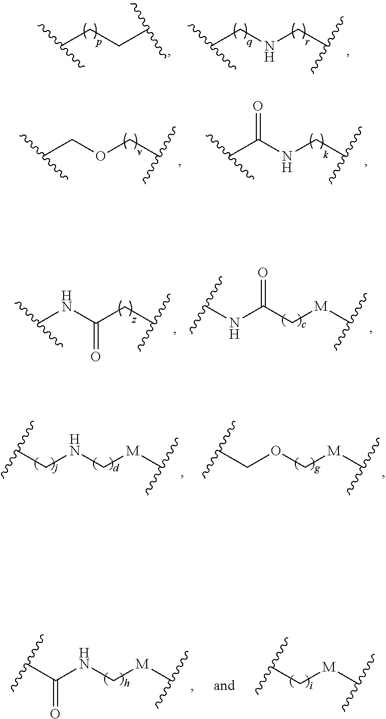

wherein k, p, q, r, and v are independently selected from integers 0, 1, 2, 3, 4, 5, 6, 7, 8, 9, and 10, z is selected from integers 1, 2, 3, 4, and 5;
c, d, e, f, g, h and i are independently selected from integers 1, 2, 3, 4, 5, 6, 7, 8, 9, and 10; j is selected from integers 0, 1, 2, 3, 4, 5, 6, 7, 8, 9, and 10;
   M is selected from —NH—, —O—, —NHC(═O)—, —C(═O)NH—, —S—, and —C(═O)—; and
   A is selected from

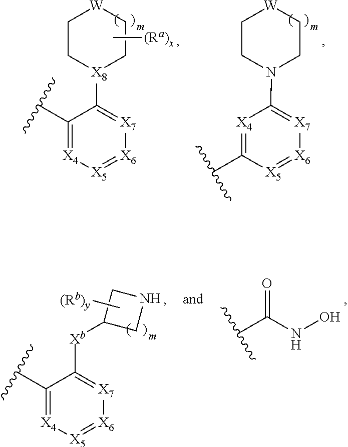

wherein X₄, X₅, X₆, and X₇, are independently selected from CR₃ and N;
X₈ is N or CH;
X^{b} is selected from 0, NH, and NCH₃;
   wherein each of R₁, R₂, and R₃ are independently selected from —H, —Cl, —Br, —I, —F, —CF₃, —CH₂F, —CHF₂, —OH, —CN, —NO₂, —NH₂, —NH(C₁-C₆ alkyl), —N(C₁-C₆ alkyl)₂, —COOH, —COO(C₁-C₆ alkyl), —CO(C₁-C₆ alkyl), —O(C₁-C₆ alkyl), —OCO(C₁-C₆ alkyl), —NCO(C₁-C₆ alkyl), —CONH(C₁-C₆ alkyl), and substituted or unsubstituted C₁-C₆ alkyl; additionally or alternatively, two R₃ on adjacent ring positions may be taken together to form a 5- or 6-membered aromatic ring having from 0 to 2 heteroatoms selected from O, S, N and NH;
   m is 1 or 2;
each R^{a} is independently selected from C₁ to C₃ alkyl, —OCH₃, —CF₃, —CH₂F, —CHF₂, halo, hydroxyl and amino; or additionally or alternatively, two R^{a} attached to the same carbon atom form an oxo group, or two R^{a} attached to different carbon atoms form a 4- to 6-membered carbocyclic ring or a 4- to 6-membered heterocyclic ring having 1 or 2 heteroatoms selected from O and NH;
x is 0, 1, 2 or 3;
each R^{b} is independently selected from C₁ to C₃ alkyl, —OCH₃, —CF₃, —CH₂F, —CHF₂, halo, hydroxyl and amino; or additionally or alternatively, two R^{b} attached to the same carbon atom form an oxo group; or two R attached to different carbon atoms form a 4- to 6-membered carbocyclic ring or a 4- to 6-membered heterocyclic ring having 1 or 2 heteroatoms selected from O and NH;
y is 0, 1, 2 or 3; and
   W is O or NR₄, wherein R₄ is selected from selected from —H, —CO(C₁-C₆ alkyl), substituted or unsubstituted C₁-C₆ alkyl, substituted or unsubstituted aryl, substituted or unsubstituted heteroaryl, substituted or unsubstituted cycloalkyl, —CO(aryl), —CO(heteroaryl), and —CO(cycloalkyl);
provided that at least two of X₁, X₂, X₃, X₄, X₅, X₆, and X₇ are N;
or a pharmaceutically acceptable salt thereof.

In embodiments of the above formula, y is 0.

In embodiments, the small molecule has the structure according to Formula II:

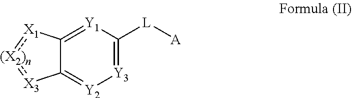

wherein
X₁, X₂, and X₃ are, in each instance, independently selected from CR₁, CHR₁, N, NH, O and S, wherein adjacent X₁, X₂, and X₃ are not simultaneously selected to be O or S; the dashed lines represent optional double bonds;
Y₁, Y₂, and Y₃ are, in each instance, independently selected from CR₂ and N;
n is 1 or 2, wherein when n is 1, only one of the dashed lines is a double bond;
L is selected from

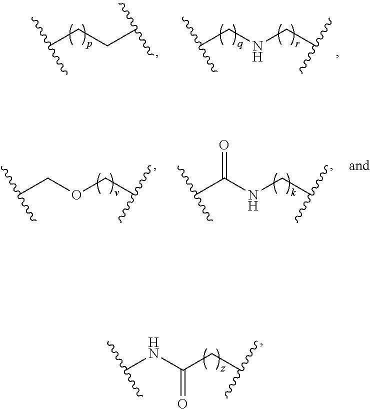

wherein k, p, q r, and v are independently selected from integers 0, 1, 2, 3, 4, 5, 6, 7, 8, 9, and 10, z is selected from integers 1, 2, 3, 4, and 5; and
A is selected from

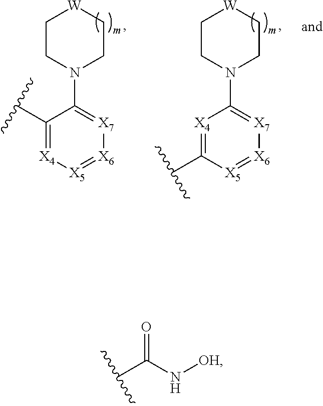

wherein X₄, X₅, X₆, and X₇, are independently selected from CR₃ and N;
   wherein each of R₁, R₂, and R₃ are independently selected from —H, —Cl, —Br, —I, —F, —CF₃, —CH₂F, —CHF₂, —OH, —CN, —NO₂, —NH₂, —NH(C₁-C₆ alkyl), —N(C₁-C₆ alkyl)₂, —COOH, —COO(C₁-C₆ alkyl), —CO(C₁-C₆ alkyl), —O(C₁-C₆ alkyl), —OCO(C₁-C₆ alkyl), —NCO(C₁-C₆ alkyl), —CONH(C₁-C₆ alkyl), and substituted or unsubstituted C₁-C₆ alkyl;
   m is 1 or 2; and
   W is O or NR₄, wherein R₄ is selected from selected from —H, —CO(C₁-C₆ alkyl), substituted or unsubstituted C₁-C₆ alkyl, substituted or unsubstituted aryl, substituted or unsubstituted heteroaryl, substituted or unsubstituted cycloalkyl, —CO(aryl), —CO(heteroaryl), and —CO(cycloalkyl);
provided that at least two of X₁, X₂, X₃, X₄, X₅, X₆, and X₇ are N;
or a pharmaceutically acceptable salt thereof.

In an embodiment of the above formula, at least one of X₁, X₂, or X₃ is N.

In an embodiment of the above formula, X₁ is N.

In an embodiment of the above formula, X₂ is N.

In an embodiment of the above formula, X₃ is N.

In an embodiment of the above formula, two of X₁, X₂, and X₃ are N.

In an embodiment of the above formula, X₁ and X₃ are N.

In an embodiment of the above formula, at least one of Y₁, Y₂, and Y₃ is N.

In an embodiment of the above formula, Y₁ is N.

In an embodiment of the above formula, Y₂ is N.

In an embodiment of the above formula, Y₃ is N.

In an embodiment of the above formula, at least one of Y₁, Y₂, and Y₃ is CR₂.

In an embodiment of the above formula, Y₁ is CR₂.

In an embodiment of the above formula, Y₂ is CR₂.

In an embodiment of the above formula, Y₃ is CR₂.

In an embodiment of the above formula, n is 2.

In embodiments, the small molecule has the structure according to Formula III:

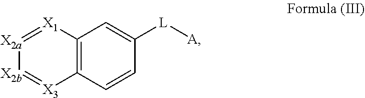

wherein
X₂ₐ and X_{2b} are independently selected from CR₁ and N;
X₁ and X₃ are independently selected from CR₁ and N;
L and A are as provided for Formula (II); and
two of X₁, X₂a, X₂b, and X₃ are N.

In embodiments, the small molecule has the structure according to formula (IV):

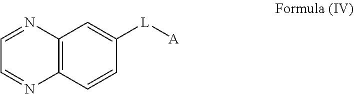

wherein
L and A are as provided for Formula (II).

In any above embodiment of the compound, L may be selected from

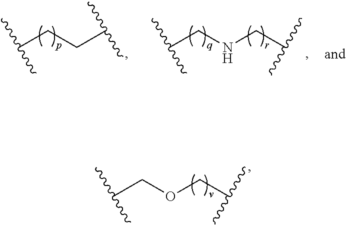

As in any above embodiment of a compound, L may be selected to be

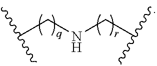

In any of the above embodiments, a compound wherein q and r are 0 or 1.

In any of the above embodiments, a compound wherein q is 1.

In any of the above embodiments, a compound wherein r is 1.

In any of the above embodiments, a compound wherein r is 0.

In any of the above embodiments, a compound wherein q and r are 1.

In any of the above embodiments, a compound wherein q is 1 and r is 0.

In any of the above embodiments, a compound wherein m is 1.

In any of the above embodiments, a compound wherein W is selected from NH, O, and N(C₁-C₆ alkyl).

In any of the above embodiments, a compound wherein W is NH.

In any of the above embodiments, a compound wherein at least one of X₄, X₅, X₆, and X₇ is N.

In any of the above embodiments, a compound wherein X₄ is N.

In any of the above embodiments, a compound wherein X₅ is N.

In any of the above embodiments, a compound wherein X₆ is N.

In any of the above embodiments, a compound wherein X₇ is N.

In any of the above embodiments, a compound wherein X₄ and X₆ are N.

In any of the above embodiments, a compound wherein X₅ and X₇ are N.

In any of the above embodiments, a compound wherein X₅ or X₆ are N, and both X₄ and X₇ are independently CR₂.

In any of the above embodiments, a compound wherein A is

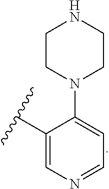 [SMILES: *c1cnccc1N1CCNCC1]

In any of the above embodiments, a compound with the structure of Formula V:

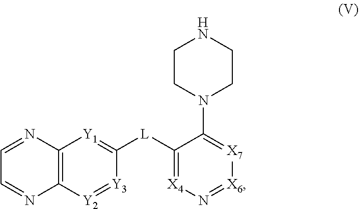

In any of the above embodiments, a compound wherein L is

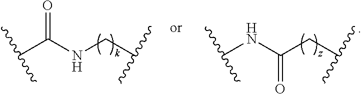

In any of the above embodiments, a compound wherein Y₁, Y₂, and Y₃ are, in each instance, independently selected from CR₂ and N, wherein R₁ is selected from —H, —Cl, —Br, —I, —F, —OH, and —NH₂.

In any of the above embodiments, a compound wherein z is 2.

In any of the above embodiments, a compound wherein Y₂ is N.

In any of the above embodiments, a compound wherein Y₂ is CR₂ and R₁ is selected from —H, —F, —OH, and —NH₂.

In any of the above embodiments, a compound wherein A is

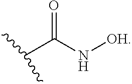 [SMILES: *C(=O)NO]

In embodiments, the small molecule has the structure according to formulas:

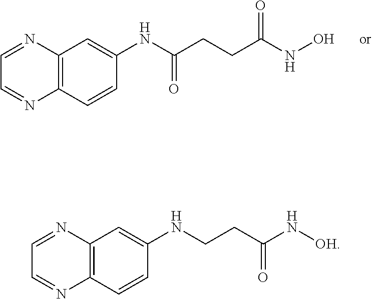 [SMILES: O=C(CCC(=O)Nc1ccc2nccnc2c1)NO ; O=C(CCNc1ccc2nccnc2c1)NO]

In other embodiments, the small molecule has the structure according to formulas:

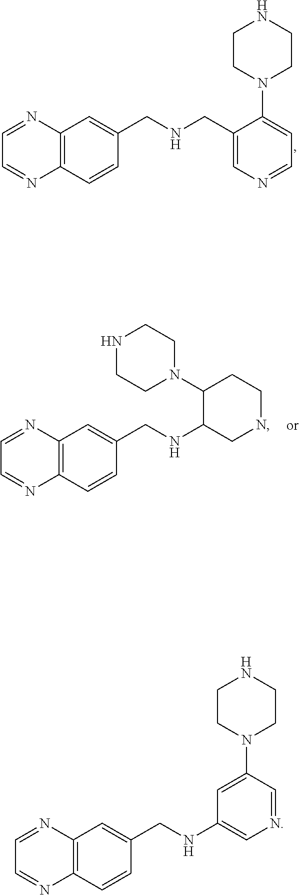

In other embodiments the small molecule has a structure of formula VI:

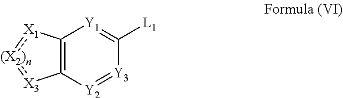

wherein
X₁, X₂, and X₃ are, in each instance, independently selected from CR₁, CHR₁, N, NH, O and S, wherein adjacent X₁, X₂, and X₃ are not simultaneously selected to be O or S; the dashed lines represent optional double bonds;
Y₁, Y₂, and Y₃ are, in each instance, independently selected from CR₂ and N;
n is 1 or 2, wherein when n is 1, only one of the dashed lines is a double bond;
L₁ is selected from

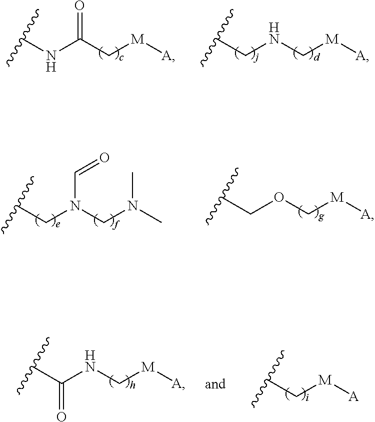

wherein c d e f g h and i are independently selected from integers 1, 2, 3, 4, 5, 6, 7, 8, 9, and 10; j is selected from integers 0, 1, 2, 3, 4, 5, 6, 7, 8, 9, and 10;
M is selected from —NH—, —O—, —NHC(═O)—, —C(═O)NH—, —S—, and —C(═O)—; and
A is selected from

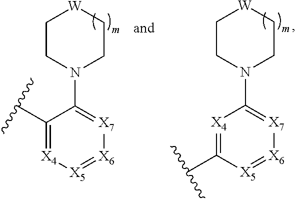

wherein X₄, X₅, X₆, and X₇, are independently selected from CR₃ and N;
wherein each of R₁, R₂, and R₃ are independently selected from —H, —Cl, —Br, —I, —F, —CF₃, —CH₂F, —CHF₂, —OH, —CN, —NO₂, —NH₂, —NH(C₁-C₆ alkyl), —N(C₁-C₆ alkyl)₂, —COOH, —COO(C₁-C₆ alkyl), —CO(C₁-C₆ alkyl), —O(C₁-C₆ alkyl), —OCO(C₁-C₆ alkyl), —NCO(C₁-C₆ alkyl), —CONH(C₁-C₆ alkyl), and substituted or unsubstituted C₁-C₆ alkyl;
m is 1 or 2; and
W is —O— or —N(R₄)—, wherein R₄ is selected from —H, —CO(C1-C₆ alkyl), substituted or unsubstituted C₁-C₆ alkyl, substituted or unsubstituted aryl, substituted or unsubstituted heteroaryl, substituted or unsubstituted cycloalkyl, —CO(aryl), —CO(heteroaryl), and —CO(cycloalkyl);
provided that at least two of X₁, X₂, X₃, X₄, X₅, X₆, and X₇ are N;
or a pharmaceutically acceptable salt thereof.

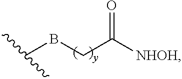

In an additional embodiment, L is, wherein B is selected from —NH— and —NHC(═O)—; and y is an integer selected from 1, 2, 3, 4, and 5.

In the above embodiments, a compound wherein at least one of X₁, X₂, or X₃ is N.

In the above embodiments, a compound wherein X₁ is N.

In the above embodiments, a compound wherein X₂ is N.

In the above embodiments, a compound wherein X₃ is N.

In the above embodiments, a compound wherein, in each instance, two of X₁, X₂, and X₃ are N.

In the above embodiments, a compound wherein X₁ and X₃ are N.

In the above embodiments, a compound wherein at least one of Y₁, Y₂, and Y₃ is N.

In the above embodiments, a compound wherein Y₁ is N.

In the above embodiments, a compound wherein Y₂ is N.

In the above embodiments, a compound wherein Y₃ is N.

In the above embodiments, a compound wherein at least one of Y₁, Y₂, and Y₃ is CR₂.

In the above embodiments, a compound wherein Y₁ is CR₂.

In the above embodiments, a compound wherein Y₂ is CR₂.

In the above embodiments, a compound wherein Y₃ is CR₂.

In the above embodiments, a compound wherein n is 2.

As in any above embodiment, a compound having the structure of formula (VII):

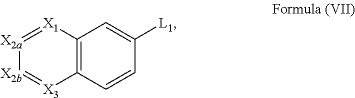

wherein
X₂ₐ and X_{2b} are independently selected from CR₁ and N;
X₁ and X₃ are independently selected from CR₁ and N;
L₁ and R₁ are as provided for Formula (I); and two of X₁, X₂a, X₂b, and X₃ are N; or a pharmaceutically acceptable salt thereof.

In the above embodiments, a compound having the structure of formula (VIII):

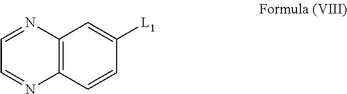

wherein
L₁ is as provided for Formula (VI); or a pharmaceutically acceptable salt thereof.

In the above embodiments, a compound wherein c, d, e, f, g, h and i are independently selected from integers 1, 2, and 3.

In the above embodiments, a compound wherein L₁ is selected from

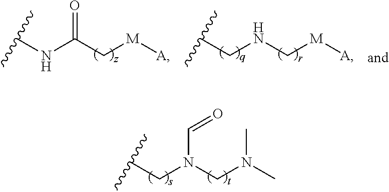

In the above embodiments, a compound wherein c, d, e, and f are independently selected from integers 1, 2, and 3.

In the above embodiments, a compound wherein c, d, and e are 1.

In the above embodiments, a compound wherein L₁ is

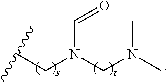

In the above embodiments, a compound wherein e and f are independently selected from 1, 2, and 3.

In the above embodiments, a compound wherein e and f are 1 or 2.

In the above embodiments, a compound wherein e is 1.

In the above embodiments, a compound wherein f is 2.

In the above embodiments, a compound wherein e is 1 and f is 2.

In the above embodiments, a compound wherein L₁ is

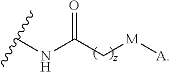

In the above embodiments, a compound wherein c is 1, 2, or 3.

In the above embodiments, a compound wherein c is 1.

In the above embodiments, a compound wherein c is 2

In the above embodiments, a compound wherein c is 3.

In the above embodiments, a compound wherein M is selected from —NH—, —O—, and —S—.

In the above embodiments, a compound wherein M is —NH—.

In the above embodiments, a compound wherein c is 1 and M is —NH—.

In the above embodiments, a compound wherein m is 1.

In the above embodiments, a compound wherein W is selected from —NH—, —O—, and —N(C₁-C₆ alkyl)-.

In the above embodiments, a compound wherein W is —NH—.

In the above embodiments, a compound wherein at least one of X₄, X₅, X₆, and X₇ is N.

In the above embodiments, a compound wherein X₄ is N.

In the above embodiments, a compound wherein X₅ is N.

In the above embodiments, a compound wherein X₆ is N.

In the above embodiments, a compound wherein X₇ is N.

In the above embodiments, a compound wherein X₄ and X₆ are N.

In the above embodiments, a compound wherein X₅ and X₇ are N.

In the above embodiments, a compound wherein X₅ or X₆ are N, and both X₄ and X₇ are independently CR₂.

In the above embodiments, a compound wherein A is

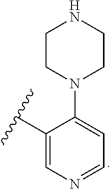 [SMILES: *c1cnccc1N1CCNCC1]

or a pharmaceutically acceptable salt thereof.

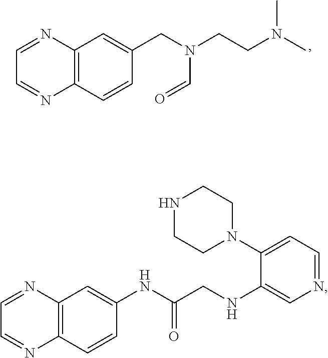 [SMILES: CN(C)CCN(C=O)Cc1ccc2nccnc2c1 ; O=C(CNc1cnccc1N1CCNCC1)Nc1ccc2nccnc2c1]

In other embodiments, the small molecule has a structure of formula (IX):

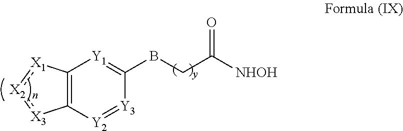

wherein X₁, X₂, and X₃ are, in each instance, independently selected from CR₁, CHR₁, N, NH, O and S, wherein adjacent X₁, X₂ and X₃ are not simultaneously selected to be O or S;
the dashed lines represent optional double bonds;
Y₁, Y₂, and Y₃ are, in each instance, independently selected from CR₂ and N;
R₁ and R₂ are independently selected from —H, —Cl, —Br, —I, —F, —CF₃, —OH, —CN, —NO₂, —NH₂, —NH(C₁-C₆ alkyl), —N(C₁-C₆ alkyl)₂, —COOH, —COO(C₁-C₆ alkyl), —CO(C₁-C₆ alkyl), —O(C₁-C₆ alkyl), —OCO(C₁-C₆ alkyl), —NCO(C₁-C₆ alkyl), —CONH(C₁-C₆ alkyl), and substituted or unsubstituted C₁-C₆ alkyl;
n is 1 or 2, wherein when n is 1, only one of the dashed lines is a double bond;
y is an integer selected from 1, 2, 3, 4, and 5; and
B is selected from —NH— and —NHC(═O)—; or a pharmaceutically acceptable salt thereof.

In the above embodiments, a compound wherein B is —NH—.

In the above embodiments, a compound wherein B is —NHC(═O)—.

In the above embodiments, a compound wherein y is an integer selected from 1, 2, and 3.

In the above embodiments, a compound wherein y is 1 or 3.

In the above embodiments, a compound wherein at least one of Y₁, Y₂, and Y₃ is N.

In the above embodiments, a compound wherein Yₜ is N.

In the above embodiments, a compound wherein Y₂ is N.

In the above embodiments, a compound wherein Y₃ is N.

In the above embodiments, a compound wherein at least one of Y₁, Y₂, and Y₃ is CR₂.

In the above embodiments, a compound wherein Yₜ is CR₂.

In the above embodiments, a compound wherein Y₂ is CR₂.

In the above embodiments, a compound wherein Y₃ is CR₂.

In the above embodiments, a compound wherein at least one of X₁, X₂, or X₃ is N.

In the above embodiments, a compound wherein, in each instance, two of X₁, X₂, and X₃ are N.

In the above embodiments, a compound wherein n is 2.

In the above embodiments, a compound with a structure of formula (X):

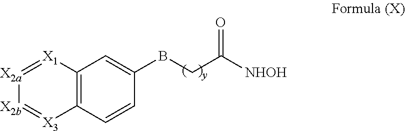

X₂ₐ and X_{2b} are independently selected from CR₁ and N;
X₁ and X₃ are independently selected from CR₁ and N;
wherein two of X₁, X₂a, X₂b, and X₃ are N; and
B, R₁ and y are as described in formula (VII); or a pharmaceutically acceptable salt thereof.

In the above embodiments, a compound having the structure of formula (XIa) or (XIb)

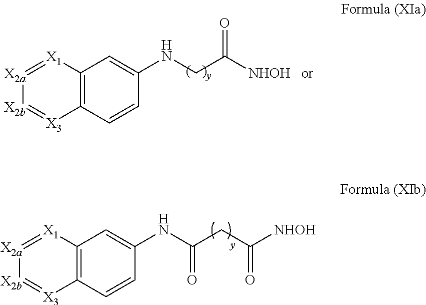

wherein
   X₂ₐ and X_{2b} are independently selected from CR₁ and N;
   X₁ and X₃ are independently selected from CR₁ and N;
   wherein two of X₁, X₂a, X₂b, and X₃ are N;
   wherein y is an integer selected from 1, 2, and 3; and R₁ is as described in formula (IX); or a pharmaceutically acceptable salt thereof.

In the above embodiments, a compound wherein y is 1.

In the above embodiments, a compound wherein y is 3.

In the above embodiments, a compound having the structure of formula (XII):

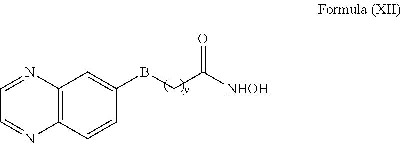

wherein B and y are as described in formula (IX); or a pharmaceutically acceptable salt thereof.

In the above embodiments, a compound wherein B is —NH—.

In the above embodiments, a compound wherein B is —NHC(═O)—.

In the above embodiments, a compound wherein said compound has the structure:

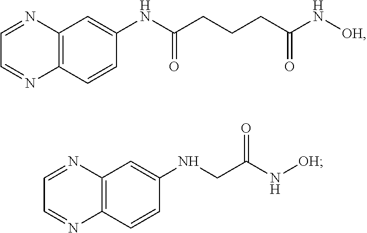 [SMILES: O=C(CCCC(=O)Nc1ccc2nccnc2c1)NO ; O=C(CNc1ccc2nccnc2c1)NO]

or a pharmaceutically acceptable salt thereof

Compounds according to the above formulas and embodiments may be prepared, for example, according to the methods provided in PCT/US2020/45022 and from U.S. provisional application Ser. No. 63/195,779, filed Jun. 2, 2021, the disclosures of which are incorporated herein by reference in their entirety.

In other embodiments, the small molecule has a structure according to formula XIII

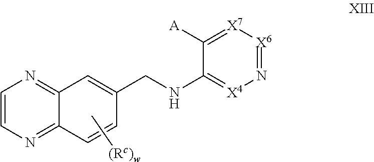

or a pharmaceutically acceptable salt thereof, wherein
X⁴ is selected from CH, CR^{d} and N;
X⁶ is selected from CH, CR^{d} and N;
X⁷ is selected from CH, CR^{d} and N;
   wherein 0 or 1 of X⁴, X⁶ or X⁷ is N;
A is selected from the group consisting of

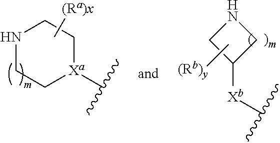

X^{a} is selected from N and CH;
X^{b} is selected from O, NH, and NCH₃;
each R^{a} is independently selected from C₁ to C₃ alkyl, —OCH₃, —CF₃, —CH₂F, —CHF₂, halo, hydroxyl and amino; or additionally or alternatively, two R^{a} attached to the same carbon atom form an oxo group; or two R^{a} attached to different carbon atoms form a 4- to 6-membered carbocyclic ring or a 4- to 6-membered heterocyclic ring having 1 or 2 heteroatoms selected from O and NH;
each R^{b} is independently selected from C₁ to C₃ alkyl, —OCH₃, —CF₃, —CH₂F, —CHF₂, halo, hydroxyl and amino; or additionally or alternatively, two R attached to the same carbon atom form an oxo group; or two R^{b} attached to different carbon atoms form a 4- to 6-membered carbocyclic ring or a 4- to 6-membered heterocyclic ring having 1 or 2 heteroatoms selected from O and NH;
m is 1 or 2;
x is 0, 1, 2 or 3;
y is 0, 1, 2 or 3;
each R^{c} is independently selected from halo, C₁ to C₃ alkyl, —OCH₃, —CF₃, —CH₂F, —CHF₂, —CN, hydroxyl and amino;
each R^{d} is independently selected from halo, C₁ to C₃ alkyl, —OCH₃, —CF₃, —CH₂F, —CHF₂, —CN, hydroxyl and amino;
alternatively, two R^{d} on adjacent ring positions may be taken together to form a 5- or 6-membered aromatic ring having from 0 to 2 heteroatoms selected from O, S, N and NH;
and w is 0, 1 or 2.

For the compounds according to formula XIII, x may be selected to be 1, 2 or 3; x may be selected to be 1 or 2; or, x may be selected to be 1. R^{a} may be selected to be methyl, fluoro or chloro; or R^{a} may be selected to be methyl. Alternatively, x may be 0.

For the compounds according to formula XIII, y may be selected to be 0 or 1. R^{b} may be selected from halo or methyl, or R^{b} may be selected to be methyl.

For the compounds according to formula XIII, w may be selected from 0 or 1. R^{c} may be selected from halo or methyl; or R^{c} may be selected from F, Cl or methyl.

For the compounds according to formula XIII, each R^{d} may be selected from halo, C₁ to C₃ alkyl, —OCH₃, —CF₃, —CH₂F, and —CHF₂; or R^{d} may be selected from CH₃, CH₂F, CHF₂, CF₃, F, Cl, Br, and OCH₃. Alternatively, two R^{d} on adjacent ring positions may be taken together to form a 5- or 6-membered aromatic ring having from 0 to 2 heteroatoms selected from O, S, N and NH.

For the compounds according to formula XIII, X^{a} may be N.

For the compounds according to formula XIII, X^{b} may be O.

In some embodiments of compounds of formula XIII, when A is selected to be

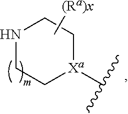

x is 1, 2 or 3; and/or
two R^{d} on adjacent ring positions are taken together to form a 5- or 6-membered aromatic ring having from 0 to 2 heteroatoms selected from O, S, N and NH.

In other embodiments, the small molecule has a structure according to formula XIV

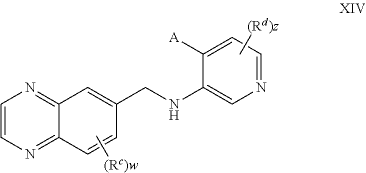

or a pharmaceutically acceptable salt thereof, wherein
A is selected from the group consisting of:

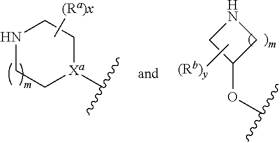

X^{a} is selected from N and CH;
each R^{a} is independently selected from C₁ to C₃ alkyl, —OCH₃, —CF₃, —CH₂F, —CHF₂, halo, hydroxyl and amino; or additionally or alternatively, two R^{a} attached to the same carbon atom form an oxo group; or two R^{a} attached to different carbon atoms form a 4- to 6-membered carbocyclic ring or a 4- to 6-membered heterocyclic ring having 1 or 2 heteroatoms selected from O and NH; each R^{b} is independently selected from C₁ to C₃ alkyl, —OCH₃, —CF₃, —CH₂F, —CHF₂, halo, hydroxyl and amino; or additionally or alternatively, two R^{b} attached to the same carbon atom form an oxo group; or two R^{b} attached to different carbon atoms form a 4- to 6-membered carbocyclic ring or a 4- to 6-membered heterocyclic ring having 1 or 2 heteroatoms selected from O and NH;
m is 1 or 2;
x is 0, 1, 2 or 3;
y is 0, 1, 2 or 3;
each R^{c} is independently selected from halo, C₁ to C₃ alkyl, —OCH₃, —CF₃, —CH₂F, —CHF₂, —CN, hydroxyl and amino;
each R^{d} is independently selected from halo, C₁ to C₃ alkyl, —OCH₃, —CF₃, —CH₂F, —CHF₂, —CN, hydroxyl and amino; alternatively, two R^{d} on adjacent ring positions may be taken together to form a 5- or 6-membered aromatic ring having from 0 to 2 heteroatoms selected from O, S, N and NH;
w is 0, 1 or 2; and
z is 0, 1 or 2.

For the compounds according to formula XIV, x may be selected to be 1, 2 or 3; x may be selected to be 1 or 2; or, x may be selected to be 1. R^{a} may be selected to be methyl, fluoro or chloro; or R^{a} may be selected to be methyl. Alternatively, x may be 0.

For the compounds according to formula XIV, y may be selected to be 0 or 1. R^{b} may be selected from halo or methyl; or R^{b} may be selected to be methyl.

For the compounds according to formula XIV, w may be selected from 0 or 1. R^{c} may be selected from halo or methyl; or R^{c} may be selected from F, Cl or methyl.

For the compounds according to formula XIV, z may be selected to be 1 or 2; or z may be selected to be 1. Each R^{d} may be independently selected from halo, C₁ to C₃ alkyl, —OCH₃, —CF₃, —CH₂F, and —CHF₂; or R^{d} may be selected from CH₃, CH₂F, CHF₂, CF₃, F, Cl, Br, and OCH₃. Alternatively, two R^{d} on adjacent ring positions may be taken together to form a 5- or 6-membered aromatic ring having from 0 to 2 heteroatoms selected from O, S, N and NH. Alternatively, z may be 0.

For the compounds according to formula XIV, X^{a} may be N.

In some embodiments of compounds of formula XIV, when A is selected to be

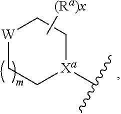

x is 1, 2 or 3; and/or
two R^{d} on adjacent ring positions are taken together to form a 5- or 6-membered aromatic ring having from 0 to 2 heteroatoms selected from O, S, N and NH.

In other embodiments, the small molecule has a structure according to formula XV

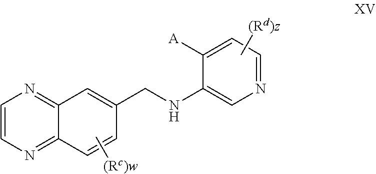

or a pharmaceutically acceptable salt thereof, wherein
A is selected from the group consisting of:

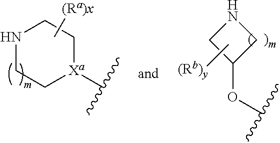

each R^{a} is independently selected from C₁ to C₃ alkyl, —OCH₃, —CF₃, —CH₂F, —CHF₂, halo, hydroxyl and amino; or additionally or alternatively, two R^{a} attached to the same carbon atom form an oxo group; or two R^{a} attached to different carbon atoms form a 4- to 6-membered carbocyclic ring or a 4- to 6-membered heterocyclic ring having 1 or 2 heteroatoms selected from O and NH;
each R^{b} is independently selected from C₁ to C₃ alkyl, —OCH₃, —CF₃, —CH₂F, —CHF₂, halo, hydroxyl and amino; or additionally or alternatively, two R^{b} attached to the same carbon atom form an oxo group; or two R^{b} attached to different carbon atoms form a 4- to 6-membered carbocyclic ring or a 4- to 6-membered heterocyclic ring having 1 or 2 heteroatoms selected from O and NH;
m is 1 or 2;
x is 0, 1, 2 or 3;
y is 0, 1, 2 or 3;
each R^{c} is independently selected from halo, C₁ to C₃ alkyl, —OCH₃, —CF₃, —CH₂F, —CHF₂, —CN, hydroxyl and amino;
each R^{d} is independently selected from halo, C₁ to C₃ alkyl, —OCH₃, —CF₃, —CH₂F, —CHF₂, —CN, hydroxyl and amino;
alternatively, two R^{d} on adjacent ring positions may be taken together to form a 5- or 6-membered aromatic ring having from 0 to 2 heteroatoms selected from O, S, N and NH;
w is 0, 1 or 2; and
z is 0, 1 or 2.

For the compounds according to formula XV, x may be selected to be 1, 2 or 3; x may be selected to be 1 or 2; or, x may be selected to be 1. R^{a} may be selected to be methyl, fluoro or chloro; or R^{a} may be selected to be methyl. Alternatively, x may be 0.

For the compounds according to formula XV, y may be selected to be 0 or 1. R^{b} may be selected from halo or methyl; or R^{b} may be selected to be methyl.

For the compounds according to formula XV, w may be selected from 0 or 1. R^{c} may be selected from halo or methyl; or R^{c} may be selected from F, Cl or methyl.

For the compounds according to formula XV, z may be selected to be 1 or 2; or z may be selected to be 1. Each R^{d} may be independently selected from halo, C₁ to C₃ alkyl, —OCH₃, —CF₃, —CH₂F, and —CHF₂; or R^{d} may be selected from CH₃, CH₂F, CHF₂, CF₃, F, Cl, Br, and OCH₃. Alternatively, two R^{d} on adjacent ring positions may be taken together to form a 5- or 6-membered aromatic ring having from 0 to 2 heteroatoms selected from O, S, N and NH. Alternatively, z may be 0.

In some embodiments of compounds of formula XV, when A is selected to be

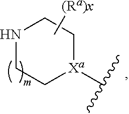

x is 1, 2 or 3; and/or
two R^{d} on adjacent ring positions are taken together to form a 5- or 6-membered aromatic ring having from 0 to 2 heteroatoms selected from O, S, N and NH.

In other embodiments, the small molecule has a structure according to formula XVI

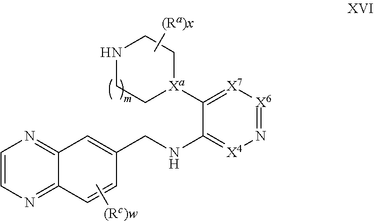

or a pharmaceutically acceptable salt thereof, wherein
X⁴ is selected from CH, CR^{d} and N;
X⁶ is selected from CH, CR^{d} and N;
X⁷ is selected from CH, CR^{d} and N;
   wherein 0 or 1 of X⁴, X⁶ or X⁷ is N;
X^{a} is selected from N and CH;
each R^{a} is independently selected from C₁ to C₃ alkyl, —OCH₃, —CF₃, —CH₂F, —CHF₂, halo, hydroxyl and amino; or additionally or alternatively, two R^{a} attached to the same carbon atom form an oxo group; or two R^{a} attached to different carbon atoms form a 4- to 6-membered carbocyclic ring or a 4- to 6-membered heterocyclic ring having 1 or 2 heteroatoms selected from O and NH;
m is 1 or 2;
x is 0, 1, 2 or 3;
each R^{c} is independently selected from halo, C₁ to C₃ alkyl, —OCH₃, —CF₃, —CH₂F, —CHF₂, —CN, hydroxyl and amino;
each R^{d} is independently selected from halo, C₁ to C₃ alkyl, —OCH₃, —CF₃, —CH₂F, —CHF₂, —CN, hydroxyl and amino;
alternatively, two R^{d} on adjacent ring positions may be taken together to form a 5- or 6-membered aromatic ring having from 0 to 2 heteroatoms selected from O, S, N and NH; and
w is 0, 1 or 2.

For the compounds according to formula XVI, x may be selected to be 1, 2 or 3; x may be selected to be 1 or 2; or, x may be selected to be 1. R^{a} may be selected to be methyl, fluoro or chloro; or R^{a} may be selected to be methyl. Alternatively, x may be 0.

For the compounds according to formula XVI, w may be selected from 0 or 1. R^{c} may be selected from halo or methyl; or R^{c} may be selected from F, Cl or methyl.

For the compounds according to formula XVI, each R^{d} may be selected from halo, C₁ to C₃ alkyl, —OCH₃, —CF₃, —CH₂F, and —CHF₂; or R^{d} may be selected from CH₃, CH₂F, CHF₂, CF₃, F, Cl, Br, and OCH₃. Alternatively, two R^{d} on adjacent ring positions may be taken together to form a 5- or 6-membered aromatic ring having from 0 to 2 heteroatoms selected from O, S, N and NH.

For the compounds according to formula XVI, X^{a} may be N.

For the compounds according to formula XVI, X^{b} may be O.

In some embodiments of compounds of formula XVI, x is 1, 2 or 3; and/or two R^{d} on adjacent ring positions are taken together to form a 5- or 6-membered aromatic ring having from 0 to 2 heteroatoms selected from O, S, N and NH.

In other embodiments, the small molecule has a structure according to formula XVII

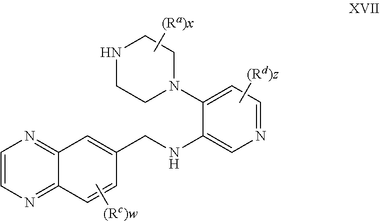

or a pharmaceutically acceptable salt thereof, wherein
each R^{a} is independently selected from C₁ to C₃ alkyl, —OCH₃, —CF₃, —CH₂F, —CHF₂, halo, hydroxyl and amino; or additionally or alternatively, two R^{a} attached to the same carbon atom form an oxo group; or two R^{a} attached to different carbon atoms form a 4- to 6-membered carbocyclic ring or a 4- to 6-membered heterocyclic ring having 1 or 2 heteroatoms selected from O and NH;
each R^{c} is independently selected from halo, C₁ to C₃ alkyl, —OCH₃, —CF₃, —CH₂F, —CHF₂, —CN, hydroxyl and amino;
each R^{d} is independently selected from halo, C₁ to C₃ alkyl, —OCH₃, —CF₃, —CH₂F, —CHF₂, —CN, hydroxyl and amino;
alternatively, two R^{d} on adjacent ring positions may be taken together to form a 5- or 6-membered aromatic ring having from 0 to 2 heteroatoms selected from O, S, N and NH;
w is 0, 1 or 2;
x is 0, 1, 2 or 3; and
z is 0, 1 or 2.

For the compounds according to formula XVII, x may be selected to be 1, 2 or 3; x may be selected to be 1 or 2; or, x may be selected to be 1. R^{a} may be selected to be methyl, fluoro or chloro; or R^{a} may be selected to be methyl. Alternatively, x may be 0.

For the compounds according to formula XVII, w may be selected from 0 or 1. Re may be selected from halo or methyl; or R^{c} may be selected from F, Cl or methyl.

For the compounds according to formula XVII, z may be selected to be 1 or 2; or z may be selected to be 1. Each R^{d} may be independently selected from halo, C₁ to C₃ alkyl, —OCH₃, —CF₃, —CH₂F, and —CHF₂; or each R^{d} may be independently selected from CH₃, CH₂F, CHF₂, CF₃, F, Cl, Br, and OCH₃. Alternatively, two R^{d} on adjacent ring positions may be taken together to form a 5- or 6-membered aromatic ring having from 0 to 2 heteroatoms selected from O, S, N and NH.

In some embodiments of compounds of formula XVII, x is 1, 2 or 3; and/or two R^{d} on adjacent ring positions are taken together to form a 5- or 6-membered aromatic ring having from 0 to 2 heteroatoms selected from O, S, N and NH.

In other embodiments, the small molecule has a structure according to formula XVIII

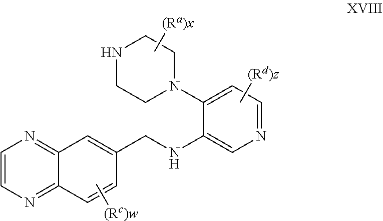

or a pharmaceutically acceptable salt thereof, wherein
each R^{a} is independently selected from methyl, halo, hydroxyl and amino;
each R^{c} is independently selected from methyl, halo, hydroxyl and amino;
each R^{d} is independently selected from methyl, halo, hydroxyl and amino;
x is 0, 1, 2 or 3;
w is 0, 1 or 2; and
z is 0, 1 or 2.

For the compounds according to formula XVIII, x may be selected to be 1, 2 or 3; x may be selected to be 1 or 2; or, x may be selected to be 1. R^{a} may be selected to be methyl, fluoro or chloro; or R^{a} may be selected to be methyl. Alternatively, x may be 0.

For the compounds according to formula XVIII, w may be selected from 0 or 1. R^{c} may be selected from halo or methyl; or R^{c} may be selected from F, Cl or methyl.

For the compounds according to formula XVIII, z may be selected to be 0 or 1; or z may be selected to be 1.

In other embodiments, the small molecule has a structure according to formula XIX:

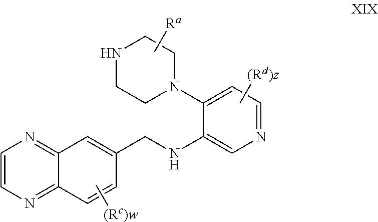

or a pharmaceutically acceptable salt thereof, wherein
R^{a} is selected from C₁ to C₃ alkyl, —OCH₃, —CF₃, —CH₂F, —CHF₂, halo, hydroxyl and amino; or additionally or alternatively;
each R^{c} is independently selected from halo, C₁ to C₃ alkyl, —OCH₃, —CF₃, —CH₂F, —CHF₂, —CN, hydroxyl and amino;
each R^{d} is independently selected from halo, C₁ to C₃ alkyl, —OCH₃, —CF₃, —CH₂F, —CHF₂, —CN, hydroxyl and amino;
alternatively, two R^{d} on adjacent ring positions may be taken together to form a 5- or 6-membered aromatic ring having from 0 to 2 heteroatoms selected from O, S, N and NH; and
w is 0, 1 or 2.
z is 0, 1 or 2.

For the compounds according to formula XIX, R^{a} may be selected from methyl, halo, hydroxyl and amino; R^{a} may be selected to be methyl, fluoro or chloro; or R^{a} may be selected to be methyl.

For the compounds according to formula XIX, each R^{c} may be independently selected from methyl, halo, hydroxyl and amino.

For the compounds according to formula XIX, each R^{d} may be independently selected from methyl, halo, hydroxyl and amino.

In other embodiments, the small molecule has a structure according to formula XX

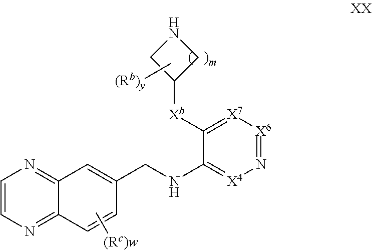

or a pharmaceutically acceptable salt thereof, wherein
X⁴ is selected from CH, CR^{d} and N;
X⁶ is selected from CH, CR^{d} and N;
X⁷ is selected from CH, CR^{d} and N;
   wherein 0 or 1 of X⁴, X⁶ or X⁷ is N;
X^{b} is selected from O, NH, and NCH₃;
each R^{b} is independently selected from C₁ to C₃ alkyl, —OCH₃, —CF₃, —CH₂F, —CHF₂, halo, hydroxyl and amino; or additionally or alternatively, two R^{b} attached to the same carbon atom form an oxo group; or two R^{b} attached to different carbon atoms form a 4- to 6-membered carbocyclic ring or a 4- to 6-membered heterocyclic ring having 1 or 2 heteroatoms selected from O and NH;
m is 1 or 2;
y is 0, 1, 2 or 3;
each R^{c} is independently selected from halo, C₁ to C₃ alkyl, —OCH₃, —CF₃, —CH₂F, —CHF₂, —CN, hydroxyl and amino;
each R^{d} is independently selected from halo, C₁ to C₃ alkyl, —OCH₃, —CF₃, —CH₂F, —CHF₂, —CN, hydroxyl and amino;
alternatively, two R^{d} on adjacent ring positions may be taken together to form a 5- or 6-membered aromatic ring having from 0 to 2 heteroatoms selected from O, S, N and NH; and
w is 0, 1 or 2.

For the compounds according to formula XX, y may be selected to be 0 or 1. R^{b} may be selected from halo or methyl; or R^{b} may be selected to be methyl.

For the compounds according to formula XX, w may be selected from 0 or 1. R^{c} may be selected from halo or methyl; or R^{c} may be selected from F, Cl or methyl.

For the compounds according to formula XX, each R^{d} may be selected from halo, C₁ to C₃ alkyl, —OCH₃, —CF₃, —CH₂F, and —CHF₂; or R^{d} may be selected from CH₃, CH₂F, CHF₂, CF₃, F, Cl, Br, and OCH₃. Alternatively, two R^{d} on adjacent ring positions may be taken together to form a 5- or 6-membered aromatic ring having from 0 to 2 heteroatoms selected from O, S, N and NH.

For the compounds according to formula XX, X^{b} may be O.

In other embodiments, the small molecule has a structure according to formula XXI

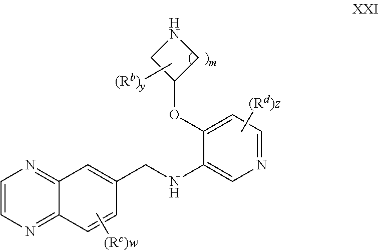

or a pharmaceutically acceptable salt thereof, wherein
each R^{b} is independently selected from C₁ to C₃ alkyl, —OCH₃, —CF₃, —CH₂F, —CHF₂, halo, hydroxyl and amino; or additionally or alternatively, two R^{b} attached to the same carbon atom form an oxo group; or two R^{b} attached to different carbon atoms form a 4- to 6-membered carbocyclic ring or a 4- to 6-membered heterocyclic ring having 1 or 2 heteroatoms selected from O and NH;
each R^{c} is independently selected from halo, C₁ to C₃ alkyl, —OCH₃, —CF₃, —CH₂F, —CHF₂, —CN, hydroxyl and amino;
each R^{d} is independently selected from halo, C₁ to C₃ alkyl, —OCH₃, —CF₃, —CH₂F, —CHF₂, —CN, hydroxyl and amino;
alternatively, two R^{d} on adjacent ring positions may be taken together to form a 5- or 6-membered aromatic ring having from 0 to 2 heteroatoms selected from O, S, N and NH;
m is 1 or 2;
w is 0, 1 or 2
y is 0, 1 or 2; and
z is 0, 1 or 2.

For the compounds according to formula XXI, y may be selected to be 0 or 1. R^{b} may be selected from halo or methyl; or R^{b} may be selected to be methyl.

For the compounds according to formula XXI, w may be selected from 0 or 1. R^{c} may be selected from halo or methyl; or R^{c} may be selected from F, Cl or methyl.

For the compounds according to formula XXI, each R^{d} may be selected from halo, C₁ to C₃ alkyl, —OCH₃, —CF₃, —CH₂F, and —CHF₂; or R^{d} may be selected from CH₃, CH₂F, CHF₂, CF₃, F, Cl, Br, and OCH₃. Alternatively, two R^{d} on adjacent ring positions may be taken together to form a 5- or 6-membered aromatic ring having from 0 to 2 heteroatoms selected from O, S, N and NH.

In other embodiments, the small molecule has a structure according to formula XXII

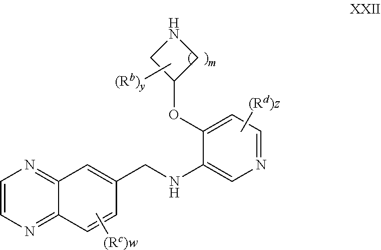

or a pharmaceutically acceptable salt thereof, wherein
each R^{b} is independently selected from C₁ to C₃ alkyl, —OCH₃, —CF₃, —CH₂F, —CHF₂, halo, hydroxyl and amino; or additionally or alternatively, two R^{b} attached to the same carbon atom form an oxo group; or two R^{b} attached to different carbon atoms form a 4- to 6-membered carbocyclic ring or a 4- to 6-membered heterocyclic ring having 1 or 2 heteroatoms selected from O and NH;
each R^{c} is independently selected from halo, C₁ to C₃ alkyl, —OCH₃, —CF₃, —CH₂F, —CHF₂, —CN, hydroxyl and amino;
each R^{d} is independently selected from halo, C₁ to C₃ alkyl, —OCH₃, —CF₃, —CH₂F, —CHF₂, —CN, hydroxyl and amino;
alternatively, two R^{d} on adjacent ring positions may be taken together to form a 5- or 6-membered aromatic ring having from 0 to 2 heteroatoms selected from O, S, N and NH;
m is 1 or 2;
w is 0, 1 or 2;
y is 0, 1 or 2; and
z is 0, 1 or 2.

For the compounds according to formula XXII, y may be selected to be 0 or 1. R^{b} may be selected from methyl, halo, hydroxyl and amino; or R^{b} may be selected from halo or methyl; or R^{b} may be selected to be methyl.

For the compounds according to formula XXII, w may be selected from 0 or 1. R^{c} may be selected from methyl, halo, hydroxyl and amino; or R^{c} may be selected from halo or methyl; or R^{c} may be selected from F, Cl or methyl.

For the compounds according to formula XXII, each R^{d} may be selected from halo, C₁ to C₃ alkyl, —OCH₃, —CF₃, —CH₂F, and —CHF₂; or R^{d} my be selected from methyl, halo, hydroxyl and amino; or R^{d} may be selected from CH₃, CH₂F, CHF₂, CF₃, F, Cl, Br, and OCH₃. Alternatively, two R^{d} on adjacent ring positions may be taken together to form a 5- or 6-membered aromatic ring having from 0 to 2 heteroatoms selected from O, S, N and NH.

In other embodiments, the small molecule has a structure according to the compounds in Table A (or a pharmaceutically acceptable salt thereof):

TABLE A

| Ref. | |
|---|---|
| No. | Structure |
| 001 | 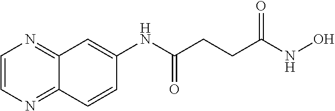 [SMILES: O=C(CCC(=O)Nc1ccc2nccnc2c1)NO] |
| 002 | 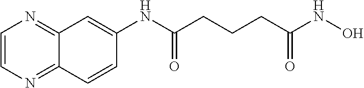 [SMILES: O=C(CCCC(=O)Nc1ccc2nccnc2c1)NO] |
| 003 | 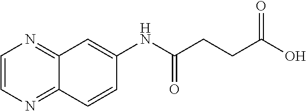 [SMILES: O=C(O)CCC(=O)Nc1ccc2nccnc2c1] |
| 004 | 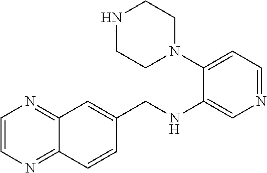 [SMILES: c1cc(N2CCNCC2)c(NCc2ccc3nccnc3c2)cn1] |
| 005 | 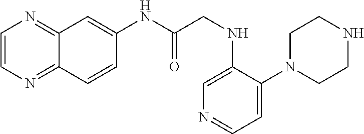 [SMILES: O=C(CNc1cnccc1N1CCNCC1)Nc1ccc2nccnc2c1] |
| 006 | 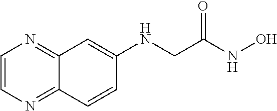 [SMILES: O=C(CNc1ccc2nccnc2c1)NO] |
| 007 | 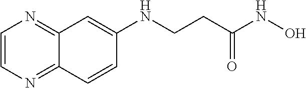 [SMILES: O=C(CCNc1ccc2nccnc2c1)NO] |
| 008 | 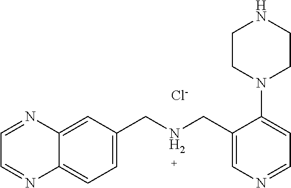 [SMILES: c1cc(N2CCNCC2)c(CNCc2ccc3nccnc3c2)cn1 ; [Cl-]] |
| 009 | 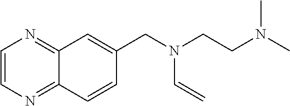 [SMILES: C=CN(CCN(C)C)Cc1ccc2nccnc2c1] |
| 010 | 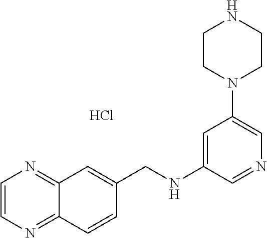 [SMILES: c1cnc2cc(CNc3cncc(N4CCNCC4)c3)ccc2n1 ; Cl] |
| 011 | 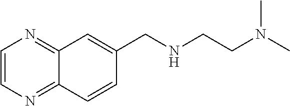 [SMILES: CN(C)CCNCc1ccc2nccnc2c1] |
| 012 | 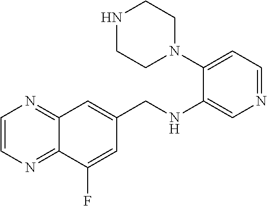 [SMILES: Fc1cc(CNc2cnccc2N2CCNCC2)cc2nccnc12] |
| 013 | 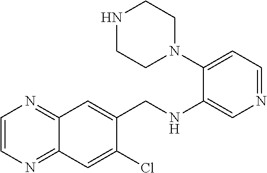 [SMILES: Clc1cc2nccnc2cc1CNc1cnccc1N1CCNCC1] |
| 014 | 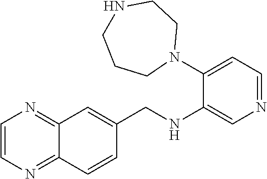 [SMILES: c1cc(N2CCCNCC2)c(NCc2ccc3nccnc3c2)cn1] |
| 015 | 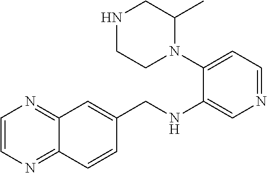 [SMILES: CC1CNCCN1c1ccncc1NCc1ccc2nccnc2c1] |
| 016 | 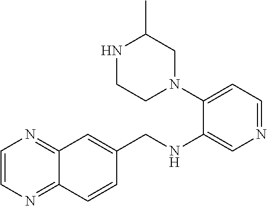 [SMILES: CC1CN(c2ccncc2NCc2ccc3nccnc3c2)CCN1] |
| 017 | 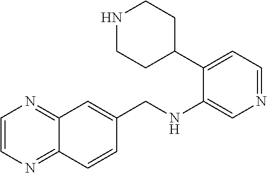 [SMILES: c1cc(C2CCNCC2)c(NCc2ccc3nccnc3c2)cn1] |
| 018 | 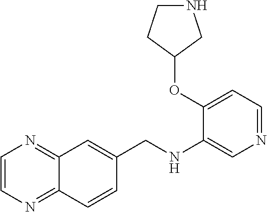 [SMILES: c1cc(OC2CCNC2)c(NCc2ccc3nccnc3c2)cn1] |
| 019 | 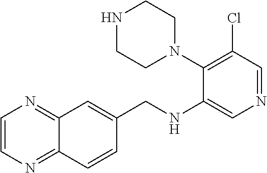 [SMILES: Clc1cncc(NCc2ccc3nccnc3c2)c1N1CCNCC1] |
| 020 | 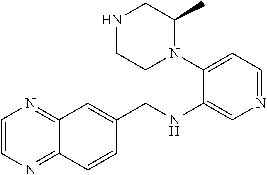 [SMILES: C[C@@H]1CNCCN1c1ccncc1NCc1ccc2nccnc2c1] |
| 021 | 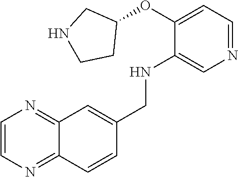 [SMILES: c1cc(O[C@@H]2CCNC2)c(NCc2ccc3nccnc3c2)cn1] |
| 022 | 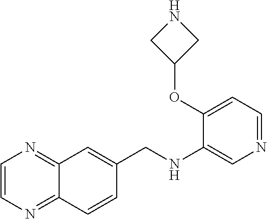 [SMILES: c1cc(OC2CNC2)c(NCc2ccc3nccnc3c2)cn1] |
| 023 | 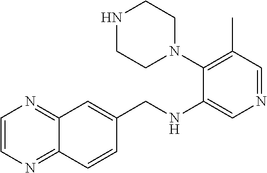 [SMILES: Cc1cncc(NCc2ccc3nccnc3c2)c1N1CCNCC1] |
| 024 | 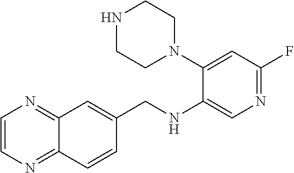 [SMILES: Fc1cc(N2CCNCC2)c(NCc2ccc3nccnc3c2)cn1] |
| 025 | 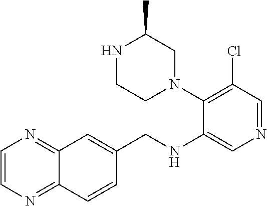 [SMILES: C[C@H]1CN(c2c(Cl)cncc2NCc2ccc3nccnc3c2)CCN1] |
| 026 | 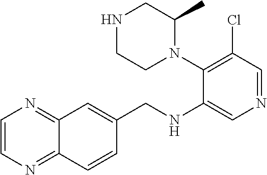 [SMILES: C[C@@H]1CNCCN1c1c(Cl)cncc1NCc1ccc2nccnc2c1] |
| 027 | 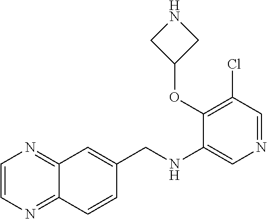 [SMILES: Clc1cncc(NCc2ccc3nccnc3c2)c1OC1CNC1] |
| 028 | 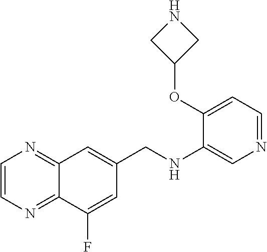 [SMILES: Fc1cc(CNc2cnccc2OC2CNC2)cc2nccnc12] |
| 029 | 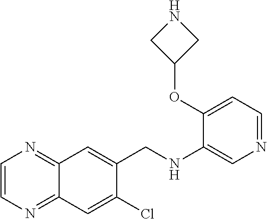 [SMILES: Clc1cc2nccnc2cc1CNc1cnccc1OC1CNC1] |
| 030 | 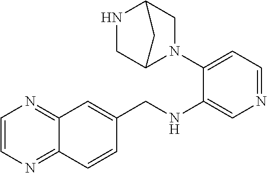 [SMILES: c1cc(N2CC3CC2CN3)c(NCc2ccc3nccnc3c2)cn1] |
| 031 | 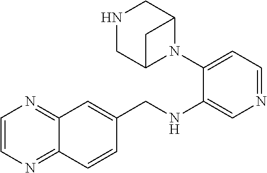 [SMILES: c1cc(N2C3CNCC2C3)c(NCc2ccc3nccnc3c2)cn1] |
| 032 | 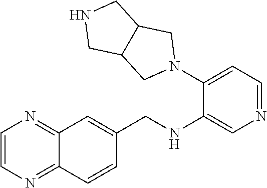 [SMILES: c1cc(N2CC3CNCC3C2)c(NCc2ccc3nccnc3c2)cn1] |
| 033 | 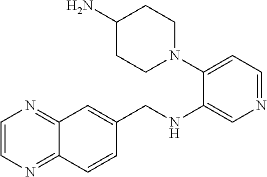 [SMILES: NC1CCN(c2ccncc2NCc2ccc3nccnc3c2)CC1] |
| 034 | 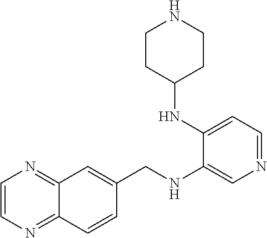 [SMILES: c1cc(NC2CCNCC2)c(NCc2ccc3nccnc3c2)cn1] |
| 035 | 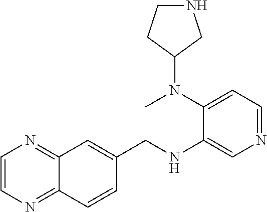 [SMILES: CN(c1ccncc1NCc1ccc2nccnc2c1)C1CCNC1] |
| 036 | 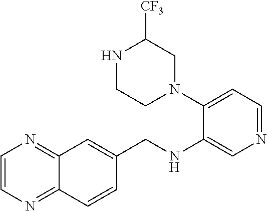 [SMILES: FC(F)(F)C1CN(c2ccncc2NCc2ccc3nccnc3c2)CCN1] |
| 037 | 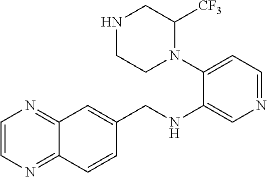 [SMILES: FC(F)(F)C1CNCCN1c1ccncc1NCc1ccc2nccnc2c1] |
| 038 | 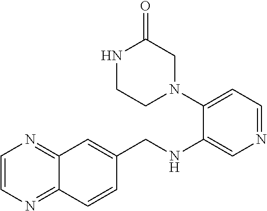 [SMILES: O=C1CN(c2ccncc2NCc2ccc3nccnc3c2)CCN1] |
| 039 | 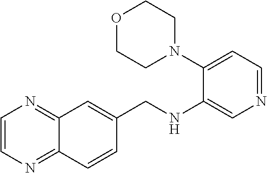 [SMILES: c1cc(N2CCOCC2)c(NCc2ccc3nccnc3c2)cn1] |
| 040 | 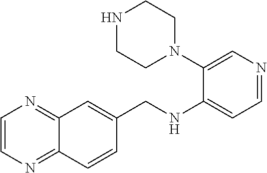 [SMILES: c1cc(NCc2ccc3nccnc3c2)c(N2CCNCC2)cn1] |
| 041 | 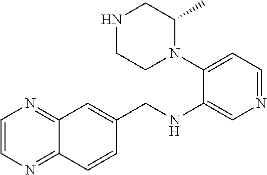 [SMILES: C[C@H]1CNCCN1c1ccncc1NCc1ccc2nccnc2c1] |
| 042 | 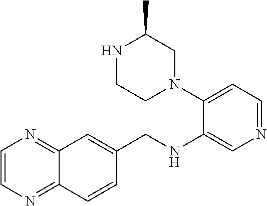 [SMILES: C[C@H]1CN(c2ccncc2NCc2ccc3nccnc3c2)CCN1] |
| 043 | 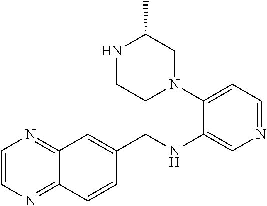 [SMILES: C[C@@H]1CN(c2ccncc2NCc2ccc3nccnc3c2)CCN1] |
| 044 | 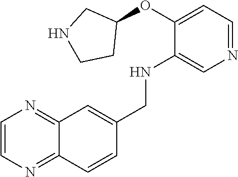 [SMILES: c1cc(O[C@H]2CCNC2)c(NCc2ccc3nccnc3c2)cn1] |
| 045 | 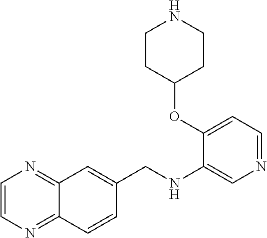 [SMILES: c1cc(OC2CCNCC2)c(NCc2ccc3nccnc3c2)cn1] |
| 046 | 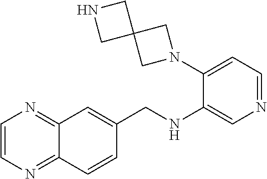 [SMILES: c1cc(N2CC3(CNC3)C2)c(NCc2ccc3nccnc3c2)cn1] |
| 047 | 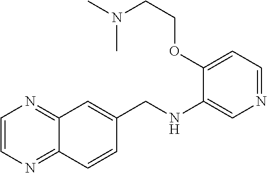 [SMILES: CN(C)CCOc1ccncc1NCc1ccc2nccnc2c1] |
| 048 | 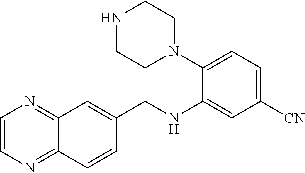 [SMILES: N#Cc1ccc(N2CCNCC2)c(NCc2ccc3nccnc3c2)c1] |
| 049 | 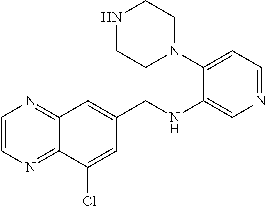 [SMILES: Clc1cc(CNc2cnccc2N2CCNCC2)cc2nccnc12] |
| 050 | 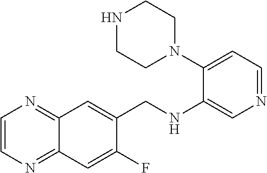 [SMILES: Fc1cc2nccnc2cc1CNc1cnccc1N1CCNCC1] |
| 051 | 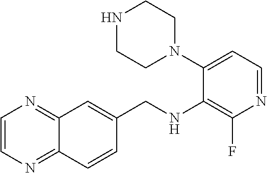 [SMILES: Fc1nccc(N2CCNCC2)c1NCc1ccc2nccnc2c1] |
| 052 | 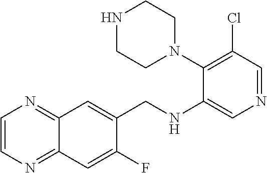 [SMILES: Fc1cc2nccnc2cc1CNc1cncc(Cl)c1N1CCNCC1] |
| 053 | 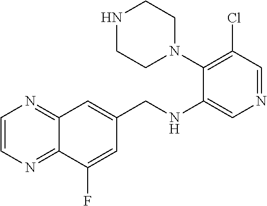 [SMILES: Fc1cc(CNc2cncc(Cl)c2N2CCNCC2)cc2nccnc12] |
| 054 | 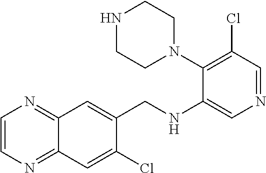 [SMILES: Clc1cc2nccnc2cc1CNc1cncc(Cl)c1N1CCNCC1] |
| 055 | 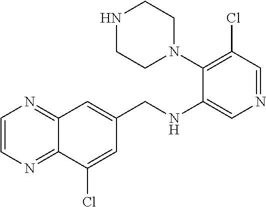 [SMILES: Clc1cncc(NCc2cc(Cl)c3nccnc3c2)c1N1CCNCC1] |
| 056 | 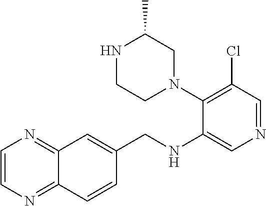 [SMILES: C[C@@H]1CN(c2c(Cl)cncc2NCc2ccc3nccnc3c2)CCN1] |
| 057 | 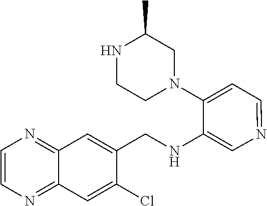 [SMILES: C[C@H]1CN(c2ccncc2NCc2cc3nccnc3cc2Cl)CCN1] |
| 058 | 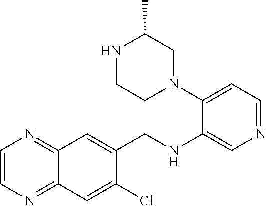 [SMILES: C[C@@H]1CN(c2ccncc2NCc2cc3nccnc3cc2Cl)CCN1] |
| 059 | 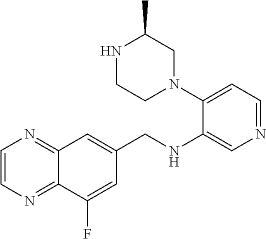 [SMILES: C[C@H]1CN(c2ccncc2NCc2cc(F)c3nccnc3c2)CCN1] |
| 060 | 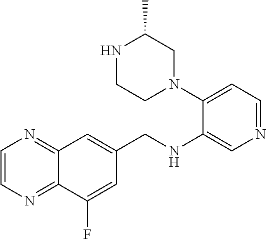 [SMILES: C[C@@H]1CN(c2ccncc2NCc2cc(F)c3nccnc3c2)CCN1] |
| 061 | 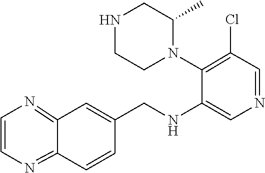 [SMILES: C[C@H]1CNCCN1c1c(Cl)cncc1NCc1ccc2nccnc2c1] |
| 062 | 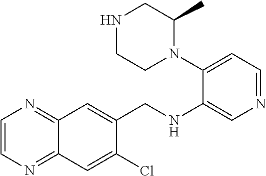 [SMILES: C[C@@H]1CNCCN1c1ccncc1NCc1cc2nccnc2cc1Cl] |
| 063 | 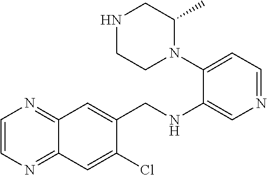 [SMILES: C[C@H]1CNCCN1c1ccncc1NCc1cc2nccnc2cc1Cl] |
| 064 | 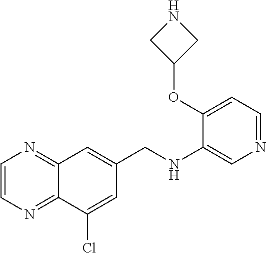 [SMILES: Clc1cc(CNc2cnccc2OC2CNC2)cc2nccnc12] |
| 065 | 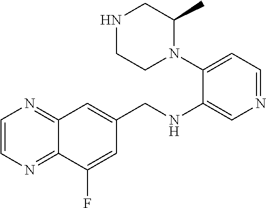 [SMILES: C[C@@H]1CNCCN1c1ccncc1NCc1cc(F)c2nccnc2c1] |
| 066 | 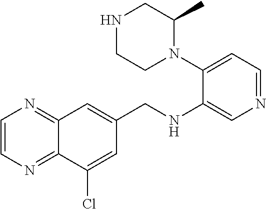 [SMILES: C[C@@H]1CNCCN1c1ccncc1NCc1cc(Cl)c2nccnc2c1] |
| 067 | 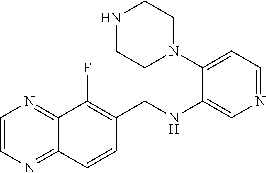 [SMILES: Fc1c(CNc2cnccc2N2CCNCC2)ccc2nccnc12] |
| 068 | 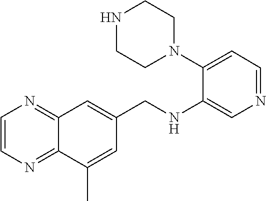 [SMILES: Cc1cc(CNc2cnccc2N2CCNCC2)cc2nccnc12] |
| 069 | 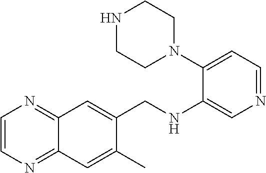 [SMILES: Cc1cc2nccnc2cc1CNc1cnccc1N1CCNCC1] |
| 070 | 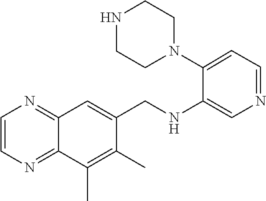 [SMILES: Cc1c(CNc2cnccc2N2CCNCC2)cc2nccnc2c1C] |
| 071 | 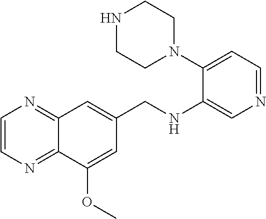 [SMILES: COc1cc(CNc2cnccc2N2CCNCC2)cc2nccnc12] |
| 072 | 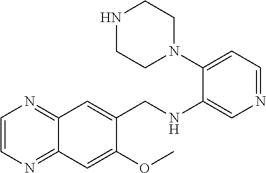 [SMILES: COc1cc2nccnc2cc1CNc1cnccc1N1CCNCC1] |
| 073 | 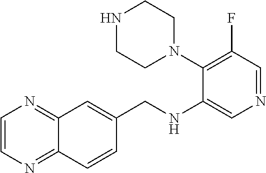 [SMILES: Fc1cncc(NCc2ccc3nccnc3c2)c1N1CCNCC1] |
| 074 | 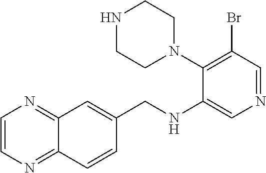 [SMILES: Brc1cncc(NCc2ccc3nccnc3c2)c1N1CCNCC1] |
| 075 | 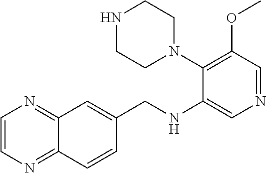 [SMILES: COc1cncc(NCc2ccc3nccnc3c2)c1N1CCNCC1] |
| 076 | 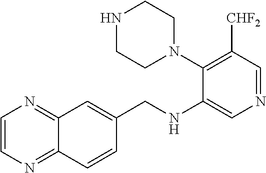 [SMILES: FC(F)c1cncc(NCc2ccc3nccnc3c2)c1N1CCNCC1] |
| 077 | 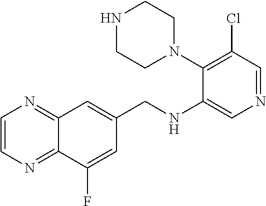 [SMILES: Fc1cc(CNc2cncc(Cl)c2N2CCNCC2)cc2nccnc12] |
| 078 | 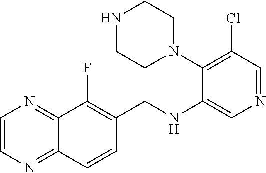 [SMILES: Fc1c(CNc2cncc(Cl)c2N2CCNCC2)ccc2nccnc12] |
| 079 | 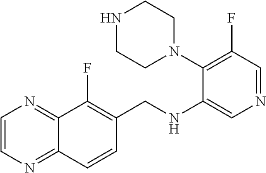 [SMILES: Fc1cncc(NCc2ccc3nccnc3c2F)c1N1CCNCC1] |
| 080 | 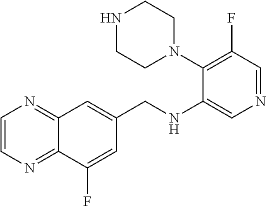 [SMILES: Fc1cncc(NCc2cc(F)c3nccnc3c2)c1N1CCNCC1] |
| 081 | 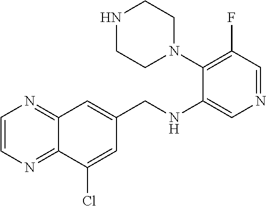 [SMILES: Fc1cncc(NCc2cc(Cl)c3nccnc3c2)c1N1CCNCC1] |
| 082 | 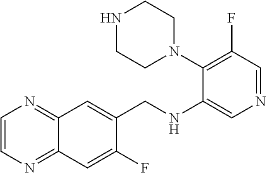 [SMILES: Fc1cc2nccnc2cc1CNc1cncc(F)c1N1CCNCC1] |
| 083 | 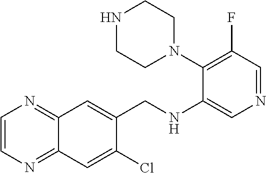 [SMILES: Fc1cncc(NCc2cc3nccnc3cc2Cl)c1N1CCNCC1] |
| 084 | 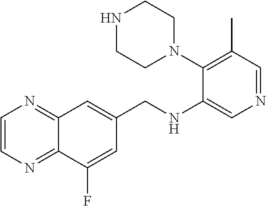 [SMILES: Cc1cncc(NCc2cc(F)c3nccnc3c2)c1N1CCNCC1] |
| 085 | 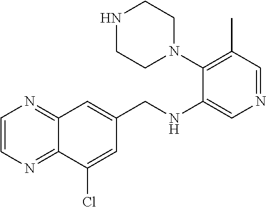 [SMILES: Cc1cncc(NCc2cc(Cl)c3nccnc3c2)c1N1CCNCC1] |
| 086 | 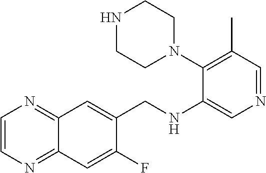 [SMILES: Cc1cncc(NCc2cc3nccnc3cc2F)c1N1CCNCC1] |
| 087 | 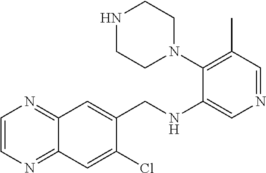 [SMILES: Cc1cncc(NCc2cc3nccnc3cc2Cl)c1N1CCNCC1] |
| 088 | 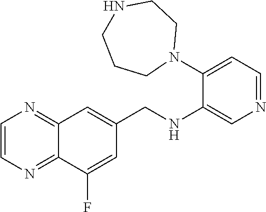 [SMILES: Fc1cc(CNc2cnccc2N2CCCNCC2)cc2nccnc12] |
| 089 | 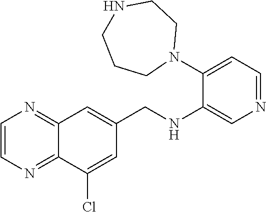 [SMILES: Clc1cc(CNc2cnccc2N2CCCNCC2)cc2nccnc12] |
| 090 | 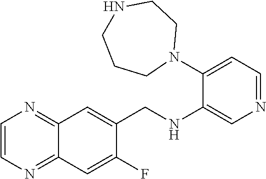 [SMILES: Fc1cc2nccnc2cc1CNc1cnccc1N1CCCNCC1] |
| 091 | 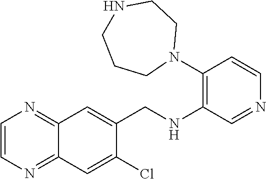 [SMILES: Clc1cc2nccnc2cc1CNc1cnccc1N1CCCNCC1] |
| 092 | 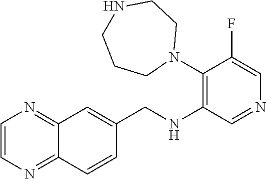 [SMILES: Fc1cncc(NCc2ccc3nccnc3c2)c1N1CCCNCC1] |
| 093 | 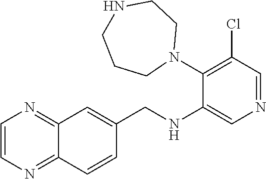 [SMILES: Clc1cncc(NCc2ccc3nccnc3c2)c1N1CCCNCC1] |
| 094 | 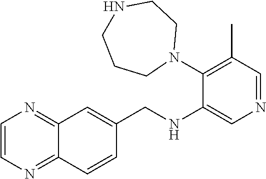 [SMILES: Cc1cncc(NCc2ccc3nccnc3c2)c1N1CCCNCC1] |
| 095 | 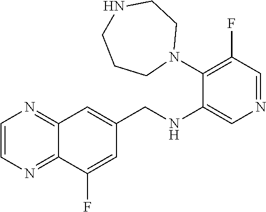 [SMILES: Fc1cncc(NCc2cc(F)c3nccnc3c2)c1N1CCCNCC1] |
| 096 | 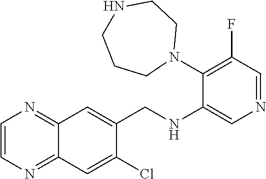 [SMILES: Fc1cncc(NCc2cc3nccnc3cc2Cl)c1N1CCCNCC1] |
| 097 | 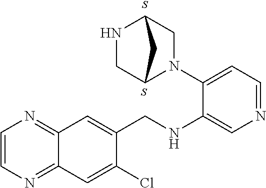 |
| 098 | 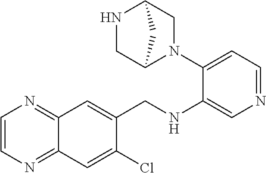 [SMILES: Clc1cc2nccnc2cc1CNc1cnccc1N1C[C@H]2C[C@@H]1CN2] |
| 099 | 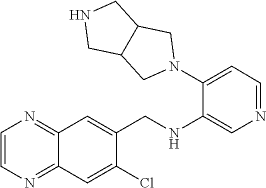 [SMILES: Clc1cc2nccnc2cc1CNc1cnccc1N1CC2CNCC2C1] |
| 100 | 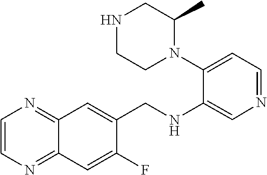 [SMILES: C[C@@H]1CNCCN1c1ccncc1NCc1cc2nccnc2cc1F] |
| 101 | 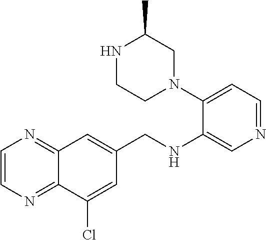 [SMILES: C[C@H]1CN(c2ccncc2NCc2cc(Cl)c3nccnc3c2)CCN1] |
| 102 | 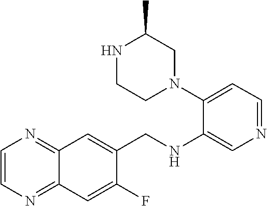 [SMILES: C[C@H]1CN(c2ccncc2NCc2cc3nccnc3cc2F)CCN1] |
| 103 | 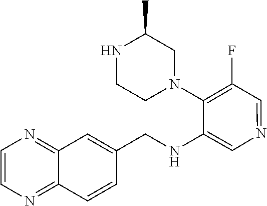 [SMILES: C[C@H]1CN(c2c(F)cncc2NCc2ccc3nccnc3c2)CCN1] |
| 104 | 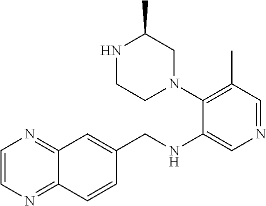 [SMILES: Cc1cncc(NCc2ccc3nccnc3c2)c1N1CCN[C@@H](C)C1] |
| 105 | 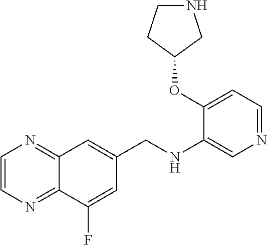 [SMILES: Fc1cc(CNc2cnccc2O[C@@H]2CCNC2)cc2nccnc12] |
| 106 | 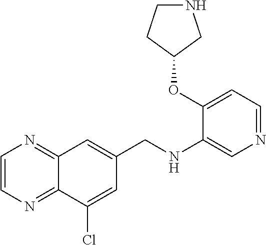 [SMILES: Clc1cc(CNc2cnccc2O[C@@H]2CCNC2)cc2nccnc12] |
| 107 | 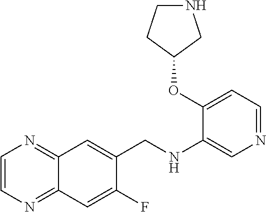 [SMILES: Fc1cc2nccnc2cc1CNc1cnccc1O[C@@H]1CCNC1] |
| 108 | 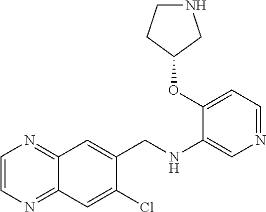 [SMILES: Clc1cc2nccnc2cc1CNc1cnccc1O[C@@H]1CCNC1] |
| 109 | 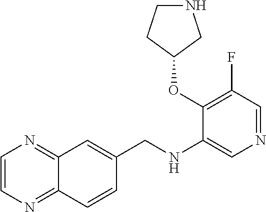 [SMILES: Fc1cncc(NCc2ccc3nccnc3c2)c1O[C@@H]1CCNC1] |
| 110 | 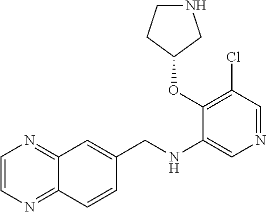 [SMILES: Clc1cncc(NCc2ccc3nccnc3c2)c1O[C@@H]1CCNC1] |
| 111 | 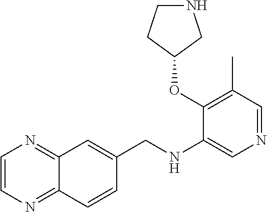 [SMILES: Cc1cncc(NCc2ccc3nccnc3c2)c1O[C@@H]1CCNC1] |
| 112 | 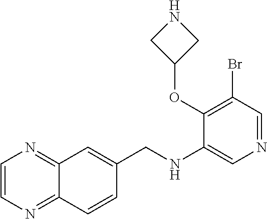 [SMILES: Brc1cncc(NCc2ccc3nccnc3c2)c1OC1CNC1] |
| 113 | 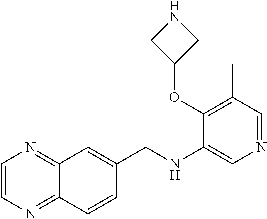 [SMILES: Cc1cncc(NCc2ccc3nccnc3c2)c1OC1CNC1] |
| 114 | 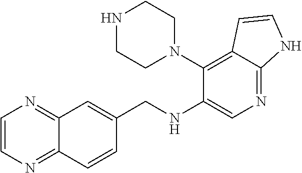 [SMILES: c1cnc2cc(CNc3cnc4[nH]ccc4c3N3CCNCC3)ccc2n1] |
| 115 | 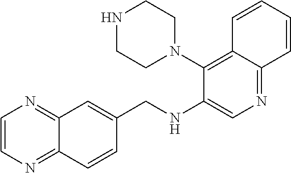 [SMILES: c1ccc2c(N3CCNCC3)c(NCc3ccc4nccnc4c3)cnc2c1] |
| 116 | 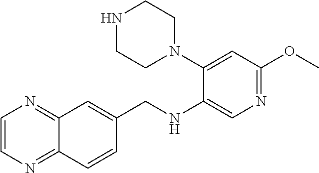 [SMILES: COc1cc(N2CCNCC2)c(NCc2ccc3nccnc3c2)cn1] |
| 117 | 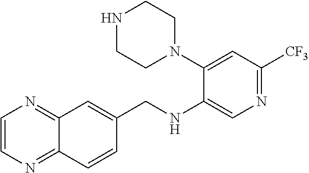 [SMILES: FC(F)(F)c1cc(N2CCNCC2)c(NCc2ccc3nccnc3c2)cn1] |
| 118 | 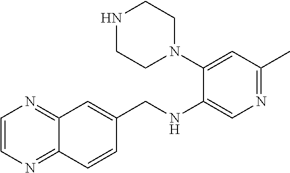 [SMILES: Cc1cc(N2CCNCC2)c(NCc2ccc3nccnc3c2)cn1] |
| 119 | 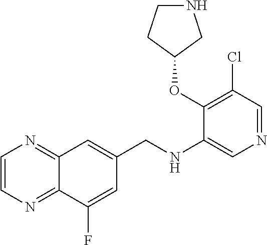 [SMILES: Fc1cc(CNc2cncc(Cl)c2O[C@@H]2CCNC2)cc2nccnc12] |
| 120 | 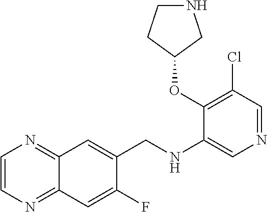 [SMILES: Fc1cc2nccnc2cc1CNc1cncc(Cl)c1O[C@@H]1CCNC1] |
| 121 | 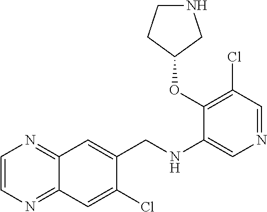 [SMILES: Clc1cc2nccnc2cc1CNc1cncc(Cl)c1O[C@@H]1CCNC1] |
| 122 | 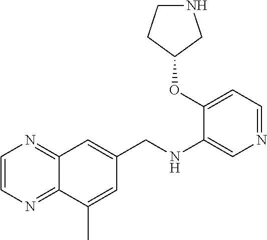 [SMILES: Cc1cc(CNc2cnccc2O[C@@H]2CCNC2)cc2nccnc12] |
| 123 | 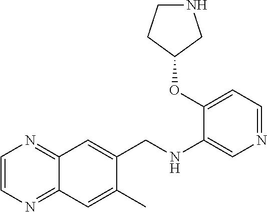 [SMILES: Cc1cc2nccnc2cc1CNc1cnccc1O[C@@H]1CCNC1] |
| 124 | 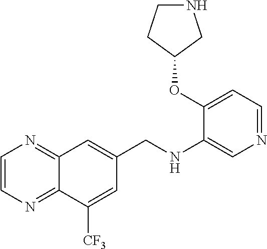 [SMILES: FC(F)(F)c1cc(CNc2cnccc2O[C@@H]2CCNC2)cc2nccnc12] |
| 125 | 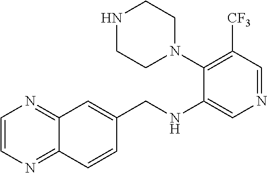 [SMILES: FC(F)(F)c1cncc(NCc2ccc3nccnc3c2)c1N1CCNCC1] |
| 126 | 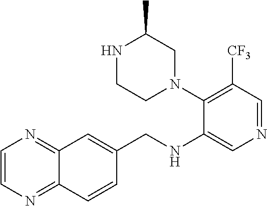 [SMILES: C[C@H]1CN(c2c(NCc3ccc4nccnc4c3)cncc2C(F)(F)F)CCN1] |
| 127 | 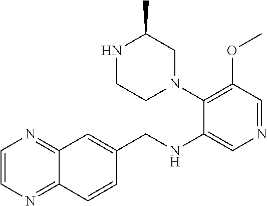 [SMILES: COc1cncc(NCc2ccc3nccnc3c2)c1N1CCN[C@@H](C)C1] |
| 128 | 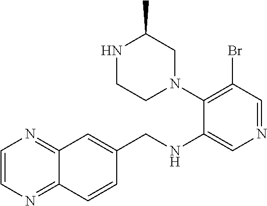 [SMILES: C[C@H]1CN(c2c(Br)cncc2NCc2ccc3nccnc3c2)CCN1] |
| 129 | 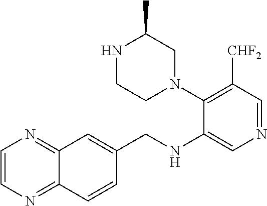 [SMILES: C[C@H]1CN(c2c(NCc3ccc4nccnc4c3)cncc2C(F)F)CCN1] |
| 130 | 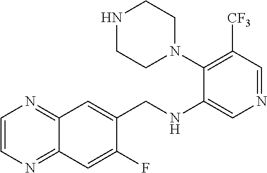 [SMILES: Fc1cc2nccnc2cc1CNc1cncc(C(F)(F)F)c1N1CCNCC1] |
| 131 | 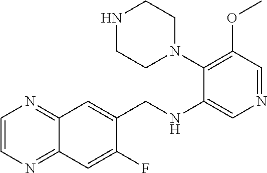 [SMILES: COc1cncc(NCc2cc3nccnc3cc2F)c1N1CCNCC1] |
| 132 | 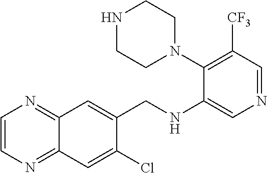 [SMILES: FC(F)(F)c1cncc(NCc2cc3nccnc3cc2Cl)c1N1CCNCC1] |
| 133 | 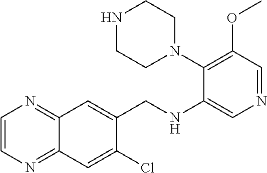 [SMILES: COc1cncc(NCc2cc3nccnc3cc2Cl)c1N1CCNCC1] |
| 134 | 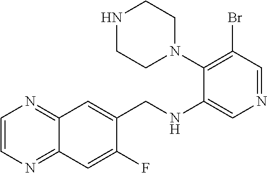 [SMILES: Fc1cc2nccnc2cc1CNc1cncc(Br)c1N1CCNCC1] |
| 135 | 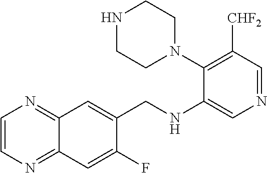 [SMILES: Fc1cc2nccnc2cc1CNc1cncc(C(F)F)c1N1CCNCC1] |
| 136 | 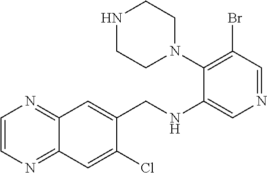 [SMILES: Clc1cc2nccnc2cc1CNc1cncc(Br)c1N1CCNCC1] |
| 137 | 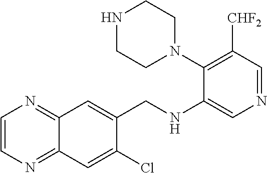 [SMILES: FC(F)c1cncc(NCc2cc3nccnc3cc2Cl)c1N1CCNCC1] |
| 138 | 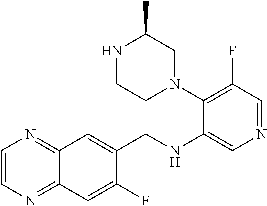 [SMILES: C[C@H]1CN(c2c(F)cncc2NCc2cc3nccnc3cc2F)CCN1] |
| 139 | 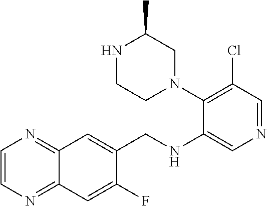 [SMILES: C[C@H]1CN(c2c(Cl)cncc2NCc2cc3nccnc3cc2F)CCN1] |
| 140 | 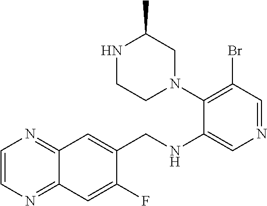 [SMILES: C[C@H]1CN(c2c(Br)cncc2NCc2cc3nccnc3cc2F)CCN1] |
| 141 | 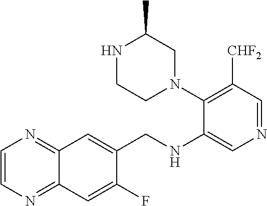 [SMILES: C[C@H]1CN(c2c(NCc3cc4nccnc4cc3F)cncc2C(F)F)CCN1] |
| 142 | 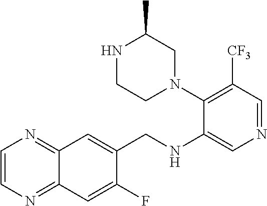 [SMILES: C[C@H]1CN(c2c(NCc3cc4nccnc4cc3F)cncc2C(F)(F)F)CCN1] |
| 143 | 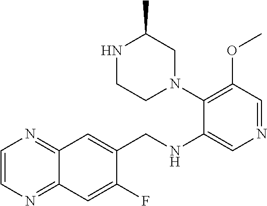 [SMILES: COc1cncc(NCc2cc3nccnc3cc2F)c1N1CCN[C@@H](C)C1] |
| 144 | 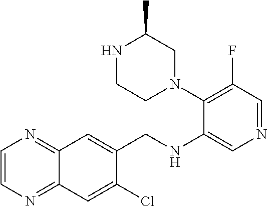 [SMILES: C[C@H]1CN(c2c(F)cncc2NCc2cc3nccnc3cc2Cl)CCN1] |
| 145 | 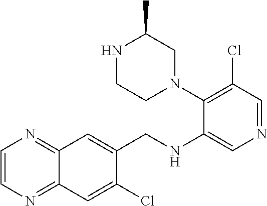 [SMILES: C[C@H]1CN(c2c(Cl)cncc2NCc2cc3nccnc3cc2Cl)CCN1] |
| 146 | 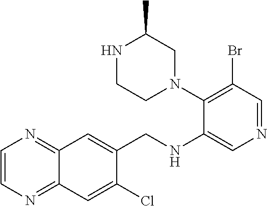 [SMILES: C[C@H]1CN(c2c(Br)cncc2NCc2cc3nccnc3cc2Cl)CCN1] |
| 147 | 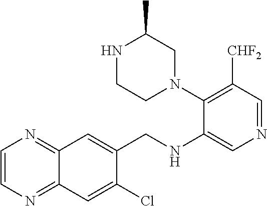 [SMILES: C[C@H]1CN(c2c(NCc3cc4nccnc4cc3Cl)cncc2C(F)F)CCN1] |
| 148 | 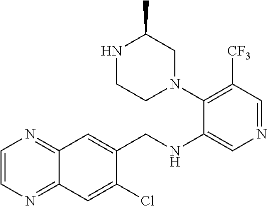 [SMILES: C[C@H]1CN(c2c(NCc3cc4nccnc4cc3Cl)cncc2C(F)(F)F)CCN1] |
| 149 | 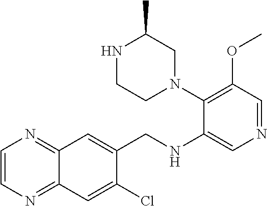 [SMILES: COc1cncc(NCc2cc3nccnc3cc2Cl)c1N1CCN[C@@H](C)C1] |
| 150 | 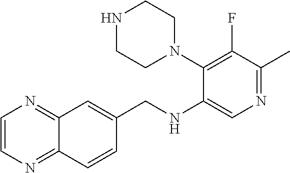 [SMILES: Cc1ncc(NCc2ccc3nccnc3c2)c(N2CCNCC2)c1F] |
| 151 | 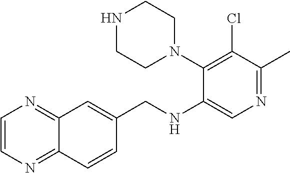 [SMILES: Cc1ncc(NCc2ccc3nccnc3c2)c(N2CCNCC2)c1Cl] |
| 152 | 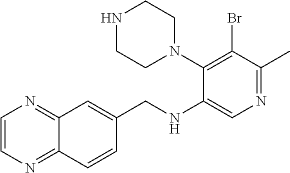 [SMILES: Cc1ncc(NCc2ccc3nccnc3c2)c(N2CCNCC2)c1Br] |
| 153 | 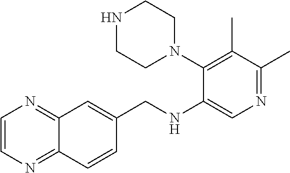 [SMILES: Cc1ncc(NCc2ccc3nccnc3c2)c(N2CCNCC2)c1C] |
| 154 | 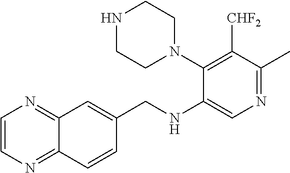 [SMILES: Cc1ncc(NCc2ccc3nccnc3c2)c(N2CCNCC2)c1C(F)F] |
| 155 | 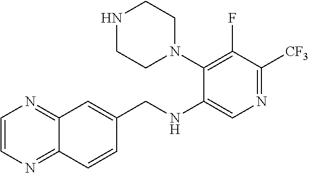 [SMILES: Fc1c(C(F)(F)F)ncc(NCc2ccc3nccnc3c2)c1N1CCNCC1] |
| 156 | 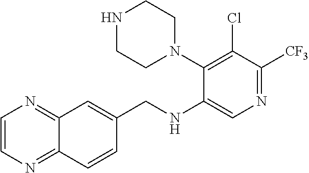 [SMILES: FC(F)(F)c1ncc(NCc2ccc3nccnc3c2)c(N2CCNCC2)c1Cl] |
| 157 | 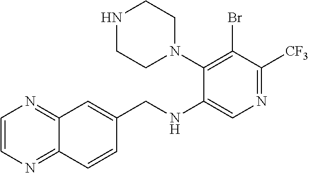 [SMILES: FC(F)(F)c1ncc(NCc2ccc3nccnc3c2)c(N2CCNCC2)c1Br] |
| 158 | 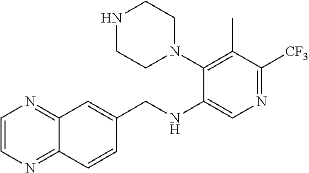 [SMILES: Cc1c(C(F)(F)F)ncc(NCc2ccc3nccnc3c2)c1N1CCNCC1] |
| 159 | 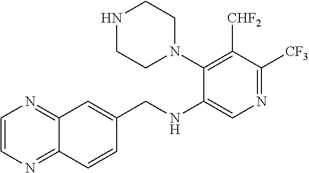 [SMILES: FC(F)c1c(C(F)(F)F)ncc(NCc2ccc3nccnc3c2)c1N1CCNCC1] |
| 160 | 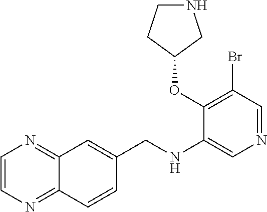 [SMILES: Brc1cncc(NCc2ccc3nccnc3c2)c1O[C@@H]1CCNC1] |
| 161 | 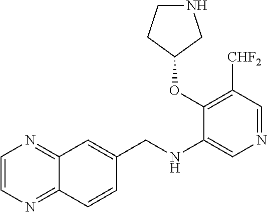 [SMILES: FC(F)c1cncc(NCc2ccc3nccnc3c2)c1O[C@@H]1CCNC1] |
| 162 | 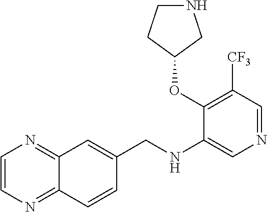 [SMILES: FC(F)(F)c1cncc(NCc2ccc3nccnc3c2)c1O[C@@H]1CCNC1] |
| 163 | 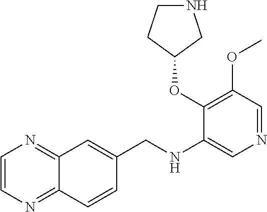 [SMILES: COc1cncc(NCc2ccc3nccnc3c2)c1O[C@@H]1CCNC1] |
| 164 | 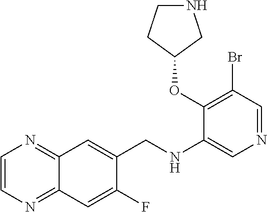 [SMILES: Fc1cc2nccnc2cc1CNc1cncc(Br)c1O[C@@H]1CCNC1] |
| 165 | 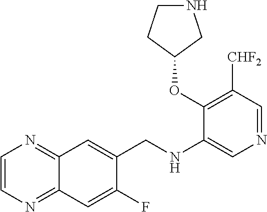 [SMILES: Fc1cc2nccnc2cc1CNc1cncc(C(F)F)c1O[C@@H]1CCNC1] |
| 166 | 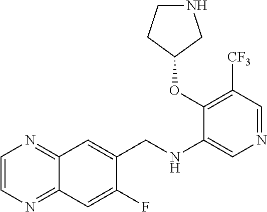 [SMILES: Fc1cc2nccnc2cc1CNc1cncc(C(F)(F)F)c1O[C@@H]1CCNC1] |
| 167 | 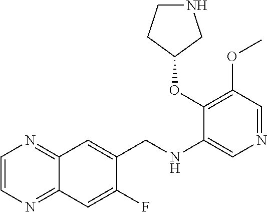 [SMILES: COc1cncc(NCc2cc3nccnc3cc2F)c1O[C@@H]1CCNC1] |
| 168 | 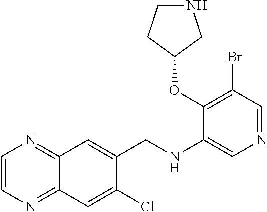 [SMILES: Clc1cc2nccnc2cc1CNc1cncc(Br)c1O[C@@H]1CCNC1] |
| 169 | 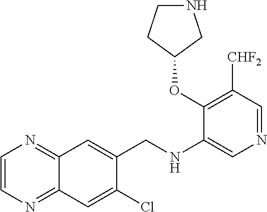 [SMILES: FC(F)c1cncc(NCc2cc3nccnc3cc2Cl)c1O[C@@H]1CCNC1] |
| 170 | 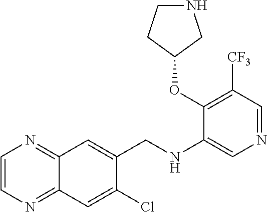 [SMILES: FC(F)(F)c1cncc(NCc2cc3nccnc3cc2Cl)c1O[C@@H]1CCNC1] |
| 171 | 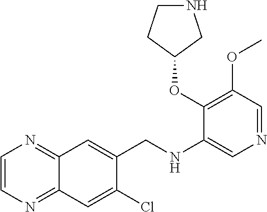 [SMILES: COc1cncc(NCc2cc3nccnc3cc2Cl)c1O[C@@H]1CCNC1] |
| 172 | 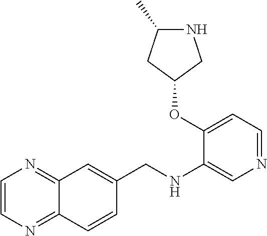 [SMILES: C[C@H]1C[C@@H](Oc2ccncc2NCc2ccc3nccnc3c2)CN1] |
| 173 | 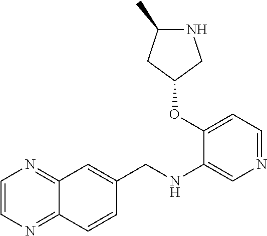 [SMILES: C[C@@H]1C[C@@H](Oc2ccncc2NCc2ccc3nccnc3c2)CN1] |
| 174 | 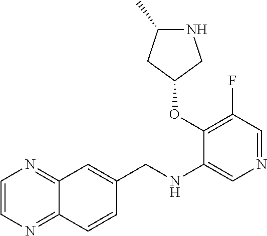 [SMILES: C[C@H]1C[C@@H](Oc2c(F)cncc2NCc2ccc3nccnc3c2)CN1] |
| 175 | 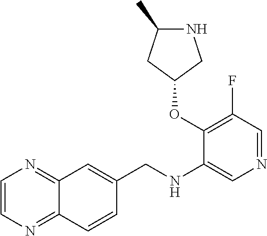 [SMILES: C[C@@H]1C[C@@H](Oc2c(F)cncc2NCc2ccc3nccnc3c2)CN1] |
| 176 | 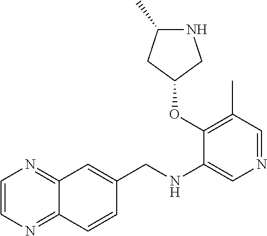 [SMILES: Cc1cncc(NCc2ccc3nccnc3c2)c1O[C@H]1CN[C@@H](C)C1] |
| 177 | 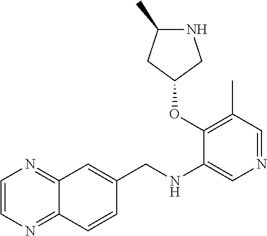 [SMILES: Cc1cncc(NCc2ccc3nccnc3c2)c1O[C@H]1CN[C@H](C)C1] |
| 178 | 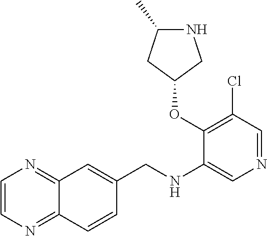 [SMILES: C[C@H]1C[C@@H](Oc2c(Cl)cncc2NCc2ccc3nccnc3c2)CN1] |
| 179 | 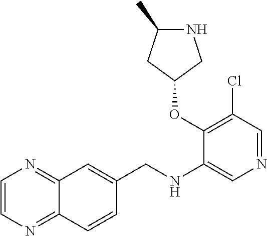 [SMILES: C[C@@H]1C[C@@H](Oc2c(Cl)cncc2NCc2ccc3nccnc3c2)CN1] |
| 180 | 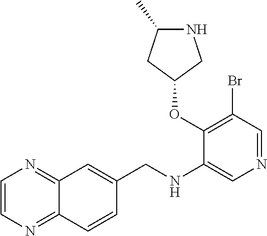 [SMILES: C[C@H]1C[C@@H](Oc2c(Br)cncc2NCc2ccc3nccnc3c2)CN1] |
| 181 | 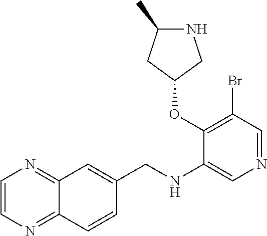 [SMILES: C[C@@H]1C[C@@H](Oc2c(Br)cncc2NCc2ccc3nccnc3c2)CN1] |
| 182 | 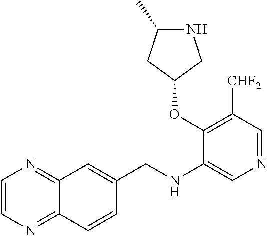 [SMILES: C[C@H]1C[C@@H](Oc2c(NCc3ccc4nccnc4c3)cncc2C(F)F)CN1] |
| 183 | 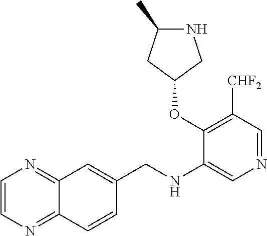 [SMILES: C[C@@H]1C[C@@H](Oc2c(NCc3ccc4nccnc4c3)cncc2C(F)F)CN1] |
| 184 | 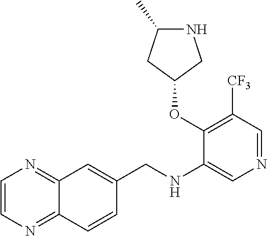 [SMILES: C[C@H]1C[C@@H](Oc2c(NCc3ccc4nccnc4c3)cncc2C(F)(F)F)CN1] |
| 185 | 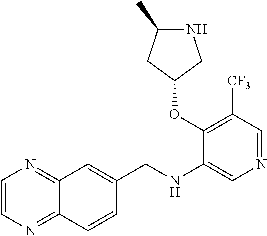 [SMILES: C[C@@H]1C[C@@H](Oc2c(NCc3ccc4nccnc4c3)cncc2C(F)(F)F)CN1] |

In embodiments, the aptamer disclosed herein binds to, or otherwise responds to the presence of one or more of the following compounds (or a pharmaceutically acceptable salt thereof):

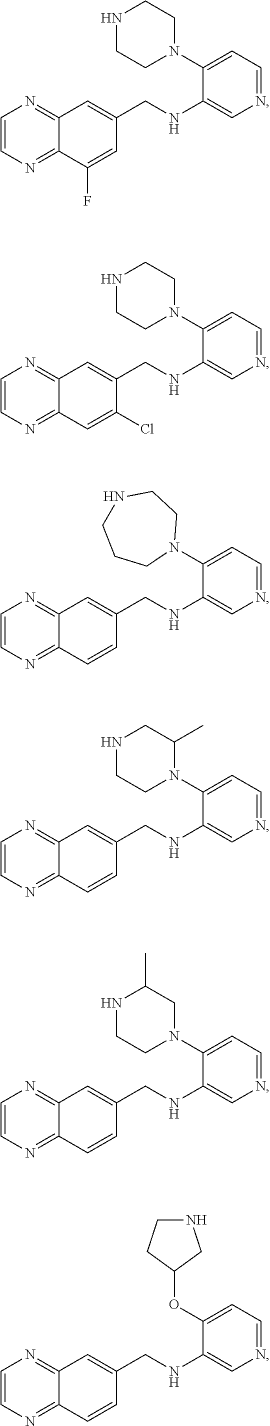 [SMILES: Fc1cc(CNc2cnccc2N2CCNCC2)cc2nccnc12 ; Clc1cc2nccnc2cc1CNc1cnccc1N1CCNCC1 ; c1cc(N2CCCNCC2)c(NCc2ccc3nccnc3c2)cn1 ; CC1CNCCN1c1ccncc1NCc1ccc2nccnc2c1 ; CC1CN(c2ccncc2NCc2ccc3nccnc3c2)CCN1 ; c1cc(OC2CCNC2)c(NCc2ccc3nccnc3c2)cn1]

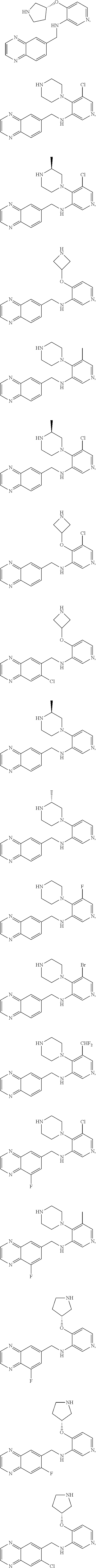

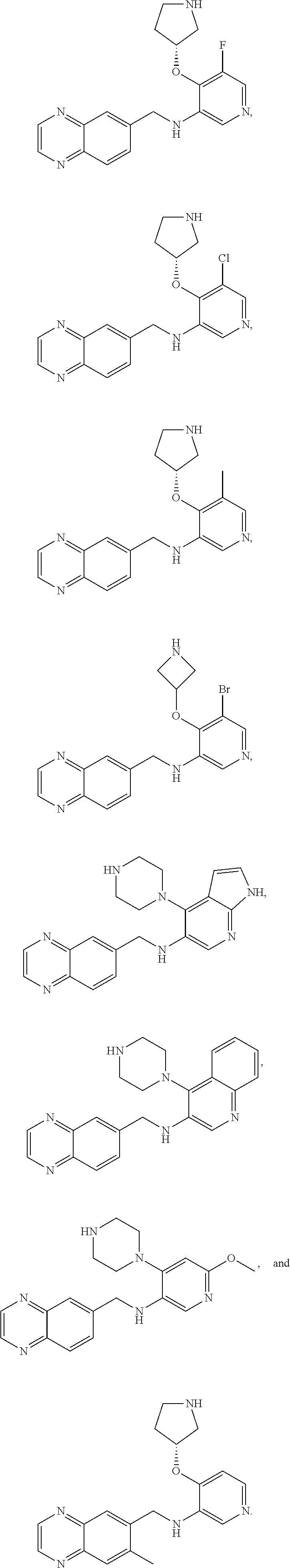

The term “alkyl” refers to the radical of saturated aliphatic groups, including straight-chain alkyl groups and branched-chain alkyl groups. In preferred embodiments, a straight chain or branched chain alkyl has 6 or fewer carbon atoms in its backbone (e.g., C₁-C₆ for straight chain, C₃-C₆ for branched chain). Alkyl groups include methyl, ethyl, propyl, isopropyl, n-butyl, iso-butyl, tert-butyl, pentyl, isopentyl, hexyl, and the like. The term “substituted alkyl” refers to an alkyl group which has from 1 to 4 substituents independently selected from halo, amino, amido, sulfonamido, OH, OCH₃, nitro and CN.

The term “cycloalkyl” refers to saturated, carbocyclic groups having from 3 to 6 carbons in the ring. Cycloalkyl groups include cyclopropyl, cyclobutyl, cyclopentyl and cyclohexyl.

The term “bicyclyl” refers to saturated carbocyclic groups having two joined ring systems, which may be fused or bridged. Bicyclic groups include bicycle[2.1.1]hexane, bicycle[2.2.1]heptane, decalin, and the like. The term “tricyclyl” refers to saturated carbocyclic groups having three joined ring systems, which may be fused and/or bridged. Tricyclic groups include adamantane and the like.

Carbocyclic refers to ring system that comprise only carbon atoms as ring atoms (i.e., the ring system does not have a heteroatom as a ring atom). Carbocyclic ring systems may be unsaturated, but preferred carbocyclic rings are not aromatic.

The term “alkenyl” refers to unsaturated aliphatic groups, including straight-chain alkenyl groups and branched-chain alkenyl groups, having at least one carbon-carbon double bond. In preferred embodiments, the alkenyl group has two to six carbon atoms (e.g., C₂-C₆ alkenyl).

As used herein, the term “halogen” or “halo” designates —F, —Cl, —Br or —I, and preferably —F, —Cl or —Br.

The terms “alkoxyl” or “alkoxy” as used herein refers to an alkyl group, as defined above, that is attached through an oxygen atom. Representative alkoxyl groups include methoxy, ethoxy, propyloxy, tert-butoxy and the like.

The terms “amine” and “amino” refer to both unsubstituted and substituted amines, e.g., a moiety that can be represented by the general formula:

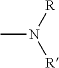

R and R′ are each independently selected from H and C₁-C₃ alkyl.

The terms “amido” refer to both unsubstituted and substituted amide substituents, e.g., a moiety that can be represented by the general formula:

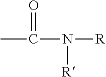

wherein R and R′ are each independently selected from H and C₁-C₃ alkyl.

The terms “sulfonamide” or “sulfonamido” refer to both unsubstituted and substituted sulfonamide substituents, e.g., a moiety that can be represented by the general formula:

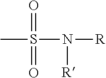

wherein R and R′ are each independently selected from H and C₁-C₃ alkyl.

The term “aryl” as used herein includes 5- and 6-membered single-ring aromatic groups that may include from zero to four heteroatoms, for example, benzene, pyrene, pyrrole, furan, thiophene, imidazole, oxazole, thiazole, triazole, pyrazole, pyridine, pyrazine, pyridazine and pyrimidine. Those aryl groups having heteroatoms in the ring structure may also be referred to as “aryl heterocycles” or “heteroaryl” groups. The term “aryl” also includes polycyclic ring systems having two or more cyclic rings in which two or more carbons are common to two adjoining rings (the rings are “fused rings”) wherein at least one of the rings is aromatic. Accordingly, aryl includes 8- to 10-membered fused bicyclic aromatic groups that may include from zero to five heteroatoms, in which one or both rings are aromatic, for example napthylene, quinolone, isoquinoline, benzo[b]thiophene, tetrahydronapthelene, and the like. Each aryl group may be unsubstituted or may be substituted with 1 to 5 substituents selected from halogen, hydroxyl, amino, cyano, amido, sulfonamide, nitro, —SH, C₁-C₆ alkyl, C₂-C₆ alkenyl, C₃-C₇ cycloalkyl, C₆-C₁₀ bicyclyl, C₁-C₆ haloalkyl, C₁-C₆ perhaloalkyl, —O—(C₁-C₆ alkyl), O—(C₃-C₇ cycloalkyl), —O—(C₁-C₆ haloalkyl), —O—(C₁-C₆ perhaloalkyl), aryl, —O-aryl, —(C₁-C₆ alkyl)-aryl, —O—(C₁-C₆ alkyl)-aryl, —S—(C₁-C₆ alkyl), —S—(C₃-C₇ cycloalkyl), —S—(C₁-C₆ haloalkyl), —S—(C₁-C₆ perhaloalkyl), —S-aryl, —S—(C₁-C₆ alkyl)-aryl, heteroaryl and hetercyclyl.

The term “heterocycle” of “heterocyclyl” refer to non-aromatic heterocycles having from 1 to 3 ring heteroatoms. Preferred heterocycles are 5- and 6-membered heterocyclic groups having from 1 to 3 heteroatoms selected from the group consisting of O, N and S.

The term “heteroatom” as used herein means an atom of any element other than carbon or hydrogen. Preferred heteroatoms are nitrogen, oxygen, and sulfur.

As used herein, the definition of each expression, e.g. alkyl, R¹, R², etc., when it occurs more than once in any structure, is intended to be independent of its definition elsewhere in the same structure.

It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc.

The aptamer ligands disclosed herein may exist in particular geometric or stereoisomeric forms well as mixtures thereof. Such geometric or stereoisomeric forms include, but not limited to, cis- and trans-isomers, R- and S-enantiomers, diastereomers, (D)-isomers, (L)-isomers, the racemic mixtures thereof, and other mixtures thereof. Additional asymmetric carbon atoms may be present in a substituent such as an alkyl group.

The compounds according to Formulas I to XXII may contain an acidic or basic functional group, and accordingly may be present in a salt form. Preferably, the salt form is a pharmaceutically acceptable salt. The term “pharmaceutically-acceptable salts” in this respect, refers to the relatively non-toxic, inorganic and organic acid and base addition salts of the compounds disclosed herein.

The compounds according to Formulas I to XXII may contain one or more basic functional group, such as amino or alkylamino, and are, thus, capable of forming pharmaceutically-acceptable salts with pharmaceutically-acceptable acids. These salts can be prepared in situ in the administration vehicle or the dosage form manufacturing process, or by separately reacting a purified compound disclosed herein in its free base form with a suitable organic or inorganic acid, and isolating the salt thus formed during subsequent purification. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like (see, e.g., Berge et al. (1977) “Pharmaceutical Salts”, J. Pharm. Sci. 66:1-19).

The pharmaceutically acceptable salts of the subject compounds include the conventional nontoxic salts or quaternary ammonium salts of the compounds, e.g., from non-toxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

In other cases, the compounds according to Formulas I to XXII may contain one or more acidic functional groups and, thus, are capable of forming pharmaceutically-acceptable salts with pharmaceutically-acceptable bases. These salts can likewise be prepared in situ in the administration vehicle or the dosage form manufacturing process, or by separately reacting the purified compound in its free acid form with a suitable base, such as the hydroxide, carbonate or bicarbonate of a pharmaceutically-acceptable metal cation, with ammonia, or with a pharmaceutically-acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like (see, e.g., Berge et al., supra).

In embodiments, the aptamers provided herein bind to, or otherwise respond to the presence of, one or more compounds of Formula I-XXII provided herein, and/or bind to, or otherwise respond to, a metabolite analog or derivative of a compound of Formula I-XXII.

The specificity of the binding of an aptamer to its ligand can be defined in terms of the comparative dissociation constants (K_{d}) of the aptamer for its ligand as compared to the dissociation constant of the aptamer for unrelated molecules. Thus, the ligand may be considered to be a molecule that binds to the aptamer with greater affinity than to unrelated material. Typically, the K_{d} for the aptamer with respect to its ligand will be at least about 10-fold less than the K_{d} for the aptamer with unrelated molecules. In other embodiments, the K_{d} will be at least about 20-fold less, at least about 50-fold less, at least about 100-fold less, and at least about 200-fold less, at least about 500-fold less, at least about 1000-fold less, or at least about 10,000-fold less than the K_{d} for the aptamer with unrelated molecules.

### Aptamers for the Regulation of Gene Expression

In some embodiments, the aptamers contemplated by the disclosure are used for the regulation of gene expression. Regulation of the expression of a target gene (e.g., a therapeutic transgene) is advantageous in a variety of situations. In the context of the therapeutic expression of genes, for example, techniques that enable regulated expression of transgenes in response to the presence of a small molecule can enhance safety and efficacy by allowing for the regulation of the level of target gene expression and its timing. In a research setting, the regulation of gene expression allows a systematic investigation of different experimental conditions.

In embodiments, the sequence encoding the aptamer is part of a gene regulation cassette that provides the ability to regulate the expression level of a target gene in response to the presence or absence of a small molecule described herein. In embodiments, the gene regulation cassette further comprises a target gene. As used herein, “target gene” refers to a transgene that is expressed in response to the presence or absence of the small molecule ligands disclosed herein due to the small molecule binding to the aptamers disclosed herein. In embodiments, the target gene comprises the coding sequence for a protein (e.g., a therapeutic protein), a miRNA, or a siRNA. The target gene is heterologous to the aptamer used for the regulation of target gene expression, is heterologous to the polynucleotide cassette used for the regulation of target gene and/or is heterologous to a portion of the polynucleotide cassette used for the regulation of target gene.

When used to regulate the expression of a target gene in response to the presence/absence of a ligand, the aptamers described herein can be part of a polynucleotide cassette that encodes the aptamer as part of a riboswitch. The terms “gene regulation cassette”, “regulatory cassette”, or “polynucleotide cassette” are used interchangeably herein.

In embodiments, the presence of a small molecule that binds to an aptamer disclosed herein leads to an increase in expression of a target gene as compared to the expression of the target gene in absence of the small molecule. In such an embodiment, the aptamer constitutes an “on” switch. In embodiments, the expression of the target gene is increased by at least 3-fold, by at least 5-fold, by at least 10-fold, by at least 15-fold, by at least 20-fold, by at least 25-fold, by at least 30-fold, by at least 40-fold, by at least 50-fold, by at least 100-fold, by at least 1000-fold, or by at least 10,000-fold in presence of the small molecule that binds to an aptamer disclosed herein as compared to in absence of the small molecule. In embodiments, the expression of the target gene is increased by between 2-fold and 10-fold, between 5-fold and 10-fold, between 5-fold and 15-fold, between 5-fold and 20-fold, between 5-fold and 25-fold, between 5-fold and 30-fold, between 10-fold and 20-fold, between 10-fold and 30-fold, between 10-fold and 40-fold, between 10-fold and 50-fold, between 10-fold and 100-fold, between 10-fold and 500-fold, between 10-fold and 1,000-fold, between 50-fold and 100-fold, between 50-fold and 500-fold, between 50-fold and 100-fold, between 50-fold and 1,000-fold, between 100-fold and 1,000-fold, or between 100-fold and 10,000-fold in presence of the small molecule that binds to an aptamer disclosed herein as compared to in absence of the small molecule.

In embodiments, the presence of a small molecule that binds to an aptamer disclosed herein leads to a decrease in expression of a target gene as compared to the expression of the target gene in the absence of the small molecule. In such embodiments, the aptamer constitutes an “off” switch. In embodiments, the expression of the target gene is decreased by at least 3-fold, by at least 5-fold, by at least 10-fold, by at least 15-fold, by at least 20-fold, by at least 25-fold, by at least 30-fold, by at least 40-fold, by at least 50-fold, by at least 100-fold, by at least 1000-fold, or by at least 10,000-fold in presence of the small molecule that binds to an aptamer disclosed herein as compared to in absence of the small molecule. In one embodiment, the expression of the target gene is decreased by between 2-fold and 10-fold, between 5-fold and 10-fold, between 5-fold and 15-fold, between 5-fold and 20-fold, between 5-fold and 25-fold, between 5-fold and 30-fold, between 10-fold and 20-fold, between 10-fold and 30-fold, between 10-fold and 40-fold, between 10-fold and 50-fold, between 10-fold and 100-fold, between 10-fold and 500-fold, between 10-fold and 1,000-fold, between 50-fold and 100-fold, between 50-fold and 500-fold, between 50-fold and 100-fold, between 50-fold and 1,000-fold, between 100-fold and 1,000-fold, or between 100-fold and 10,000-fold in presence of the small molecule that binds to an aptamer disclosed herein as compared to in absence of the small molecule.

In embodiments, the aptamer is part of a riboswitch. Riboswitches are regulatory segments of an RNA polynucleotide that regulate the stability of the RNA polynucleotide and/or regulate the production of a protein from the RNA polynucleotide in response to the presence or absence of aptamer-specific ligand molecules. In embodiments, the riboswitch comprises a sensor region (e.g., the aptamer region) and an effector region that together are responsible for sensing the presence of a ligand (e.g., a small molecule) and causing an effect that leads to increased or decreased expression of the target gene. The riboswitches described herein are recombinant, utilizing polynucleotides from two or more sources. In embodiments, the sensor and effector regions are joined by a polynucleotide linker. In embodiments, the polynucleotide linker forms a RNA stem or paired region (i.e., a region of the RNA polynucleotide that is double-stranded). In embodiments, the paired region linking the aptamer to the effector region comprises all, or some of an aptamer stem (e.g., for example all, or some of the aptamer P1 stem).

Riboswitches comprising aptamer sequences may be used, for example, to control the formation of rho-independent transcription termination hairpins leading to premature transcription termination. Riboswitches comprising aptamer sequences may also induce structural changes in the RNA, leading to sequestration for the ribosome binding site and inhibition of translation. Alternative riboswitch structures comprising the aptamer sequences disclosed herein can further affect the splicing of mRNA in response to the presence of the small molecule ligand.

### Alternative Splicing Riboswitch

In one embodiment, the aptamers described herein are encoded as part of a gene regulation cassette for the regulation of a target gene by aptamer/ligand mediated alternative splicing of the resulting RNA (e.g., pre-mRNA). In this context, the gene regulation cassette comprises a riboswitch comprising a sensor region (e.g., the aptamers described herein) and an effector region that together are responsible for sensing the presence of a small molecule ligand and altering splicing to an alternative exon. Splicing refers to the process by which an intronic sequence is removed from the nascent pre-messenger RNA (pre-mRNA) and the exons are joined together to form the mRNA. Splice sites are junctions between exons and introns, and are defined by different consensus sequences at the 5′ and 3′ ends of the intron (i.e., the splice donor and splice acceptor sites, respectively). Splicing is carried out by a large multi-component structure called the spliceosome, which is a collection of small nuclear ribonucleoproteins (snRNPs) and a diverse array of auxiliary proteins. By recognizing various cis regulatory sequences, the spliceosome defines exon/intron boundaries, removes intronic sequences, and splices together the exons into a final message (e.g., the mRNA). In the case of alternative splicing, certain exons can be included or excluded to vary the final coding message thereby changing the resulting expressed protein.

In one embodiment, the regulation of target gene expression is achieved by using any of the DNA constructs disclosed in WO2016/126747, which is hereby incorporated by reference in its entirety. In embodiments of the present disclosure, the riboswitches and polynucleotide cassettes disclosed in WO2016/126747 comprise an aptamer encoding sequence described herein in place of the aptamer sequence disclosed in WO2016/126747.

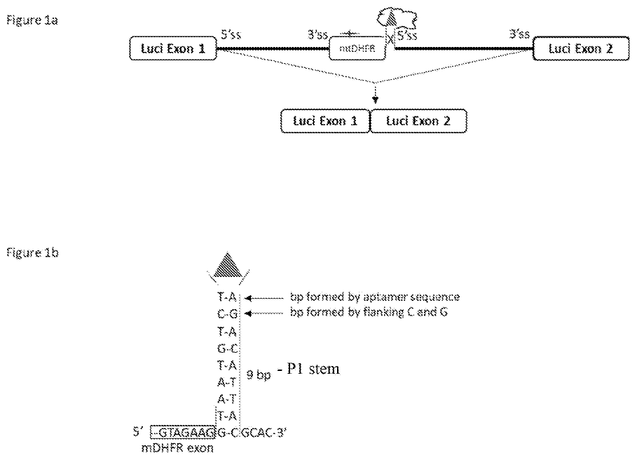

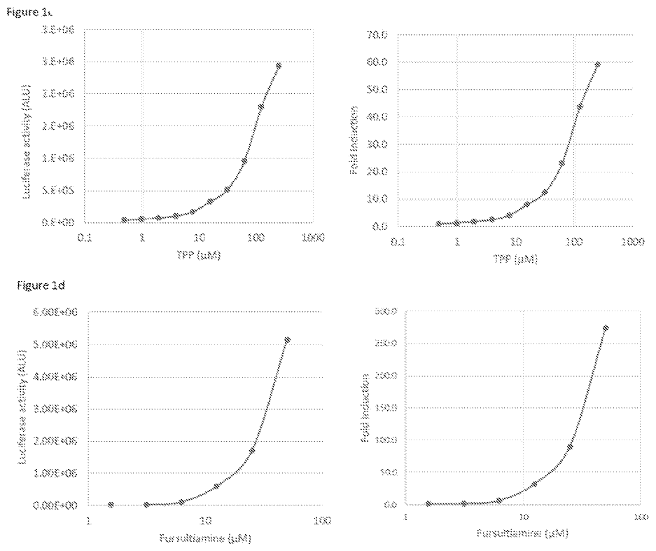

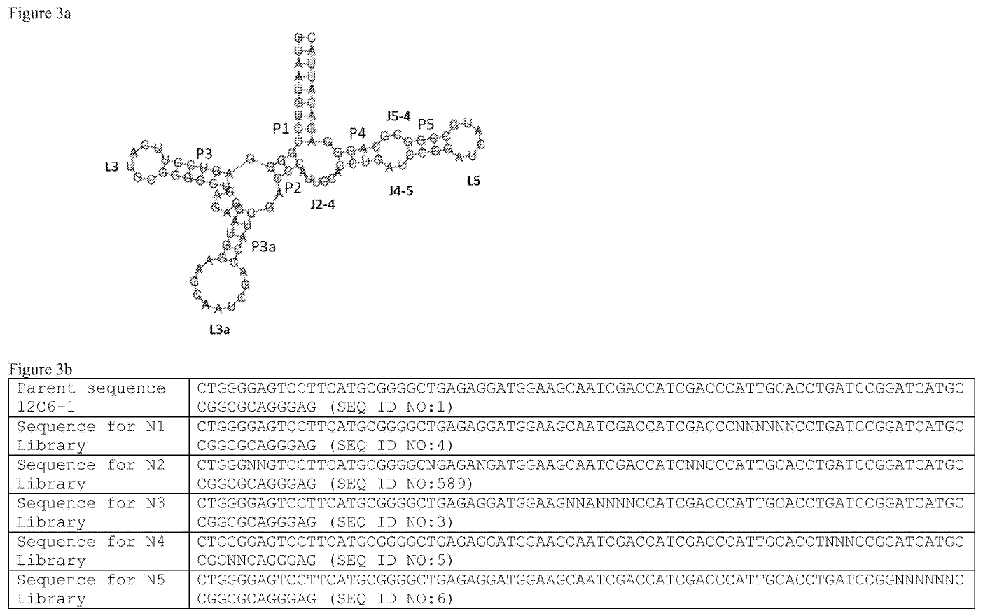

In one embodiment, the polynucleotide cassette comprises (a) a riboswitch and (b) an alternatively-spliced exon, flanked by a 5′ intron and a 3′ intron, wherein the riboswitch comprises (i) an effector region comprising a stem forming sequence that includes the 5′ splice site sequence of the 3′ intron (and sequence complementary thereto), and (ii) an aptamer disclosed herein. In embodiments, the effector region is a stem forming region that forms the P1 stem of the aptamer (see, e.g., FIG. 1b and FIG. 3a where the 12C6-1 aptamer sequence is flanked by additional sequence that forms the P1 stem of the aptamer and contains the splice site sequence and sequence complementary thereto). Thus, in embodiments, the effector stem is, or comprises, the P1 stem of the aptamers disclosed herein. In other words, the effector stem comprises a first sequence that is linked to the 5′ end of the aptamers disclosed herein and a second sequence that is linked to the 3′ end of the aptamers disclosed herein, wherein the first or second sequence includes the 5′ splice site sequence of the 3′ intron and the other includes sequence complementary to the 5′ splice site sequence of the 3′ intron. In embodiments, the effector region comprises the intronic 5′ splice site (“5′ ss”) sequence of the intron that is immediately 3′ of the alternative exon, as well as the sequence complimentary to the 5′ ss sequence of the 3′ intron.

5′ splice site sequences are well known in the art. There is some variability among different 5′ splice site sequences, and this variability is also well understood in the art. For example, Shapiro and Senapathy (Shapiro M B, Senapathy P. RNA splice junctions of different classes of eukaryotes: sequence statistics and functional implications in gene expression. Nucleic Acids Res. 1987 Sep. 11; 15(17):7155-74 or Zhang M Q. Statistical features of human exons and their flanking regions. Hum Mol Genet. 1998 May; 7(5):919-32, which is incorporated in its entirety herein) describe for a variety of eukaryotes which positions of the splice site sequence have some variability, and which positions are fixed. Likewise, Zhang (Zhang M Q. Statistical features of human exons and their flanking regions. Hum Mol Genet. 1998 May; 7(5):919-32, which is incorporated in its entirety herein) also shows which positions of the splice site sequence may have some variability, and which positions are fixed. As such, a person skilled in the art can easily recognize a splice site sequence based on the known consensus sequence and based on its location relative to the exon/intron boundary. Exemplary splice site sequences include, but are not limited to: A G G∥G T G A G T; A A A∥G T A A G C; G C A∥G T A A G T; G A G∥G T G T G G; A/C A G∥G T A/G A G T; N A G∥G T A/G A G T; N A G∥G T A A G T; A/C A/T G∥G T A N G T; and N A G/A∥G T A A G T (where ∥ denotes the exon/intron boundary and N represents A, G, C, or T).

When the aptamer binds its ligand, the effector region forms a stem and thus prevents splicing to the splice donor site at the 3′ end of the alternative exon. Under certain conditions (for example, when the aptamer is not bound to its ligand), the effector region is in a context that provides access to the splice donor site at the 3′ end of the alternative exon, leading to inclusion of the alternative exon in the target gene mRNA. In some embodiments, the polynucleotide cassette is placed in the target gene to regulate expression of the target gene in response to a ligand. In one embodiment, the alternatively-spliced exon comprises a stop codon that is in-frame with the target gene when the alternatively-spliced exon is spliced into the target gene mRNA.

In one embodiment, the gene regulation cassette comprises the sequence of SEQ ID NO:676, wherein —X— represents an aptamer encoding sequence disclosed herein. Lower case letters indicate paired stem sequence linking the aptamer to the remainder of the riboswitch. In one embodiment, the alternative exon (underlined in SEQ ID NO:676, below) is replaced with another alternative exon sequence.

SEQ ID NO: 676:
GTGAGTCTATGGGACCCTTGATGTTTTCTTTCCCCTTCTTTTCTATGGT
TAAGTTCATGTCATAGGAAGGGGAGAAGTAACAGGGTACACATATTGAC
CAAATCAGGGTAATTTTGCATTTGTAATTTTAAAAAATGCTTTCTTCTT
TTAATATACTTTTTTGTTTATCTTATTTCTAATACTTTCCCTAATCTCT
TTCTTTCAGGGCAATAATGATACAATGTATCATGCCGAGTAACGCTGTT
TCTCTAACTTGTAGGAATGAATTCAGATATTTCCAGAGAATGAAAAAAA
AATCTTCAGTAGAAGgtaatgt-X-acattacGCACCATTCTAAAGAAT
AACAGTGATAATTTCTGGGTTAAGGCAATAGCAATATTTCTGCATATAA
ATATTTCTGCATATAAATTGTAACTGATGTAAGAGGTTTCATATTGCTA
ATAGCAGCTACAATCCAGCTACCATTCTGCTTTTATTTTATGGTTGGGA
TAAGGCTGGATTATTCTGAGTCCAAGCTAGGCCCTTTTGCTAATCATGT
TCATACCTCTTATCTTCCTCCCACAG.

The alternative exon is flanked by 5′ and 3′ intronic sequences. The 5′ and 3′ intronic sequences that can be used in the gene regulation cassettes disclosed herein can be any sequence that can be spliced out of the target gene creating either the target gene mRNA or the target gene comprising the alternative exon in the mRNA, depending upon the presence or absence of a ligand that binds the aptamer. The 5′ and 3′ intronic sequences each have the sequences necessary for splicing to occur, i.e., splice donor, splice acceptor and branch point sequences. In one embodiment, the 5′ and 3′ intronic sequences of the gene regulation cassette are derived from one or more naturally occurring introns or portions thereof. In one embodiment, the 5′ and 3′ intronic sequences are derived from a truncated human beta-globin intron 2 (IVS2A), from intron 2 of the human 03-globin gene, from the SV40 mRNA intron (used in pCMV-LacZ vector from Clontech Laboratories, Inc.), from intron 6 of human triose phosphate isomerase (TPI) gene (Nott Ajit, et al. RNA. 2003, 9:6070617), from an intron from human factor IX (Sumiko Kurachi, et al. J. Bio. Chem. 1995, 270(10), 5276), from the target gene's own endogenous intron, or from any genomic fragment or synthetic introns (Yi Lai, et al. Hum Gene Ther. 2006:17(10): 1036) that contain elements that are sufficient for regulated splicing (Thomas A. Cooper, Methods 2005 (37):331).

In one embodiment, the alternative exon and riboswitch are engineered to be in an endogenous intron of a target gene. That is, the intron (or a substantially similar intronic sequence) naturally occurs at that position of the target gene. In this case, the intronic sequence immediately upstream of the alternative exon is referred to as the 5′ intron or 5′ intronic sequence, and the intronic sequence immediately downstream of the alternative exon is referred to as the 3′ intron or 3′ intronic sequence. In this case, the endogenous intron is modified to contain a splice acceptor sequence and splice donor sequence flanking the 5′ and 3′ ends of the alternative exon. In one embodiment, the 5′ and/or 3′ introns are exogenous to the target gene.

The splice donor and splice acceptor sites in the alternative splicing gene regulation cassette can be modified to be strengthened or weakened. That is, the splice sites can be modified to be closer to the consensus for a splice donor or acceptor by standard cloning methods, site directed mutagenesis, and the like. Splice sites that are more similar to the splice consensus tend to promote splicing and are thus strengthened. Splice sites that are less similar to the splice consensus tend to hinder splicing and are thus weakened. The consensus for the splice donor of the most common class of introns (U2) is A/C A G∥G T A/G A G T (where ∥ denotes the exon/intron boundary). The consensus for the splice acceptor is C A G∥G (where ∥ denotes the exon/intron boundary). The frequency of particular nucleotides at the splice donor and acceptor sites are described in the art (see, e.g., Zhang, M. Q., Hum Mol Genet. 1988. 7(5):919-932). The strength of 5′ and 3′ splice sites can be adjusted to modulate splicing of the alternative exon.

Additional modifications to 5′ and 3′ introns present in the alternative splicing gene regulation cassette that can be made to modulate splicing include modifying, deleting, and/or adding intronic splicing enhancer elements, intronic splicing suppressor elements and or splice sites, and/or modifying the branch site sequence.

In one embodiment, the 5′ intron has been modified to contain a stop codon that will be in frame with the target gene. The 5′ and 3′ intronic sequences can also be modified to remove cryptic slice sites, which can be identified with publicly available software (see, e.g., Kapustin, Y. et al. Nucl. Acids Res. 2011. 1-8).

The lengths of the 5′ and 3′ intronic sequences can be adjusted in order to, for example, meet the size requirements for viral expression constructs. In one embodiment, the 5′ and/or 3′ intronic sequences are about 50 to about 300 nucleotides in length. In one embodiment, the 5′ and/or 3′ intronic sequences are about 125 to about 240 nucleotides in length.

The stem portion of the effector region should be of a sufficient length (and GC content) to substantially prevent alternative splicing of the alternative exon upon ligand binding the aptamer, while also allowing access to the splice site when the ligand is not present in sufficient quantities. In embodiments, the stem portion of the effector region comprises a stem sequence in addition to the 5′ splice site sequence of the 3′ intron and its complementary sequence of the 5′ splice site sequence. In embodiments, this additional stem sequence comprises a sequence from the aptamer stem. The length and sequence of the stem portion can be modified using known techniques in order to identify stems that allow acceptable background expression of the target gene when no ligand is present and acceptable expression levels of the target gene when the ligand is present. In one embodiment, the effector region stem of the riboswitch is about 7 to about 20 base pairs in length. In one embodiment, the effector region stem is 8 to 11 base pairs in length. In addition to the length of the stem, the GC base pair content of the stem can be altered to modify the stability of the stem.

In one embodiment, the alternative exon that is part of the alternative splicing gene regulation cassettes disclosed herein is a polynucleotide sequence capable of being transcribed to a pre-mRNA and alternatively spliced into the mRNA of the target gene. In one embodiment, the alternative exon contains at least one sequence that inhibits translation such that when the alternative exon is included in the target gene mRNA, expression of the target gene from that mRNA is prevented or reduced. In a preferred embodiment, the alternative exon contains a stop codon (TGA, TAA, TAG) that is in frame with the target gene when the alternative exon is included in the target gene mRNA by splicing. In embodiments, the alternative exon comprises, in addition to a stop codon, or as an alternative to a stop codon, another sequence that reduces or substantially prevents translation when the alternative exon is incorporated by splicing into the target gene mRNA including, e.g., a microRNA binding site, which leads to degradation of the mRNA. In one embodiment, the alternative exon comprises a miRNA binding sequence that results in degradation of the mRNA. In one embodiment, the alternative exon encodes a polypeptide sequence which reduces the stability of the protein containing this polypeptide sequence. In one embodiment, the alternative exon encodes a polypeptide sequence which directs the protein containing this polypeptide sequence for degradation.

The basal or background level of splicing of the alternative exon can be optimized by altering exon splice enhancer (ESE) sequences and exon splice suppressor (ESS) sequences and/or by introducing ESE or ESS sequences into the alternative exon. Such changes to the sequence of the alternative exon can be accomplished using methods known in the art, including, but not limited to site directed mutagenesis. Alternatively, oligonucleotides of a desired sequence (e.g., comprising all or part of the alternative exon) can be obtained from commercial sources and cloned into the gene regulation cassette. Identification of ESS and ESE sequences can be accomplished by methods known in the art, including, for example using ESEfinder 3.0 (Cartegni, L. et al. ESEfinder: a web resource to identify exonic splicing enhancers. Nucleic Acid Research, 2003, 31(13): 3568-3571) and/or other available resources.

In one embodiment, the alternative exon is a naturally-occurring exon. In another embodiment, the alternative exon is derived from all or part of a known exon. In this context, “derived” refers to the alternative exon containing sequence that is substantially homologous to a naturally occurring exon, or a portion thereof, but may contain various mutations, such a mutations generated by altering exon splice enhancer (ESE) sequences and exon splice suppressor (ESS) sequences and/or by introducing ESE or ESS sequences into the alternative exon. “Homology” and “homologous” as used herein refer to the percent of identity between two polynucleotide sequences or between two polypeptide sequences. The correspondence between one sequence to another can be determined by techniques known in the art. For example, homology can be determined by a direct comparison of two polypeptide molecules by aligning their sequences and using readily available computer programs. Alternatively, homology can be determined by hybridization of polynucleotides under conditions which form stable duplexes between homologous regions, followed by digestion with single-stranded-specific nuclease(s), and size determination of the digested fragments. Two polynucleotide or two polypeptide sequences are “substantially homologous” to each other when, after optimally aligned with appropriate insertions or deletions, at least about 80%, at least about 85%, at least about 90%, and at least about 95% of the nucleotides or amino acids, respectively, match over a defined length of the molecules, as determined using the methods above.

In one embodiment, the alternative exon is exogenous to the target gene, although it may be derived from a sequence originating from the organism where the target gene will be expressed. As used herein, “exogenous” means derived from a genotypically distinct entity from that of the rest of the entity to which it is compared or into which it is introduced or incorporated. For example, a polynucleotide introduced by genetic engineering techniques into a different cell type is a heterologous polynucleotide (and, when expressed, can encode a heterologous polypeptide). In one embodiment, the alternatively-spliced exon is derived from exon 2 of the human dihydrofolate reductase gene (DHFR), mutant human Wilms tumor 1 exon 5, mouse calcium/calmodulin-dependent protein kinase II delta exon 16, or SIRT1 exon 6. In embodiments, the alternatively-spliced exon is, or comprises, the modified DHFR exon 2 in SEQ ID NO:677.

(GAATGAATTCAGATATTTCCAGAGAATGAAAAAAAAATCTTCAGTAGAAG). In embodiments, the alternatively-spliced exon is, or comprises, the modified DHFR exon 2 in

SEQ ID NO: 678
(GAATGAATTCAGATATTTCCAGAGAATGAAAA
AAAATCTTCAGTAGAAG).

### Aptamer-Mediated Cleavage by Self-Cleaving Ribozymes

In one embodiment, the aptamer-mediated expression of the target gene is regulated by an aptamer-mediated modulation of small endonucleolytic ribozymes. A ribozyme is an RNA enzyme that catalyzes a chemical reaction. In the nucleic acids and methods disclosed herein, a ribozyme may be any small endonucleolytic ribozyme that will self-cleave in the target cell type including, but not limited to a hammerhead, hairpin, the hepatitis delta virus, the Varkud satellite, twister, twister sister, pistol or hatchet ribozyme. Accordingly, in one embodiment, provided is a riboswitch, and a gene expression cassette comprising the riboswitch that contains a ribozyme linked to an aptamer disclosed herein. WO2017/136608, which is incorporated in its entirety by reference herein, describes such riboswitches that activate ribozyme self-cleavage in the presence of aptamer ligand (“off” switch) or riboswitches that inhibit ribozyme self-cleavage in the presence of aptamer (“on” switch).

In an “off” switch scenario, aptamer/ligand binding increases the ribonuclease function of the ribozyme, leading to cleavage of the target gene RNA that contains the polynucleotide cassette, thereby reducing target gene expression. Examples of such an off switch include a polynucleotide cassette for the regulation of the expression of a target gene comprising a riboswitch that comprises a twister ribozyme linked by a stem to an aptamer, wherein the stem linking the twister ribozyme to the aptamer attaches to the ribozyme at the location of the P3 stem of the twister ribozyme and wherein the target gene is linked to the P1 stem of the twister ribozyme (see, e.g. FIG. 1a, 1b, or 3a of WO2017/136608 and the associated text, incorporated herein by reference).

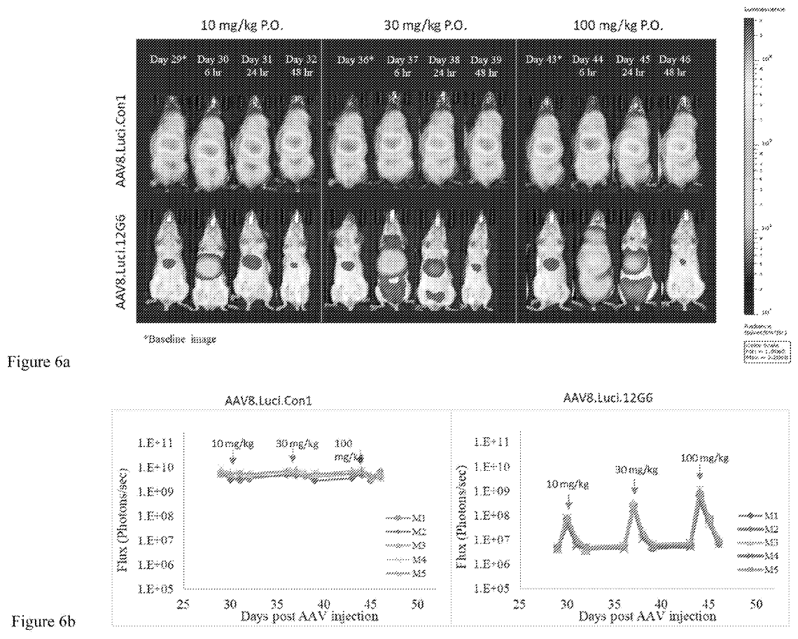

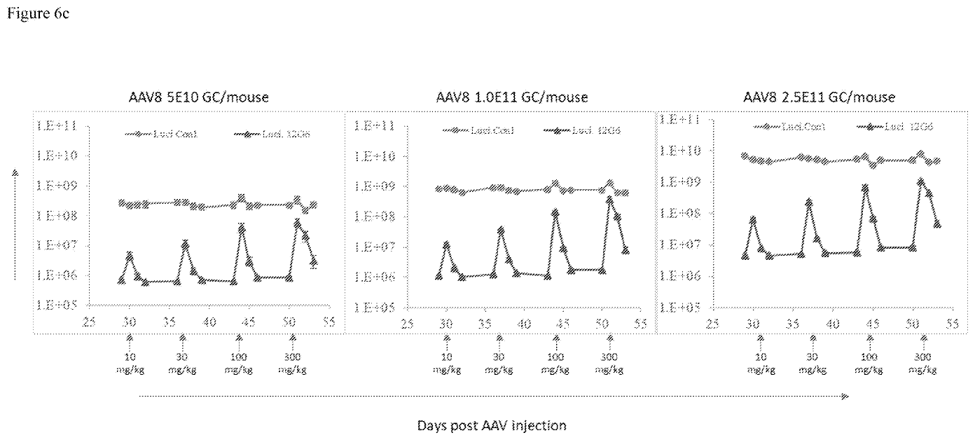

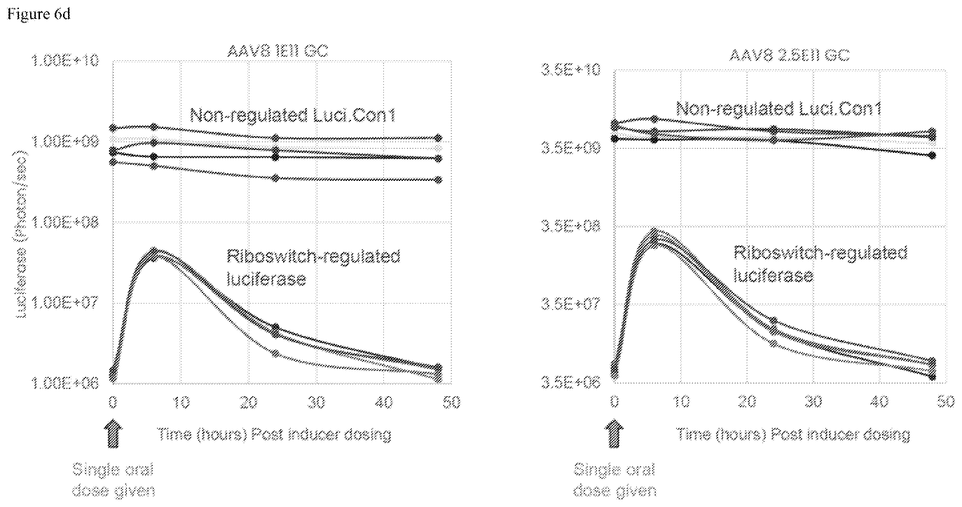

In an “on” switch scenario, aptamer/ligand binding inhibits the ribonuclease function of the ribozyme, decreasing cleavage of the target gene RNA that contains the polynucleotide cassette, thereby increasing target gene expression in the presence of ligand. Examples of an on switch include a riboswitch that comprises a twister ribozyme linked to an aptamer, wherein the aptamer is linked to the 3′ or 5′ end of the twister ribozyme P1 stem, wherein when the aptamer is linked to the 3′ end of the twister ribozyme P1 stem, a portion of the 3′ arm of the twister ribozyme P1 stem is alternatively the 5′ arm of the aptamer P1 stem, and wherein when the aptamer is linked to the 5′ end of the twister ribozyme P1 stem, a portion of the 5′ arm of the twister ribozyme P1 stem is alternatively the 3′ arm of the aptamer P1 stem (see, e.g., FIGS. 6a-6b of WO2017/136608 and the associated text, incorporated herein by reference).

### Aptamer Modulation of Polyadenylation

In embodiments, the expression of a target gene is regulated by aptamer-modulated polyadenylation. The 3′ end of almost all eukaryotic mRNAs comprises a poly(A) tail—a homopolymer of 20 to 250 adenosine residues. Because addition of the poly(A) tail to mRNA protects it from degradation, expression of a gene can be influenced by modulating the polyadenylation the corresponding mRNA.

In one embodiment, the expression of the target gene is regulated through aptamer-modulated accessibility of polyadenylation sequences as described in and WO2018/156658, which is incorporated in its entirety by reference herein. In such embodiments, the riboswitch comprises an effector stem-loop and an aptamer described herein, wherein the effector stem-loop comprises a polyadenylation signal, and wherein the aptamer and effector stem-loop are linked by an alternatively shared stem arm comprising a sequence that is complementary to the unshared arm of the aptamer stem (e.g., the aptamer P1 stem) and to the unshared arm of the effector stem loop (see, e.g., FIGS. 1a, 1b, 2a, and 5a of WO2018/156658 and the associated text, incorporated herein by reference). In one embodiment, the effector stem-loop is positioned 3′ of the aptamer such that the alternatively shared stem arm comprises all or a portion of the 3′ aptamer stem arm and all or a portion of the 5′ arm of the effector stem. In one embodiment, the effector stem-loop is positioned 5′ of the aptamer such that the alternatively shared stem arm comprises all or a portion of the 5′ aptamer stem arm and all or a portion of the 3′ arm of the effector stem. In one embodiment, the polyadenylation signal comprises AATAA or ATTAA. In one embodiment, the polyadenylation signal is AATAAA or ATTAAA. In embodiments, the polyadenylation signal is a downstream element (DSE). In one embodiment, the polyadenylation signal is an upstream sequence element (USE). In one embodiment, the polynucleotide cassette comprises two riboswitches, wherein the effector stem loop of the first riboswitch comprises all or part of the polyadenylation signal AATAAA or ATTAAA and the effector stem loop of the second riboswitch comprises all or part of the downstream element (DSE). In one embodiment, the two riboswitches each comprise aptamers that bind the same ligand. In one embodiment, the two riboswitches comprise different aptamers that bind different ligands.

In some embodiments, the riboswitch comprises a sensing region (e.g., an aptamer described herein) and an effector region comprising a binding site for the small nuclear ribonucleoprotein (snRNP) U1, which is part of the spliceosome. WO2017/136591 describes riboswitches wherein the effector region comprises a U1 snRNP binding site (and sequence complementary thereto), and is incorporated herein by reference in its entirety. When the aptamer binds its ligand, the effector region forms a stem and sequesters the U1 snRNP binding site from binding a U1 snRNP. Under certain conditions (for example, when the aptamer is not bound to its ligand), the effector region is in a context that provides access to the U1 snRNP binding site, allowing U1 snRNP to bind the mRNA and inhibit polyadenylation leading to degradation of the message. The U1 snRNP binding site can be any polynucleotide sequence that is capable of binding the U1 snRNP, thereby recruiting the U1 snRNP to the 3′ UTR of a target gene and suppressing polyadenylation of the target gene message. In one embodiment, the U1 snRNP binding site is CAGGTAAGTA, (CAGGUAAGUA, when in the mRNA). In some embodiments, the U1 snRNP binding site is a variation of this consensus sequence, including for example sequences that are shorter or have one or more nucleotides changed from the consensus sequence. In one embodiment, the U1 snRNP binding site contains the sequence CAGGTAAG. In some embodiments, the binding site is encoded by the sequence selected from CAGGTAAGTA, CAGGTAAGT, and CAGGTAAG. The UT snRNP binding site can be any 5′ splice site sequence from a gene, e.g., the 5′ splice site from human DHFR exon 2.

### Aptamer-Mediated Modulation of Ribonuclease Cleavage

In one embodiment, the expression of the target gene is regulated through aptamer-modulated ribonuclease cleavage. Ribonucleases (RNases) recognize and cleave specific ribonuclease substrate sequences. Provided herein are recombinant DNA constructs that, when incorporated into the DNA of a target gene, provide the ability to regulate expression of the target gene by aptamer/ligand mediated ribonuclease cleavage of the resulting RNA. In some embodiments, the aptamer encoding sequence described herein is part of a construct that contains or encodes a ribonuclease substrate sequence and a riboswitch comprising an effector region and the aptamer such that when the aptamer binds a ligand, target gene expression occurs (as described in WO2018/161053, which is incorporated in its entirety by reference herein). In embodiments, an RNase P substrate sequence is linked to a riboswitch wherein the riboswitch comprises an effector region and an aptamer described herein, wherein the effector region comprises a sequence complimentary to a portion of the RNase P substrate sequence. Binding of a suitable ligand to the aptamer induces structural changes in the aptamer and effector region, altering the accessibility of the ribonuclease substrate sequence for cleavage by the ribonuclease.

In one embodiment, the aptamer sequence is located 5′ to the RNase P substrate sequence and the effector region comprises all or part of the leader sequence and all or part of the 5′ acceptor stem sequence of the RNase P substrate sequence. See, e.g., FIGS. 1a, 1b, and 3b of WO2018/161053 and the associated text, incorporated herein by reference. In further embodiments, the acceptor stem of the RNase P substrate and the riboswitch effector region are separated by 0, 1, 2, 3, or 4 nucleotides. In other embodiments, the effector region stem includes, in addition to leader sequence (and its complement), one or more nucleotides of the acceptor stem of the RNase P substrate, and sequence complementary to the one or more nucleotides of the acceptor stem.

In one embodiment, the aptamer sequence of the polynucleotide cassette is located 3′ to the RNase P substrate sequence and the effector region comprises sequence complimentary to the all or part of the 3′ acceptor stem of the RNase P substrate sequence. See, e.g., FIG. 3a of WO2018/161053 and the associated text, incorporated herein by reference. In further embodiments, the effector region sequence complimentary to the 3′ acceptor stem of the RNase P substrate is 1 to 7 nucleotides. In other words, the effector region stem includes 1 to 7 nucleotides of the acceptor stem and includes sequence that is complementary to this 1 to 7 nucleotides of the acceptor stem. In embodiments, the riboswitch is located 3′ of the RNase P substrate so the effector region stem and the acceptor stem of the RNase P substrate do not overlap. In embodiments, the effector region and the acceptor stem of the RNase P substrate are immediately adjacent (i.e., not overlapping). In other embodiments, the effector region and the acceptor stem of the RNase P substrate are separated by 1, 2, 3, 4, 5 or more nucleotides.

### Target Gene

The aptamers and gene regulation cassettes disclosed herein can be used to regulate the expression of any target gene that can be expressed in a target cell, tissue or organism. The term “target gene” refers to a polynucleotide that is introduced into a cell and is capable of being transcribed into RNA and translated and/or expressed under appropriate conditions. Alternatively, the target gene is endogenous to the target cell and the gene regulation cassette is positioned into the target gene (for example into an existing untranslated region or intron of the endogenous target gene).

An example of a target gene is a polynucleotide encoding a therapeutic polypeptide. In one embodiment, the target gene is exogenous to the cell in which the recombinant DNA construct is to be transcribed. In another embodiment, the target gene is endogenous to the cell in which the recombinant DNA construct is to be transcribed. The target gene may be a gene encoding a protein, or a sequence encoding a non-protein coding RNA. The target gene may be, for example, a gene encoding a structural protein, an enzyme, a cell signaling protein, a mitochondrial protein, a zinc finger protein, a hormone, a transport protein, a growth factor, a cytokine, an intracellular protein, an extracellular protein, a transmembrane protein, a cytoplasmic protein, a nuclear protein, a receptor molecule, an RNA binding protein, a DNA binding protein, a transcription factor, translational machinery, a channel protein, a motor protein, a cell adhesion molecule, a mitochondrial protein, a metabolic enzyme, a kinase, a phosphatase, exchange factors, a chaperone protein, and modulators of any of these. In embodiments, the target gene encodes erythropoietin (Epo), human growth hormone (hGH), transcription activator-like effector nucleases (TALEN), human insulin, CRISPR associated protein 9 (cas9), or an immunoglobulin (or portion thereof), including, e.g., a therapeutic antibody.

In embodiments, the target gene is Cas9 or CasRx and the expression construct further comprises a sequence encoding a guide RNA (gRNA), for example a gRNA targeting PCSK9, which can be used to regulate expression of the gRNA target.

In embodiments, the target gene is PTH. In embodiments, the target gene is insulin (e.g., comprising sequence comprising the A chain, B chain and C peptide) for use in regulating insulin levels in response to a small molecule for treating diabetes.

In embodiments, the target gene is a therapeutic antibody including an anti-PCSK9 antibody, anti-VEGFR2 antibody (e.g., for ophthalmological applications), anti-amyloid App3-42 antibody, anti-IL-17 antibody, anti-PD1 antibody, and anti-HER2 antibody. In embodiments when the target gene is an antibody, the heavy and light chains can be expressed from a single message separated by a protein cleave site (furan, etc.) or peptide self-leaving site (e.g., 2A peptide such as T2A or P2A).

In embodiments, the target gene encodes an antibody against the SARS-CoV-2 viral proteins or antigens (such as the spike protein)(e.g., casirivimab and/or imdevimab (Regeneron), or bamlanivimab and/or etesevimab (Eli Lilly)). In embodiments, the target gene encodes all or a portion of a SARS-CoV-2 spike protein, where induction of expression produces mRNA and thus functions like an inducible mRNA vaccine (mRNA-1273, Moderna or Comirnaty, Pfizer-BioNTech).

In embodiments, the aptamers and gene regulation cassettes disclosed herein are used to regulate the expression of a target gene in eukaryotic cells for example, mammalian cells and more particularly human cells. In embodiments, the aptamers and gene regulation cassettes disclosed herein are used to regulate the expression of a target gene in the eye (including cornea and retina), central nervous system (including the brain), liver, kidney, pancreas, heart, airway, muscle, skin, lung, cartilage, testes, arteries, thymus, bone marrow, or in tumors.

In one aspect, provided are recombinant vectors and their use for the introduction of a polynucleotide comprising a target gene and a gene regulation cassette, wherein the gene regulation cassette comprises an aptamer disclosed herein. In some embodiments, the recombinant DNA constructs include additional DNA elements including DNA segments that provide for the replication of the DNA in a host cell and expression of the target gene in target cells at appropriate levels. The ordinarily skilled artisan appreciates that expression control sequences (promoters, enhancers, and the like) are selected based on their ability to promote expression of the target gene in the target cell. “Vector” means a recombinant plasmid, yeast artificial chromosome (YAC), mini chromosome, DNA mini-circle or virus (including virus derived sequences) that comprises a polynucleotide to be delivered into a host cell, either in vitro or in vivo. In one embodiment, the recombinant vector is a viral vector or a combination of multiple viral vectors.

Viral vectors for the expression of a target gene in a target cell, tissue, or organism are known in the art and include adenoviral (AV) vectors, adeno-associated virus (AAV) vectors, retroviral and lentiviral vectors, and Herpes simplex type 1 (HSV1) vectors.

Adenoviral vectors include, for example, those based on human adenovirus type 2 and human adenovirus type 5 that have been made replication defective through deletions in the E1 and E3 regions. The transcriptional cassette can be inserted into the E1 region, yielding a recombinant E1/E3-deleted AV vector. Adenoviral vectors also include helper-dependent high-capacity adenoviral vectors (also known as high-capacity, “gutless” or “gutted” vectors), which do not contain viral coding sequences. These vectors, contain the cis-acting elements needed for viral DNA replication and packaging, mainly the inverted terminal repeat sequences (ITR) and the packaging signal (CY). These helper-dependent AV vector genomes have the potential to carry from a few hundred base pairs up to approximately 36 kb of foreign DNA.

Recombinant adeno-associated virus “rAAV” vectors include any vector derived from any adeno-associated virus serotype, including, without limitation, AAV-1, AAV-2, AAV-3, AAV-4, AAV-5, AAV-7 and AAV-8, AAV-9, AAV-10, and the like. rAAV vectors can have one or more of the AAV wild-type genes deleted in whole or in part, preferably the Rep and/or Cap genes, but retain functional flanking ITR sequences. Functional ITR sequences are retained for the rescue, replication, packaging and potential chromosomal integration of the AAV genome. The ITRs need not be the wild-type nucleotide sequences, and may be altered (e.g., by the insertion, deletion or substitution of nucleotides) so long as the sequences provide for functional rescue, replication and packaging.

Alternatively, other systems such as lentiviral vectors can be used. Lentiviral-based systems can transduce nondividing as well as dividing cells making them useful for applications targeting, for examples, the nondividing cells of the CNS. Lentiviral vectors are derived from the human immunodeficiency virus and, like that virus, integrate into the host genome providing the potential for very long-term gene expression.

Polynucleotides, including plasmids, YACs, minichromosomes and minicircles, carrying the target gene containing the gene regulation cassette can also be introduced into a cell or organism by nonviral vector systems using, for example, cationic lipids, polymers, or both as carriers. Conjugated poly-L-lysine (PLL) polymer and polyethylenimine (PEI) polymer systems can also be used to deliver the vector to cells. Other methods for delivering the vector to cells includes hydrodynamic injection and electroporation and use of ultrasound, both for cell culture and for organisms. For a review of viral and non-viral delivery systems for gene delivery see Nayerossadat, N. et al. (Adv Biomed Res. 2012; 1:27) incorporated herein by reference.

In one aspect, this disclosure provides a method of modulating the expression of a target gene (e.g., a therapeutic gene) comprising (a) inserting the polynucleotide cassette comprising an aptamer disclosed herein into the target gene, (b) introducing the target gene comprising the polynucleotide cassette into a cell, and (c) exposing the cell to a small molecule ligand that specifically binds the aptamer in an amount effective to induce expression of the target gene. In aspects, expression of the target gene in target cells confers a desired property to a cell into which it was introduced, or otherwise leads to a desired therapeutic outcome.

In one embodiment, a gene regulation cassette comprising an aptamer disclosed herein is inserted into the protein coding sequence of the target gene (rather than in the 5′ or 3′ untranslated regions). In one embodiment, a single gene regulation cassette comprising an aptamer disclosed herein is inserted into the target gene. In other embodiments 2, 3, 4, or more gene regulation cassettes are inserted in the target gene, wherein one or more gene regulation cassettes comprise an aptamer disclosed herein. In one embodiment, two gene regulation cassettes are inserted into the target gene, wherein one or both gene regulation cassettes comprise an aptamer disclosed herein. When multiple gene regulation cassettes are inserted into a target gene, they each can contain the same aptamer such that a single ligand can be used to modulate target gene expression. In other embodiments, multiple gene regulation cassettes are inserted into a target gene, each can contain a different aptamer so that exposure to multiple different small molecule ligands modulates target gene expression.

### Methods of Treatment and Pharmaceutical Compositions

In one aspect, provided is a method of regulating the level of a therapeutic protein delivered by gene therapy. The therapeutic gene sequence containing a regulatory cassette comprising an aptamer disclosed herein is delivered to the target cells in the body, e.g., by a vector. The cell specificity of the target gene expression may be controlled by a promoter and/or other elements within the vector and/or by the capsid of the viral vector. Delivery of the vector construct containing the target gene, and the transfection of the target tissues resulting in stable transfection of the regulated target gene, is the first step in producing the therapeutic protein. However, due to an aptamer within the target gene sequence, the target gene is not expressed at significant levels, i.e., it is in the “off state” in the absence of the specific ligand that binds to the aptamer contained within in the regulatory cassette riboswitch. Only when the aptamer specific ligand is administered is the target gene expression activated.

The delivery of the vector construct containing the target gene and the delivery of the activating ligand generally are separated in time. The delivery of the activating ligand will control when the target gene is expressed, as well as the level of protein expression. The ligand may be delivered by a number of routes including, but not limited to, intravitreal, intraocular, inhalation, subcutaneous, intramuscular, intradermal, intralesion, topical, intraperitoneal, intravenous (IV), intra-arterial, perivascular, intracerebral, intracerebroventricular, oral, sublingual, sublabial, buccal, nasal, intrathoracic, intracardiac, intrathecal, epidural, intraosseous, or intraarticular.

The timing of delivery of the ligand will depend on the requirement for activation of the target gene. For example, if the therapeutic protein encoded by the target gene is required constantly, an oral small molecule ligand may be delivered daily, or multiple times a day, to ensure continual activation of the target gene, and thus continual expression of the therapeutic protein. If the target gene has a long acting effect, the inducing ligand may be dosed less frequently, for example, once a week, every other week, once a month.

This aptamers described herein in the context of a gene regulation cassette comprising a riboswitch allow the expression of a therapeutic transgene to be controlled temporally, in a manner determined by the temporal dosing of the ligand specific to the aptamer. The expression of the therapeutic transgene only on ligand administration, increases the safety of a gene therapy treatment by allowing the target gene to be off in the absence of the ligand.

Different aptamers can be used in multiple riboswitches to allow different ligands to up-regulate or down-regulate the expression of a target gene. In certain embodiments, each therapeutic gene containing a regulatory cassette will have a specific aptamer within the cassette that will be activated by a specific small molecule. This means that each therapeutic gene can be activated only by the ligand specific to the aptamer housed within it. In these embodiments, each ligand will only activate one therapeutic gene. This allows for the possibility that several different “target genes” may be delivered to one individual and each will be activated on delivery of the specific ligand for the aptamer contained within the regulatory cassette housed in each target gene.

The aptamers disclosed herein in the context of a riboswitch allow any therapeutic protein whose gene can be delivered to the body (such as erythropoietin (EPO) or a therapeutic antibody) to be produced by the body when the activating ligand is delivered. This method of therapeutic protein delivery may replace the manufacture of such therapeutic proteins outside of the body which are then injected or infused, e.g., antibodies used in cancer or to block inflammatory or autoimmune disease. The body containing the regulated target gene becomes the biologics manufacturing factory, which is switched on when the gene-specific ligand is administered.

In one embodiment, the target protein may be a nuclease that can target and edit a particular DNA sequence. Such nucleases include CasRx, Cas9, zinc finger containing nucleases, or TALENs. In the case of these nucleases, the nuclease protein may be required for only a short period of time that is sufficient to edit the target endogenous genes. However, if an unregulated nuclease gene is delivered to the body, this protein may be present for the rest of the life of the cell. In the case of nucleases, there is an increasing risk of off-target editing the longer the nuclease is present. Regulation of expression of such proteins has a significant safety advantage. In this case, vector containing the nuclease target gene containing a regulatory cassette could be delivered to the appropriate cells in the body. The target gene is in the “off” state in the absence of the cassette-specific ligand, so no nuclease is produced. Only when the activating ligand is administered, is the nuclease produced. When sufficient time has elapsed allowing sufficient editing to occur, the ligand will be withdrawn and not administered again. Thus the nuclease gene is thereafter in the “off” state and no further nuclease is produced and editing stops. This approach may be used to correct genetic conditions, including a number of inherited retinopathies such as LCA10 caused by mutations in CEP290 and Stargardts disease caused by mutations in ABCA4.

Administration of a regulated target gene encoding a therapeutic protein which is activated only on specific ligand administration may be used to regulate therapeutic genes to treat many different types of diseases, e.g., cancer with therapeutic antibodies, immune disorders with immune modulatory proteins or antibodies, metabolic diseases, rare diseases such as PNH with anti-C5 antibodies or antibody fragments as the regulated gene, or ocular angiogenesis with therapeutic antibodies, and dry AMD with immune modulatory proteins.

A wide variety of specific target genes, allowing for the treatment of a wide variety of specific diseases and conditions, are suitable for use as a target gene whose expression can be regulated using an aptamer/ligand described herein. For example, insulin or an insulin analog (preferably human insulin or an analog of human insulin) may be used as the target gene to treat type I diabetes, type II diabetes, or metabolic syndrome; human growth hormone may be used as the target gene to treat children with growth disorders or growth hormone-deficient adults; erythropoietin (preferably human erythropoietin) may be used as the target gene to treat anemia due to chronic kidney disease, anemia due to myelodysplasia, or anemia due to cancer chemotherapy. Additional target genes compatibles with the aptamers and gene expression cassettes disclosed herein include, but are not limited to, cyclic nucleotide-gated cation channel alpha-3 (CNGA3) and cyclic nucleotide-gated cation channel beta-3 (CNGB3) for the treatment of achromatopsia, retinoid isomerohydrolase (RPE65) for the treatment of retinitis pigmentosa or Leber's congential amaurosis, X-linked retinitis pigmentosa GTPase regulator (RPGR) for the treatment of X-linked retinitis pigmentosa, glutamic acid decarboxylase (GAD) including for the treatment of Parkinson's disease, regulator of nonsense transcripts 1 (UPF1) for the treatment amyotrophic lateral sclerosis, and aquaporin for the treatment of radiation-induced xerostomia and Sjogren's syndrome. Additional target genes include ArchT (archaerhodopsin from Halorubrum strain TP009), Jaws (cruxhalorhodopsin derived from Haloarcula (Halobacterium) salinarum (strain Shark)), iC1C2 (a variant of a C1C2 chimaera between channel rhodopsins ChR1 and ChR2 from Chlamydomonas reinhardiii), or Rgs9-anchor protein (R9AP), a critical component of GTPase complex that mediates the deactivation of phototransduction cascade.

The expression constructs comprising an aptamer disclosed herein may be especially suitable for treating diseases caused by single gene defects such as cystic fibrosis, hemophilia, muscular dystrophy, thalassemia, or sickle cell anemia. Thus, human β-, γ-, δ-, or ζ-globin may be used as the target gene to treat β-thalassemia or sickle cell anemia; human Factor VIII or Factor IX may be used as the target gene to treat hemophilia A or hemophilia B.

In embodiments, the expression constructs/small molecules disclosed herein may be used to treat, prevent, or lessen the severity of a viral disease. In embodiments, the disclosure provides a method for treating, preventing, or lessening the severity of COVID-19 by expressing antibodies against the SARS-CoV-2 viral proteins or antigens (e.g., spike protein) in response to administration of a small molecule ligand. In embodiments, the disclosure provides a method for preventing (or lessening the severity of) infection by SARS-CoV-2 by expressing the spike protein (or multiple serotype spike proteins) or portions thereof, using the gene regulation cassettes described herein and administering ligand. In embodiments, the target gene is an antibody against the SARS-CoV-2 viral proteins or antigens (such as the spike protein). In other embodiments, the target gene encodes all or a portion of one or more SARS-CoV-2 spike proteins, where induction of expression produces mRNA and thus functions like an inducible mRNA vaccine. In embodiments, the expression construct is part of an AAV viral genome and the AAV vector comprising the expression construct is administered to, e.g., the muscle of a subject followed by administration of the ligand.

In embodiments, the disclosure provides a method for restoring hemocrit and a method of treating anemia by expression of Epo from a gene regulation construct described herein, wherein a vector comprising an Epo gene regulation construct is administered to the subject in need thereof followed by administration of a small molecule ligand described herein. In embodiments, the anemia is due to chronic kidney disease in the subject.

In embodiments, the disclosure provides a method for restoring hemocrit and a method of treating chronic kidney disease by expression of Epo from a gene regulation construct described herein, wherein a vector comprising an Epo gene regulation construct is administered to the subject in need thereof followed by administration of a small molecule ligand described herein.

The small molecules described herein are generally combined with one or more pharmaceutically acceptable carriers to form pharmaceutical compositions suitable for administration to a patient. Pharmaceutically acceptable carriers include solvents, binders, diluents, disintegrants, lubricants, dispersion media, coatings, antibacterial and antifungal agents, isotonic and absorption delaying agents, and the like, generally used in the pharmaceutical arts. Pharmaceutical compositions may be in the form of tablets, pills, capsules, troches, eye drops, and the like, and are formulated to be compatible with their intended route of administration. Examples of routes of administration include parenteral, e.g., intravenous, intradermal, intranasal, subcutaneous, oral, inhalation, transdermal (topical), transmucosal, and ocular.

The pharmaceutical compositions comprising compounds of I-XVI are administered to a patient in a dosing schedule such that an amount of the compound sufficient to desirably regulate the target gene is delivered to the patient. When the dosage form is a tablet, pill, or the like, preferably the pharmaceutical composition comprises from 0.1 mg to 10 g of the compound; from 0.5 mg to 5 g of the compound; from 1 mg to 1 g of the compound; from 2 mg to 750 mg of the compound; from 5 mg to 500 mg of the compound; from 10 mg to 250 mg of the compound; or from 150 mg to 300 mg of the compound.

The pharmaceutical compositions may be dosed once per day or multiple times per day (e.g., 2, 3, 4, 5, or more times per day). Alternatively, pharmaceutical compositions may be dosed less often than once per day, e.g., once every 2, 3, 4, 5, 6, 7, 8, 9, 10, 11, 12, 13, or 14 days, or once a month or once every few months. In some embodiments, the pharmaceutical compositions may be administered to a patient only a small number of times, e.g., once, twice, three times, etc.

Provided herein is a method of treating a patient in need of increased expression of a therapeutic protein encoded by a target gene, the method comprising administering to the patient a pharmaceutical composition comprising a ligand, which an aptamer disclosed herein binds to or otherwise responds to, wherein the patient previously had been administered a recombinant DNA comprising the target gene, and where the target gene contains a gene regulation cassette disclosed herein that provides the ability to regulate expression of the target gene by the ligand of the aptamer. Provided herein is a pharmaceutical composition comprising a ligand, which an aptamer disclosed herein binds to or otherwise responds to, for use in a method of treating a patient in need of increased expression of a therapeutic protein encoded by a target gene, wherein the patient previously had been administered a recombinant DNA comprising the target gene, and where the target gene contains a gene regulation cassette disclosed herein that provides the ability to regulate expression of the target gene by the ligand of the aptamer.

Aptamers for Detection and/or Diagnostic Uses

A wide range of detection and diagnostic agents can be linked to aptamers through chimerical or physical conjugation. Further, aptamers can be incorporated in biosensors, microfluidic devices and other detection platforms. In some embodiments, the aptamer is conjugated to a polyalkylene glycol moiety, including, but not limited to, polyethylene glycol (PEG), polypropylene glycol (PPG), polyoxyethylated glycerol (POG) and other polyoxyethylated polyols, polyvinyl alcohol (PVA) and other polyalkylene oxides, polyoxyethylated sorbitol, or polyoxyethylated glucose.

In some embodiments, the aptamer is conjugated to a detectable moiety, including, but not limited to, fluorescent moieties or labels, imaging agents, radioisotopic moieties, radiopaque moieties, and the like, e.g. detectable labels such as biotin, fluorophores, chromophores, spin resonance probes, nanoparticles (including, but not limited to gold, magnetic, and superparamagnetic nanoparticles), quantum dots, radiolabels. Exemplary fluorophores include fluorescent dyes (e.g. fluorescein, rhodamine, and the like) and other luminescent molecules (e.g. luminal). A fluorophore may be environmentally-sensitive such that its fluorescence changes if it is located close to one or more residues in the modified protein that undergo structural changes upon binding a substrate (e.g. dansyl probes). Exemplary radiolabels include small molecules containing atoms with one or more low sensitivity nuclei (¹³C, ¹⁵N, ²H, ¹²⁵I, ¹²³I, ⁹⁹Tc, ⁴³K, ⁵²Fe, ⁶⁷Ga, ⁶⁸Ga, ¹¹¹In and the like). Other useful moieties are known in the art.

In some embodiments, the aptamer is conjugated to a therapeutic moiety, including, but not limited to, an anti-inflammatory agent, anti-cancer agent, anti-neurodegenerative agent, anti-infective agent, or generally a therapeutic agent.

Methods for Identifying an Aptamer that Binds to a Compound

Disclosed herein are methods for identifying an aptamer that binds to a compound of Formula I-XXII, or otherwise modulates target gene expression when part of a riboswitch, in response to the addition of, or exposure to, the compound of Formula I-XXII. In one embodiment, the method comprises the steps of
(i) selecting a parent aptamer sequence;
(ii) generating an aptamer library comprising sequence encoding the aptamer selected in (i), wherein one or more nucleotides in the aptamer encoding sequence are randomly mutated at one or more positions that correspond to one or more unpaired regions in the aptamer, wherein the mutated aptamer sequences are in the context of a riboswitch that controls the expression of a reporter gene;
(iii) screening the library from (ii) for aptamers having increased regulation (e.g., higher fold induction or repression) of the target gene expression in response to a compound disclosed herein compared to the parent aptamer sequence;
(iv) optionally repeating steps (ii) and (iii) on an aptamer identified in step (iii) rather than an aptamer selected in step (i).

The parent aptamer sequence may be a TPP aptamer, including known TPP aptamer sequence or may be a putative TPP aptamer identified by searching for homologous sequences in available databases. The parent aptamer sequence may be an aptamer sequence disclosed herein, e.g.,

(12C6-1; SEQ ID NO: 1)
CTGGGGAGTCCTTCATGCGGGGCTGAGAGGATGGAAGCAATCGAC
CATCGACCCATTGCACCTGATCCGGATCATGCCGGCGCAGGGAG.

The step of selecting a parent aptamer sequence can involve, for example, (i) identifying a putative TPP aptamer; (ii) inserting the aptamer into a riboswitch that modulates the expression of a target gene (for example a reporter gene); and (iii) exposing the riboswitch/target gene construct to a thiamine or TPP analog or derivative (e.g., the compounds described herein).

Putative TPP aptamers can be identified from an appropriate sequence database such as the Rfam database, which is a collection of RNA families, each represented by multiple sequence alignments, consensus secondary structures and covariance models (CMs). In embodiments, the putative TPP aptamer is identified from the Rfam TPP riboswitch family RF00059. In embodiments, the putative TPP aptamer has a sequence that is at least 85%, at least 90%, at least 95%, at least 96%, at least 97% at least 98% or at least 99% identical to

thiC
(GUAAUGUGUCGGAGUGCCUUAGGGAUUAUUCCCCUAAAGC
UGAGACCGCAUUGCGGGAUCCGUUGAACCUGAUCAGGCUAA
UACCUGCGAAGGGAACACAUUAC, SEQ ID NO: 679)
or
thiM
(GUAAUGUCUCGGGGUGCCCUUCUGCGUGAAGGCUGAGAA
AUACCCGUAUCACCUGAUCUGGAUAAUGCCAGCGUAGGG
AAGACAUUAC, SEQ ID NO: 680).

The putative TPP aptamer can be inserted into a riboswitch using techniques known to the ordinarily skilled artisan. The responsiveness of the aptamer to the presence of TPP and one or more thiamine or TPP analogs or derivatives (e.g., the compounds described herein) can be tested in cell culture and/or in a cell-free system. In particular, the cell culture system is a eukaryotic cell culture including, e.g., a mammalian, a plant, or an insect cell culture.

In order to identify aptamers that respond to a compound described herein, one or more nucleotide positions of the sequence encoding the aptamer (i.e., the parent aptamer) are randomized. Areas of the sequence that can be randomized include J2-4; L3a; P4/J4-5 to J5-4; and L5.

The nucleotide positions for randomization can be selected based on the structure of the parent aptamer sequence. The predicted secondary structure can be obtained using available programs such as RNAfold (http://rna.tbi.univie.ac.at/cgi-bin/RNAWebSuite/RNAfold.cgi) and/or by comparison to the crystal structure of a related aptamer (e.g., the E. coli thiM riboswitch in Edwards, T E & Ferré-D'Amaré, A R, Structure. 2006 September; 14(9):1459-68). For example, unpaired regions of the aptamer, including loop (L) regions (e.g., L3 and/or L5) and joining (J) regions (e.g., J3-2 (joining paired regions P3 and P2), J2-4, and/or J4-5), can be identified, and one or more nucleotides in one or more unpaired regions can be randomized to generate a library of aptamers. In embodiments, one or more nucleotides adjacent to one or more unpaired regions are randomized. Additionally, one or more nucleotides in a paired (P) region can be randomized. Further, one or more nucleotides in an unpaired or paired region can be added or deleted. The mutagenized aptamer sequences can be provided as a library of aptamer sequences in the context of a riboswitch. In embodiments, the aptamer library is provided in the context of a riboswitch as part of a gene expression cassette disclosed herein.

The aptamer encoding sequences containing one or more mutations can be tested for responsiveness to the presence of one or more compounds described herein.

Aptamers that are responsive to the desired compound, can be further mutagenized by randomizing nucleotides. The nucleotides at selected positions, for example unpaired regions, can be randomized and a library created as described above.

Reporter proteins encoded by the reporter genes used in the methods disclosed herein are proteins that can be assayed by detecting characteristics of the reporter protein, such as enzymatic activity or spectrophotometric characteristics, or indirectly, such as with antibody-based assays. Examples of reporter gene products that are readily detectable include, but are not limited to, puromycin resistance marker (pac), 3-galactosidase, luciferase, orotidine 5′-phosphate decarboxylase (URA3), arginine permease CAN1, galactokinase (GAL1), beta-galactosidase (LacZ), or chloramphenicol acetyl transferase (CAT). Other examples of detectable signals include cell surface markers, including, but not limited to CD4. Reporter genes suitable for the use in the methods for identifying aptamers disclosed herein also include fluorescent proteins (e.g., green fluorescent protein (GFP) and its derivatives), or proteins fused to a fluorescent tag. Examples of fluorescent tags and proteins include, but are not limited to, (3-F)Tyr-EGFP, A44-KR, aacuGFP1, aacuGFP2, aceGFP, aceGFP-G222E-Y220L, aceGFP-h, AcGFP1, AdRed, AdRed-C148S, aeurGFP, afraGFP, alajGFP1, alajGFP2, alajGFP3, amCyanl, amFP486, amFP495, amFP506, amFP515, amilFP484, amilFP490, amilFP497, amilFP504, amilFP512, amilFP513, amilFP593, amilFP597, anm1GFP1, anm1GFP2, anm2CP, anobCFP1, anobCFP2, anobGFP, apulFP483, AQ14, AQ143, Aquamarine, asCP562, asFP499, AsRed2, asulCP, atenFP, avGFP, avGFP454, avGFP480, avGFP509, avGFP510, avGFP514, avGFP523, AzamiGreen, Azurite, BDFP1.6, bfloGFPal, bfloGFPcl, BFP, BFP.A5, BFP5, bsDronpa (On), ccalGFPl, ccalGFP3, ccalOFP1, ccalRFP1, ccalYFP1, cEGFP, cerFP505, Cerulean, CFP, cFP484, cfSGFP2, cgfmKate2, CGFP, cgfTagRFP, cgigGFP, cgreGFP, CheGFP1, CheGFP2, CheGFP4, Citrine, Citrine2, Clomeleon, Clover, cp-mKate, cpCitrine, cpT-Sapphire174-173, CyOFP1, CyPet, CyRFP1 (CyRFP1), d-RFP618, D10, dlEosFP (Green), d1EosFP (Red), d2EosFP (Green), d2EosFP (Red), deGFP1, deGFP2, deGFP3, deGFP4, dendFP (Green), dendFP (Red), Dendra (Green), Dendra (Red), Dendra2 (Green), Dendra2 (Red), Dendra2-M159A (Green), Dendra2-M159A (Orange), Dendra2-T69A (Green), Dendra2-T69A (Orange), dfGFP, dimer1, dimer2, dis2RFP, dis3GFP, dKeima, dKeima570, dLanYFP, DrCBD, Dreiklang (On), Dronpa (On), Dronpa-2 (On), Dronpa-3 (On), dsFP483, DspR1, DsRed, DsRed-Express, DsRed-Express2, DsRed-Max, DsRed.M1, DsRed.T3, DsRed.T4, DsRed2, DstC1, dTFPO.1, dTFPO.2, dTG, dTomato, dVFP, E2-Crimson, E2-Orange, E2-Red/Green, EaGFP, EBFP, EBFP1.2, EBFP1.5, EBFP2, ECFP, ECFPH148D, ECGFP, eechGFP1, eechGFP2, eechGFP3, eechRFP, efasCFP, efasGFP, eforCP, EGFP, eGFP203C, eGFP205C, Emerald, Enhanced Cyan-Emitting GFP, EosFP (Green), EosFP (Red), eqFP578, eqFP611, eqFP611V124T, eqFP650, eqFP670, EYFP, EYFP-Q69K, fabdGFP, ffDronpa (On), FoldingReporterGFP, FP586, FPrfl2.3, FR-1, FusionRed, FusionRed-M, G1, G2, G3, Gamillus (On), Gamillus0.1, Gamillus0.2, Gamillus0.3, Gamillus0.4, GCaMP2, gfasGFP, GFP(S65T), GFP-151pyTyrCu, GFP-Tyrl5lpyz, GFPmut2, GFPmut3, GFPxm16, GFPxm161, GFPxm162, GFPxm163, GFPxm18, GFPxm181uv, GFPxm18uv, GFPxm19, GFPxml9luv, GFPxml9uv, H9, HcRed, HcRed-Tandem, HcRed7, hcriGFP, hmGFP, HriCFP, HriGFP, iFP1.4, iFP2.0, iLov, iq-EBFP2, iq-mApple, iq-mCerulean3, iq-mEmerald, iq-mKate2, iq-mVenus, iRFP670, iRFP682, iRFP702, iRFP713, iRFP720, IrisFP (Green), IrisFP (Orange), IrisFP-M159A (Green), Jred, Kaede (Green), Kaede (Red), Katushka, Katushka-9-5, Katushka2S, KCY, KCY-G4219, KCY-G4219-38L, KCY—R¹, KCY-R1-158A, KCY-R1-38H, KCY-R1-38L, KFP1 (On), KikGR1 (Green), KikGR1 (Red), KillerOrange, KillerRed, KO, Kohinoor (On), laesGFP, laGFP, LanFP1, LanFP2, lanRFP-AS831, LanYFP, laRFP, LSS-mKatel, LSS-mKate2, LSSmOrange, M355NA, mAmetrine, mApple, Maroon0.1, mAzamiGreen, mBanana, mBeRFP, mBlueberryl, mBlueberry2, mc1, mc2, mc3, mc4, mc5, mc6, McaG1, McaGlea, McaG2, mCardinal, mCarmine, mcavFP, mcavGFP, mcavRFP, mcCFP, mCerulean, mCerulean.B, mCerulean.B2, mCerulean.B24, mCerulean2, mCerulean2.D3, mCerulean2.N, mCerulean2.N(T65S), mCerulean3, mCherry, mCherry2, mCitrine, mClavGR2 (Green), mClavGR2 (Red), mClover3, mCyRFP1, mECFP, meffCFP, meffGFP, meffRFP, mEGFP, meleCFP, meleRFP, mEmerald, mEos2 (Green), mEos2 (Red), mEos2-A69T (Green), mEos2-A69T (Orange), mEos3.1 (Green), mEos3.1 (Red), mEos3.2 (Green), mEos3.2 (Red), mEos4a (Green), mEos4a (Red), mEos4b (Green), mEos4b (Red), mEosFP (Green), mEosFP (Red), mEosFP-F173S (Green), mEosFP-F173S (Red), mEosFP-M159A (Green), mEYFP, MfaGl, mGarnet, mGarnet2, mGeos-C(On), mGeos-E (On), mGeos-F (On), mGeos-L (On), mGeos-M (On), mGeos-S(On), mGingerl, mGinger2, mGrapel, mGrape2, mGrape3, mHoneydew, MiCy, mIFP, miniSOG, miniSOGQ103V, miniSOG2, miRFP, miRFP670, miRFP670nano, miRFP670vl, miRFP703, miRFP709, miRFP720, mIrisFP (Green), mIrisFP (Red), mK-GO (Early), mK-GO (Late), mKalama1, mKate, mKateM41GS158C, mKateS158A, mKateS158C, mKate2, mKeima, mKelly1, mKelly2, mKG, mKikGR (Green), mKikGR (Red), mKillerOrange, mKO, mKO2, mKOκ, mLumin, mMaple (Green), mMaple (Red), mMaple2 (Green), mMaple2 (Red), mMaple3 (Green), mMaple3 (Red), mMaroonl, mmGFP, mMiCy, mmilCFP, mNectarine, mNeonGreen, mNeptune, mNeptune2, mNeptune2.5, mNeptune681, mNeptune684, Montiporasp. #20-9115, mOrange, mOrange2, moxBFP, moxCerulean3, moxDendra2 (Green), moxDendra2 (Red), moxGFP, moxMaple3 (Green), moxMaple3 (Red), moxNeonGreen, moxVenus, mPapaya, mPapaya0.7, mPlum, mPlum-E16P, mRaspberry, mRed7, mRed7Q1, mRed7Q1S1, mRed7Q1S1BM, mRFP1, mRFP1-Q66C, mRFP1-Q66S, mRFP1-Q66T, mRFP1.1, mRFP1.2, mRojoA, mRojoB, mRouge, mRtms5, mRuby, mRuby2, mRuby3, mScarlet, mScarlet-H, mScarlet-I, mStable, mStrawberry, mT-Sapphire, mTagBFP2, mTangerine, mTFP0.3, mTFP0.7 (On), mTFP1, mTFP1-Y67W, mTurquoise, mTurquoise2, muGFP, mUkG, mVenus, mVenus-Q69M, mVFP, mVFP1, mWasabi, Neptune, NijiFP (Green), NijiFP (Orange), NowGFP, obeCFP, obeGFP, obeYFP, OFP, OFPxm, oxBFP, oxCerulean, oxGFP, oxVenus, P11, P4, P4-1, P4-3E, P9, PA-GFP (On), Padron (On), Padron(star) (On), Padron0.9 (On), PAmCherry 1 (On), PAmCherry2 (On), PAmCherry3 (On), PAmKate (On), PATagRFP (On), PATagRFP1297 (On), PATagRFP1314 (On), pcDronpa (Green), pcDronpa (Red), pcDronpa2 (Green), pcDronpa2 (Red), PdaC1, pdaelGFP, phiYFP, phiYFPv, pHluorin,ecliptic, pHluorin,ecliptic (acidic), pHluorin, ratiometric (acidic), pHluorin, ratiometric (alkaline), pHluorin2 (acidic), pHluorin2 (alkaline), pHuji, PlamGFP, pmeaGFP1, pmeaGFP2, pmimGFP1, pmimGFP2, Pp2FbFP, Pp2FbFPL30M, ppluGFP1, ppluGFP2, pporGFP, pporRFP, PS—CFP (Cyan), PS—CFP (Green), PS—CFP2 (Cyan), PS—CFP2 (Green), psamCFP, PSmOrange (Far-red), PSmOrange (Orange), PSmOrange2 (Far-red), PSmOrange2 (Orange), ptilGFP, R3-2+PCB, RCaMP, RDSmCherry0.1, RDSmCherry0.2, RDSmCherry0.5, RDSmCherry1, rfloGFP, rfloRFP, RFP611, RFP618, RFP630, RFP637, RFP639, roGFP1, roGFP1-R1, roGFP1-R8, roGFP2, rrenGFP, RRvT, rsCherry (On), rsCherryRev (On), rsCherryRevl.4 (On), rsEGFP (On), rsEGFP2 (On), rsFastLime (On), rsFolder (Green), rsFolder2 (Green), rsFusionRedl (On), rsFusionRed2 (On), rsFusionRed3 (On), rsTagRFP (ON), Sandercyanin, Sapphire, sarcGFP, SBFP1, SBFP2, SCFP1, SCFP2, SCFP3A, SCFP3B, scubGFP1, scubGFP2, scubRFP, secBFP2, SEYFP, sgl1, sgl2, sg25, sg42, sg50, SGFP1, SGFP2, SGFP2(206A), SGFP2(E222Q), SGFP2(T65G), SHardonnay, shBFP, shBFP-N158S/L173I, ShG24, Sirius, SiriusGFP, Skylan-NS (On), Skylan-S(On), smURFP, SNIFP, SOPP, SOPP2, SOPP3, SPOON (on), stylGFP, SuperfolderGFP, SuperfoldermTurquoise2, SuperfoldermTurquoise2ox, SuperNovaGreen, SuperNovaRed, SYFP2, T-Sapphire, TagBFP, TagCFP, TagGFP, TagGFP2, TagRFP, TagRFP-T, TagRFP657, TagRFP675, TagYFP, td-RFP611, td-RFP639, tdimer2(12), tdKatushka2, TDsmURFP, tdTomato, tKeima, Topaz, TurboGFP, TurboGFP-V197L, TurboRFP, Turquoise-GL, Ultramarine, UnaG, usGFP, Venus, VFP, vsfGFP-0, vsfGFP-9, WiC, W2, W7, WasCFP, Wi-Phy, YPet, zFP538, zoan2RFP, ZsGreen, ZsYellow1, αGFP, 10B, 22G, 5B, 6C, Ala, aacuCP, acanFP, ahyaCP, amilCP, amilCP580, amilCP586, amilCP604, apulCP584, BFPsol, Blue102, CFP4, cgigCP, CheGFP3, Clover1.5, cpasCP, Cyl1.5, dClavGR1.6, dClover2, dClover2A206K, dhorGFP, dhorRFP, dPapaya0.1, Dronpa-C62S, DsRed-Timer, echFP, echiFP, EYFP-F46L, fcFP, fcomFP, Fpaagar, Fpag_frag, Fpcondchrom, FPmann, FPmcavgr7.7, Gamillus0.5, gdjiCP, gfasCP, GFPhal, gtenCP, hcriCP, hfriFP, KikG, LEA, mcFP497, mcFP503, mcFP506, mCherry1.5, mClavGRl, mClavGR1.1, mClavGR1.8, mCloverl.5, mcRFP, meffCP, mEos2-NA, meruFP, mKate2.5, mOFP.T.12, mOFP.T.8, montFP, moxEos3.2, mPA-GFP, mPapaya0.3, mPapaya0.6, mRFP1.3, mRFP1.4, mRFP1.5, mTFP0.4, mTFP0.5, mTFP0.6, mTFP0.8, mTFP0.9, mTFP1-Y67H, mTurquoise-146G, mTurquoise-146S, mTurquoise-DR, mTurquoise-GL, mTurquoise-GV, mTurquoise-RA, mTurquoise2-G, NpR3784g, PDM1-4, psupFP, Q80R, rfloGFP2, RpBphPl, RpBphP2, RpBphP6, rrGFP, RSGFP1, RSGFP2, RSGFP3, RSGFP4, RSGFP6, RSGFP7, Rtms5, scleFP1, scleFP2, spisCP, stylCP, sympFP, TeAPCa, tPapaya0.01, Trp-lessGFP, vsGFP, Xpa, yEGFP, YFP3, zGFP, and zRFP.

Methods for screening an aptamer library disclosed herein may include measuring the activity of the reporter gene under the control of the aptamer and/or comparing the activity of the reporter gene in presence of the thiamine or TPP analog used for the screen as compared to the activity of the reporter gene in absence of the thiamine or TPP analog used for the screen.

### Articles of Manufacture and Kits

Also provided are kits or articles of manufacture for use in the methods described herein. In aspects, the kits comprise the compositions described herein (e.g., compositions for delivery of a vector comprising the target gene containing the gene regulation cassette) in suitable packaging. Suitable packaging for compositions (such as ocular compositions for injection) described herein are known in the art, and include, for example, vials (such as sealed vials), vessels, ampules, bottles, jars, flexible packaging (e.g., sealed Mylar or plastic bags), and the like. These articles of manufacture may further be sterilized and/or sealed.

Also provided are kits comprising the compositions described herein. These kits may further comprise instruction(s) on methods of using the composition, such as uses described herein. The kits described herein may further include other materials desirable from a commercial and user standpoint, including buffers, diluents, filters, needles, syringes, and package inserts with instructions for performing the administration of the composition or performing any methods described herein. For example, in some embodiments, the kit comprises an rAAV for the expression of a target gene comprising a gene regulation cassette containing an aptamer sequence described herein, a pharmaceutically acceptable carrier suitable for injection, and one or more of: a buffer, a diluent, a filter, a needle, a syringe, and a package insert with instructions for performing the injections. In some embodiments, the kit is suitable for intraocular injection, intramuscular injection, intravenous injection and the like.

It is to be understood and expected that variations of the compositions of matter and methods herein disclosed can be made by one skilled in the art and it is intended that such modifications are to be included within the scope of the present disclosure. The following Examples further illustrate the invention, but should not be construed to limit the scope of the invention in any way.

All references cited herein are hereby incorporated by reference in their entirety. All nucleotide sequences provided herein are in a 5′ to 3′ orientation unless stated otherwise. A Sequence Listing is filed herewith, the contents of which are incorporated herein by reference in its entirety.

### EXAMPLES

### Example 1: A TPP Aptamer Homologous Sequence Regulates Gene Expression in Mammalian Cells in Response to Thiamine Pyrophosphate (TPP) and Vitamin B1 Analogs

### Experimental Procedures:

Riboswitch construct: Aptamers were synthesized by Integrated DNA Technologies, Inc. The synthesized aptamer sequence, here referred to as aptamer sequence 12C6-1, contains a putative TPP aptamer sequence (AP008955.1/944373-944459; CP030117.1/954080-954166; CP023474.1/977011-977097) with C at 5′ end and a complementary G at 3′ end flanking the putative TPP aptamer sequence: CTGGGGAGTCCTTCATGCGGGGCTGAGAGGATGGAAGCAATCGACCATCGACCC ATTGCACCTGATCCGGATCATGCCGGCGCAGGGAG (SEQ ID NO: 1). Golden Gate cloning strategy (New England Biolabs, NEB) was used to clone the synthesized aptamer sequences into an intron-exon-intron cassette to replace the guanine aptamer in the G17 riboswitch cassette (see SEQ ID NO: 15 recited in WO 2016/126747, which is incorporated herein in its entirety) to generate riboswitch construct Luci-12C6-1.

Transfection: 3.5×10⁴ human embryonic kidney (HEK) 293 cells were plated in a 96-well flat bottom plate the day before transfection. Plasmid DNA (500 ng) was added to a tube or a 96-well U-bottom plate. Separately, TransIT-293 reagent (Mirus; 1.4 L) was added to 50 μL Optimum I media (Life Technologies) and allowed to sit for 5 minutes at room temperature (RT). Then, 50 μL of this diluted transfection reagent was added to the DNA, mixed, and incubated at RT for 20 min. Finally, 7 μL of this solution was added to a well of cells in the 96-well plate. Four hours after transfection, medium containing transfection solution was replaced by medium with either TPP or fursultiamine as aptamer ligands.

Firefly luciferase assay of cultured cells: Twenty-four hours after media change, plates were removed from the incubator, and equilibrated to RT for several minutes on a lab bench, then aspirated. Glo-lysis buffer (Promega, 100 μL, RT) was added, and the plates allowed to remain at RT for at least 5 minutes. Then, the well contents were mixed by 50 μL trituration, and 20 μL of each sample was mixed with 20 μL of bright-glo reagent (Promega) that had been diluted to 10% in glo-lysis buffer. 96 wells were spaced on an opaque white 384-well plate. Following a 5 min incubation at RT, luminescence was measured using a Tecan machine with 500 ms read time. The luciferase activity was expressed as mean arbitrary light units (ALU)±S.D., and fold induction was calculated as the quotient of the luciferase activity obtained from cells with TPP or analog compound treatment divided by the luciferase activity obtained from cells without TPP or analog compound treatment.

### Results:

A TPP aptamer homologous sequence (AP008955.1/944373-944459; CP030117.1/954080-954166; CP023474.1/977011-977097) was identified from a RNA family database RF00059 (http://rfam.xfam.org/family/RF00059), and was tested in the alternative splicing based synthetic aptamer riboswitch system for regulation of target gene expression in response to TPP treatment. This synthetic riboswitch system, as described in WO2016/126747 (incorporated herein by reference in its entirety), contains an intron-alternative exon-riboswitch-intron cassette in which ligand binding to the aptamer portion of the riboswitch controls the accessibility of the 5′ splice site of the 3′ intron, therefore allowing for regulation of the expression of a target gene through modulating alternative splicing (FIG. 1a). The putative TPP aptamer sequence, together with the flanking C at 5′ end and G at 3′ end, was inserted into the intron downstream of the alternative exon containing an in-frame stop codon, generating riboswitch 12C6-1. In this configuration (FIG. 1b), ligand binding presumably brings close the 5′ and 3′ ends of the aptamers sequence which includes the adjacent U1 binding site and its complementary sequence, stabilizing a 9 bp stem structure that sequesters the accessibility of the 5′ splice site, allowing splicing occur between the exons of the transgene and subsequence transgene gene expression. HEK 293 cells were transfected with luciferase construct containing 12C6-1 riboswitch (Luci-12C6-1) and treated with TPP for increased luciferase expression. As shown in FIG. 1c, cells transfected with Luci-12C6-1 construct expressed increased luciferase expression upon treatment with TPP when compared with luciferase expression from cells without TPP treatment. The luciferase expression increased along with the increased concentration of TPP, demonstrating a dose-dependent response to TPP treatment. These results indicate that the putative TPP aptamer indeed responds to TPP treatment and regulates gene expression in a synthetic riboswitch cassette in mammalian cells.

We previously found that TPP responsive aptamers also respond to vitamin B1 analogs (as described in 62/994,135 PTC application). Similarly, we found that the 12C6-1 riboswitch also responded to B1 analogs, such as fursultiamine, and induced luciferase gene expression in a dose-dependent manner (FIG. 1d). Thus, using this TPP aptamer homologous sequence, we have constructed a synthetic mammalian aptamer riboswitch that can regulate transgene expression in the presence or absence of TPP as well as synthetic Vitamin B1 analogs.

### Example 2: Synthetic Riboswitches Comprising Thiamine Pyrophosphate (TPP)-Responsive Aptamers Regulate Gene Expression in Response to Comp. 004

### Experimental Procedure: As Described in Example 1.

### Results

To identify additional synthetic small molecules that potentially bind and activate 12C6-1 riboswitch in mammalian cells, we tested a novel TPP aptamer binding compound, Comp. 004 (KW-62, PCT application number or publication to cite), which was generated by Weeks et al using a fragment-based aptamer ligand discovery approach.

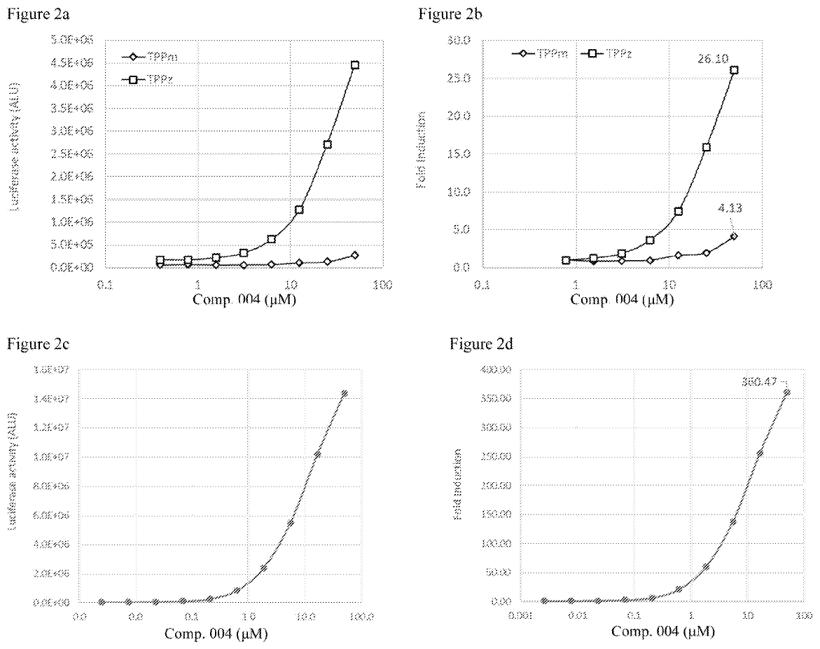

First, the E. coli thiM TPP aptamer, the aptamer that was used in Weeks' work in generating the Comp. 004, was tested in TPPm riboswitch construct (SEQ ID No. 87 as described in 62/994,135) for its response to Comp. 004 in inducing gene expression. To evaluate whether this novel TPP aptamer binder could bind a different TPP aptamer, TPP aptamer from Alishewanella tabrizica thiC gene (Microbiol Res. 2017 January; 195:71-80) was tested in TPPz riboswitch construct (SEQ ID No. 86 as described in 62/994,135). As shown in FIG. 2, both TPPm and TPPz riboswitches regulate luciferase expression in responding to Comp. 004 treatment in dose-dependent manner. This observation indicates that Comp. 004 binds TPP aptamer in mammalian cells. However, these two riboswitches have different Comp. 004-induced riboswitch activity, with TPPz riboswitch construct showing much higher gene regulation activity in responding to Comp. 004 treatment. As shown in FIG. 2a, TPPz riboswitch construct generated 26-fold increase in luciferase expression when treated with 50 μM Comp. 004, whereas TPPm construct expressed only 4.1 increase in luciferase expression at the same concentration of Comp. 004. These results demonstrate that Comp. 004 can activate TPP aptamer riboswitches. Further, the higher dynamic range of TPPz in regulating gene expression suggest that Comp. 004 binds Alishewanella tabrizica thiC aptamer in TPPz riboswitch with higher affinity than with E. Coli thiM TPP aptamer in TPPm riboswitch.

Next, Comp. 004 was tested on 12C6-1 riboswitch in regulating gene expression in mammalian cells. HEK 293 cells were transfected with 12C6-1 riboswitch constructs and treated with Comp. 004 at various concentrations. As shown in FIGS. 2c and 2d, luciferase expression increased upon Comp. 004 treatment, and the induced expression of luciferase is in a dose-dependent manner. The dynamic range of induced gene expression from Luci-12C6-1 is even higher than that of TPPz riboswitch construct, rendering 360-fold increase in luciferase expression in the presence of 50 uM Comp. 004. These results indicate that Comp. 004 can activate this newly developed riboswitch 12C6-1 in regulating gene expression in mammalian cells with high dynamic range.

### Example 3: Generation of Riboswitches Comprising Re-Engineered Aptamer Sequences that have Enhanced Reactivity to Comp. 004

### Experimental Procedure:

Cloning of riboswitch constructs containing 12C6-1 variant aptamer sequences: 12C6-1 aptamer sequence was used as template, and nucleobases were randomized at certain position in the sequence. Aptamers incorporating random mutagenesis were synthesized by Integrated DNA Technologies, Inc. Golden Gate cloning strategy (New England Biolabs, NEB) was used to clone the synthesized aptamer sequences into intron-exon-intron cassette to replace the 12C6-1 aptamer in the Luci-12C6-1 riboswitch construct, generating riboswitch constructs containing variant aptamer sequences.

### Results

To further improve the riboswitch activity in responding to Comp. 004 and related compounds, the aptamer sequence 12C6-1 was subject to mutagenesis to generate aptamer variants, and the riboswitches containing the variant aptamers were screened against Comp. 004 for the ones that have improved dynamic range of induced gene expression (the fold induction), in comparison with the fold induction by parental riboswitch construct Luci-12C6-1. As shown in FIG. 3a in the predicted secondary structure (RNAfold, http://ma.tbi.univie.ac.at/cgi-bin/RNAWebSuite/RNAfold.cgi) of the 12C6-1 aptamer, loop regions or junction regions that do not appear to be involved in helical formation may participate in tertiary structure upon ligand binding. These loop or junction region alone or together with stem region in proximity were chosen for random mutagenesis to generate riboswitches with re-engineered aptamer sequences with improved activity.

Five aptamer libraries N1, N2, N3, N4 and N5 were generated by randomizing nucleotides at positions in J2-4, J2-3/J3-3a/J3a-2/P3, L3a, J4-5/J5-4/P4 and L5 regions of the parent 12C6-1 sequence, respectively (see FIGS. 3a and 3b). Single bacterial colonies were picked and plasmids containing variant riboswitch constructs were screened in HEK 293 cells for improved gene regulation activity in response to 25 M Comp. 004 as compared to parent riboswitch construct Luci-12C6-1.

Nucleobases in the junction region (J2-4) that links P2 and P4 were randomized, generating 4096 variant sequences in library N1. Eighty-two variant aptamers were identified and screened against Comp. 004 (see Table 1 for variant sequences in J2-4). Approximately 93.9% of the identified riboswitch constructs showed decreased riboswitch activity (<250-fold induction) and 17.1% of these 82 riboswitch constructs showed minimum (2- to 2.5-fold induction) or no riboswitch activity (no induction), in inducing luciferase gene expression in comparison with parental 12C6-1, which has an average fold induction of about 300. Constructs with aptamers N1_1F1_2 and N1_2H3 generated more than 300-fold increase in luciferase gene expression, indicating enhanced riboswitch activity compared to parental 12C6-1 (Table 1).

Nucleobases at 6 positions in J2-3/J3-3a/J3a-2/P3, the region that link P2, P3 and P3a, were randomized, generating 4096 variant sequences in library N2. 192 variants were screened for riboswitch activity, with no construct identified as showing riboswitch activity to induce luciferase expression in response to Comp. 004 treatment (see Table 5 for sequence variants in J2-3/J3-3a/J3a-2/P3). Therefore, changes in the selected region did not generate riboswitches with enhanced gene regulation activity, but rather abolished the riboswitch activity in response to Comp. 004.

Nucleobases at 6 positions in the L3a region were randomized, generating 4096 variant sequences in library N3. 85 variant riboswitches were identified and screened against Comp. 004 (see Table 2 for variant sequences in L3a), 94% of which showed decreased riboswitch activity in inducing luciferase gene expression in comparison with parental 12C6-1, and 37.4% of which showed minimum (2- to 2.5-fold induction) or no riboswitch activity (no induction). 1 (N3_G6) out of 85 constructs exhibited 858-fold, and 2 out of 85 showed greater than 400-fold induction in luciferase gene expression, indicating enhanced riboswitch activity than parental 12C6-1 (see Table 2).

Nucleobases at 5 positions in P4/J4-5/J5-4 region were randomized, generating 1024 variant aptamer sequences in library N4. In partial library screening, 864 riboswitches were screened against Comp. 004 treatment, with approximately 46.2% of the screened riboswitch constructs inducing greater than 500-fold increase in luciferase expression in response to Comp. 004 treatment. Among the 183 sequence-verified unique variants, 1 riboswitch (N4-1C11) induced greater than 2000-fold and 19 riboswitches induced greater than 1000-fold increase in luciferase gene expression in response to Comp. 004 treatment, whereas 33 riboswitch constructs showed reduced riboswitch activity in comparison with parental 12C6-1, which provides an average fold induction of about 300 (see Table 3 for variant sequences in P4/J4-5/J5-4).

Nucleobases at 6 positions in L₅ region were randomized, generating 4096 variant sequences in library N5. In partial N5 library screening, 1222 riboswitches were screened against Comp. 004 treatment, with approximately 77.1% of the screened riboswitch constructs inducing greater than 500-fold increase in luciferase expression in response to Comp. 004 treatment. Among the 231 unique variant sequences identified, 5 riboswitches induced greater than 2000-fold and 89 riboswitches induced greater than 1000-fold increase in luciferase gene expression in response to Comp. 004 treatment, whereas 10 riboswitch constructs showed reduced riboswitch activity in comparison with parental 12C6-1 (see Table 4 for variant sequences in L5).

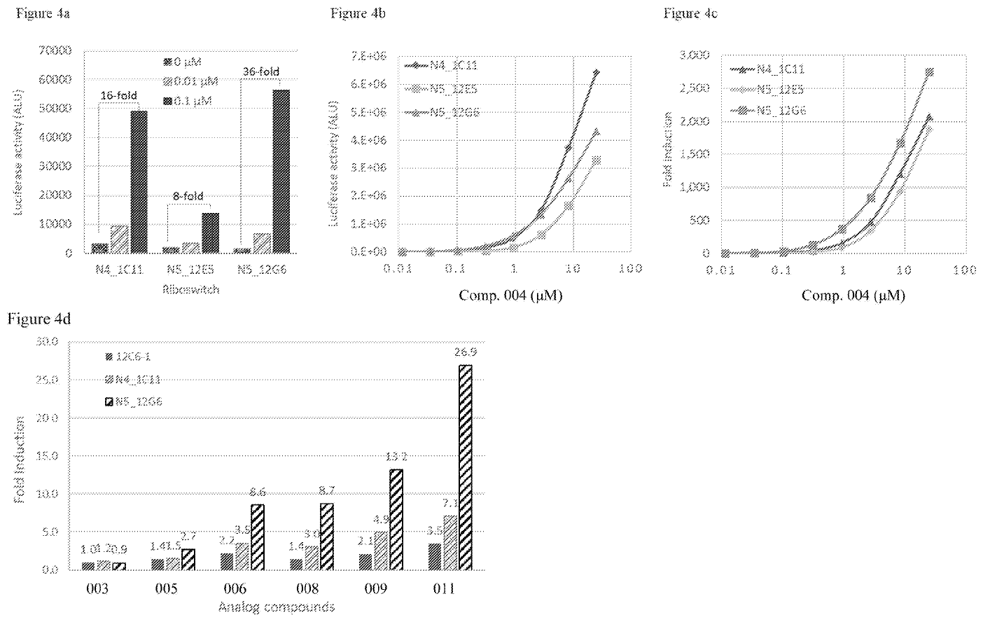

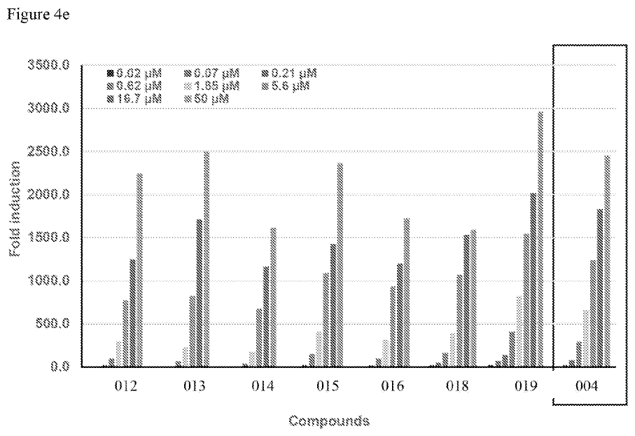

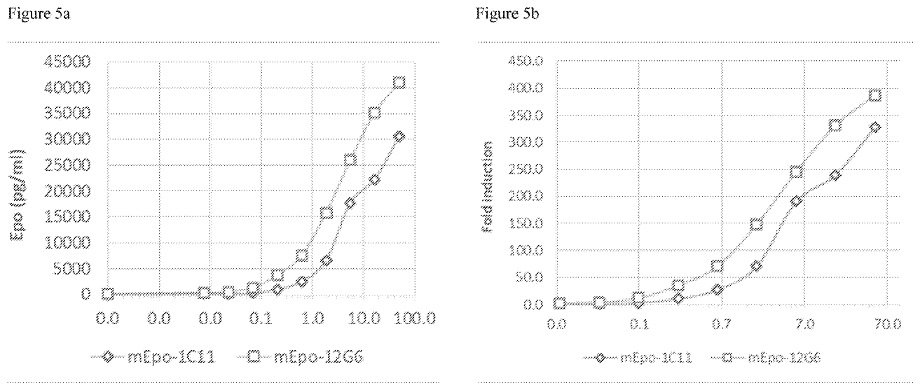

Riboswitch constructs containing re-engineered aptamer sequences N4-1C11, N5-12E5 and N5-12G6 were further validated for their enhanced riboswitch activity. As shown in FIG. 4a, all three riboswitches increased luciferase activity when treated with 0.01 μM Comp. 004, and induced 16-, 8- and 36-fold, respectively, increase in luciferase expression, in response to 0.1 μM Comp. 004 treatment. The induced expression of luciferase increased in a dose-dependent manner (FIG. 4b, 4c).

The parental riboswitch 12C-1 and its derivatives also respond to a series of compounds that are analogous to Comp. 004, with the N5-12G6 riboswitch showing stronger response (FIG. 4d). Additional analogues to Comp. 004 were tested against the 12G6 riboswitch (FIG. 4e and Table B, below) for induction of luciferase expression in HEK293 cells. The structures for these compounds are provided in Table A, and synthesis is described herein.

TABLE B

| | | Fold induction | | |
|---|---|---|---|---|
| Ref. | | 25 μM | 3.3 μM | 2 μM |
| No. | Structure | (HEK) | (HEK) | (HEK) |
| 012 | 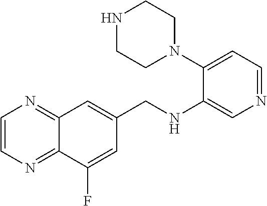 [SMILES: Fc1cc(CNc2cnccc2N2CCNCC2)cc2nccnc12] | 383.1 | | |
| 013 | 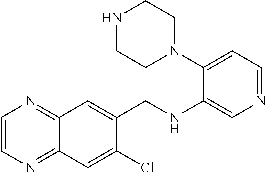 [SMILES: Clc1cc2nccnc2cc1CNc1cnccc1N1CCNCC1] | 314.1 | | |
| 014 | 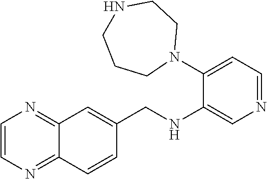 [SMILES: c1cc(N2CCCNCC2)c(NCc2ccc3nccnc3c2)cn1] | 251.1 | | |
| 015 | 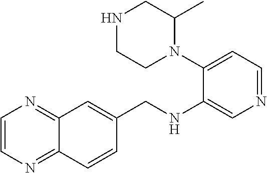 [SMILES: CC1CNCCN1c1ccncc1NCc1ccc2nccnc2c1] | 329.4 | | |
| 016 | 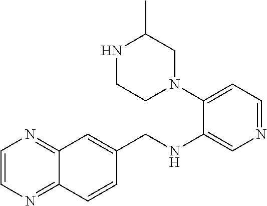 [SMILES: CC1CN(c2ccncc2NCc2ccc3nccnc3c2)CCN1] | 287.1 | | |
| 017 | 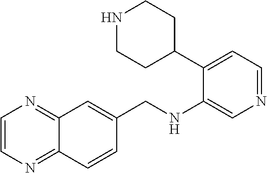 [SMILES: c1cc(C2CCNCC2)c(NCc2ccc3nccnc3c2)cn1] | 130.3 | | |
| 018 | 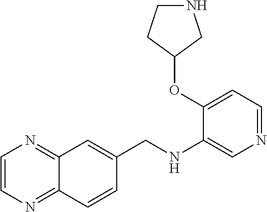 [SMILES: c1cc(OC2CCNC2)c(NCc2ccc3nccnc3c2)cn1] | 286.0 | | |
| 019 | 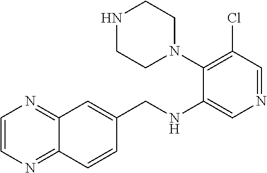 [SMILES: Clc1cncc(NCc2ccc3nccnc3c2)c1N1CCNCC1] | 452.6 | | 60.2 |
| 020 | 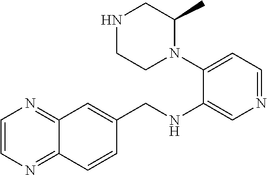 [SMILES: C[C@@H]1CNCCN1c1ccncc1NCc1ccc2nccnc2c1] | | 52 | 31.2 |
| 021 | 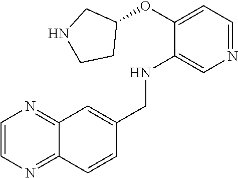 [SMILES: c1cc(O[C@@H]2CCNC2)c(NCc2ccc3nccnc3c2)cn1] | | 62 | 68.4 |
| 022 | 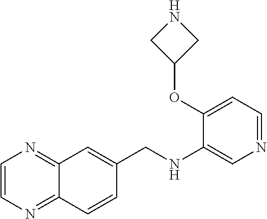 [SMILES: c1cc(OC2CNC2)c(NCc2ccc3nccnc3c2)cn1] | | | 43.2 |
| 023 | 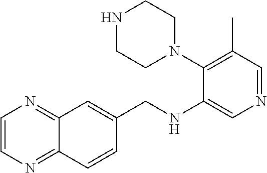 [SMILES: Cc1cncc(NCc2ccc3nccnc3c2)c1N1CCNCC1] | | | 67.4 |
| 024 | 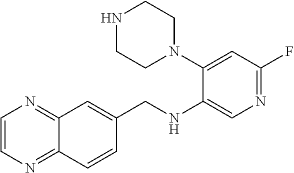 [SMILES: Fc1cc(N2CCNCC2)c(NCc2ccc3nccnc3c2)cn1] | | | 35.1 |
| 025 | 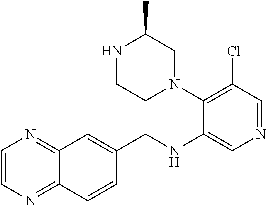 [SMILES: C[C@H]1CN(c2c(Cl)cncc2NCc2ccc3nccnc3c2)CCN1] | | | 42.6 |
| 026 | 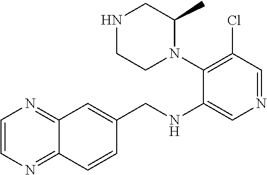 [SMILES: C[C@@H]1CNCCN1c1c(Cl)cncc1NCc1ccc2nccnc2c1] | | | 27.2 |
| 027 | 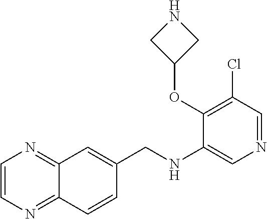 [SMILES: Clc1cncc(NCc2ccc3nccnc3c2)c1OC1CNC1] | | | 49.4 |
| 028 | 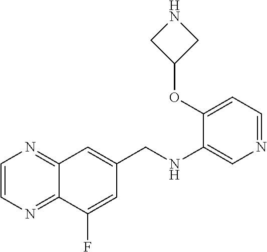 [SMILES: Fc1cc(CNc2cnccc2OC2CNC2)cc2nccnc12] | | | 33.0 |
| 029 | 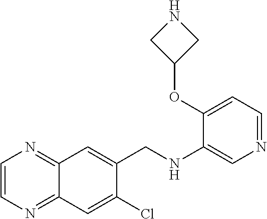 [SMILES: Clc1cc2nccnc2cc1CNc1cnccc1OC1CNC1] | | | 41.3 |
| 030 | 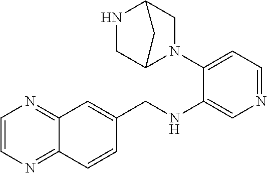 [SMILES: c1cc(N2CC3CC2CN3)c(NCc2ccc3nccnc3c2)cn1] | 82.9 | | |
| 031 | 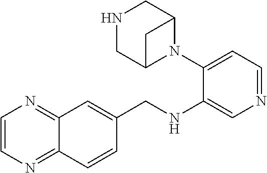 [SMILES: c1cc(N2C3CNCC2C3)c(NCc2ccc3nccnc3c2)cn1] | 17.9 | | |
| 032 | 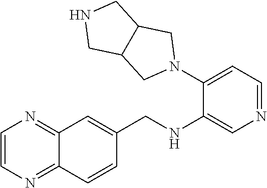 [SMILES: c1cc(N2CC3CNCC3C2)c(NCc2ccc3nccnc3c2)cn1] | 69.4 | | |
| 033 | 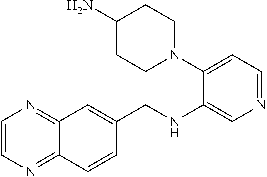 [SMILES: NC1CCN(c2ccncc2NCc2ccc3nccnc3c2)CC1] | 7.8 | | |
| 034 | 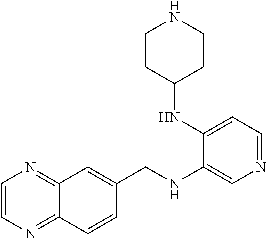 [SMILES: c1cc(NC2CCNCC2)c(NCc2ccc3nccnc3c2)cn1] | 53.2 | | |
| 035 | 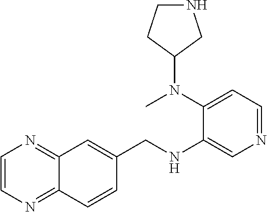 [SMILES: CN(c1ccncc1NCc1ccc2nccnc2c1)C1CCNC1] | 86.4 | | |
| 036 | 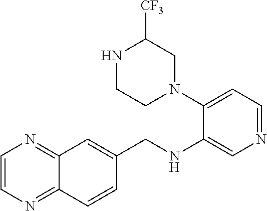 [SMILES: FC(F)(F)C1CN(c2ccncc2NCc2ccc3nccnc3c2)CCN1] | 8.2 | | |
| 037 | 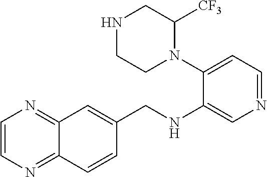 [SMILES: FC(F)(F)C1CNCCN1c1ccncc1NCc1ccc2nccnc2c1] | 6.1 | | |
| 038 | 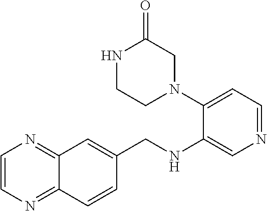 [SMILES: O=C1CN(c2ccncc2NCc2ccc3nccnc3c2)CCN1] | 2.9 | | |
| 039 | 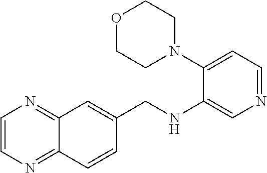 [SMILES: c1cc(N2CCOCC2)c(NCc2ccc3nccnc3c2)cn1] | 6 | | |
| 040 | 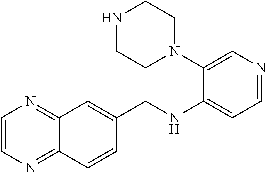 [SMILES: c1cc(NCc2ccc3nccnc3c2)c(N2CCNCC2)cn1] | 69 | | |
| 041 | 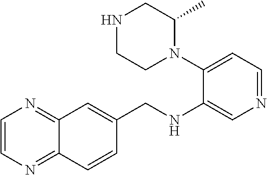 [SMILES: C[C@H]1CNCCN1c1ccncc1NCc1ccc2nccnc2c1] | | 5 @ 3.3 uM | |
| 042 | 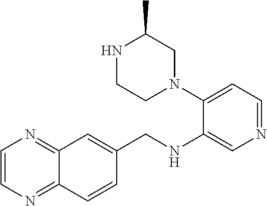 [SMILES: C[C@H]1CN(c2ccncc2NCc2ccc3nccnc3c2)CCN1] | | 29 @ 3.3 uM | |
| 043 | 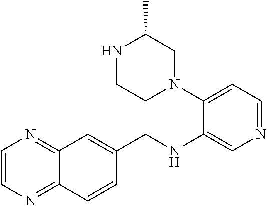 [SMILES: C[C@@H]1CN(c2ccncc2NCc2ccc3nccnc3c2)CCN1] | | 31 @ 3.3 uM | |
| 044 | 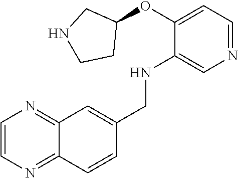 [SMILES: c1cc(O[C@H]2CCNC2)c(NCc2ccc3nccnc3c2)cn1] | | 22 @ 3.3 uM | |
| 045 | 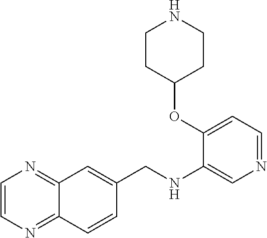 [SMILES: c1cc(OC2CCNCC2)c(NCc2ccc3nccnc3c2)cn1] | | | 12.0 |
| 046 | 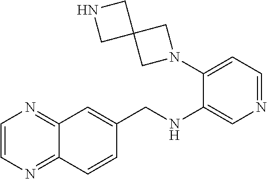 [SMILES: c1cc(N2CC3(CNC3)C2)c(NCc2ccc3nccnc3c2)cn1] | | | 1.6 |
| 047 | 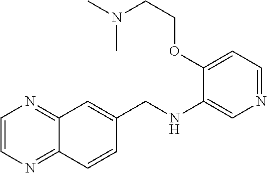 [SMILES: CN(C)CCOc1ccncc1NCc1ccc2nccnc2c1] | | | 2.7 |
| 048 | 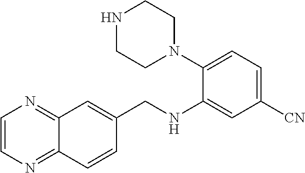 [SMILES: N#Cc1ccc(N2CCNCC2)c(NCc2ccc3nccnc3c2)c1] | | | 1.0 |
| 049 | 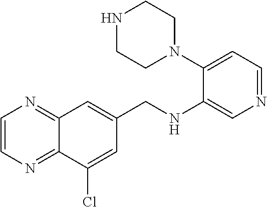 [SMILES: Clc1cc(CNc2cnccc2N2CCNCC2)cc2nccnc12] | | | 5.7 |
| 050 | 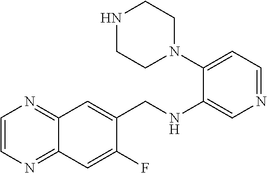 [SMILES: Fc1cc2nccnc2cc1CNc1cnccc1N1CCNCC1] | | | 23.5 |
| 051 | 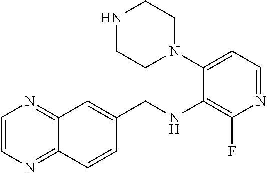 [SMILES: Fc1nccc(N2CCNCC2)c1NCc1ccc2nccnc2c1] | | | 2.7 |
| 052 | 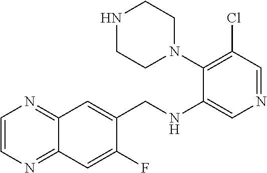 [SMILES: Fc1cc2nccnc2cc1CNc1cncc(Cl)c1N1CCNCC1] | | | 18.9 |
| 053 | 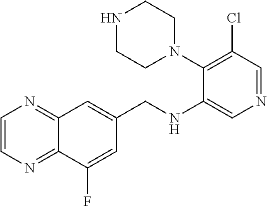 [SMILES: Fc1cc(CNc2cncc(Cl)c2N2CCNCC2)cc2nccnc12] | | | 16.8 |
| 054 | 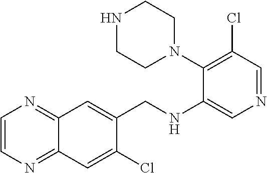 [SMILES: Clc1cc2nccnc2cc1CNc1cncc(Cl)c1N1CCNCC1] | | | 10.6 |
| 055 | 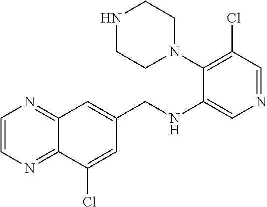 [SMILES: Clc1cncc(NCc2cc(Cl)c3nccnc3c2)c1N1CCNCC1] | | | 4.1 |
| 056 | 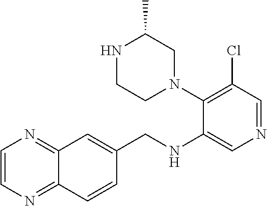 [SMILES: C[C@@H]1CN(c2c(Cl)cncc2NCc2ccc3nccnc3c2)CCN1] | | | 12.9 |
| 057 | 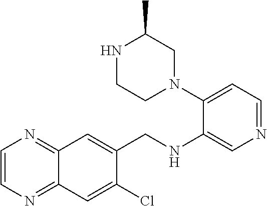 [SMILES: C[C@H]1CN(c2ccncc2NCc2cc3nccnc3cc2Cl)CCN1] | | | 22.3 |
| 058 | 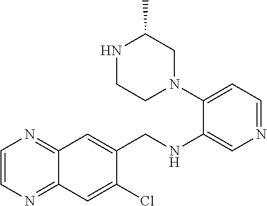 [SMILES: C[C@@H]1CN(c2ccncc2NCc2cc3nccnc3cc2Cl)CCN1] | | | 6.1 |
| 059 | 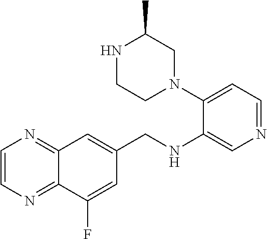 [SMILES: C[C@H]1CN(c2ccncc2NCc2cc(F)c3nccnc3c2)CCN1] | | | 15.8 |
| 060 | 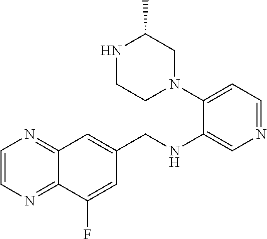 [SMILES: C[C@@H]1CN(c2ccncc2NCc2cc(F)c3nccnc3c2)CCN1] | | | 1.7 |
| 061 | 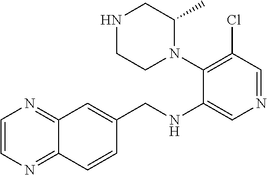 [SMILES: C[C@H]1CNCCN1c1c(Cl)cncc1NCc1ccc2nccnc2c1] | | | 6.0 |
| 062 | 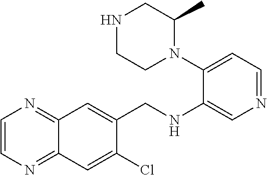 [SMILES: C[C@@H]1CNCCN1c1ccncc1NCc1cc2nccnc2cc1Cl] | | | 8.0 |
| 063 | 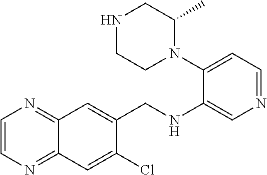 [SMILES: C[C@H]1CNCCN1c1ccncc1NCc1cc2nccnc2cc1Cl] | | | 2.7 |
| 064 | 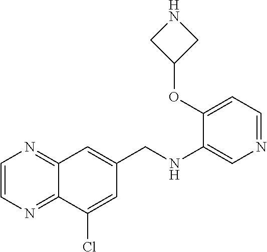 [SMILES: Clc1cc(CNc2cnccc2OC2CNC2)cc2nccnc12] | | | 15.0 |
| 065 | 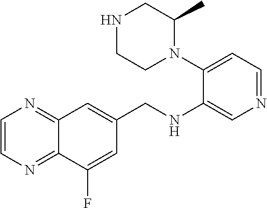 [SMILES: C[C@@H]1CNCCN1c1ccncc1NCc1cc(F)c2nccnc2c1] | | | 21.7 |
| 066 | 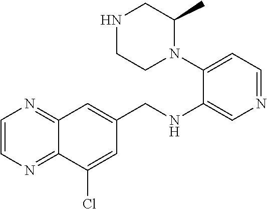 [SMILES: C[C@@H]1CNCCN1c1ccncc1NCc1cc(Cl)c2nccnc2c1] | | | 2.0 |
| 067 | 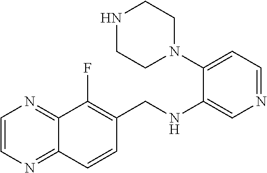 [SMILES: Fc1c(CNc2cnccc2N2CCNCC2)ccc2nccnc12] | | | 3.4 |
| 068 | 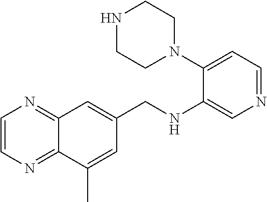 [SMILES: Cc1cc(CNc2cnccc2N2CCNCC2)cc2nccnc12] | | | 4.4 |
| 069 | 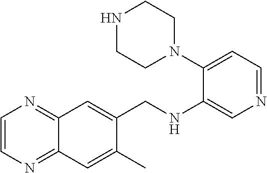 [SMILES: Cc1cc2nccnc2cc1CNc1cnccc1N1CCNCC1] | | | 28.3 |
| 070 | 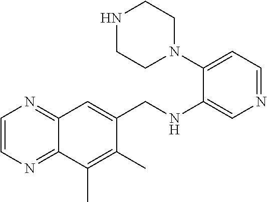 [SMILES: Cc1c(CNc2cnccc2N2CCNCC2)cc2nccnc2c1C] | | | 2.9 |
| 071 | 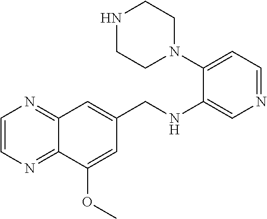 [SMILES: COc1cc(CNc2cnccc2N2CCNCC2)cc2nccnc12] | | | 6.1 |
| 072 | 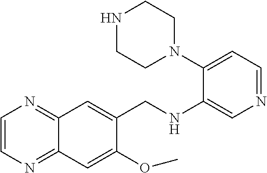 [SMILES: COc1cc2nccnc2cc1CNc1cnccc1N1CCNCC1] | | | 19.6 |
| 073 | 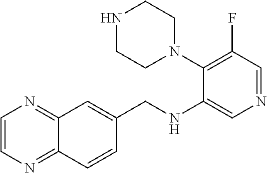 [SMILES: Fc1cncc(NCc2ccc3nccnc3c2)c1N1CCNCC1] | | | 43.9 |
| 074 | 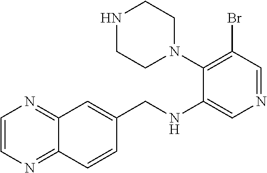 [SMILES: Brc1cncc(NCc2ccc3nccnc3c2)c1N1CCNCC1] | | | 48.6 |
| 075 | 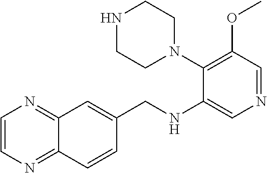 [SMILES: COc1cncc(NCc2ccc3nccnc3c2)c1N1CCNCC1] | | | 18.6 |
| 076 | 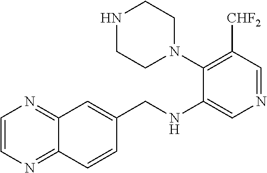 [SMILES: FC(F)c1cncc(NCc2ccc3nccnc3c2)c1N1CCNCC1] | | | 117.4 |
| 077 | 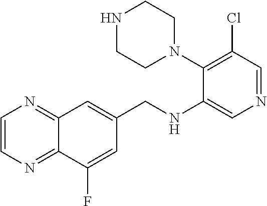 [SMILES: Fc1cc(CNc2cncc(Cl)c2N2CCNCC2)cc2nccnc12] | | | 43.5 |
| 078 | 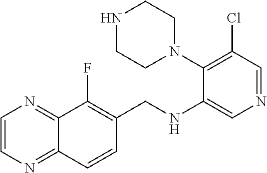 [SMILES: Fc1c(CNc2cncc(Cl)c2N2CCNCC2)ccc2nccnc12] | | | 6.1 |
| 079 | 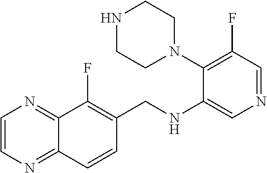 [SMILES: Fc1cncc(NCc2ccc3nccnc3c2F)c1N1CCNCC1] | | | 4.4 |
| 080 | 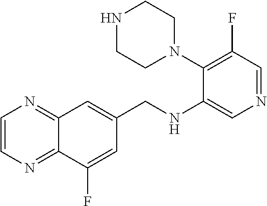 [SMILES: Fc1cncc(NCc2cc(F)c3nccnc3c2)c1N1CCNCC1] | | | 31.2 |
| 081 | 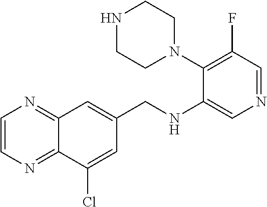 [SMILES: Fc1cncc(NCc2cc(Cl)c3nccnc3c2)c1N1CCNCC1] | | | 3.9 |
| 082 | 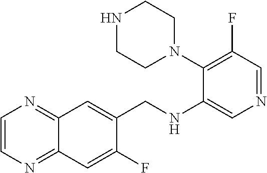 [SMILES: Fc1cc2nccnc2cc1CNc1cncc(F)c1N1CCNCC1] | | | 24.7 |
| 083 | 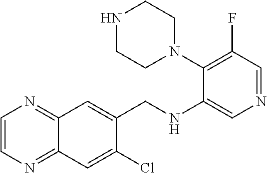 [SMILES: Fc1cncc(NCc2cc3nccnc3cc2Cl)c1N1CCNCC1] | | | 12.8 |
| 084 | 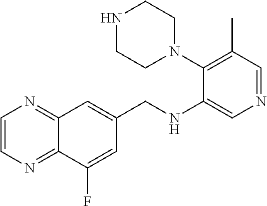 [SMILES: Cc1cncc(NCc2cc(F)c3nccnc3c2)c1N1CCNCC1] | | | 66.4 |
| 085 | 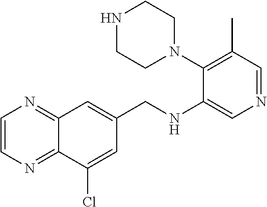 [SMILES: Cc1cncc(NCc2cc(Cl)c3nccnc3c2)c1N1CCNCC1] | | | 5.4 |
| 086 | 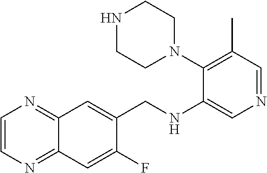 [SMILES: Cc1cncc(NCc2cc3nccnc3cc2F)c1N1CCNCC1] | | | 17.3 |
| 087 | 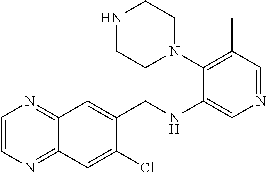 [SMILES: Cc1cncc(NCc2cc3nccnc3cc2Cl)c1N1CCNCC1] | | | 20.9 |
| 088 | 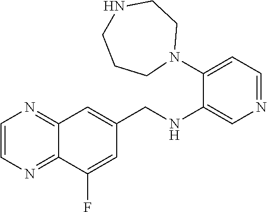 [SMILES: Fc1cc(CNc2cnccc2N2CCCNCC2)cc2nccnc12] | | | 2.6 |
| 089 | 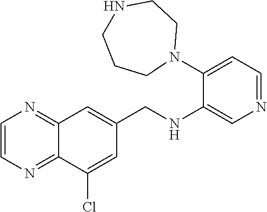 [SMILES: Clc1cc(CNc2cnccc2N2CCCNCC2)cc2nccnc12] | | | 4.8 |
| 090 | 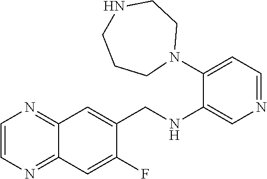 [SMILES: Fc1cc2nccnc2cc1CNc1cnccc1N1CCCNCC1] | | | 15.0 |
| 091 | 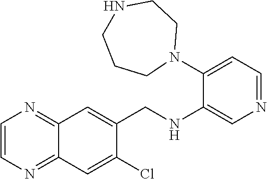 [SMILES: Clc1cc2nccnc2cc1CNc1cnccc1N1CCCNCC1] | | | 28.4 |
| 092 | 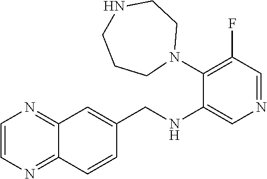 [SMILES: Fc1cncc(NCc2ccc3nccnc3c2)c1N1CCCNCC1] | | | 34.9 |
| 093 | 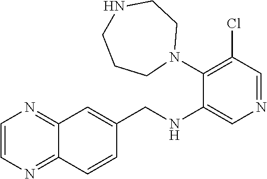 [SMILES: Clc1cncc(NCc2ccc3nccnc3c2)c1N1CCCNCC1] | | | 30.7 |
| 094 | 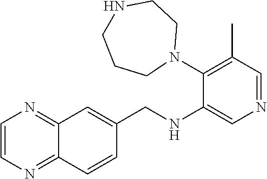 [SMILES: Cc1cncc(NCc2ccc3nccnc3c2)c1N1CCCNCC1] | | | 35.1 |
| 095 | 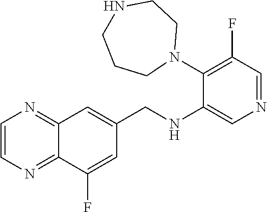 [SMILES: Fc1cncc(NCc2cc(F)c3nccnc3c2)c1N1CCCNCC1] | | | 8.9 |
| 096 | 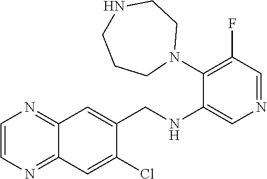 [SMILES: Fc1cncc(NCc2cc3nccnc3cc2Cl)c1N1CCCNCC1] | | | 16.2 |
| 097 | 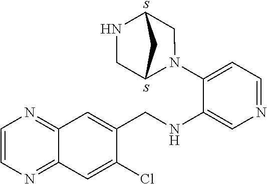 | | | 2.6 |
| 098 | 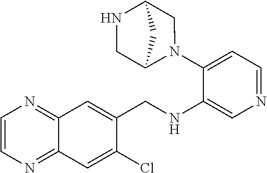 [SMILES: Clc1cc2nccnc2cc1CNc1cnccc1N1C[C@H]2C[C@@H]1CN2] | | | 4.5 |
| 099 | 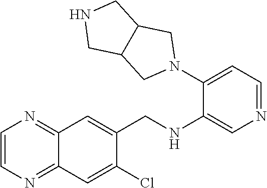 [SMILES: Clc1cc2nccnc2cc1CNc1cnccc1N1CC2CNCC2C1] | | | 8.2 |
| 100 | 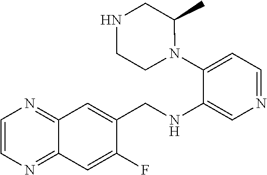 [SMILES: C[C@@H]1CNCCN1c1ccncc1NCc1cc2nccnc2cc1F] | | | 3.5 |
| 101 | 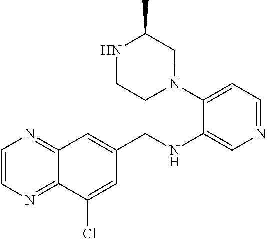 [SMILES: C[C@H]1CN(c2ccncc2NCc2cc(Cl)c3nccnc3c2)CCN1] | | | 2.2 |
| 102 | 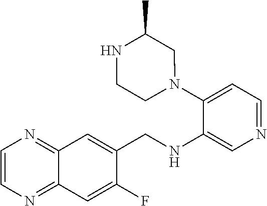 [SMILES: C[C@H]1CN(c2ccncc2NCc2cc3nccnc3cc2F)CCN1] | | | 13.6 |
| 103 | 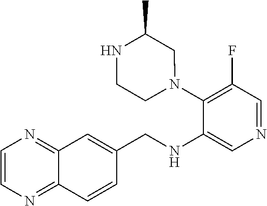 [SMILES: C[C@H]1CN(c2c(F)cncc2NCc2ccc3nccnc3c2)CCN1] | | | 23.3 |
| 104 | 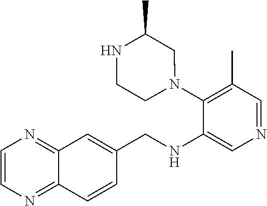 [SMILES: Cc1cncc(NCc2ccc3nccnc3c2)c1N1CCN[C@@H](C)C1] | | | 33.3 |
| 105 | 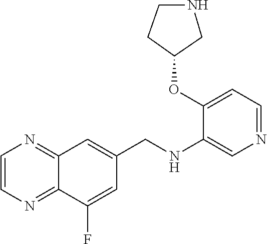 [SMILES: Fc1cc(CNc2cnccc2O[C@@H]2CCNC2)cc2nccnc12] | | | 37.7 |
| 106 | 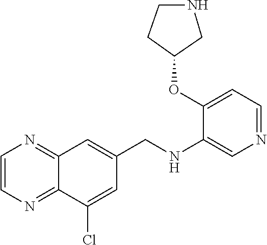 [SMILES: Clc1cc(CNc2cnccc2O[C@@H]2CCNC2)cc2nccnc12] | | | 22.2 |
| 107 | 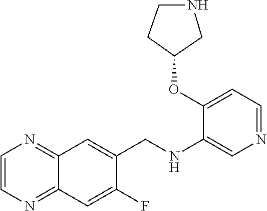 [SMILES: Fc1cc2nccnc2cc1CNc1cnccc1O[C@@H]1CCNC1] | | | 48.7 |
| 108 | 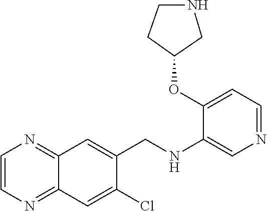 [SMILES: Clc1cc2nccnc2cc1CNc1cnccc1O[C@@H]1CCNC1] | | | 37.5 |
| 109 | 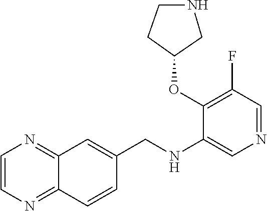 [SMILES: Fc1cncc(NCc2ccc3nccnc3c2)c1O[C@@H]1CCNC1] | | | 54.6 |
| 110 | 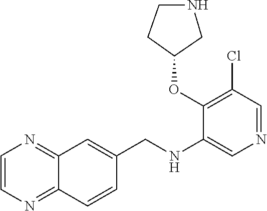 [SMILES: Clc1cncc(NCc2ccc3nccnc3c2)c1O[C@@H]1CCNC1] | | | 43.1 |
| 111 | 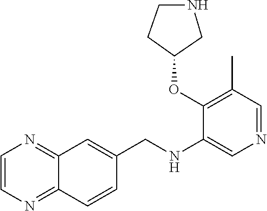 [SMILES: Cc1cncc(NCc2ccc3nccnc3c2)c1O[C@@H]1CCNC1] | | | 74.0 |
| 112 | 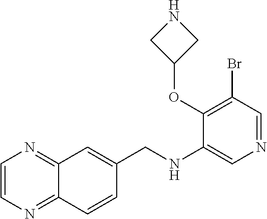 [SMILES: Brc1cncc(NCc2ccc3nccnc3c2)c1OC1CNC1] | | | 40.9 |
| 113 | 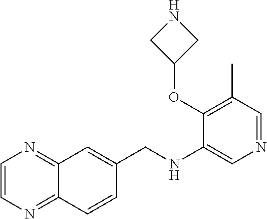 [SMILES: Cc1cncc(NCc2ccc3nccnc3c2)c1OC1CNC1] | | | 31.9 |
| 114 | 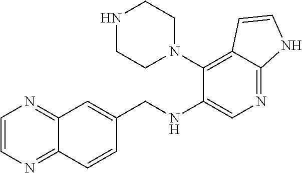 [SMILES: c1cnc2cc(CNc3cnc4[nH]ccc4c3N3CCNCC3)ccc2n1] | | | 60.1 |
| 115 | 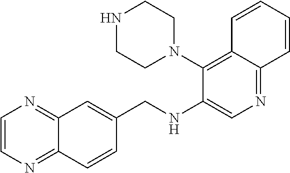 [SMILES: c1ccc2c(N3CCNCC3)c(NCc3ccc4nccnc4c3)cnc2c1] | | | 79.0 |
| 116 | 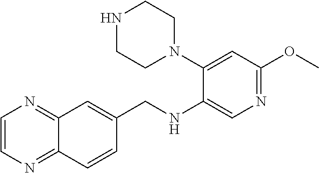 [SMILES: COc1cc(N2CCNCC2)c(NCc2ccc3nccnc3c2)cn1] | | | 50.0 |
| 117 | 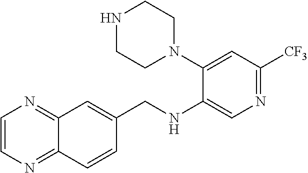 [SMILES: FC(F)(F)c1cc(N2CCNCC2)c(NCc2ccc3nccnc3c2)cn1] | | | 30.6 |
| 118 | 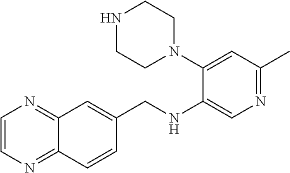 [SMILES: Cc1cc(N2CCNCC2)c(NCc2ccc3nccnc3c2)cn1] | | | 26.2 |
| 119 | 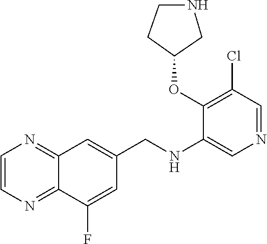 [SMILES: Fc1cc(CNc2cncc(Cl)c2O[C@@H]2CCNC2)cc2nccnc12] | | | 32.5 |
| 120 | 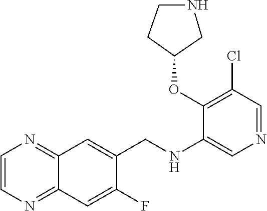 [SMILES: Fc1cc2nccnc2cc1CNc1cncc(Cl)c1O[C@@H]1CCNC1] | | | 24.7 |
| 121 | 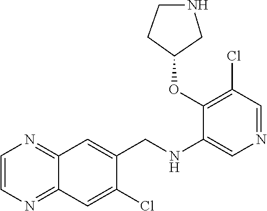 [SMILES: Clc1cc2nccnc2cc1CNc1cncc(Cl)c1O[C@@H]1CCNC1] | | | 28.2 |
| 122 | 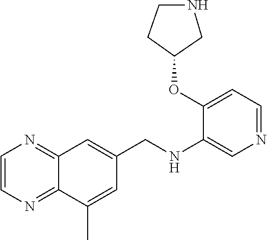 [SMILES: Cc1cc(CNc2cnccc2O[C@@H]2CCNC2)cc2nccnc12] | | | 35.8 |
| 123 | 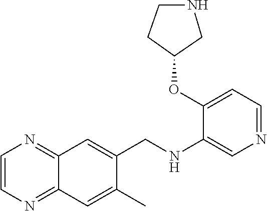 [SMILES: Cc1cc2nccnc2cc1CNc1cnccc1O[C@@H]1CCNC1] | | | 83.9 |
| 124 | 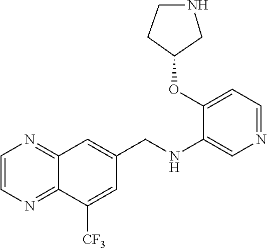 [SMILES: FC(F)(F)c1cc(CNc2cnccc2O[C@@H]2CCNC2)cc2nccnc12] | | | 3.0 |

These results indicate that sequence changes introduced in P4/J4-5/J5-4 or in L5 region significantly improved the riboswitch activity against Comp. 004. The observation that wide range of changes improved riboswitch activity (46.2% in N4 library and 77.1% in N5 library exhibited greater than 500-fold induction) suggests that nucleobases in these regions are not in direct contact with ligand, but rather involved in forming tertiary structure. Thus, through random mutagenesis in selected region of natural sequence, we have developed riboswitches with re-engineered aptamers sequences that are highly responsive to synthetic Comp. 004 and its analog compounds and regulate gene expression with high dynamic range in mammalian cells.

### Example 4: Synthetic Riboswitch Regulates Expression of Various Target Genes in Response to Comp. 004 in Mammalian Cells

### Experimental Procedures:

Riboswitch constructs: The alternative splicing riboswitch cassette containing aptamers N5-12G6 or N4-1C11 was inserted between nucleotide position 307 and 308 in the mouse erythropoietin cDNA sequence, generating constructs mEpo-12G6 and mEpo-1C11. Expression of the erythropoietin gene was driven by CASI promoter. The intron-exon-intron cassette without aptamer sequence was inserted at the same position in the cDNA of mEpo gene to create construct mEpo-Con1, serving as a control for constitutive target gene expression. N5-12G6 riboswitch cassette was inserted between nucleotide position 424 and 425 in the cDNA of human growth hormone (hGH) gene driven under CMV promoter.

Enzyme-linked immunosorbent assay (ELISA) for mouse erythropoietin (mEpo): AML-12 cells or C2C12 cells were transfected as described in Example 1 with TransIT-X₂ transfection reagent (Mirus Bio). Four hours after transfection, the transfected cells were treated with or without Comp. 004 at the indicated doses. The supernatants from the transfected cells were collected 24 hours after compound treatment and were subjected to ELISA for the detection of mEpo in the supernatant following the manufacturer's instruction (R&D).

ELISA for human growth hormone (hGH): HEK 293 cells were transfected as described in Example 1 with TransIT-293 transfection reagent (Mirus Bio). Four hours after transfection, the transfected cells were treated with or without Comp. 004 at the indicated doses. The supernatants from the transfected cells were collected 24 hours after Comp. 004 treatment and were subjected to ELISA for the detection of hGH in the supernatant following the manufacturer's instruction (R&D Systems).

### Results

As discussed in Example 3, isolated riboswitches comprising re-engineered aptamer sequences efficiently regulate expression of the reporter protein luciferase in response to various concentration of Comp. 004. To test the ability of the newly isolated aptamer riboswitches to regulate expression of other target genes, riboswitch cassette containing re-engineered aptamer sequences N5-12G6 and N4-1C11 were inserted into the cDNA sequence of murine erythropoietin (mEpo) and the cDNA sequence of human growth hormone gene (hGH), generating regulatable constructs for these two genes.

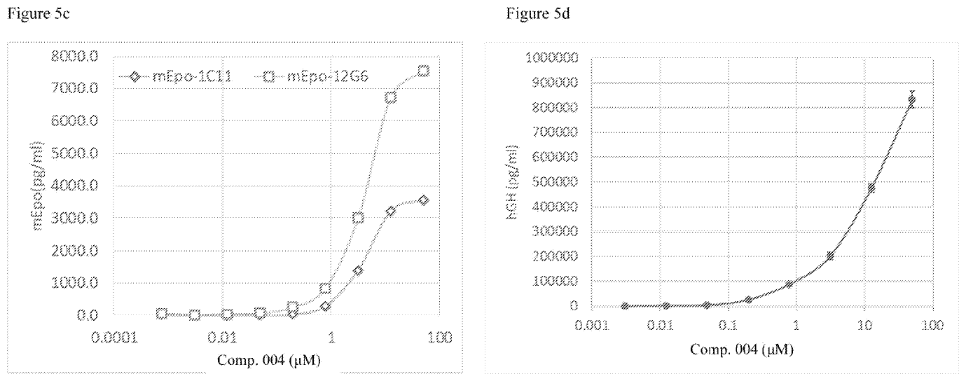

First, the ability of riboswitches comprising aptamers N5-12G6 and N4-1C11 to regulate mEpo expression was examined in the mouse liver cell line AML12. As shown in FIG. 5a, in the absence of Comp. 004, cells transfected with construct mEpo-12G6 or construct mEpo-1C11 expressed very low levels of mEpo. However, upon treatment with Comp. 004, expression of mEpo was enhanced in AML12 cells in a dose-dependent manner. In response to treatment of 1.85 μM of Comp. 004, expression of mEpo was induced by about 148-fold from cells expressing construct mEpo-12G6 and 71-fold from cells expressing construct mEpo-1C11, when compared to expression in absence of Comp. 004 (see FIG. 5b). The riboswitch regulatable mEpo constructs were also tested in mouse myoblast cell line C2C12 cells. Consistent with the observation in AML12 cells, the mEpo expression was very low in the absence of Comp. 004 and was induced upon treatment of Comp. 004 in a dose dependent manner (FIG. 5c).

The riboswitch activity in regulating transgene expression was further tested in human growth hormone (hGH) gene in HEK 293 cells. In the absence of Comp. 004, cells transfected with hGH-12G6 construct expressed about 0.83 ng/ml of hGH. In contrast in the transfected cells that were treated with Comp. 004, the level of hGH expression is significantly increased. With 3.1 μM Comp. 004 treatment, cells expressed 202 ng/ml of hGH, approximately 243-fold of the hGH expression from cells without compound treatment (FIG. 5d). This enhanced expression increased along with the increase of the concentration of Comp. 004, indicating the dose-dependence in the riboswitch regulated gene expression in human cells.

These results demonstrate that the ability of riboswitches comprising re-engineered aptamer sequences to induce gene expression in response to small molecules is not restricted to specific target gene sequences or to a specific cell type, indicating a general applicability of these aptamer riboswitches in regulating target gene expression.

### Example 5: Synthetic Riboswitches Regulate Gene Expression In Vivo in Mice

To assess the ability of engineered aptamers to induce and regulate gene expression in vivo, mice were transduced with an adeno-associated viral vector (AAV) carrying an engineered riboswitch, which was inserted into the gene for the reporter protein luciferase.

### Experimental Procedures:

AAV2.8 viralparticle production: The AAV8 particles used for the transduction of mice comprised a viral genome derived from AAV2 and a capsid derived from AAV8. The firefly luciferase gene containing an intron-exon-intron cassette with (1) a non-regulatable intron cassette without aptamer (“Luci-Con1”), (2) a riboswitch cassette comprising aptamer N5-12G6 (“Luci-12G6”), respectively, was cloned into an AAV2 plasmid vector. Expression of the luciferase gene was driven by CAS promoter which includes CMV and ubiquitin C enhancer elements and the chicken β-actin promoter. The viral vector was packaged into AAV8 capsid and produced following manufacture's protocol (Vigene Biosciences).

Animal studies: For inducible luciferase study, female Balb/c mice received a single tail vein injection or single intra-muscular injection in hind limb quadricep of 5×10¹⁰, 1.0×10¹¹ or 2.5×10¹¹ genome copies (GC) of the receptive AAV8 viral particle. Comp. 004 was formulated in 0.5% methylcellulose (MC): 0.25% Tween® 80 in deionized (DI) water for oral administration. 30 days after AAV vector delivery, mice were treated orally via oral gavage with 10 mg/kg Comp. 004. Luciferase activity was measured the day prior to drug dosing, as well as 6 h, 24 h, 48 h after drug dosing. After the first oral administration of Comp. 004, the mice were subjected to two additional rounds of dosing and imaging cycles as follows: Day 37 (post AAV administration): 30 mg/kg; day 44: 100 mg/kg.

For regulated mouse erythropoietin (mEpo) study, each female Balb/c mouse was injected in the quadricep muscle with 1.0×10¹¹, 5×10¹⁰, 1×10¹⁰, or 5×10⁹ GC of AAV8 vectors containing riboswitch N5-12G6 regulated mEpo gene (AAV8.mEpo.12G6). 5 weeks post AAV injection, mice were treated with Comp. 004 formulated in 0.5% methylcellulose (MC): 0.25% Tween® 80 in deionized (DI) water via oral gavage. 16 hours post oral dosing, mice were subjected to submandibular blood collection. 10 fold diluted serum was used to measure mouse serum Epo using ELISA (Invitrogen).

Chronic kidney disease-associated anima: male C57Bl/6 mice were injected intramuscularly with 2.5×10¹⁰ vg or 1.0×10¹⁰ vg of AAV8.mEpo.12G6 per mouse. One week post AAV injection, mice were treated daily with 50 mg/kg adenine (Sigma) via oral gavage for total 28 treatment in 5 weeks. Hematocrit was measured after Adenine treatment and before small molecule inducer treatment and monitored every 7 to 10 days post daily small molecule inducer oral dosing.

Noninvasive live animal bioluminescence imaging: Before imaging, mice were anesthetized with 2% isoflurane and injected with 150 mg/kg body weight of D-luciferin luciferase substrate. At the indicated time point post drug dosing, images were taken within 10 minutes after luciferin injection using IVIS® SpectrumCT (Perkin Elmer, MA). Luciferase activity was expressed as mean photon/s±S.D. (n=5). The fold induction of luciferase gene expression was calculated as the quotient of photon/s obtained from mice treated with Comp. 004 divided by the value obtained from mice the day before compound treatment.

### Results

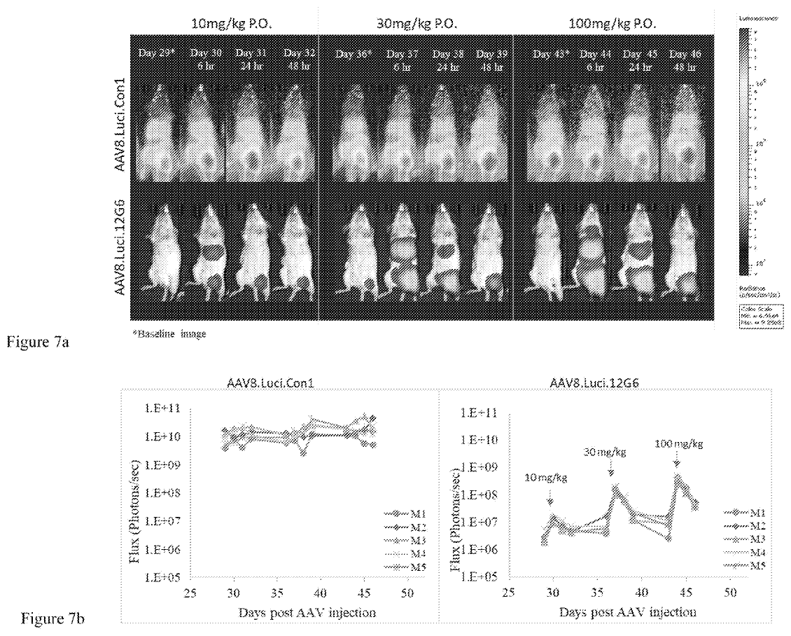

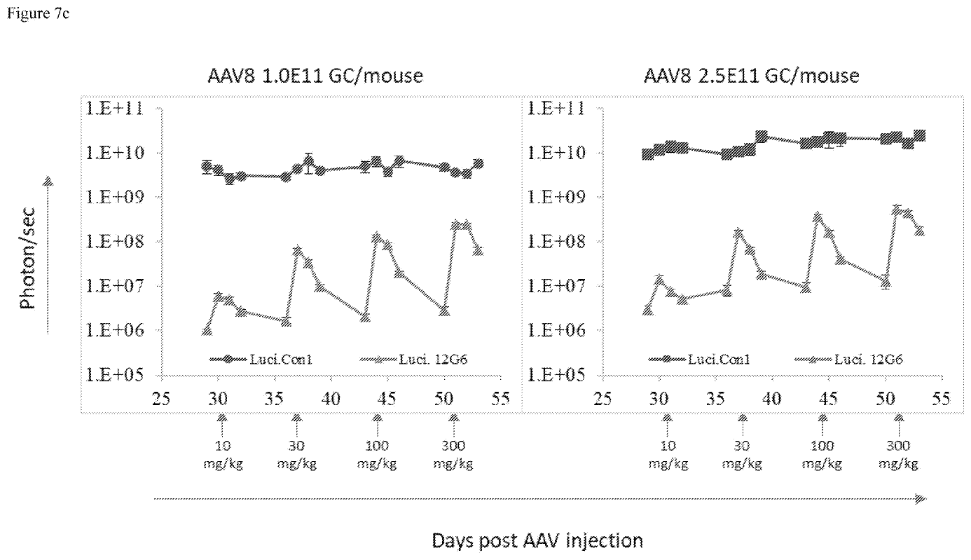

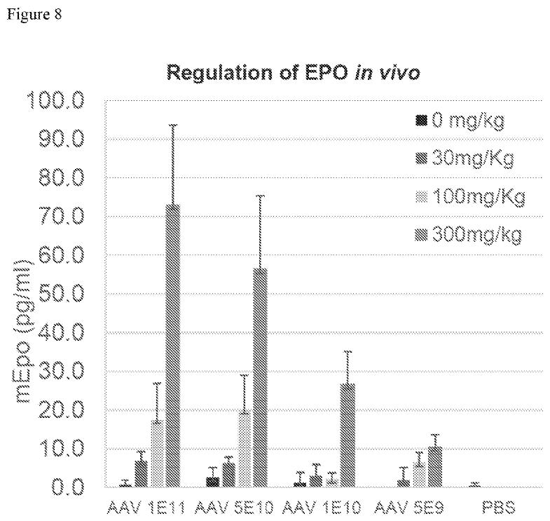

To test the riboswitch in regulating gene expression in animals, AAV8 vectors harboring luciferase gene with or without riboswitch were delivered into mice intravenously. Mice were treated with compound via oral gavage 4 weeks post AAV injection. 6 hours after a single dose of compound (10 mg/kg) treatment, luciferase activity was significantly increased in mice injected AAV vectors containing a luciferase gene comprising riboswitch 12G6 when compared with the luciferase signal prior to compound treatment, whereas the luciferase expression did not change significantly after compound administration in the group of mice injected with the same dose of non-regulatable control vector Con1 (see FIGS. 6a and 6b). With single administration of the compound inducer, the induced luciferase activity was highest at 6 hr post dosing, and decreased at 24 hr. By 48 hr, the luciferase signal returned to baseline level (prior to dosing), indicating the on-and-off state of transgene expression in the presence and absence of the compound inducer and the reversibility of the riboswitch gene regulation system. Subsequent dosing with higher doses in the same mice induced further elevated luciferase signal, indicating dose dependency. Similar results were observed in the mice injected intramuscularly with AAV8.Luci.12G6 vector (FIGS. 7a and 7b).

Luciferase expression from the AAV8.Luci.12G6 exhibited tighter regulation with lower background expression levels in absence of Comp. 004, while luciferase expression from the AAV8.Luci.1B6 exhibited looser regulation with higher background expression levels in absence of Comp. 004, but also higher peak luciferase expression in response to Comp. 004 (FIGS. 6c and 7c).

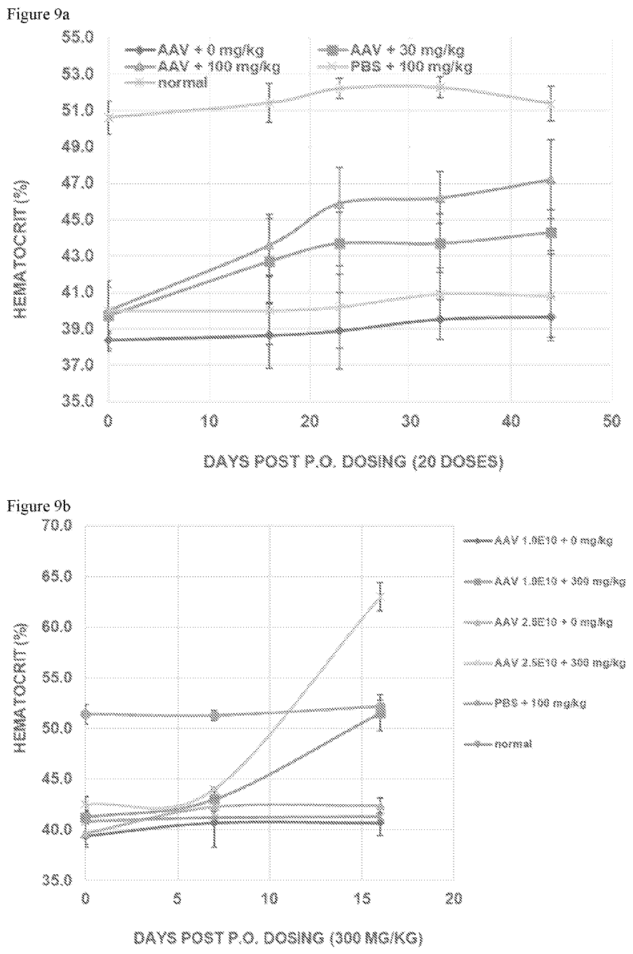

The ability of riboswitch in regulating gene expression in animal was further evaluated using mouse erythropoietin gene (mEpo). Mice were injected in the muscle with 1×10¹¹ GC of AAV8 vectors containing the mEpo gene with 12G6 riboswitch cassette. In mice treated with 30 mg/kg Compd. 004, the serum vector-expressed mEpo was elevated when compared to mice without compound dosing. Moreover, the serum vector expressed mEpo level was further elevated with higher doses of compound treatment and amount of AAV administered, indicating a dose-dependent increase in transgene expression along the increase of the compound inducer (FIG. 8). The effect of riboswitch-regulated expression of Epo on hematocrit was evaluated in a mouse model of chronic kidney disease (CKD)-associated anemia. After 20 doses of compound 004 by oral administration, the hematocrit of anemic mice was increased, with the biggest increase in the 100 mg/kg dose group. However, the hematocrits of anemic mice injected with AAV8.mEpo.12G6 but were not treated with compound inducer did not increase, remaining the same hematocrit as that from anemic mice without delivered AAV8.mEpo.12G6 (FIG. 9a). When mice treated with higher compound dose at 300 mg/kg for 15 days and 10 doses, the hematocrit was restored to normal level in the mouse group injected with lower AAV dose (1×10¹⁰ vg per mouse). In contrast, the hematocrits of mice injected with relatively higher AAV dose (2.5×10¹⁰ vg per mouse) exceeded the normal hematocrit level. These results indicate that Epo was induced from the delivered AAV vector after riboswitch inducer treatment and the induced Epo stimulated erythropoiesis leading to hematocrit increase in anemic animal.

These results demonstrate that riboswitches comprising re-engineered aptamer sequences can regulate target gene expression through orally administered small molecule inducer in a dose-dependent manner in vivo in liver and in muscle These results further demonstrate that the newly developed aptamer riboswitches function in regulating therapeutic genes such as erythropoietin.

### Example 6. Synthetic Riboswitches Regulate Parathyroid Hormone In Vivo in Mice

### Experimental Procedures:

Riboswitch constructs: Alternative splicing riboswitch cassette containing aptamers N5-12G6 was inserted between nucleotide position 181 and 182 in human parathyroid hormone (hPTH) cDNA sequence, generating constructs hPTH-12G6. Expression of the erythropoietin gene was driven by CASI promoter.

Enzyme-linked immunosorbent assay (ELISA) for human PTH: HEK 293 cells were transfected as described in Example 1 with TransIT-293 transfection reagent (Mirus Bio). Four hours after transfection, the transfected cells were treated with or without Compound 004 at the indicated doses. The supernatants from the transfected cells were collected 48 hours after compound treatment and were subjected to ELISA for the detection of human PTH in the supernatant following the manufacturer's instruction (Abcam).

AAV2.9 viral particle production: The AAV9 particles used for the transduction of mice comprised a viral genome derived from AAV2 (ITR) and a capsid derived from AAV9. The hPTH-12G6 was cloned into AAV plasmid backbone with CASI promoter, and the AAV plasmid was packaged into AAV9 capsid, generating vector AAV9.hPTH-12G6 (Signagen)

Animal study: C57BL/6 mice were injected intramuscularly with AAV9.hPTH-12G6 at 2.5×10¹¹ viral genome (VG) per mouse of the AAV9 viral particle into both quadriceps. Compound 004 was formulated in 0.5% methylcellulose (MC): 0.25% Tween® 80 in deionized (DI) water for oral administration. 30 days after AAV vector delivery, mice were treated orally via oral gavage with 0 mg/kg, 30 mg/kg, 100 mg/kg or 300 mg/kg Comp. 004 for 3 days.

### Results

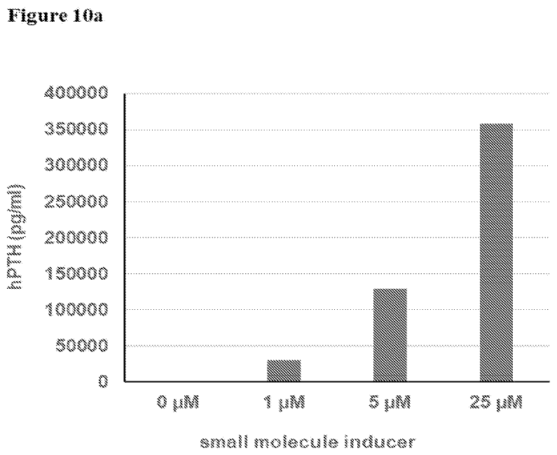

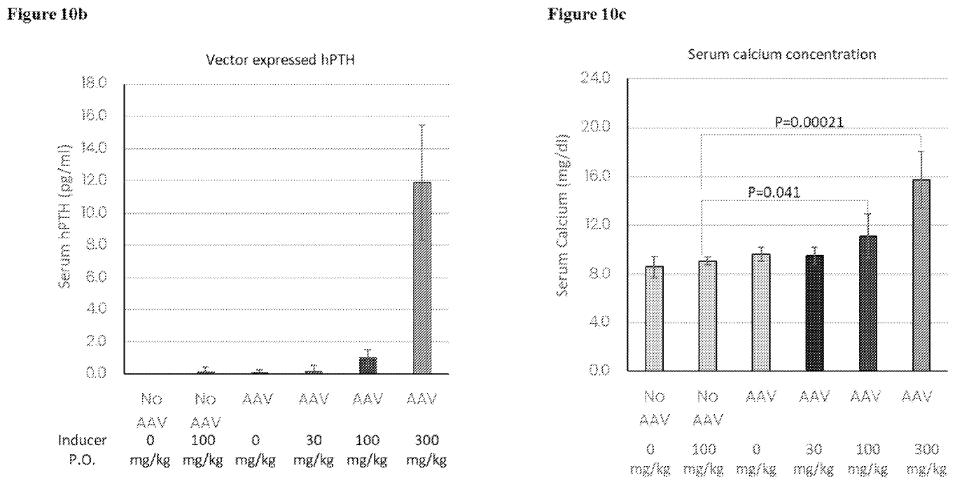

As with luciferase gene or Epo gene, riboswitch 12G6 regulated hPTH expression in dose dependent manner (FIG. 10a). When this regulated hPTH was delivered into mice via AAV vector, Compound 004 treatment induced dose-dependent production of hPTH (FIG. 10b) in mice and accordingly inducing the increase in the serum calcium concentration (FIG. 10c). These results, together with the regulated Epo expression in CKD-anemia, demonstrate that a riboswitch comprising an aptamer disclosed herein can control the expression of therapeutic genes in animal in response to a small molecule ligand (inducer) disclosed herein.

### Examples 7 to 24

### Experimental

All solvents and reagents were obtained commercially and used as received. ¹H NMR spectra were recorded on a Bruker instrument (300 MHz or 400 MHz) in the cited deuterated solvents. Chemical shifts are given in ppm, and coupling constants are in hertz. All final compounds were purified by flash chromatography using 220-400 mesh silica gel or reverse-phase HPLC with CH₃CN/water as the solvents. Thin-layer chromatography was done on silica gel 60 F-254 (0.25-nm thickness) plates. Visualization was accomplished with UV light and/or 10% phosphomolybdic acid in ethanol. Nominal (low resolution) mass spectra were acquired on either a Waters LCT or an Applied Biosystems API 3000 mass spectrometer. High resolution mass spectra (HRMS) were acquired on either a Waters LCT or an Agilent TOF mass spectrometer. All other LC-MS experiments were done on an Agilent 1100 HPLC coupled with an Agilent single quadrupole mass spectrometer. Compound purity was determined by a LC-MS with 230 nM and 254 nM wavelengths. All final compounds reported here have purity≥95%.

### Example 7

### N-((8-Fluoroquinoxalin-6-yl)methyl)-4-(piperazin-1-yl)pyridin-3-amine (Comp. 012)

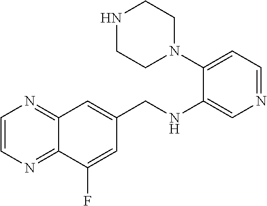 [SMILES: Fc1cc(CNc2cnccc2N2CCNCC2)cc2nccnc12]

### Step 1. 5-Fluoro-7-vinylquinoxaline

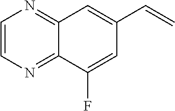

A mixture of 7-bromo-5-fluoroquinoxaline (814 mg, 3.59 mmol, 1.00 equiv), potassium trifluoro (vinyl) boranuide (961 mg, 7.17 mmol, 2.00 equiv), Pd(dppf)Cl₂·CH₂Cl₂ (586 mg, 717 μmol, 0.20 equiv), Cs₂CO₃ (2.34 g, 7.17 mmol, 2.00 equiv) in dioxane (8.00 mL) and H₂O (1.60 mL) was degassed and purged with N₂ for 3 times, and then the mixture was stirred at 100° C. for 1 h under N₂ atmosphere, quenched with water (5.00 mL), and extracted with EtOAc (5.00 mL×2). The reaction organic layers were washed with brine (5.00 mL), dried with Na₂SO₄, filtered, and concentrated in vacuum. The residue was purified by column chromatography (SiO₂; petroleum ether:ethyl acetate=1:0 to 5:1, R_{f}=0.60) to provide the title compound (0.536 g, 85.8%) as a white solid. MS (ES+) m/e 175.1 (M+H)⁺.

### Step 2. 8-Fluoroquinoxaline-6-carbaldehyde

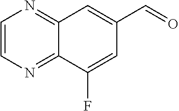 [SMILES: O=Cc1cc(F)c2nccnc2c1]

To a solution of 5-fluoro-7-vinylquinoxaline (536 mg, 3.08 mmol, 1.00 equiv) in THE (10.7 mL) and H₂O (5.36 mL) was added OsO₄ (117 mg, 462 μmol, 24.0 μL, 0.15 equiv) and NaIO₄ (3.29 g, 15.4 mmol, 853 μL, 5.00 equiv). The mixture was stirred at 15° C. for 2 h. Insoluble precipitate was removed by passing through a celite column, and the filtrate was extracted with ethyl acetate (5.00 mL×3). The combined organic layers was washed with brine (5.00 mL), dried with Na₂SO₄ and concentrated under vacuum to give the residue. The residue was purified by column chromatography (SiO₂, petroleum ether:ethyl acetate=1:0 to 5:1) to provide the title compound (516 mg, 95.2%) as a yellow solid. MS (ES+) m/e 177.1 (M+H)⁺.

### Step 3. tert-Butyl (E)-4-(3-(((8-fluoroquinoxalin-6-yl)methylene)amino)pyridin-4-yl)piperazine-1-carboxylate

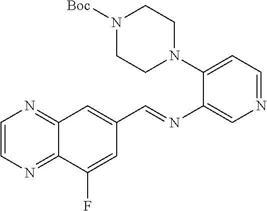 [SMILES: CC(C)(C)OC(=O)N1CCN(c2ccncc2/N=C/c2cc(F)c3nccnc3c2)CC1]

To a solution of 8-fluoroquinoxaline-6-carbaldehyde (250 mg, 1.42 mmol, 1.00 equiv) in EtOH (10 mL) was added tert-butyl 4-(3-aminopyridin-4-yl)piperazine-1-carboxylate (435 mg, 1.56 mmol, 1.10 equiv), CH₃COOH (128 mg, 2.13 mmol, 122 μL, 1.50 equiv) and 4A MS (400 mg). The mixture was stirred at 80° C. for 3 h and was concentrated under reduced pressure to remove AcOH and EtOH to provide the title compound (619 mg, crude) as a yellow oil. MS (ES+) m/e 437.2 (M+H)⁺.

### Step 4. tert-Butyl 4-(3-(((8-fluoroquinoxalin-6-yl)methyl)amino)pyridin-4-yl)piperazine-1-carboxylate

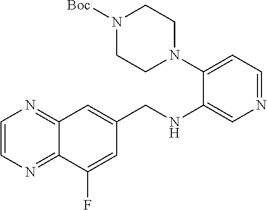 [SMILES: CC(C)(C)OC(=O)N1CCN(c2ccncc2NCc2cc(F)c3nccnc3c2)CC1]

To a solution of tert-butyl (E)-4-(3-(((8-fluoroquinoxalin-6-yl)methylene)amino)pyridin-4-yl)piperazine-1-carboxylate (619 mg, 1.42 mmol, 1.00 equiv) in MeOH (10 mL) was added NaBH₄ (107 mg, 2.84 mmol, 2.00 equiv). The mixture was stirred at 0° C.˜5° C. for 0.5 h and was quenched with sat. NH₄Cl (10.0 mL). The filtrate was concentrated under vacuum. The residue was extracted with ethyl acetate (10.0 mL×3). The combined organic layers was washed with brine (5 mL), dried with Na₂SO₄ and concentrated under vacuum to give the residue. The residue was purified by prep-HPLC (column: YMC Triart C18 250×50 mm×7 um; mobile phase: [water (FA)-ACN]; B %: 22%-52%, 10 min). The title compound (400 mg, 64.3%) was obtained as a white solid. MS (ES+) m/e 439.2 (M+H)⁺.

### Step 5. N-((8-Fluoroquinoxalin-6-yl)methyl)-4-(piperazin-1-yl)pyridin-3-amine

To tert-butyl 4-(3-(((8-fluoroquinoxalin-6-yl)methyl)amino)pyridin-4-yl)piperazine-1-carboxylate (200 mg, 456.1 umol, 1.00 equiv) in MeOH (4.00 mL) was added HCl/MeOH (4 M, 4.00 mL) dropwise at 20° C. The mixture was stirred for 3 h and was then concentrated under reduced pressure to give the title compound (174 mg, 96.6%) as a dark solid. ¹H NMR (400 MHz, D₂O) δ 9.62 (br s, 2H), 8.99 (dd, J=14.4, 1.8 Hz, 2H), 8.09 (d, J=6.4 Hz, 1H), 7.92 (s, 1H), 7.84-7.77 (m, 2H), 7.40 (d, J=6.4 Hz, 1H), 6.87 (br s, 1H), 4.74 (br d, J=4.8 Hz, 2H), 3.50 (br s, 4H), 3.41 (br s, 4H). MS (ES+) m/e 339.1 (M+H)⁺.

### Example 8

### N-((7-Chloroquinoxalin-6-yl)methyl)-4-(piperazin-1-yl)pyridin-3-amine (Comp. 013)

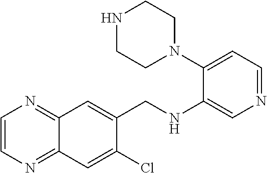 [SMILES: Clc1cc2nccnc2cc1CNc1cnccc1N1CCNCC1]

### Step 1. 4-Bromo-5-chlorobenzene-1,2-diamine

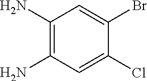

To a solution of 4-bromo-5-chloro-2-nitroaniline (5.00 g, 20.0 mmol, 1.00 equiv) in EtOH (120 mL) was added SnCl₂ (18.0 g, 79.5 mmol, 4.00 equiv). The mixture was stirred at 70° C. for 3 h, cooled to room temperature and poured into ice water (200 mL). The pH of the mixture was adjusted to basic with addition of saturated NaOH (200 mL) and the mixture was then extracted with EtOAc (200 mL×2). The combined organic phases were washed with brine, dried (Na₂SO₄), filtered, and concentrated in vacuo to provide the title compound (4.11 g, crude) as a white solid. MS (ES+) m/e 222.9 (M+H)⁺.

### Step 2. 6-Bromo-7-chloro uinoxaline

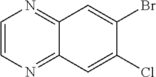 [SMILES: Clc1cc2nccnc2cc1Br]

To a solution of 4-bromo-5-chlorocyclohexa-3,5-diene-1,2-diamine (4.11 g, 18.6 mmol, 1.00 equiv) in EtOH (164 mL) was added oxaldehyde (5.38 g, 37.1 mmol, 40% purity, 2.00 equiv). The mixture was stirred at 15° C. for 12 h, cooled to 15° C., and filtered. The filter cake was washed with EtOH (10 mL×2) and dried to provide the title compound (2.70 g, crude) as a yellow solid. MS (ES+) m/e 452.0 (M+H)⁺.

### Step 3. 6-Chloro-7-vinylquinoxaline

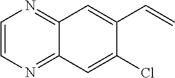

A mixture of 6-bromo-7-chloroquinoxaline (1.00 g, 4.11 mmol, 1.00 equiv), potassium trifluoro (vinyl) boranuide (1.10 g, 8.21 mmol, 2.00 equiv), Pd (dppf)Cl₂—CH₂Cl₂ (671 mg, 821 μmol, 0.200 equiv), and Cs₂CO₃ (2.68 g, 8.21 mmol, 2.00 equiv) in dioxane (10.0 mL) and H₂O (2.00 mL) was degassed, purged with N₂ for 3 times, and stirred at 100° C. for 1 h under N₂ atmosphere, quenched with water (5.00 mL), and extracted with EtOAc (5.00 mL×2). The organic layers were washed with brine (5.00 mL), dried by Na₂SO₄, filtered, and concentrated in vacuum. The residue was purified by prep-TLC (SiO₂; petroleum ether:ethyl acetate=5:1, R_{f}=0.60) to provide the title compound (0.634 g, 81.0%) as a yellow oil. MS (ES+) m/e 191.1 (M+H)⁺.

### Step 4. 7-Chloroquinoxaline-6-carbaldehyde

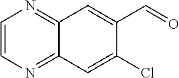 [SMILES: O=Cc1cc2nccnc2cc1Cl]

To a solution of 6-chloro-7-vinylquinoxaline (534 mg, 2.80 mmol, 1.00 equiv) in THF (10.7 mL) and H₂O (5.34 mL) was added OsO₄ (107 mg, 420 μmol, 21.80 μL, 0.15 equiv) and NaIO₄ (3.00 g, 14.0 mmol, 776 μL, 5.00 equiv). The mixture was stirred at 15° C. for 0.5 h. The insoluble was removed through a celite column, and the filtrate was extracted with ethyl acetate (5.00 mL×3). The combined organic layers were washed with brine (5.00 mL), dried with Na₂SO₄ and concentrated under vacuum to give the residue. The residue was purified by prep-TLC (SiO₂, petroleum ether:ethyl acetate=1:1, R_{f}=0.4) to provide the title compound (164 mg, 30.4%) as a white solid. MS (ES+) m/e 193.2 (M+H)⁺.

### Step 5. tert-Butyl (E)-4-(3-(((7-chloroquinoxalin-6-yl)methylene)amino)pyridin-4-yl)piperazine-1-carboxylate

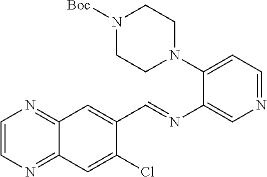 [SMILES: CC(C)(C)OC(=O)N1CCN(c2ccncc2/N=C/c2cc3nccnc3cc2Cl)CC1]

To a solution of 7-chloroquinoxaline-6-carbaldehyde (208 mg, 1.08 mmol, 1.00 equiv) in EtOH (8.30 mL) was added tert-butyl 4-(3-aminopyridin-4-yl)piperazine-1-carboxylate (331 mg, 1.19 mmol, 1.10 equiv), CH₃COOH (97.3 mg, 1.62 mmol, 92.6 μL, 1.50 equiv) and 4A MS (594 mg). The mixture was stirred at 80° C. for 3 h and was concentrated under reduced pressure to remove AcOH and EtOH to provide the title compound (489 mg, crude) as a yellow oil. MS (ES+) m/e 453.3 (M+H)⁺.

### Step 6. tert-Butyl 4-(3-(((7-chloroquinoxalin-6-yl)methyl)amino)pyridin-4-yl)piperazine-1-carboxylate

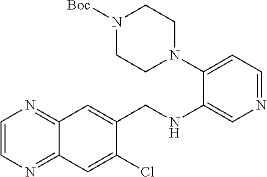 [SMILES: CC(C)(C)OC(=O)N1CCN(c2ccncc2NCc2cc3nccnc3cc2Cl)CC1]

To a solution of tert-butyl (E)-4-(3-(((7-chloroquinoxalin-6-yl)methylene)amino)pyridin-4-yl)piperazine-1-carboxylate (489 mg, 1.08 mmol, 1.00 equiv) in MeOH (8.0 mL) was added NaBH₄ (81.7 mg, 2.16 mmol, 2.00 equiv). The mixture was stirred at 0˜5° C. for 0.5 h, quenched with sat. NH₄Cl (10.0 mL) and filtered to give the filtrate. The filtrate was concentrated under vacuum. The residue was extracted with EtOAc (10 mL×3). The combined organic layers were washed with brine (5 mL), dried with Na₂SO₄ and concentrated to give the residue. The residue was purified by prep-HPLC (Waters xbridge 150×25 mm×10 um; mobile phase: [water (NH₄HCO₃)-ACN]; B %: 35%-65%, 11 min) to provide the title compound (170 mg, 34.6%) as a yellow solid. MS (ES+) m/e 455.2 (M+H)⁺.

### Step 7. N-((7-Chloroquinoxalin-6-yl)methyl)-4-(piperazin-1-yl)pyridin-3-amine

To a solution of tert-butyl 4-(3-(((7-chloroquinoxalin-6-yl)methyl)amino)pyridin-4-yl)piperazine-1-carboxylate (170 mg, 374 mol, 1.00 equiv) in dioxane (2.00 mL) was added HCl/dioxane (4 M, 157 L, 1.68 equiv). The mixture was stirred at 15° C. for 0.5 h and was filtered. The filter cake was concentrated in vacuo to provide the title compound (64.3 mg, 40.9%) as a brown solid. ¹H NMR (400 MHz, D₂O) δ 8.86-8.79 (m, 2H), 8.20-8.13 (m, 1H), 8.04-7.96 (m, 1H), 7.88 (br s, 1H), 7.69 (d, J=0.88 Hz, 1H), 7.41 (d, J=6.4 Hz, 1H), 4.76 (s, 2H), 3.68-3.60 (m, 4H), 3.56-3.52 (m, 4H). MS (ES+) m/e 355.2 (M+H)⁺.

### Example 9

### 4-(1,4-Diazepan-1-yl)-N-(quinoxalin-6-ylmethyl)pyridin-3-amine (Comp. 014)

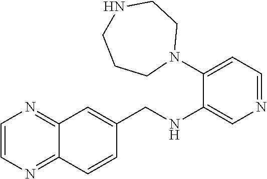 [SMILES: c1cc(N2CCCNCC2)c(NCc2ccc3nccnc3c2)cn1]

### Step 1. 4-Bromo-N-(quinoxalin-6-ylmethyl)pyridin-3-amine

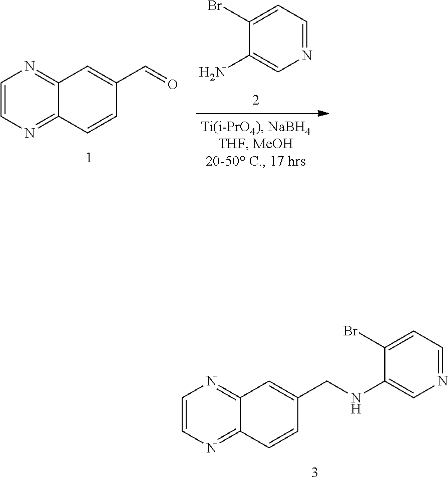 [SMILES: O=Cc1ccc2nccnc2c1 ; Nc1cnccc1Br ; Brc1ccncc1NCc1ccc2nccnc2c1]

To a solution of quinoxaline-6-carbaldehyde (5.00 g, 28.9 mmol, 1.00 equiv) and 4-bromopyridin-3-amine (5.94 g, 37.6 mmol, 1.30 equiv) in THF (100 mL) was added Ti(i-PrO)₄ (16.4 g, 57.8 mmol, 17.1 mL, 2.00 eq). The reaction mixture was stirred at 50° C. for 16 h and cooled to 20° C. MeOH (100 mL) and NaBH₄ (4.37 g, 115.6 mmol, 4.00 equiv) was added and the resulting solution was stirred at 20° C. for 1 h, quenched with addition ice water (400 mL) at 0° C. and extracted with EtOAc (200 mL×3). The combined organic layers were washed with brine 200 mL, dried over Na₂SO₄, filtered and concentrated under reduced pressure to give a residue. The crude product was triturated with EtOAc (50.0 mL) at 20° C. for 30 min, then filtered and the yellow solid was collected. The title compound (6.00 g, 65.8%) was obtained as a yellow solid. 1H NMR (400 MHz, D2O) δ 8.94-8.90 (m, 2H), 8.09 (d, J=8.8 Hz, 1H), 8.03 (d, J=0.8 Hz, 1H), 7.91-7.86 (m, 2H), 7.64 (d, J=5.2 Hz, 1H), 7.49 (d, J=5.2 Hz, 1H), 6.52 (t, J=6.4 Hz, 1H), 4.77 (d, J=6.4 Hz, 2H). MS (ES+) m/e 315.1 (M+H)⁺.

### Step 2. 4-(1,4-Diazepan-1-yl)-N-(quinoxalin-6-ylmethyl)pyridin-3-amine (M173)

To a solution of 4-bromo-N-(quinoxalin-6-ylmethyl)pyridin-3-amine (200 mg, 634.6 μmol, 1.00 equiv) and tert-butyl 1,4-diazepane-1-carboxylate (1.90 mmol, 3.00 equiv) in NMP (2.00 mL) was added DIPEA (328.1 mg, 2.54 mmol, 442.1 μL, 4.00 equiv). The mixture was stirred at 180° C. for 8 h. The reaction mixture was directly purified by Pre-HPLC (HCl condition) without workup. The purified product was dissolved in MeOH (1.00 mL) followed by addition of HCl/MeOH (4.0 M, 1.00 mL, 35.7 equiv). The mixture was stirred at 20° C. for 1 h and was purified by prep-HPLC (HCl condition) to give the title compound (116.8 mg, 36.5%) as a brown solid. ¹H NMR (400 MHz, D₂O) δ 8.81 (s, 2H), 8.05-8.03 (m, 1H), 7.97 (s, 1H), 7.87-7.84 (m, 2H), 7.58 (s, 1H), 7.26-7.24 (m, 1H), 4.63 (s, 2H), 3.88-3.86 (m, 2H), 3.66-3.57 (m, 4H), 3.47-3.45 (m, 2H), 3.44-3.40 (m, 2H), 2.20-2.17 (m, 2H). MS (ES+) m/e 435.2 (M+H)⁺.

### Example 10

### 4-(2-Methylpiperazin-1-yl)-N-(quinoxalin-6-ylmethyl)pyridin-3-amine (Comp. 015)

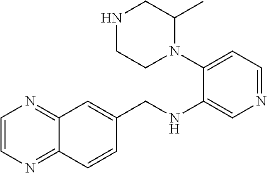 [SMILES: CC1CNCCN1c1ccncc1NCc1ccc2nccnc2c1]

To a solution of 4-bromo-N-(quinoxalin-6-ylmethyl)pyridin-3-amine (200 mg, 634 μmol, 1.00 equiv) and tert-butyl 3-methylpiperazine-1-carboxylate (1.90 mmol, 3.00 equiv) in NMP (2.00 mL) was added DIPEA (328 mg, 2.54 mmol, 442.1 μL, 4.00 equiv). The mixture was stirred at 180° C. for 8 h. The reaction mixture was directly purified without workup to provide the title compound (94.0 mg, 43.3%) as a brown solid. ¹H NMR (400 MHz, DMSO-d₆) δ 8.84 (s, 2H), 8.13-8.11 (m, 1H), 8.07 (s, 1H), 8.01-7.99 (m, 1H), 7.89 (s, 1H), 7.80-7.78 (m, 1H), 6.87 (d, J=5.6 Hz, 1H), 4.78-4.75 (m, 1H), 4.68-4.66 (m, 2H), 4.02-3.90 (m, 1H), 3.28-3.22 (m, 3H), 3.18-3.04 (m, 1H), 2.77-2.74 (m, 1H), 2.50-2.44 (m, 1H), 1.18 (d, J=6.4 Hz, 3H). MS (ES+) m/e 335.3 (M+H)⁺.

### Example 11

### 4-(3-Methylpiperazin-1-yl)-N-(quinoxalin-6-ylmethyl)pyridin-3-amine (Comp. 016)

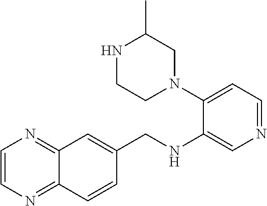 [SMILES: CC1CN(c2ccncc2NCc2ccc3nccnc3c2)CCN1]

To a solution of 4-bromo-N-(quinoxalin-6-ylmethyl)pyridin-3-amine (500 mg, 1.59 mmol, 1.00 equiv) and tert-butyl 2-methylpiperazine-1-carboxylate (476 mg, 2.38 mmol, 1.50 equiv) in NMP (2.00) was added DIPEA (328 mg, 2.54 mmol, 442 L, 4.00 equiv). The mixture was stirred at 180° C. for 8 h. The reaction mixture was directly purified without workup to provide the title compound (403.5 mg, 39.6%) as a brown solid. ¹H NMR (400 MHz, DMSO-d₆) δ 8.85-8.83 (m, 2H), 8.08-7.99 (m, 1H), 7.97-7.95 (m, 2H), 7.89-7.86 (m, 1H), 7.67 (s, 1H), 7.37 (d, J=6.4 Hz, 1H), 4.76-4.73 (m, 2H), 3.94-3.91 (m, 2H), 3.71-3.64 (m, 1H), 3.61-3.60 (m, 1H), 3.47-3.44 (m, 1H), 3.26-3.24 (m, 1H), 3.10-3.04 (m, 1H), 1.40 (d, J=6.4 Hz, 3H). MS (ES+) m/e 335.2 (M+H)⁺.

### Example 12

### 4-(Piperidin-4-yl)-N-(quinoxalin-6-ylmethyl)pyridin-3-amine (Comp. 017)

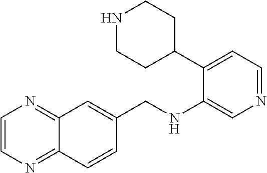 [SMILES: c1cc(C2CCNCC2)c(NCc2ccc3nccnc3c2)cn1]

### Step 1. tert-Butyl 3′-amino-3,6-dihydro-[4,4′-bipyridine]-1(2H)-carboxylate

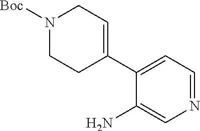 [SMILES: CC(C)(C)OC(=O)N1CC=C(c2ccncc2N)CC1]

A mixture of 4-bromopyridin-3-amine (1.00 g, 5.78 mmol, 1.00 equiv), tert-butyl 4-(4,4,5,5-tetramethyl-1,3,2-dioxaborolan-2-yl)-3,6-dihydropyridine-1(2H)-carboxylate (1.97 g, 6.36 mmol, 1.10 equiv), Pd(OAc)₂ (129 mg, 578 μmol, 0.10 equiv), Xantphos (668 mg, 1.16 mmol, 0.20 equiv), and K₃PO₄ (1.60 g, 11.6 mmol, 2.00 equiv) in dioxane (10.0 mL) and H₂O (2.00 mL) was stirred at 80° C. for 12 h and then at 110° C. for 12 h. The reaction mixture was quenched with water (50.0 mL) and extracted with EtOAc (50.0 mL×2). The combined organic layers were dried with Na₂SO₄, filtered, and concentrated. The residue was purified by silica gel chromatography (eluted with petroleum ether:EtOAc=1:1˜0:1, R_{f}0.3) to provide the title compound (0.70 g, 43.9%) as a yellow oil. MS (ES+) m/e 276.2 (M+H)⁺.

### Step 2. tert-Butyl 4-(3-aminopyridin-4-yl)piperidine-1-carboxylate

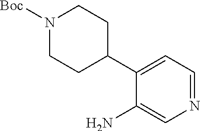 [SMILES: CC(C)(C)OC(=O)N1CCC(c2ccncc2N)CC1]

A mixture of tert-butyl 3′-amino-3,6-dihydro-[4,4′-bipyridine]-1(2H)-carboxylate (0.70 g, 2.54 mmol, 1.00 equiv) and Pd/C (0.10 g, 2.54 mmol, 10% purity, 1.00 equiv) in MeOH (10.0 mL) was stirred at 25° C. for 2 h under H₂ (15 psi). The mixture was filtered and washed with MeOH (10 mL). The filtrate was concentrated to provide the title compound (550 mg, 78.0%) as a yellow oil. MS (ES+) m/e 278.2 (M+H)⁺.

### Step 3. tert-Butyl 4-(3-((quinoxalin-6-ylmethyl)amino)pyridin-4-yl)piperidine-1-carboxylate

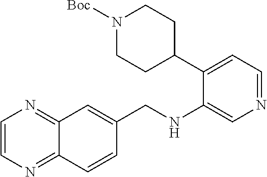 [SMILES: CC(C)(C)OC(=O)N1CCC(c2ccncc2NCc2ccc3nccnc3c2)CC1]

A mixture of tert-butyl 4-(3-aminopyridin-4-yl)piperidine-1-carboxylate (200 mg, 718.5 μmol, 1.00 equiv), quinoxaline-6-carbaldehyde (113.6 mg, 718.5 μmol, 1.00 equiv), and Ti(i-PrO)₄ (224 mg, 790 μmol, 233 μL, 1.10 equiv) in THF (5.00 mL) was stirred at 70° C. for 36 h. NaBH₃CN (90.3 mg, 1.44 mmol, 2.00 equiv) was added and the mixture was stirred at 25° C. for 0.5 h and was poured into sat. NaHCO₃ (20.0 mL). The resulting solution was extracted with EtOAc (10.0 mL×2). The organic layers were washed with water (20.0 mL×2), dried with Na₂SO₄ and concentrated. The residue was purified by reverse phase HPLC (formic acid condition) to provide the title compound (62.0 mg, 20.5%) as a yellow solid. MS (ES+) m/e 421.2 (M+H)⁺.

### Step 4. 4-(Piperidin-4-yl)-N-(quinoxalin-6-ylmethyl)pyridin-3-amine

To a solution of tert-butyl 4-(3-((quinoxalin-6-ylmethyl)amino)pyridin-4-yl)piperidine-1-carboxylate (48.3 mg, 115 μmol, 1.00 equiv) in MeOH (1.00 mL) was added HCl/MeOH (4.00 M, 1.00 mL, 34.7 equiv). The mixture was stirred at 20° C. for 1 h and concentrated to provide the title compound (35.0 mg, 82.0%) as a brown oil. ¹H NMR (400 MHz, D₂O) δ 8.85 (s, 2H), 8.10-8.08 (m, 1H), 7.99 (s, 1H), 7.94-7.92 (m, 1H), 7.89-7.87 (m, 1H), 7.70-7.66 (m, 2H), 4.82 (m, 2H), 3.65-3.62 (m, 2H), 3.31-3.22 (m, 3H), 2.33-2.30 (m, 2H), 1.99-1.95 (m, 2H). MS (ES+) m/e 320.1 (M+H)⁺.

### Example 13

### 4-(Pyrrolidin-3-yloxy)-N-(quinoxalin-6-ylmethyl)pyridin-3-amine (Comp. 018)

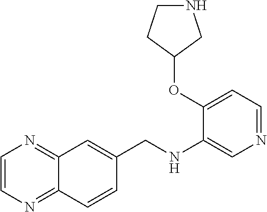 [SMILES: c1cc(OC2CCNC2)c(NCc2ccc3nccnc3c2)cn1]

### Step 1. tert-Butyl 3-((3-nitropyridin-4-yl)oxy)pyrrolidine-1-carboxylate

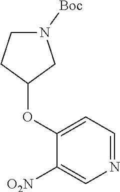 [SMILES: CC(C)(C)OC(=O)N1CCC(Oc2ccncc2[N+](=O)[O-])C1]

To a mixture of 4-chloro-3-nitropyridine (1.00 g, 6.31 mmol, 1.00 equiv) and tert-butyl 3-hydroxypyrrolidine-1-carboxylate (1.18 g, 6.31 mmol, 1.00 equiv) in THF (10.0 mL) was added t-BuOK (2.12 g, 18.9 mmol, 3.00 equiv) at 0° C. The mixture was stirred at 25° C. for 12 h, quenched with NH₄Cl (30 mL), and extracted with EtOAc (30.0 mL×2). The combined organic layers were washed with water (30.0 mL), dried by Na₂SO₄, filtered, and concentrated in vacuum to provide the title compound (1.50 g, crude) as a yellow solid. MS (ES+) m/e 310.1 (M+H)⁺.

### Step 2. tert-Butyl 3-((3-aminopyridin-4-yl)oxy)pyrrolidine-1-carboxylate

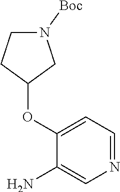 [SMILES: CC(C)(C)OC(=O)N1CCC(Oc2ccncc2N)C1]

To a solution of tert-butyl 3-((3-nitropyridin-4-yl)oxy)pyrrolidine-1-carboxylate (1.50 g, 4.85 mmol, 1.00 equiv) and NH₄C1 (1.30 g, 24.3 mmol, 5.00 equiv) in EtOH (25.0 mL) and H₂O (25.0 mL) was added Fe (1.35 g, 24.3 mmol, 5.00 equiv). The mixture was stirred at 45° C. for 1 h and was filtered. The filtrate was extracted with EtOAc (100 mL×2). The combined organic layers were washed with water (100 mL), dried by Na₂SO₄, filtered, and concentrated to provide the title compound (900 mg, crude) as a brown solid. MS (ES+) m/e 280.2 (M+H)⁺.

### Step 3. tert-Butyl 3-((3-((quinoxalin-6-ylmethyl)amino)pyridin-4-yl)oxy)pyrrolidine-1-carboxylate

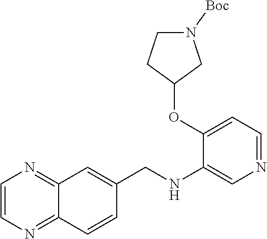 [SMILES: CC(C)(C)OC(=O)N1CCC(Oc2ccncc2NCc2ccc3nccnc3c2)C1]

A mixture of tert-butyl 3-((3-aminopyridin-4-yl)oxy)pyrrolidine-1-carboxylate (450 mg, 1.61 mmol, 1.00 equiv), quinoxaline-6-carbaldehyde (254 mg, 1.61 mmol, 1.00 equiv), AcOH (145 mg, 2.42 mmol, 138 μL, 1.50 equiv) and 4A MS (1.00 g, 1.61 mmol, 1.00 equiv) in EtOH (2.00 mL) was stirred at 80° C. for 12 h. NaBH(OAc)₃ (1.50 g) was added and the mixture was stirred 12 h at 25° C., quenched with NaHCO₃ (40.0 mL), and extracted with DCM (30.0 mL×2). The combined organic layers were dried with Na₂SO₄, filtered, and concentrated to provide the title compound (500 mg, crude) as a yellow oil. MS (ES+) m/e 422.2 (M+H)⁺.

### Step 4. 4-(Pyrrolidin-3-yloxy)-N-(quinoxalin-6-ylmethyl)pyridin-3-amine

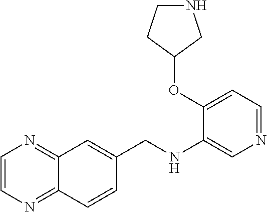 [SMILES: c1cc(OC2CCNC2)c(NCc2ccc3nccnc3c2)cn1]

To a solution of t-butyl 3-((3-((quinoxalin-6-ylmethyl)amino)pyridin-4-yl)oxy)pyrrolidine-1-carboxylate (100 mg, 237 mol, 1.00 equiv) in dioxane (2.00 mL) was added HCl/dioxane (2.00 mL). The mixture was stirred at 20° C. for 1 h and was concentrated to provide the title compound (50.0 mg, 64.2%) as a dark solid. ¹H NMR (400 MHz, D₂O) δ 8.87-8.86 (m, 2H), 8.10-8.07 (m, 1H), 8.0-7.98 (m, 2H), 7.96-7.89 (m, 1H), 7.63 (s, 1H), 7.37-7.35 (m, 1H), 5.64 (s, 1H), 3.84-3.58 (m, 6H), 2.53-2.48 (m, 2H). MS (ES+) m/e 322.2 (M+H)⁺.

### Example 14

### 5-Chloro-4-(piperazin-1-yl)-N-(quinoxalin-6-ylmethyl)pyridin-3-amine (Comp. 019)

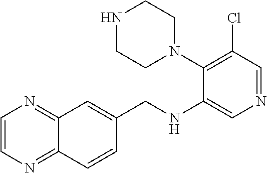 [SMILES: Clc1cncc(NCc2ccc3nccnc3c2)c1N1CCNCC1]

### Step 1. tert-Butyl 4-(3-chloro-5-nitropyridin-4-yl)piperazine-1-carboxylate

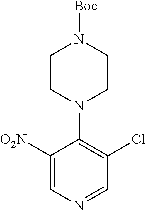 [SMILES: CC(C)(C)OC(=O)N1CCN(c2c(Cl)cncc2[N+](=O)[O-])CC1]

A mixture of 3,4-dichloro-5-nitropyridine (1.00 g, 5.18 mmol, 1.00 equiv), tert-butyl piperazine-1-carboxylate (965 mg, 5.18 mmol, 1.00 equiv) and DIEA (736 mg, 5.70 mmol, 992 μL, 1.10 equiv) in i-PrOH (10.0 mL) was stirred at 25° C. for 12 h. The reaction solution was concentrated to provide the title compound (1.78 g, crude) as a yellow solid. MS (ES+) m/e 343.1 (M+H)⁺.

### Step 2. tert-butyl 4-(3-amino-5-chloropyridin-4-yl)piperazine-1-carboxylate

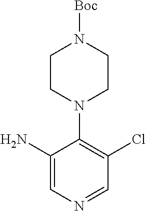 [SMILES: CC(C)(C)OC(=O)N1CCN(c2c(N)cncc2Cl)CC1]

To a solution of tert-butyl 4-(3-chloro-5-nitropyridin-4-yl)piperazine-1-carboxylate (1.78 g, 5.19 mmol, 1.00 equiv) and NH₄Cl (4.17 g, 77.9 mmol, 15.0 equiv) in EtOH (20.0 mL) and H₂O (15.0 mL) was added Fe (1.45 g, 25.9 mmol, 5.00 equiv). The mixture was stirred at 25° C. for 4 and was filtered. The filtrate was extracted with DCM (100 mL×3). The combined organic layers were washed with water (50.0 mL), dried with Na₂SO₄, filtered, and concentrated to provide the title compound (1.53 g, 94.1%) as a yellow solid. MS (ES+) m/e 355.1 (M+H)⁺.

### Step 3. tert-Butyl 4-(3-chloro-5-((quinoxalin-6-ylmethyl)amino)pyridin-4-yl)piperazine-1-carboxylate

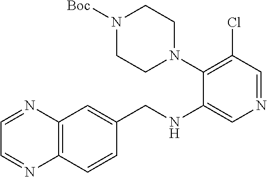 [SMILES: CC(C)(C)OC(=O)N1CCN(c2c(Cl)cncc2NCc2ccc3nccnc3c2)CC1]

A mixture of tert-butyl 4-(3-amino-5-chloropyridin-4-yl)piperazine-1-carboxylate (200 mg, 639 μmol, 1.00 equiv), quinoxaline-6-carbaldehyde (101 mg, 639 μmol, 1.00 equiv), AcOH (57.6 mg, 959 μmol, 54.8 μL, 1.50 equiv) and 4A MS (0.5 g) in EtOH (1.00 mL) was stirred at 80° C. for 12 h. NaBH₃CN (90.3 mg, 1.44 mmol, 2.00 equiv) was added and the mixture was stirred at 25° C. for 1 h. The reaction solution was concentrated to provide the title compound (250 mg, crude) as a yellow solid.

### Step 4. 5-chloro-4-(piperazin-1-yl)-N-(quinoxalin-6-ylmethyl)pyridin-3-amine

To a solution of tert-butyl 4-(3-chloro-5-((quinoxalin-6-ylmethyl)amino)pyridin-4-yl)piperazine-1-carboxylate (250 mg, 549 μmol, 1.00 equiv) in dioxane (5.00 mL) was added HCl/dioxane (4.00 M, 1.00 mL, 7.28 equiv). The mixture was stirred at 25° C. for 12 h. The solids formed was collected by filtration, washed with dioxane (1.00 mL) and dried to provide the title compound (180 mg, 83.7%) as a brown solid. ¹H NMR (400 MHz, DMSO-d₆) δ 9.5 (br s, 2H), 8.95-8.90 (m, 2H), 8.11-8.06 (m, 2H), 7.93-7.90 (m, 1H), 7.87 (s, 2H), 7.25 (s, 1H), 4.79 (s, 2H), 3.43 (br s, 8H). MS (ES+) m/e 355.1 (M+H)⁺.

The following compounds were synthesized using essentially the same procedures described for the previous compounds with appropriate starting materials.

### Example 15

### 4-(Azetidin-3-yloxy)-N-(quinoxalin-6-ylmethyl)pyridin-3-amine (Comp. 022)

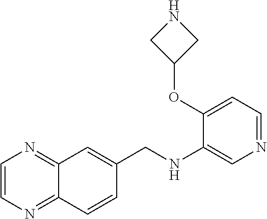 [SMILES: c1cc(OC2CNC2)c(NCc2ccc3nccnc3c2)cn1]

¹H NMR (400 MHz, DMSO-d₆) δ 8.90 (q, J=2.0 Hz, 2H), 8.07 (d, J=8.8 Hz, 1H), 8.02 (d, J=1.2 Hz, 1H), 7.89 (dd, J₁=1.6 Hz, J₂=8.4 Hz, 1H), 6.57 (d, J=5.2 Hz, 1H), 6.16 (br s, 1H), 5.14-5.06 (m, 1H), 4.66 (d, J=6.0 Hz, 2H), 4.41-4.17 (m, 1H), 3.82-3.41 (m, 6H). MS (ES+) m/e 308.2 (M+H)⁺.

### Example 16

### 5-Methyl-4-(piperazin-1-yl)-N-(quinoxalin-6-ylmethyl)pyridin-3-amine (Comp. 023)

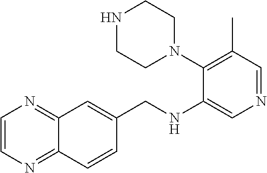 [SMILES: Cc1cncc(NCc2ccc3nccnc3c2)c1N1CCNCC1]

¹H NMR (400 MHz, DMSO-d₆) δ 8.84 (s, 2H), 8.12 (d, J=8.80 Hz, 1H), 8.05 (s, 1H), 7.82-7.76 (m, 2H), 7.74 (br s, 1H), 5.53 (br t, J=5.80 Hz, 1H), 4.66 (d, J=5.60 Hz, 2H), 3.45-3.23 (m, 2H), 3.22-2.78 (m, 7H), 2.35 (s, 3H). MS (ES+) m/e 335.1 (M+H)⁺.

### Example 17

### 6-Fluoro-4-(piperazin-1-yl)-N-(quinoxalin-6-ylmethyl)pyridin-3-amine (Comp. 024)

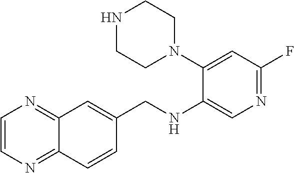 [SMILES: Fc1cc(N2CCNCC2)c(NCc2ccc3nccnc3c2)cn1]

¹H NMR (400 MHz, DMSO-d₆) δ 8.86 (s, 2H), 8.14 (d, J=8.80 Hz, 1H), 8.07 (s, 1H), 7.85-7.74 (m, 1H), 7.37 (s, 1H), 6.50 (s, 1H), 4.63 (br d, J=5.50 Hz, 2H), 4.48 (br t, J 4.90 Hz, 1H), 3.11 (s, 8H). MS (ES+) m/e 339.1 (M+H)⁺.

### Example 18

### (S)-5-Chloro-4-(3-methylpiperazin-1-yl)-N-(quinoxalin-6-ylmethyl)pyridin-3-amine (Comp. 025)

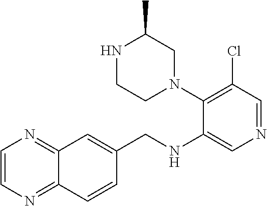 [SMILES: C[C@H]1CN(c2c(Cl)cncc2NCc2ccc3nccnc3c2)CCN1]

¹H NMR (400 MHz, DMSO-d₆) δ 8.85 (s, 2H), 8.13 (d, J=8.40 Hz, 1H), 8.03 (s, 1H), 7.87 (s, 1H), 7.80 (s, 1H), 7.76 (dd, J=1.80, 8.80 Hz, 1H), 5.70 (br s, 1H), 4.68 (d, J 6.00 Hz, 2H), 3.58-3.45 (m, 1H), 3.22-3.11 (m, 2H), 3.03-2.81 (m, 4H), 1.12 (br d, J=6.00 Hz, 3H). MS (ES+) m/e 369.0 (M+H)⁺.

### Example 19

### (R)-5-Chloro-4-(2-methylpiperazin-1-yl)-N-(quinoxalin-6-ylmethyl)pyridin-3-amine (Comp. 026)

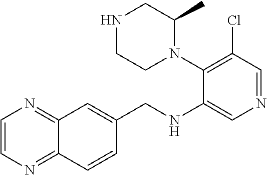 [SMILES: C[C@@H]1CNCCN1c1c(Cl)cncc1NCc1ccc2nccnc2c1]

¹H NMR (400 MHz, DMSO-d₆) δ 8.85 (s, 2H), 8.12 (d, J=8.80 Hz, 1H), 8.02 (s, 1H), 7.86 (s, 1H), 7.81 (s, 1H), 7.74 (dd, J=1.60, 8.80 Hz, 1H), 5.94 (br t, J=5.80 Hz, 1H), 4.69 (br d, J=6.00 Hz, 2H), 3.87-3.76 (m, 1H), 3.51 (br t, J=10.60 Hz, 1H), 3.23-3.06 (m, 2H), 2.95 (br t, J=11.00 Hz, 1H), 2.85-2.70 (m, 1H), 2.57 (br t, J=10.40 Hz, 1H), 0.88 (d, J=6.00 Hz, 3H). MS (ES+) m/e 369.0 (M+H)⁺.

### Example 20

### 4-(Azetidin-3-yloxy)-5-chloro-N-(quinoxalin-6-ylmethyl)pyridin-3-amine (Comp. 027)

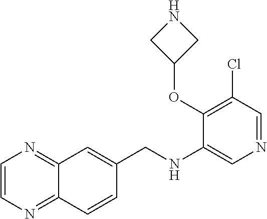 [SMILES: Clc1cncc(NCc2ccc3nccnc3c2)c1OC1CNC1]

¹H NMR (400 MHz, DMSO-d₆) δ 8.90-8.74 (m, 2H), 8.14-8.01 (m, 2H), 7.97 (br s, 1H), 7.85 (br d, J=7.2 Hz, 1H), 7.74 (s, 1H), 5.61 (br t, J=6.0 Hz, 1H), 4.79-4.77 (m, 2H), 4.69-4.57 (m, 4H). MS (ES+) m/e 342.1 (M+H)⁺.

### Example 21

### 4-(Azetidin-3-yloxy)-N-((8-fluoroquinoxalin-6-yl)methyl)pyridin-3-amine (Comp. 028)

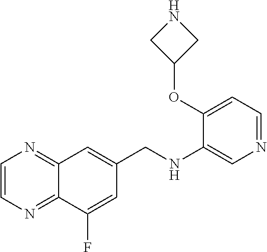 [SMILES: Fc1cc(CNc2cnccc2OC2CNC2)cc2nccnc12]

¹H NMR (400 MHz, DMSO-d₆) δ 9.48-9.10 (m, 2H), 9.00 (dd, J₁=1.6 Hz, J₂=14.0 Hz, 2H), 8.12 (d, J=6.4 Hz, 1H), 7.93 (s, 2H), 7.74 (dd, J₁=1.6 Hz, J₂=11.2 Hz, 1H), 7.45-7.31 (m, 1H), 7.22 (d, J=6.4 Hz, 1H), 5.45 (br s, 1H), 4.76 (br d, J=6.0 Hz, 2H), 4.66-4.49 (m, 2H), 4.29 (br d, J=8.8 Hz, 2H). MS (ES+) m/e 426.2 (M+H)⁺.

### Example 22

### 4-(Azetidin-3-yloxy)-N-((7-chloroquinoxalin-6-yl)methyl)pyridin-3-amine (Comp. 029)

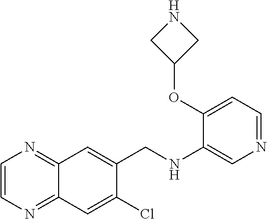 [SMILES: Clc1cc2nccnc2cc1CNc1cnccc1OC1CNC1]

¹H NMR (400 MHz, DMSO-d₆) δ 9.57 (s, 2H), 8.95 (d, J=10.0 Hz, 2H), 8.29 (s, 1H), 8.19 (d, J=6.0 Hz, 1H), 7.99 (s, 1H), 7.94 (s, 1H) 7.33-7.32 (m, 2H), 5.53-5.51 (m, 1H), 4.77 (d, J=4.8 Hz, 2H), 4.62-4.58 (m, 2H), 4.33 (d, J=8.4 Hz, 2H). MS (ES+) m/e 342.0 (M+H)⁺.

### Example 23

### (R)-4-(2-Methylpiperazin-1-yl)-N-(quinoxalin-6-ylmethyl)pyridin-3-amine (Comp. 020)

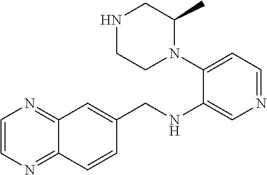 [SMILES: C[C@@H]1CNCCN1c1ccncc1NCc1ccc2nccnc2c1]

¹H NMR (400 MHz, DMSO-d₆) δ 8.89 (s, 2H), 8.07 (d, J=8.4 Hz, 1H), 7.97 (s, 1H), 7.86 (dd, J=1.6, 8.4 Hz, 1H), 7.80-7.73 (m, 2H), 6.95 (d, J=5.2 Hz, 1H), 6.01 (br t, J=6.0 Hz, 1H), 4.83-4.59 (m, 2H), 3.20 (br s, 1H), 3.05-2.91 (m, 3H), 2.91-2.81 (m, 1H), 0.82 (d, J=6.4 Hz, 3H). MS (ES+) m/e 335.2 (M+H)⁺.

### Example 24

### (R)-4-(pyrrolidin-3-yloxy)-N-(quinoxalin-6-ylmethyl)pyridin-3-amine (Comp. 021)

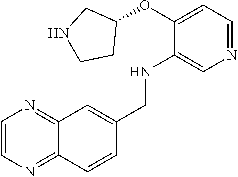 [SMILES: c1cc(O[C@@H]2CCNC2)c(NCc2ccc3nccnc3c2)cn1]

¹H NMR (400 MHz, DMSO-d₆) δ 8.81 (s, 2H), 8.03-7.91 (m, 3H), 7.83 (d, J=8.8 Hz, 1H), 7.59 (s, 1H), 7.33 (d, J=6.4 Hz, 1H), 5.61 (br d, J=2.4 Hz, 1H), 4.71 (s, 1H), 3.85-3.77 (m, 1H), 3.75-3.66 (m, 1H), 3.59 (t, J=7.6 Hz, 2H), 2.52-2.43 (m, 2H). MS (ES+) m/e 322.3 (M+H)⁺.

### Example 25

### 4-(2,5-diazabicyclo[2.2.1]heptan-2-yl)-N-(quinoxalin-6-ylmethyl)pyridin-3-amine (Comp. 030)

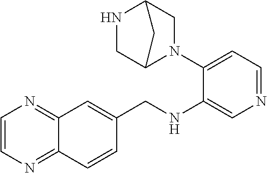 [SMILES: c1cc(N2CC3CC2CN3)c(NCc2ccc3nccnc3c2)cn1]

¹H NMR (400 MHz, D₂O) δ 8.85 (s, 2H), 8.10-8.08 (m, 1H), 8.02 (s, 1H), 7.91-7.89 (m, 1H), 7.86-7.84 (m, 1H), 7.56 (s, 1H), 7.01-7.00 (m, 1H), 5.14 (s, 1H), 4.65-4.59 (m, 3H), 4.20-4.16 (m, 1H), 3.87-3.84 (m, 1H), 3.65-3.62 (m, 1H), 3.53-3.50 (m, 1H), 2.35-2.32 (m, 1H), 2.18-2.15 (m, 1H). MS (ES+) m/e 333 (M+H)⁺.

### Example 26

### 4-(3,6-diazabicyclo[3.1.1]heptan-6-yl)-N-(quinoxalin-6-ylmethyl)pyridin-3-amine (Comp. 031)

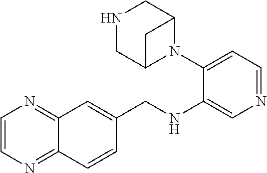 [SMILES: c1cc(N2C3CNCC2C3)c(NCc2ccc3nccnc3c2)cn1]

¹H NMR (400 MHz, D₂O) δ 8.79 (s, 2H), 8.00 (d, J=8.8 Hz, 1H), 7.92 (s, 1H), 7.86 (d, J=6.4 Hz, 1H), 7.81 (dd, J=8.8, 1.60 Hz, 1H), 7.60 (s, 1H), 6.82 (d, J=6.8 Hz, 1H), 4.93 (d, J=6.8 Hz, 2H), 4.53 (s, 2H), 3.77 (d, J=13.6 Hz, 2H), 3.60 (d, J=13.2 Hz, 2H), 3.15-3.09 (m, 1H), 1.98 (d, J=10.4 Hz, 1H). MS (ES+) m/e 333 (M+H)⁺.

### Example 27

### 4-(hexahydropyrrolo[3,4-c]pyrrol-2(1H)-yl)-N-(quinoxalin-6-ylmethyl)pyridin-3-amine (Comp. 032)

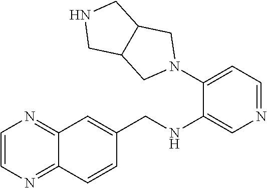 [SMILES: c1cc(N2CC3CNCC3C2)c(NCc2ccc3nccnc3c2)cn1]

¹H NMR (400 MHz, D₂O) δ 8.85 (s, 2H), 8.10-8.08 (m, 1H), 8.03 (s, 1H), 7.92-7.90 (m, 1H), 7.93-7.81 (m, 1H), 7.51 (s, 1H), 7.02-7.00 (m, 1H), 4.65 (s, 2H), 3.78-3.75 (m, 4H), 3.63-3.62 (m, 2H), 3.32-3.30 (m, 4H). MS (ES+) m/e 347 (M+H)⁺.

### Example 28

### 4-(4-aminopiperidin-1-yl)-N-(quinoxalin-6-ylmethyl)pyridin-3-amine (Comp. 033)

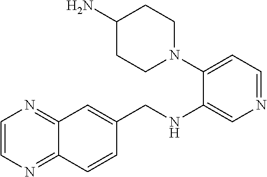 [SMILES: NC1CCN(c2ccncc2NCc2ccc3nccnc3c2)CC1]

¹H NMR (400 MHz, D₂O) δ 8.86 (s, 2H), 8.09-8.07 (m, 1H), 7.99 (s, 1H), 7.91-7.87 (m, 2H), 7.56 (s, 1H), 7.28-7.27 (m, 1H), 4.77-4.72 (m, 2H), 3.89-3.86 (m, 2H), 3.53-3.46 (m, 1H), 3.00 (t, J=12.0 Hz, 2H), 2.21-2.18 (m, 2H), 1.93-1.84 (m, 2H). MS (ES+) m/e 335 (M+H)⁺.

### Example 29

### N⁴-(piperidin-4-yl)-N³-(quinoxalin-6-ylmethyl)pyridine-3,4-diamine (Comp. 034)

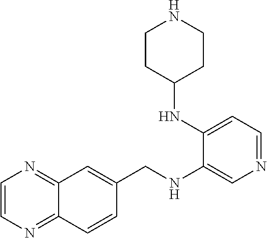 [SMILES: c1cc(NC2CCNCC2)c(NCc2ccc3nccnc3c2)cn1]

¹H NMR (400 MHz, D₂O) δ 8.85 (s, 2H), 8.08-8.06 (m, 1H), 8.01 (s, 1H), 7.90-7.87 (m, 1H), 7.79-7.77 (m, 1H), 7.41 (s, 1H), 6.91-6.89 (m, 1H), 4.66 (s, 2H), 4.04-3.98 (m, 1H), 3.56-3.52 (m, 2H), 3.22-3.15 (m, 2H), 2.34-2.30 (m, 2H), 1.88-1.85 (m, 2H). MS (ES+) m/e 335 (M+H)⁺.

### Example 30

### N⁴-methyl-N⁴-(pyrrolidin-3-yl)-N³-(quinoxalin-6-ylmethyl)pyridine-3,4-diamine (Comp. 035)

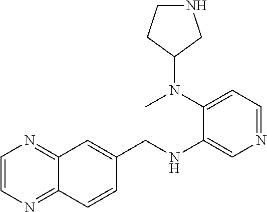 [SMILES: CN(c1ccncc1NCc1ccc2nccnc2c1)C1CCNC1]

¹H NMR (400 MHz, D₂O) δ 8.83 (s, 2H), 8.06-8.04 (m, 1H), 8.00 (s, 1H), 7.90-7.87 (m, 1H), 7.83-7.81 (m, 1H), 7.52 (s, 1H), 6.94-6.92 (m, 1H), 4.70-4.69 (m, 2H), 4.09-3.95 (m, 4H), 3.74-3.70 (m, 1H), 2.77 (s, 3H), 2.58-2.51 (m, 1H), 2.30-2.25 (m, 1H). MS (ES+) m/e 335 (M+H)⁺.

### Example 31

### N-(quinoxalin-6-ylmethyl)-4-(3-(trifluoromethyl)piperazin-1-yl)pyridin-3-amine (Comp. 036)

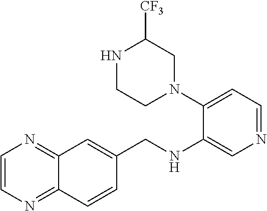 [SMILES: FC(F)(F)C1CN(c2ccncc2NCc2ccc3nccnc3c2)CCN1]

¹H NMR (400 MHz, CDCl₃) δ 8.85 (s, 2H), 8.15-8.12 (m, 1H), 8.06-8.03 (m, 2H), 7.94 (s, 1H), 7.81-7.78 (m, 1H), 6.93-6.91 (m, 1H), 4.76-4.72 (m, 1H), 4.68-4.67 (m, 2H), 3.51-3.48 (m, 1H), 3.41-3.37 (m, 1H), 3.29-3.27 (m, 2H), 3.05-2.99 (m, 1H), 2.97-2.94 (m, 1H), 2.86-2.84 (m, 1H). MS (ES+) m/e 389 (M+H)⁺.

### Example 32

### N-(quinoxalin-6-ylmethyl)-4-(2-(trifluoromethyl)piperazin-1-yl)pyridin-3-amine (Comp. 037)

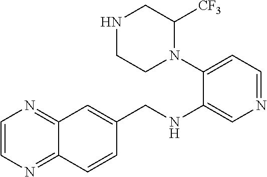 [SMILES: FC(F)(F)C1CNCCN1c1ccncc1NCc1ccc2nccnc2c1]

¹H NMR (400 MHz, CDCl₃) δ 8.85 (s, 2H), 8.15-8.13 (m, 1H), 8.05-8.03 (m, 2H), 7.93 (s, 1H), 7.80-7.78 (m, 1H), 6.94-6.92 (m, 1H), 4.74-4.73 (m, 1H), 4.68-4.67 (m, 2H), 3.51-3.48 (m, 1H), 3.41-3.37 (m, 1H), 3.29-3.27 (m, 2H), 3.05-2.95 (m, 2H), 2.88-2.85 (m, 1H), 1.98-1.96 (m, 1H). MS (ES+) m/e 389 (M+H)⁺.

### Example 33

### 4-(3-((quinoxalin-6-ylmethyl)amino)pyridin-4-yl)piperazin-2-one (Comp. 038)

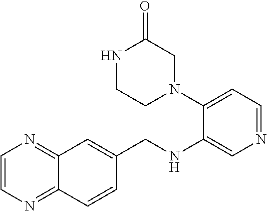 [SMILES: O=C1CN(c2ccncc2NCc2ccc3nccnc3c2)CCN1]

¹H NMR (400 MHz, D₂O) δ 8.87 (s, 2H), 8.14-8.11 (m, 1H), 8.02-8.00 (m, 1H), 7.94 (s, 1H), 7.84-7.82 (m, 2H), 7.10-7.08 (m, 1H), 5.48 (s, 2H), 4.63 (s, 2H), 3.94 (t, J=6.8 Hz, 2H), 3.40 (t, J=6.8 Hz, 2H). MS (ES+) m/e 335 (M+H)⁺.

### Example 34

### 4-morpholino-N-(quinoxalin-6-ylmethyl)pyridin-3-amine (Comp. 039)

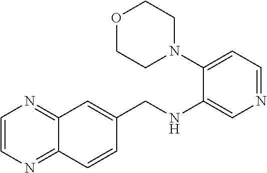 [SMILES: c1cc(N2CCOCC2)c(NCc2ccc3nccnc3c2)cn1]

¹H NMR (400 MHz, CDCl₃) δ 8.85-8.84 (m, 2H), 8.14-8.12 (m, 1H), 8.06-8.05 (m, 1H), 8.03-8.02 (m, 1H), 7.89 (s, 1H), 7.80-7.78 (m, 1H), 6.91-6.90 (m, 1H), 4.79-4.78 (m, 1H), 4.68-4.67 (m, 2H), 3.90-3.87 (m, 4H), 3.09-3.07 (m, 4H). MS (ES+) m/e 322 (M+H)⁺.

### Example 35

### 3-(piperazin-1-yl)-N-(quinoxalin-6-ylmethyl)pyridin-4-amine (Comp. 040)

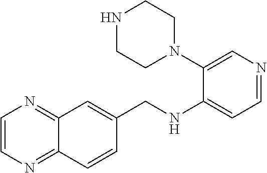 [SMILES: c1cc(NCc2ccc3nccnc3c2)c(N2CCNCC2)cn1]

¹H NMR (400 MHz, D₂O) δ 8.86 (s, 2H), 8.09 (m, 2H), 7.95 (s, 1H), 7.88-7.83 (m, 2H), 6.82-6.80 (m, 1H), 4.94 (s, 2H), 3.49-3.42 (m, 4H), 3.27-3.18 (m, 4H). MS (ES+) m/e 321 (M+H)⁺.

### Example 36

### (S)-4-(2-methylpiperazin-1-yl)-N-(quinoxalin-6-ylmethyl)pyridin-3-amine (Comp. 041)

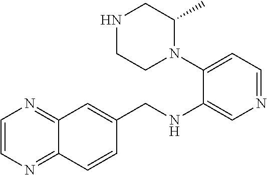 [SMILES: C[C@H]1CNCCN1c1ccncc1NCc1ccc2nccnc2c1]

¹H NMR (400 MHz, DMSO-d₆) δ 8.90 (s, 2H), 8.08 (d, J=8.8 Hz, 1H), 7.98 (s, 1H), 7.90-7.85 (m, 1H), 7.79-7.74 (m, 1H), 6.96 (d, J=4.8 Hz, 1H), 6.00 (t, J=6.0 Hz, 1H), 4.78-4.61 (m, 2H), 3.24-3.16 (m, 1H), 3.06-2.82 (m, 4H), 2.58-2.52 (m, 2H), 2.50-2.46 (m, 2H), 0.83 (d, J=6.0 Hz, 3H). MS (ES+) m/e 335 (M+H)⁺.

### Example 37

### (S)-4-(3-methylpiperazin-1-yl)-N-(quinoxalin-6-ylmethyl)pyridin-3-amine (Comp. 042)

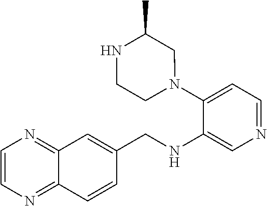 [SMILES: C[C@H]1CN(c2ccncc2NCc2ccc3nccnc3c2)CCN1]

1H NMR (400 MHz, DMSO-d6) δ 8.90 (d, J=1.6 Hz, 2H), 8.08 (d, J=8.8 Hz, 1H), 8.00 (d, J=1.2 Hz, 1H), 7.88 (dd, J1=1.6 Hz, J2=8.8 Hz, 1H), 7.77 (d, J=4.8 Hz, 1H), 7.69 (s, 1H), 6.84 (d, J=5.2 Hz, 1H), 5.65 (t, J=6.0 Hz, 1H), 4.68 (d, J=6.0 Hz, 2H), 3.21-3.12 (m, 2H), 3.02-2.92 (m, 3H), 2.52 (br s, 1H), 2.21 (t, J=10.6 Hz, 1H), 1.01 (d, J=6.4 Hz, 3H). MS (ES+) m/e 335 (M+H)⁺.

### Example 38

### (R)-4-(3-methylpiperazin-1-yl)-N-(quinoxalin-6-ylmethyl)pyridin-3-amine (Comp. 043)

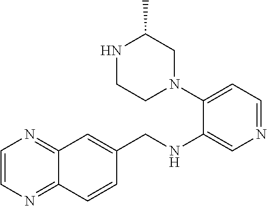 [SMILES: C[C@@H]1CN(c2ccncc2NCc2ccc3nccnc3c2)CCN1]

¹H NMR (400 MHz, DMSO-d₆) δ 8.90 (s, 2H), 8.08 (d, J=8.4 Hz, 1H), 8.00 (s, 1H), 7.88 (d, J=8.8 Hz, 1H), 7.77 (d, J=5.2 Hz, 1H), 7.70 (s, 1H), 6.83 (d, J=5.2 Hz, 1H), 5.65 (t, J=6.0 Hz, 1H), 4.68 (br d, J=6.0 Hz, 2H), 3.17 (br t, J=8.8 Hz, 2H), 3.03-2.92 (m, 3H), 2.57-2.52 (m, 1H), 2.22 (t, J=10.4 Hz, 1H), 1.01 (d, J=6.4 Hz, 3H). MS (ES+) m/e 335 (M+H)⁺.

### Example 39

### (S)-4-(pyrrolidin-3-yloxy)-N-(quinoxalin-6-ylmethyl)pyridin-3-amine (Comp. 044)

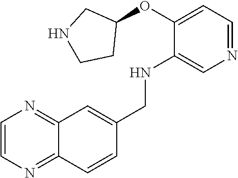 [SMILES: c1cc(O[C@H]2CCNC2)c(NCc2ccc3nccnc3c2)cn1]

¹H NMR (400 MHz, DMSO-d₆) δ 8.85 (s, 2H), 8.07 (d, J=8.8 Hz, 1H), 8.01-7.94 (m, 2H), 7.87 (d, J=8.8 Hz, 1H), 7.63 (s, 1H), 7.36 (d, J=6.4 Hz, 1H), 5.65-5.61 (m, 1H), 4.86-4.81 (m, 1H), 3.85-3.78 (m, 1H), 3.77-3.68 (m, 1H), 3.59 (t, J=7.6 Hz, 2H), 2.53-2.46 (m, 2H). MS (ES+) m/e 322 (M+H)⁺.

### Example 40

### 4-(piperidin-4-yloxy)-N-(quinoxalin-6-ylmethyl)pyridin-3-amine (Comp. 045)

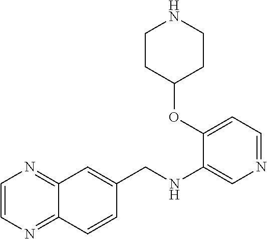 [SMILES: c1cc(OC2CCNCC2)c(NCc2ccc3nccnc3c2)cn1]

¹H NMR (400 MHz, DMSO-d₆) δ 8.96-8.84 (m, 2H), 8.07 (d, J=8.8 Hz, 1H), 8.00 (s, 1H), 7.88 (dd, J1=2.0 Hz, J2=8.8 Hz, 1H), 7.69 (d, J=5.2 Hz, 1H), 7.65 (s, 1H), 6.90 (br d, J=4.8 Hz, 1H), 6.07-5.82 (m, 1H), 4.66 (d, J=6.4 Hz, 3H), 3.76-3.65 (m, 1H), 3.34-3.26 (m, 1H), 3.02 (br s, 1H), 2.74-2.59 (m, 1H), 2.06-1.81 (m, 2H), 1.62 (br s, 2H). MS (ES+) m/e 336 (M+H)⁺.

### Example 41

### N-(quinoxalin-6-ylmethyl)-4-(2,6-diazaspiro[3.3]heptan-2-yl)pyridin-3-amine (Comp. 046)

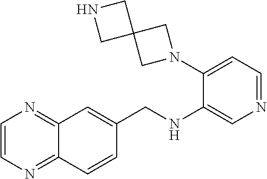 [SMILES: c1cc(N2CC3(CNC3)C2)c(NCc2ccc3nccnc3c2)cn1]

¹H NMR (400 MHz, DMSO-d₆) δ 8.94 (s, 2H), 8.72 (br s, 2H), 8.15-8.05 (m, 2H), 7.94-7.87 (m, 2H), 7.49 (s, 1H), 6.59 (d, J=6.4 Hz, 1H), 6.00 (br t, J=5.2 Hz, 1H), 4.62 (s, 4H), 4.58 (br d, J=5.2 Hz, 2H), 4.21 (br s, 4H). MS (ES+) m/e 333 (M+H)⁺.

### Example 42

### 4-(2-(dimethylamino)ethoxy)-N-(quinoxalin-6-ylmethyl)pyridin-3-amine (Comp. 047)

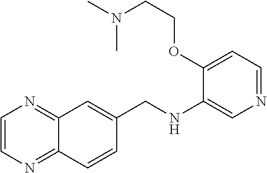 [SMILES: CN(C)CCOc1ccncc1NCc1ccc2nccnc2c1]

¹H NMR (400 MHz, DMSO-d₆) δ 8.91 (s, 2H), 8.27 (br s, 1H), 8.14-8.08 (m, 2H), 8.02-7.95 (m, 1H), 7.81 (s, 1H), 7.42 (d, J=6.4 Hz, 1H), 4.89-4.61 (m, 4H), 3.66 (br d, J=4.4 Hz, 3H), 2.88 (d, J=4.8 Hz, 6H). MS (ES+) m/e 324 (M+H)⁺.

### Example 43

### 4-(piperazin-1-yl)-3-((quinoxalin-6-ylmethyl)amino)benzonitrile (Comp. 048)

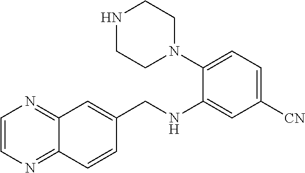 [SMILES: N#Cc1ccc(N2CCNCC2)c(NCc2ccc3nccnc3c2)c1]

### Example 44

### N-((8-chloroquinoxalin-6-yl)methyl)-4-(piperazin-1-yl)pyridin-3-amine (Comp. 049)

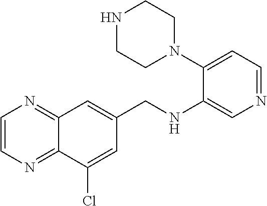 [SMILES: Clc1cc(CNc2cnccc2N2CCNCC2)cc2nccnc12]

¹H NMR (400 MHz, DMSO-d₆) δ 9.03 (dd, J=2.00, 7.20 Hz, 2H), 8.94 (br s, 2H), 8.12 (d, J=1.60 Hz, 1H), 8.07 (d, J=6.00 Hz, 1H), 8.01 (s, 1H), 7.82 (s, 1H), 7.33 (d, J=6.00 Hz, 1H), 6.67-6.48 (m, 1H), 4.73 (br d, J=6.00 Hz, 2H), 3.41 (br s, 8H). MS (ES+) m/e 355 (M+H)⁺.

### Example 45

### N-((7-fluoroquinoxalin-6-yl)methyl)-4-(piperazin-1-yl)pyridin-3-amine (Comp. 050)

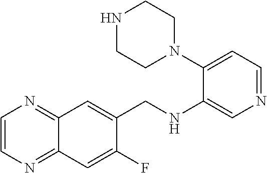 [SMILES: Fc1cc2nccnc2cc1CNc1cnccc1N1CCNCC1]

¹H NMR (400 MHz, CDCl₃) δ 8.83 (d, J=1.60 Hz, 1H), 8.80 (d, J=2.00 Hz, 1H), 8.07 (d, J=7.60 Hz, 1H), 8.02 (d, J=5.20 Hz, 1H), 7.89 (s, 1H), 7.79 (d, J=10.40 Hz, 1H), 6.88 (d, J=5.20 Hz, 1H), 4.84 (br d, J=6.00 Hz, 1H), 4.72 (d, J=6.00 Hz, 2H), 3.11-2.95 (m, 9H). MS (ES+) m/e 339 (M+H)⁺.

### Example 46

### 2-fluoro-4-(piperazin-1-yl)-N-(quinoxalin-6-ylmethyl)pyridin-3-amine (Comp. 051)

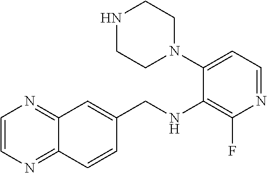 [SMILES: Fc1nccc(N2CCNCC2)c1NCc1ccc2nccnc2c1]

¹H NMR (400 MHz, CDCl₃) δ 8.84 (s, 2H), 8.08 (d, J=8.40 Hz, 1H), 8.01 (s, 1H), 7.74 (dd, J=2.00, 8.80 Hz, 1H), 7.59 (d, J=0.80, 4.40 Hz, 1H), 6.76 (d, J=5.20 Hz, 1H), 4.74 (d, J=8.40 Hz, 2H), 4.41 (br d, J=2.80 Hz, 1H), 3.18-3.00 (m, 8H). MS (ES+) m/e 339 (M+H)⁺.

### Example 47

### 5-chloro-N-((7-fluoroquinoxalin-6-yl)methyl)-4-(piperazin-1-yl)pyridin-3-amine (Comp. 052)

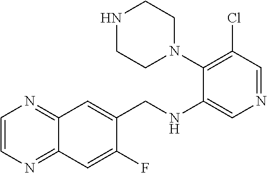 [SMILES: Fc1cc2nccnc2cc1CNc1cncc(Cl)c1N1CCNCC1]

¹H NMR (400 MHz, CDCl₃) δ 8.87-8.77 (m, 2H), 8.02 (d, J=7.60 Hz, 1H), 7.87 (s, 1H), 7.83 (s, 1H), 7.78 (d, J=10.40 Hz, 1H), 5.77 (br t, J=6.00 Hz, 1H), 4.73 (br d, J=6.40 Hz, 2H), 3.76-2.70 (m, 9H). MS (ES+) m/e 373 (M+H)⁺.

### Example 48

### 5-chloro-N-((8-fluoroquinoxalin-6-yl)methyl)-4-(piperazin-1-yl)pyridin-3-amine (Comp. 053)

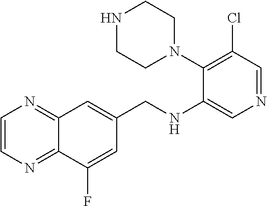 [SMILES: Fc1cc(CNc2cncc(Cl)c2N2CCNCC2)cc2nccnc12]

¹H NMR (400 MHz, CDCl₃) δ 8.89 (d, J=3.60 Hz, 2H), 7.91-7.83 (m, 2H), 7.76 (s, 1H), 7.46 (d, J=10.40 Hz, 1H), 5.77 (br t, J=6.00 Hz, 1H), 4.67 (d, J=6.00 Hz, 2H), 3.64 (br s, 2H), 3.15 (br s, 2H), 2.99 (br s, 2H), 2.90 (br s, 2H). MS (ES+) m/e 373 (M+H)⁺.

### Example 49

### 5-chloro-N-((7-chloroquinoxalin-6-yl)methyl)-4-(piperazin-1-yl)pyridin-3-amine (Comp. 054)

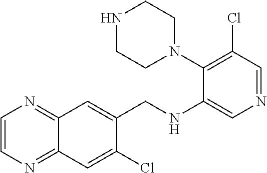 [SMILES: Clc1cc2nccnc2cc1CNc1cncc(Cl)c1N1CCNCC1]

¹H NMR (400 MHz, D₂O) δ 8.85 (d, J=4.80 Hz, 2H), 8.21 (s, 1H), 8.08 (s, 1H), 7.93 (s, 1H), 7.78 (s, 1H), 4.82 (s, 2H), 3.69 (br s, 4H), 3.51 (br t, J=4.80 Hz, 4H). MS (ES+) m/e 389 (M+H)⁺.

### Example 50

### 5-chloro-N-((8-chloroquinoxalin-6-yl)methyl)-4-(piperazin-1-yl)pyridin-3-amine (Comp. 055)

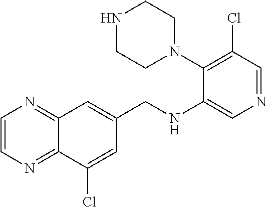 [SMILES: Clc1cncc(NCc2cc(Cl)c3nccnc3c2)c1N1CCNCC1]

¹H NMR (400 MHz, CDCl₃) δ 8.96 (d, J=1.60 Hz, 1H), 8.91 (d, J=1.20 Hz, 1H), 7.98 (s, 1H), 7.89 (s, 2H), 7.77 (s, 1H), 5.78 (br t, J=5.80 Hz, 1H), 4.67 (d, J=6.40 Hz, 2H), 3.68-3.52 (m, 2H), 3.21-2.75 (m, 7H). MS (ES+) m/e 389 (M+H)⁺.

### Example 51

### (R)-5-chloro-4-(3-methylpiperazin-1-yl)-N-(quinoxalin-6-ylmethyl)pyridin-3-amine (Comp. 056)

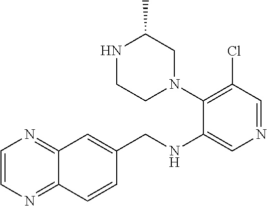 [SMILES: C[C@@H]1CN(c2c(Cl)cncc2NCc2ccc3nccnc3c2)CCN1]

¹H NMR (400 MHz, CDCl₃) δ 8.95-8.80 (m, 2H), 8.13 (d, J=8.40 Hz, 1H), 8.03 (s, 1H), 7.87 (br s, 1H), 7.83-7.70 (m, 2H), 5.70 (br s, 1H), 4.67 (br d, J=6.00 Hz, 2H), 3.54 (br t, J=10.80 Hz, 1H), 3.36-2.75 (m, 7H), 1.13 (br d, J=5.60 Hz, 3H). MS (ES+) m/e 369 (M+H)⁺.

### Example 52

### (S)—N-((7-chloroquinoxalin-6-yl)methyl)-4-(3-methylpiperazin-1-yl)pyridin-3-amine (Comp. 057)

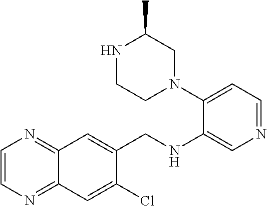 [SMILES: C[C@H]1CN(c2ccncc2NCc2cc3nccnc3cc2Cl)CCN1]

¹H NMR (400 MHz, CDCl₃) δ 8.84 (d, J=2.00 Hz, 1H), 8.82 (d, J=1.60 Hz, 1H), 8.22 (s, 1H), 8.08 (s, 1H), 8.02 (d, J=4.80 Hz, 1H), 7.86 (s, 1H), 6.89 (d, J=5.20 Hz, 1H), 4.94-4.85 (m, 1H), 4.73 (d, J=5.60 Hz, 2H), 3.32-3.19 (m, 3H), 3.18-3.05 (m, 2H), 2.90-2.77 (m, 1H), 2.58-2.45 (m, 1H), 1.22 (d, J=6.40 Hz, 3H). MS (ES+) m/e 369 (M+H)⁺.

### Example 53

### (R)—N-((7-chloroquinoxalin-6-yl)methyl)-4-(3-methylpiperazin-1-yl)pyridin-3-amine (Comp. 058)

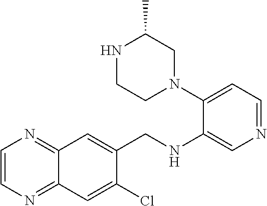 [SMILES: C[C@@H]1CN(c2ccncc2NCc2cc3nccnc3cc2Cl)CCN1]

¹H NMR (400 MHz, CDCl₃) δ 8.83 (dd, J=1.60, 9.60 Hz, 2H), 8.22 (s, 1H), 8.10-8.06 (m, 1H), 8.01 (d, J=5.20 Hz, 1H), 7.87 (s, 1H), 6.89 (d, J=5.20 Hz, 1H), 4.94-4.86 (m, 1H), 4.73 (d, J=6.00 Hz, 2H), 3.32-3.19 (m, 3H), 3.18-3.07 (m, 2H), 2.89-2.79 (m, 1H), 2.52 (br t, J=10.8 Hz, 1H), 1.22 (d, J=6.40 Hz, 3H). MS (ES+) m/e 369 (M+H)⁺.

### Example 54

### (S)—N-((8-fluoroquinoxalin-6-yl)methyl)-4-(3-methylpiperazin-1-yl)pyridin-3-amine (Comp. 059)

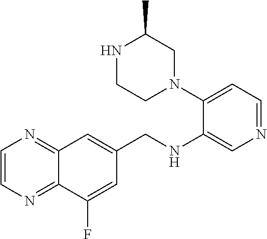 [SMILES: C[C@H]1CN(c2ccncc2NCc2cc(F)c3nccnc3c2)CCN1]

¹H NMR (400 MHz, CDCl₃) δ 8.90 (dd, J=2.00, 6.00 Hz, 1H), 8.95-8.76 (m, 1H), 8.02 (d, J=5.20 Hz, 1H), 7.94-7.84 (m, 2H), 7.52 (dd, J=1.20, 10.40 Hz, 1H), 6.92-6.85 (m, 1H), 4.85-4.78 (m, 1H), 4.66 (d, J=6.00 Hz, 2H), 3.24 (br d, J=12.00 Hz, 3H), 3.15-3.05 (m, 2H), 2.87-2.70 (m, 1H), 2.49 (br t, J=10.40 Hz, 1H), 1.20 (d, J=6.40 Hz, 3H). MS (ES+) m/e 353 (M+H)⁺.

### Example 55

### (R)—N-((8-fluoroquinoxalin-6-yl)methyl)-4-(3-methylpiperazin-1-yl)pyridin-3-amine (Comp. 060)

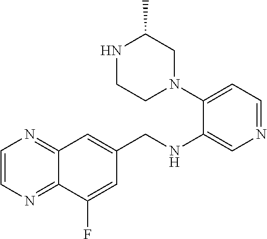 [SMILES: C[C@@H]1CN(c2ccncc2NCc2cc(F)c3nccnc3c2)CCN1]

¹H NMR (400 MHz, CDCl₃) δ 8.95-8.85 (m, 2H), 8.03 (d, J=4.80 Hz, 1H), 7.91 (s, 1H), 7.88 (s, 1H), 7.51 (dd, J=1.20, 10.40 Hz, 1H), 6.89 (d, J=5.20 Hz, 1H), 4.84-4.75 (m, 1H), 4.66 (d, J=6.00 Hz, 2H), 3.34-3.24 (m, 3H), 3.22-3.09 (m, 2H), 2.94-2.85 (m, 1H), 2.60 (br t, J=10.80 Hz, 1H), 1.28 (d, J=6.40 Hz, 3H). MS (ES+) m/e 353 (M+H)⁺.

### Example 56

### (S)-5-chloro-4-(2-methylpiperazin-1-yl)-N-(quinoxalin-6-ylmethyl)pyridin-3-amine (Comp. 061)

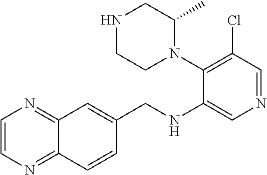 [SMILES: C[C@H]1CNCCN1c1c(Cl)cncc1NCc1ccc2nccnc2c1]

¹H NMR (400 MHz, CDCl₃) δ 8.84 (s, 2H), 8.12 (d, J=8.40 Hz, 1H), 8.02 (s, 1H), 7.85 (s, 1H), 7.80 (s, 1H), 7.74 (dd, J=2.00, 8.80 Hz, 1H), 5.95 (br t, J=6.00 Hz, 1H), 4.69 (d, J=6.40 Hz, 2H), 3.83-3.79 (m, 1H), 3.49 (dt, J=2.80, 11.60 Hz, 1H), 3.12 (br t, J=10.40 Hz, 2H), 2.94 (br d, J=2.80 Hz, 1H), 2.78 (br d, J=12.00 Hz, 1H), 2.56 (br t, J=11.20 Hz, 1H), 0.87 (d, J=6.40 Hz, 3H). MS (ES+) m/e 369 (M+H)⁺.

### Example 57

### (R)—N-((7-chloroquinoxalin-6-yl)methyl)-4-(2-methylpiperazin-1-yl)pyridin-3-amine (Comp. 062)

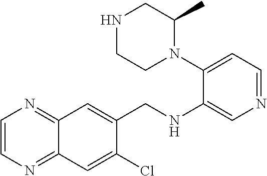 [SMILES: C[C@@H]1CNCCN1c1ccncc1NCc1cc2nccnc2cc1Cl]

¹H NMR (400 MHz, CDCl₃) δ 8.83 (dd, J=2.00, 9.50 Hz, 2H), 8.21 (s, 1H), 8.07-8.00 (m, 2H), 7.98 (s, 1H), 7.04-6.99 (m, 1H), 5.43-5.34 (m, 1H), 4.80-4.67 (m, 2H), 3.52-3.44 (m, 1H), 3.35 (br s, 2H), 3.18 (s, 2H), 3.01-2.94 (m, 1H), 2.89-2.83 (m, 1H), 1.00 (d, J=6.00 Hz, 3H). MS (ES+) m/e 369 (M+H)⁺.

### Example 58

### (S)—N-((7-chloroquinoxalin-6-yl)methyl)-4-(2-methylpiperazin-1-yl)pyridin-3-amine (Comp. 063)

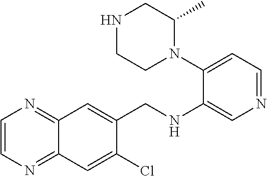 [SMILES: C[C@H]1CNCCN1c1ccncc1NCc1cc2nccnc2cc1Cl]

¹H NMR (400 MHz, CDCl₃) δ 8.88-8.77 (m, 2H), 8.20 (s, 1H), 8.06 (s, 1H), 8.01 (d, J=5.20 Hz, 1H), 7.92 (s, 1H), 6.98 (d, J=5.20 Hz, 1H), 5.46 (br t, J=6.40 Hz, 1H), 4.73 (t, J=6.40 Hz, 2H), 3.30-3.22 (m, 1H), 3.19 (br dd, J=2.40, 12.40 Hz, 1H), 3.16-3.08 (m, 2H), 3.07-3.00 (m, 1H), 2.72 (br dd, J=9.20, 11.20 Hz, 2H), 0.94 (d, J=6.00 Hz, 3H). MS (ES+) m/e 369 (M+H)⁺.

### Example 59

### 4-(azetidin-3-yloxy)-N-((8-chloroquinoxalin-6-yl)methyl)pyridin-3-amine (Comp. 064)

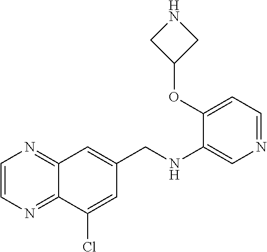 [SMILES: Clc1cc(CNc2cnccc2OC2CNC2)cc2nccnc12]

¹H NMR (400 MHz, DMSO-d₆) δ 9.00 (s, 2H), 8.09 (s, 1H), 8.02 (s, 1H), 7.73-7.65 (m, 2H), 6.59-6.53 (m, 1H), 6.16 (br s, 1H), 5.09 (br d, J=6.00 Hz, 1H), 4.65 (br d, J=6.40 Hz, 2H), 3.84 (br s, 2H), 3.67-3.58 (m, 2H). MS (ES+) m/e 342 (M+H)⁺.

### Example 60

### (R)—N-((8-fluoroquinoxalin-6-yl)methyl)-4-(2-methylpiperazin-1-yl)pyridin-3-amine (Comp. 065)

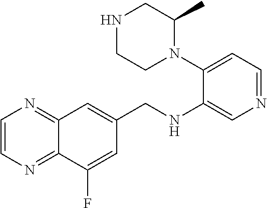 [SMILES: C[C@@H]1CNCCN1c1ccncc1NCc1cc(F)c2nccnc2c1]

¹H NMR (400 MHz, CDCl₃) δ 8.89 (d, J=5.60 Hz, 2H), 8.01 (br d, J=4.00 Hz, 1H), 7.90 (s, 2H), 7.49 (d, J=10.40 Hz, 1H), 6.98 (d, J=4.80 Hz, 1H), 5.38 (br t, J=5.60 Hz, 1H), 4.67 (t, J=5.60 Hz, 2H), 3.33-2.88 (m, 5H), 2.68 (br d, J=10.00 Hz, 2H), 0.94 (d, J=6.00 Hz, 3H). MS (ES+) m/e 353 (M+H)⁺.

### Example 61

### (R)—N-((8-chloroquinoxalin-6-yl)methyl)-4-(2-methylpiperazin-1-yl)pyridin-3-amine (Comp. 066)

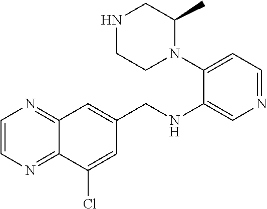 [SMILES: C[C@@H]1CNCCN1c1ccncc1NCc1cc(Cl)c2nccnc2c1]

¹H NMR (400 MHz, CDCl₃) δ 8.95 (d, J=1.60 Hz, 1H), 8.90 (d, J=2.00 Hz, 1H), 8.07-7.97 (m, 2H), 7.95-7.86 (m, 2H), 6.99 (br d, J=4.80 Hz, 1H), 5.37 (br t, J=5.20 Hz, 1H), 4.67 (t, J=5.20 Hz, 2H), 3.41-3.26 (m, 1H), 3.25-2.96 (m, 4H), 2.84-2.66 (m, 2H), 1.02-0.92 (m, 3H). MS (ES+) m/e 369 (M+H)⁺.

### Example 62

### N-((5-fluoroquinoxalin-6-yl)methyl)-4-(piperazin-1-yl)pyridin-3-amine (Comp. 067)

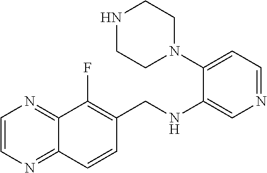 [SMILES: Fc1c(CNc2cnccc2N2CCNCC2)ccc2nccnc12]

¹H NMR (400 MHz, DMSO-d₆) δ 9.74 (s, 2H), 9.03 (s, 2H), 8.12 (d, J=6.4 Hz, 1H), 7.94 (d, J=8.8 Hz, 1H), 7.82-7.87 (m, 2H), 7.41 (d, J=6.4 Hz, 1H), 6.79 (s, 1H), 4.78 (d, J=4 Hz, 2H), 3.49 (s, 4H), 3.39 (s, 4H). MS (ES+) m/e 339 (M+H)⁺.

### Example 63

### N-((8-methylquinoxalin-6-yl)methyl)-4-(piperazin-1-yl)pyridin-3-amine (Comp. 068)

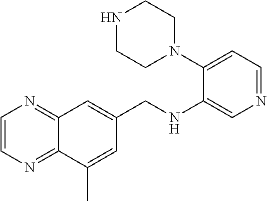 [SMILES: Cc1cc(CNc2cnccc2N2CCNCC2)cc2nccnc12]

¹H NMR (400 MHz, DMSO-d₆) δ 15.8-14.5 (m, 1H), 9.77 (s, 2H), 9.08 (d, J=6.4 Hz, 2H), 8.55 (s, 1H), 8.15 (d, J=6.4 Hz, 1H), 8.02 (s, 1H), 7.68 (s, 1H), 7.47 (d, J=6.4 Hz, 1H), 6.92 (m, 1H), 4.84-4.83 (m, 2H), 3.54 (s, 4H), 3.41 (s, 4H), 2.73 (s, 3H). MS (ES+) m/e 335 (M+H)⁺.

### Example 64

### N-((7-methylquinoxalin-6-yl)methyl)-4-(piperazin-1-yl)pyridin-3-amine (Comp. 069)

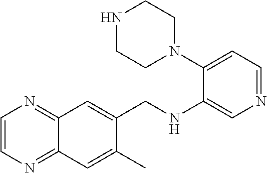 [SMILES: Cc1cc2nccnc2cc1CNc1cnccc1N1CCNCC1]

¹H NMR (400 MHz, DMSO-d₆) δ 9.61 (br d, J=2.1 Hz, 1H), 8.88 (d, J=1.8 Hz, 1H), 8.82 (d, J=1.8 Hz, 1H), 8.12 (d, J=6.4 Hz, 1H), 7.98 (s, 1H), 7.78 (s, 1H), 7.70 (s, 1H), 7.45 (d, J=6.2 Hz, 1H), 6.75 (br t, J=5.4 Hz, 1H), 4.68 (br d, J=5.0 Hz, 2H), 3.52 (br d, J=4.9 Hz, 4H), 3.41 (br s, 4H), 3.16 (s, 2H), 2.62 (s, 3H). MS (ES+) m/e 335 (M+H)⁺.

### Example 65

### N-((7,8-dimethylquinoxalin-6-yl)methyl)-4-(piperazin-1-yl)pyridin-3-amine (Comp. 070)

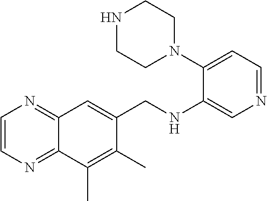 [SMILES: Cc1c(CNc2cnccc2N2CCNCC2)cc2nccnc2c1C]

¹H NMR (400 MHz, DMSO-d₆) δ 8.68 (dd, J₁=14.8 Hz, J₁=1.6 Hz 2H), 8.00 (d, J=6.4 Hz, 1H), 7.60 (s, 1H), 7.98 (s, 1H), 7.50 (s, 1H), 7.42 (d, J=6.4 Hz, 1H), 4.60 (s, 2H), 3.60-3.54 (m, 8H), 2.48 (s, 3H), 2.38 (s, 3H). MS (ES+) m/e 349 (M+H)⁺.

### Example 66

### N-((8-methoxyquinoxalin-6-yl)methyl)-4-(piperazin-1-yl)pyridin-3-amine (Comp. 071)

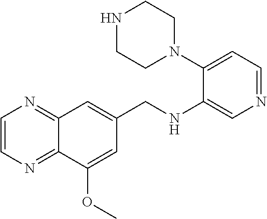 [SMILES: COc1cc(CNc2cnccc2N2CCNCC2)cc2nccnc12]

¹H NMR (400 MHz, DMSO-d₆) δ 14.65-15.30 (m, 1H) 9.74 (br s, 2H) 8.73-9.07 (m, 2H) 8.08 (br d, J=6 Hz, 1H) 7.78 (s, 1H) 7.55 (s, 1H) 7.40 (br d, J=4.89 Hz, 2H) 6.87 (br s, 1H) 4.71 (br s, 2H) 4.01 (s, 3H) 3.34-3.62 (m, 8H). MS (ES+) m/e 351 (M+H)⁺.

### Example 67

### N-((7-methoxyquinoxalin-6-yl)methyl)-4-(piperazin-1-yl)pyridin-3-amine (Comp. 072)

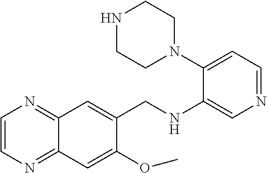 [SMILES: COc1cc2nccnc2cc1CNc1cnccc1N1CCNCC1]

¹H NMR (400 MHz, DMSO-d₆) δ 9.79 (s, 2H), 8.84 (s, 1H), 8.73 (s, 1H), 8.09 (d, J=6 Hz, 1H), 7.75 (d, J=9.2 Hz, 2H), 7.54 (s, 1H), 7.42 (d, J=6 Hz, 1H), 6.73 (br s, 1H), 4.64 (s, 2H), 3.51 (s, 4H), 3.40 (s, 4H). MS (ES+) m/e 351 (M+H)⁺.

### Example 68

### 5-fluoro-4-(piperazin-1-yl)-N-(quinoxalin-6-ylmethyl)pyridin-3-amine (Comp. 073)

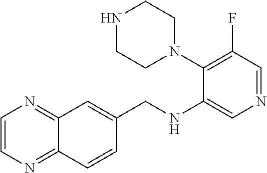 [SMILES: Fc1cncc(NCc2ccc3nccnc3c2)c1N1CCNCC1]

¹H NMR (400 MHz, DMSO-d₆) δ 9.77 (br s, 2H), 8.93 (s, 2H), 8.34 (d, J=5.0 Hz, 1H), 8.14-8.02 (m, 2H), 7.92 (dd, J=1.6, 8.6 Hz, 1H), 7.81 (s, 1H), 7.45-7.16 (m, 1H), 4.81 (s, 2H), 3.66-3.22 (m, 9H). MS (ES+) m/e 339 (M+H)⁺.

### Example 69

### 5-bromo-4-(piperazin-1-yl)-N-(quinoxalin-6-ylmethyl)pyridin-3-amine (Comp. 074)

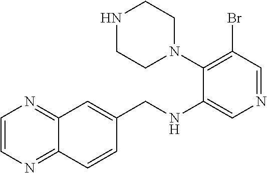 [SMILES: Brc1cncc(NCc2ccc3nccnc3c2)c1N1CCNCC1]

¹H NMR (400 MHz, DMSO-d₆) δ 9.03-8.88 (m, 2H), 8.28 (d, J=1.0 Hz, 1H), 8.17 (d, J=8.6 Hz, 1H), 8.09 (d, J=1.0 Hz, 1H), 8.02-7.96 (m, 1H), 7.92 (d, J=0.9 Hz, 1H), 4.91-4.91 (m, 2H), 3.74 (br s, 4H), 3.62 (br s, 4H). MS (ES+) m/e 390 (M+H)⁺.

### Example 70

### 5-methoxy-4-(piperazin-1-yl)-N-(quinoxalin-6-ylmethyl)pyridin-3-amine (Comp. 075)

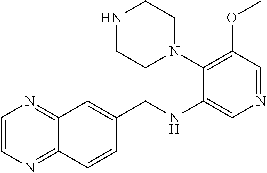 [SMILES: COc1cncc(NCc2ccc3nccnc3c2)c1N1CCNCC1]

¹H NMR (400 MHz, DMSO-d₆) δ 9.57 (br d, J=3.1 Hz, 2H), 8.92 (s, 2H), 8.09 (d, J=8.7 Hz, 1H), 8.03 (s, 1H), 7.96 (s, 1H), 7.90 (dd, J=1.8, 8.7 Hz, 1H), 7.72 (s, 1H), 7.28 (br s, 1H), 4.79 (br s, 2H), 3.93 (s, 3H), 3.37 (br s, 8H). MS (ES+) m/e 351 (M+H)⁺.

### Example 71

### 5-(difluoromethyl)-4-(piperazin-1-yl)-N-(quinoxalin-6-ylmethyl)pyridin-3-amine (Comp. 076)

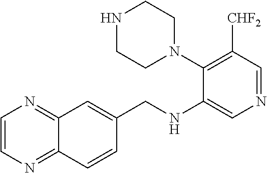 [SMILES: FC(F)c1cncc(NCc2ccc3nccnc3c2)c1N1CCNCC1]

¹H NMR (400 MHz, DMSO-d₆) δ 9.67 (br s, 2H) 8.93 (s, 2H) 8.22 (s, 1H) 8.06-8.12 (m, 2H) 8.04 (s, 1H) 7.93 (dd, J=8.62, 1.77 Hz, 1H) 7.26-7.63 (m, 1H) 7.18 (br s, 1H) 4.84 (br s, 2H) 3.40-3.52 (m, 8H). MS (ES+) m/e 371 (M+H)⁺.

### Example 72

### 5-chloro-N-((8-fluoroquinoxalin-6-yl)methyl)-4-(piperazin-1-yl)pyridin-3-amine (Comp. 077)

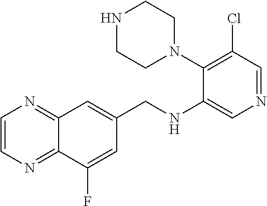 [SMILES: Fc1cc(CNc2cncc(Cl)c2N2CCNCC2)cc2nccnc12]

¹H NMR (400 MHz, DMSO-d₆) δ 9.84-9.38 (m, 2H), 9.01 (d, J=1.6 Hz, 1H), 8.97 (d, J=1.6 Hz, 1H), 8.13 (s, 1H), 7.94 (s, 1H), 7.88 (s, 1H), 7.80 (d, J=11.2 Hz, 1H), 7.54-7.12 (m, 1H), 4.78 (s, 2H), 3.45 (br s, 8H). MS (ES+) m/e 373 (M+H)⁺.

### Example 73

### 5-chloro-N-((5-fluoroquinoxalin-6-yl)methyl)-4-(piperazin-1-yl)pyridin-3-amine (Comp. 078)

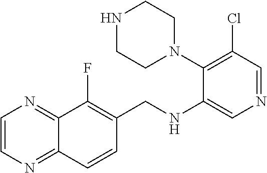 [SMILES: Fc1c(CNc2cncc(Cl)c2N2CCNCC2)ccc2nccnc12]

¹H NMR (400 MHz, DMSO-d₆) δ 8.84-8.87 (m, 2H), 8.06 (s, 1H), 7.78-7.80 (m, 3H), 4.81 (s, 2H), 3.64 (s, 4H), 3.49-3.52 (m, 4H). MS (ES+) m/e 373 (M+H)⁺.

### Example 74

### 5-fluoro-N-((5-fluoroquinoxalin-6-yl)methyl)-4-(piperazin-1-yl)pyridin-3-amine (Comp. 079)

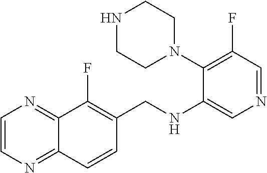 [SMILES: Fc1cncc(NCc2ccc3nccnc3c2F)c1N1CCNCC1]

¹H NMR (400 MHz, DMSO-d₆) δ 9.36 (s, 2H), 9.03 (s, 2H), 8.19 (d, J=4.0 Hz, 1H), 7.93 (d, J=8.8 Hz, 1H), 7.86 (d, J=7.6 Hz, 1H), 7.83 (s, 1H), 6.94 (s, 1H), 4.80 (s, 2H), 3.38 (s, 8H). MS (ES+) m/e 357 (M+H)⁺.

### Example 75

### 5-fluoro-N-((8-fluoroquinoxalin-6-yl)methyl)-4-(piperazin-1-yl)pyridin-3-amine (Comp. 080)

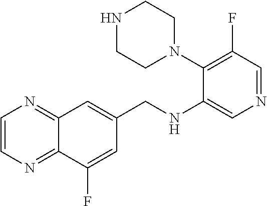 [SMILES: Fc1cncc(NCc2cc(F)c3nccnc3c2)c1N1CCNCC1]

¹H NMR (400 MHz, DMSO-d₆) δ 9.77 (s, 2H), 9.01 (d, J=1.6 Hz, 1H), 8.97 (s, 1H), 8.35 (d, J=5.2 Hz, 1H), 7.94 (s, 1H), 7.80-7.83 (m, 2H), 7.33 (s, 1H), 4.79 (s, 2H), 3.49 (s, 4H), 3.41 (s, 4H). MS (ES+) m/e 357 (M+H)⁺.

### Example 76

### N-((8-chloroquinoxalin-6-yl)methyl)-5-fluoro-4-(piperazin-1-yl)pyridin-3-amine (Comp. 081)

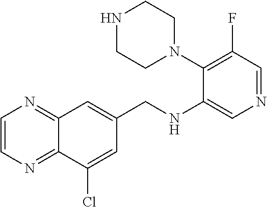 [SMILES: Fc1cncc(NCc2cc(Cl)c3nccnc3c2)c1N1CCNCC1]

¹H NMR (400 MHz, DMSO-d₆) δ 9.73 (s, 2H), 9.03 (d, J=6.4 Hz, 2H), 8.33 (d, J=5.2 Hz, 1H), 8.16 (d, J=1.2 Hz, 1H), 8.04 (s, 1H), 7.85 (s, 1H), 7.29 (s, 1H), 4.80 (s, 2H), 3.48-3.41 (m, 8H). MS (ES+) m/e 373 (M+H)⁺.

### Example 77

### 5-fluoro-N-((7-fluoroquinoxalin-6-yl)methyl)-4-(piperazin-1-yl)pyridin-3-amine (Comp. 082)

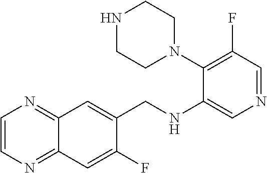 [SMILES: Fc1cc2nccnc2cc1CNc1cncc(F)c1N1CCNCC1]

1H NMR (400 MHz, DMSO-d6) δ 9.69 (br s, 2H), 8.93 (dd, J=1.8, 15.9 Hz, 2H), 8.38 (d, J=5.0 Hz, 1H), 8.02-7.88 (m, 3H), 7.22-7.06 (m, 1H), 4.82 (br s, 2H), 3.56-3.31 (m, 8H). MS (ES+) m/e 357 (M+H)⁺.

### Example 78

### N-((7-chloroquinoxalin-6-yl)methyl)-5-fluoro-4-(piperazin-1-yl)pyridin-3-amine (Comp. 083)

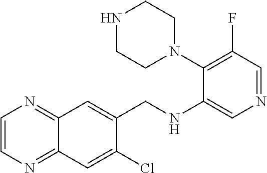 [SMILES: Fc1cncc(NCc2cc3nccnc3cc2Cl)c1N1CCNCC1]

¹H NMR (400 MHz, DMSO-d₆) δ 9.62 (br s, 2H), 8.96 (dd, J=1.7, 10.4 Hz, 2H), 8.36 (d, J=4.8 Hz, 1H), 8.30-8.30 (m, 1H), 8.31 (s, 1H), 7.96 (s, 1H), 7.89 (s, 1H), 7.12 (br s, 1H), 4.79 (br s, 2H), 3.50 (br s, 4H), 3.38 (br s, 4H) MS (ES+) m/e 373 (M+H)⁺.

### Example 79

### N-((8-fluoroquinoxalin-6-yl)methyl)-5-methyl-4-(piperazin-1-yl)pyridin-3-amine (Comp. 084)

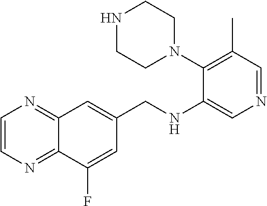 [SMILES: Cc1cncc(NCc2cc(F)c3nccnc3c2)c1N1CCNCC1]

¹H NMR (400 MHz, DMSO-d₆) δ 9.74 (br d, J=1.8 Hz, 2H), 9.07-8.92 (m, 2H), 8.04-7.88 (m, 2H), 7.81 (br d, J=7.8 Hz, 2H), 7.18 (br s, 1H), 4.77 (br s, 2H), 3.46 (br s, 9H), 2.45-2.34 (m, 3H). MS (ES+) m/e 353 (M+H)⁺.

### Example 80

### N-((8-chloroquinoxalin-6-yl)methyl)-5-methyl-4-(piperazin-1-yl)pyridin-3-amine (Comp. 085)

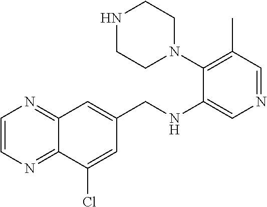 [SMILES: Cc1cncc(NCc2cc(Cl)c3nccnc3c2)c1N1CCNCC1]

¹H NMR (400 MHz, DMSO-d₆) δ 9.90 (s, 2H), 9.01-9.03 (m, 2H), 8.15-8.17 (m, 1H), 8.00-8.02 (s, 1H), 7.95-7.95 (m, 1H), 7.82-7.784 (m, 1H), 7.24 (s, 1H), 4.78 (s, 2H), 3.47 (s, 8H), 2.39-2.42 (m, 3H). MS (ES+) m/e 369 (M+H)⁺.

### Example 81

### N-((7-fluoroquinoxalin-6-yl)methyl)-5-methyl-4-(piperazin-1-yl)pyridin-3-amine (Comp. 086)

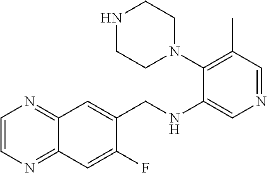 [SMILES: Cc1cncc(NCc2cc3nccnc3cc2F)c1N1CCNCC1]

¹H NMR (400 MHz, DMSO-d₆) δ 15.86-15.10 (m, 1H), 9.74 (br s, 2H), 8.92 (dd, J=1.7, 17.6 Hz, 2H), 8.09-7.81 (m, 4H), 7.03 (br s, 1H), 4.80 (br d, J=3.9 Hz, 2H), 3.61-3.22 (m, 8H), 2.42 (s, 3H). MS (ES+) m/e 353 (M+H)⁺.

### Example 82

### N-((7-chloroquinoxalin-6-yl)methyl)-5-methyl-4-(piperazin-1-yl)pyridin-3-amine (Comp. 087)

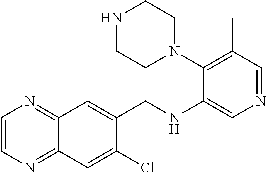 [SMILES: Cc1cncc(NCc2cc3nccnc3cc2Cl)c1N1CCNCC1]

¹H NMR (400 MHz, DMSO-d₆) δ 9.82-9.44 (m, 2H), 8.96 (dd, J=1.9, 12.6 Hz, 2H), 8.31 (s, 1H), 8.03 (s, 1H), 7.90 (d, J=3.8 Hz, 2H), 7.00 (br t, J=5.4 Hz, 1H), 4.77 (br d, J=5.0 Hz, 2H), 3.59-3.30 (m, 8H), 2.44 (s, 3H). MS (ES+) m/e 369 (M+H)⁺.

### Example 83

### 4-(1,4-diazepan-1-yl)-N-((8-fluoroquinoxalin-6-yl)methyl)pyridin-3-amine (Comp. 088)

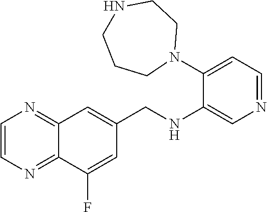 [SMILES: Fc1cc(CNc2cnccc2N2CCCNCC2)cc2nccnc12]

¹H NMR (400 MHz, DMSO-d₆) δ 14.99 (s, 1H), 9.88 (s, 2H), 8.98 (d, J=16 Hz, 2H), 8.02 (d, J=6 Hz, 1H), 7.95 (s, 1H), 7.85 (d, J=10.8 Hz, 1H), 7.74 (s, 1H), 7.33 (d, J=6.4 Hz, 1H), 4.68 (s, 2H), 3.81 (s, 2H), 3.55 (s, 2H), 3.37 (s, 2H), 3.29 (s, 2H), 2.20 (s, 2H). MS (ES+) m/e 353 (M+H)⁺.

### Example 84

### N-((8-chloroquinoxalin-6-yl)methyl)-4-(1,4-diazepan-1-yl)pyridin-3-amine (Comp. 089)

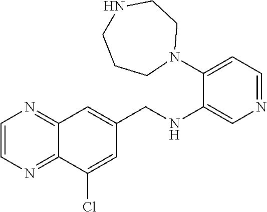 [SMILES: Clc1cc(CNc2cnccc2N2CCCNCC2)cc2nccnc12]

¹H NMR (400 MHz, DMSO-d₆) δ 9.00-8.94 (m, 2H), 8.11 (d, J=1.8 Hz, 1H), 8.07 (d, J=1.5 Hz, 1H), 8.03 (dd, J=1.1, 6.5 Hz, 1H), 7.79 (d, J=1.0 Hz, 1H), 7.43 (d, J=6.5 Hz, 1H), 4.75 (s, 2H), 4.01-3.92 (m, 2H), 3.77-3.69 (m, 2H), 3.63-3.55 (m, 2H), 3.53-3.45 (m, 2H), 2.38-2.30 (m, 2H). MS (ES+) m/e 369 (M+H)⁺.

### Example 85

### 4-(1,4-diazepan-1-yl)-N-((7-fluoroquinoxalin-6-yl)methyl)pyridin-3-amine (Comp. 090)

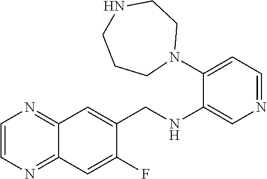 [SMILES: Fc1cc2nccnc2cc1CNc1cnccc1N1CCCNCC1]

¹H NMR (400 MHz, DMSO-d₆) δ 15.05-14.55 (m, 1H), 9.69 (br s, 2H), 8.93 (dd, J=2.0, 15.6 Hz, 2H), 8.06 (dd, J=7.2, 17.2 Hz, 2H), 7.95 (d, J=10.8 Hz, 1H), 7.88 (s, 1H), 7.36 (d, J=6.4 Hz, 1H), 6.68 (br s, 1H), 4.69 (br s, 2H), 3.93-3.76 (m, 2H), 3.57 (br t, J=5.6 Hz, 2H), 3.41-3.18 (m, 4H), 2.18 (s, 2H). MS (ES+) m/e 284 (M+H)⁺.

### Example 86

### N-((7-chloroquinoxalin-6-yl)methyl)-4-(1,4-diazepan-1-yl)pyridin-3-amine (Comp. 091)

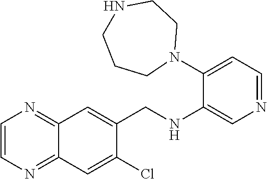 [SMILES: Clc1cc2nccnc2cc1CNc1cnccc1N1CCCNCC1]

¹H NMR (400 MHz, DMSO-d₆) δ 14.77-14.04 (m, 1H), 9.65-9.36 (m, 2H), 8.97 (dd, J=1.8, 10.8 Hz, 2H), 8.32 (s, 1H), 8.11 (d, J=6.4 Hz, 1H), 8.02 (s, 1H), 7.83 (s, 1H), 7.37 (d, J=6.6 Hz, 1H), 6.63 (br t, J=5.4 Hz, 1H), 4.72-4.62 (m, 2H), 3.84 (br s, 2H), 3.63-3.59 (m, 2H), 3.41-3.21 (m, 5H), 2.17 (br s, 2H). MS (ES+) m/e 369 (M+H)⁺.

### Example 87

### 4-(1,4-diazepan-1-yl)-5-fluoro-N-(quinoxalin-6-ylmethyl)pyridin-3-amine (Comp. 092)

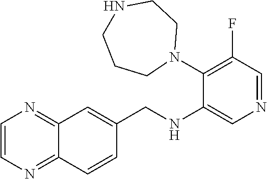 [SMILES: Fc1cncc(NCc2ccc3nccnc3c2)c1N1CCCNCC1]

¹H NMR (400 MHz, DMSO-d₆) δ 9.85 (br s, 2H) 8.92 (q, J=1.83 Hz, 2H) 8.30 (d, J=4.28 Hz, 1H) 8.09 (dd, J=4.83, 3.61 Hz, 2H) 7.97 (dd, J=8.68, 1.83 Hz, 1H) 7.83 (s, 1H) 4.82 (s, 2H) 3.57 (br d, J=4.40 Hz, 2H) 3.26-3.39 (m, 6H) 2.21 (br s, 2H). MS (ES+) m/e 353 (M+H)⁺.

### Example 88

### 5-chloro-4-(1,4-diazepan-1-yl)-N-(quinoxalin-6-ylmethyl)pyridin-3-amine (Comp. 093)

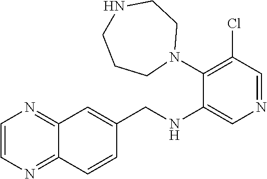 [SMILES: Clc1cncc(NCc2ccc3nccnc3c2)c1N1CCCNCC1]

¹H NMR (400 MHz, DMSO-d₆) δ 10.19-9.80 (m, 2H), 8.93-8.90 (m, 2H), 8.22 (s, 1H), 8.08 (d, J=8.8 Hz, 2H), 7.97 (dd, J=1.6, 8.7 Hz, 1H), 7.92 (s, 1H), 4.86 (s, 2H), 3.57 (br t, J=5.2 Hz, 2H), 3.30 (br s, 6H), 2.24 (br s, 2H). MS (ES+) m/e 369 (M+H)⁺.

### Example 89

### 4-(1,4-diazepan-1-yl)-5-methyl-N-(quinoxalin-6-ylmethyl)pyridin-3-amine (Comp. 094)

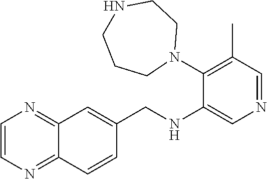 [SMILES: Cc1cncc(NCc2ccc3nccnc3c2)c1N1CCCNCC1]

¹H NMR (400 MHz, DMSO-d₆) δ 14.77 (s, 1H), 10.33-9.60 (m, 2H), 8.91 (s, 2H), 8.11-8.03 (m, 2H), 7.99-7.91 (m, 2H), 7.78 (s, 1H), 7.62 (br t, J=5.6 Hz, 1H), 7.70-7.51 (m, 1H), 5.05 (br s, 8H), 4.82 (br d, J=4.8 Hz, 2H), 3.53 (br t, J=5.1 Hz, 2H), 2.34 (s, 3H). MS (ES+) m/e 349 (M+H)⁺.

### Example 90

### 4-(1,4-diazepan-1-yl)-5-fluoro-N-((8-fluoroquinoxalin-6-yl)methyl)pyridin-3-amine (Comp. 095)

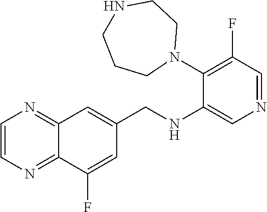 [SMILES: Fc1cncc(NCc2cc(F)c3nccnc3c2)c1N1CCCNCC1]

¹H NMR (400 MHz, D₂O) δ 8.81-8.96 (m, 2H) 8.02 (d, J=4.77 Hz, 1H) 7.82 (br s, 1H) 7.55-7.70 (m, 2H) 4.86-4.96 (m, 2H) 3.68-3.83 (m, 2H) 3.50 (q, J=5.50 Hz, 6H) 2.23 (quin, J=5.59 Hz, 2H). MS (ES+) m/e 371 (M+H)⁺.

### Example 91

### N-((7-chloroquinoxalin-6-yl)methyl)-4-(1,4-diazepan-1-yl)-5-fluoropyridin-3-amine (Comp. 096)

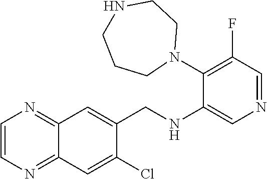 [SMILES: Fc1cncc(NCc2cc3nccnc3cc2Cl)c1N1CCCNCC1]

¹H NMR (400 MHz, DMSO-d₆) δ 8.83 (dd, J=9.17, 1.83 Hz, 2H) 8.21 (s, 1H) 8.03 (br d, J=1.22 Hz, 1H) 7.89 (s, 1H) 7.60-7.71 (m, 1H) 4.72 (s, 1H) 4.70-4.73 (m, 1H) 3.60 (br s, 2H) 3.34 (br d, J=5.50 Hz, 6H) 2.00-2.18 (m, 1H) 1.99-2.20 (m, 1H). MS (ES+) m/e 387 (M+H)⁺.

### Example 92

### 4-((1S,4S)-2,5-diazabicyclo[2.2.1]heptan-2-yl)-N-((7-chloroquinoxalin-6-yl)methyl)pyridin-3-amine (Comp. 097)

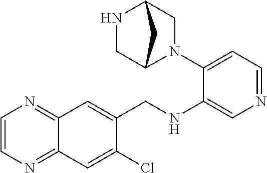 [SMILES: Clc1cc2nccnc2cc1CNc1cnccc1N1C[C@@H]2C[C@H]1CN2]

¹H NMR (400 MHz, DMSO-d₆) δ 8.93 (dd, J=1.6, 9.2 Hz, 2H), 8.27 (s, 1H), 8.02 (s, 1H), 7.71 (d, J=5.4 Hz, 1H), 7.49 (s, 1H), 6.67 (d, J=5.2 Hz, 1H), 5.30 (br t, J=6.0 Hz, 1H), 4.56 (br dd, J=6.0, 9.6 Hz, 2H), 4.28 (s, 1H), 3.70 (dd, J=2.0, 8.8 Hz, 1H), 3.61 (br s, 1H), 3.15 (br d, J=8.8 Hz, 1H), 3.08 (br d, J=10.0 Hz, 1H), 2.86 (br d, J=8.4 Hz, 1H), 1.79 (br d, J=8.8 Hz, 1H), 1.65 (br d, J=9.2 Hz, 1H). MS (ES+) m/e 467 (M+H)⁺.

### Example 93

### 4-((1R,4R)-2,5-diazabicyclo[2.2.1]heptan-2-yl)-N-((7-chloroquinoxalin-6-yl)methyl)pyridin-3-amine (Comp. 098)

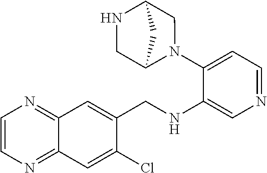 [SMILES: Clc1cc2nccnc2cc1CNc1cnccc1N1C[C@H]2C[C@@H]1CN2]

¹H NMR (400 MHz, CD₃OD) δ 8.91 (dd, J=1.8, 10.4 Hz, 2H), 8.26 (s, 1H), 8.12 (s, 1H), 8.04-7.95 (m, 1H), 7.77-7.67 (m, 1H), 7.26-7.14 (m, 1H), 5.14 (s, 1H), 4.87-4.86 (m, 2H), 4.78-4.58 (m, 3H), 4.30 (dd, J=2.6, 11.7 Hz, 1H), 3.93 (dd, J=1.2, 11.6 Hz, 1H), 3.73-3.63 (m, 1H), 3.57-3.48 (m, 1H), 2.40 (br d, J=11.7 Hz, 1H), 2.28-2.13 (m, 1H). MS (ES+) m/e 367 (M+H)⁺.

### Example 94

### N-((7-chloroquinoxalin-6-yl)methyl)-4-(hexahydropyrrolo[3,4-c]pyrrol-2(1H)-yl)pyridin-3-amine (Comp. 099)

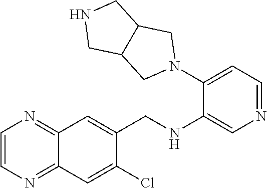 [SMILES: Clc1cc2nccnc2cc1CNc1cnccc1N1CC2CNCC2C1]

¹H NMR (400 MHz, D₂O) δ 8.88-8.77 (m, 2H), 8.15 (s, 1H), 7.93 (s, 1H), 7.89-7.81 (m, 1H), 7.51 (s, 1H), 7.03 (br d, J=6.7 Hz, 1H), 4.64 (s, 2H), 3.80 (br s, 4H), 3.64 (br d, J=4.6 Hz, 2H), 3.33 (br d, J=8.6 Hz, 4H). MS (ES+) m/e 381 (M+H)⁺.

### Example 95

### (R)—N-((7-fluoroquinoxalin-6-yl)methyl)-4-(2-methylpiperazin-1-yl)pyridin-3-amine (Comp. 100)

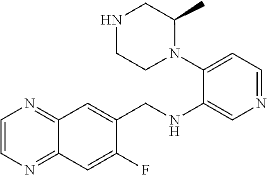 [SMILES: C[C@@H]1CNCCN1c1ccncc1NCc1cc2nccnc2cc1F]

¹H NMR (400 MHz, DMSO-d₆) δ 9.79-10.02 (m, 2H), 8.94 (d, J=1.6 Hz, 1H), 8.90 (s, 1H), 8.10 (d, J=6 Hz, 1H), 7.99 (s, 1H), 7.95 (d, J=10.8 Hz, 1H), 7.87 (d, J=8 Hz, 1H), 7.53 (d, J=6 Hz, 1H), 7.09 (s, 1H), 4.45-4.87 (m, 2H), 4.06 (s, 1H), 3.61-3.51 (m, 1H), 3.46-3.49 (m, 2H), 3.16-3.22 (m, 2H), 3.14 (d, J=6 Hz, 1H), 1.10 (d, J=6 Hz, 3H). MS (ES+) m/e 353 (M+H)⁺.

### Example 96

### (S)—N-((8-chloroquinoxalin-6-yl)methyl)-4-(3-methylpiperazin-1-yl)pyridin-3-amine (Comp. 101)

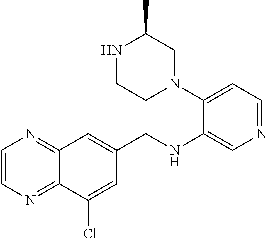 [SMILES: C[C@H]1CN(c2ccncc2NCc2cc(Cl)c3nccnc3c2)CCN1]

¹H NMR (400 MHz, DMSO-d₆) δ 15.19 (s, 1H), 10.18 (d, J=8.4 Hz, 1H), 9.80 (d, J=8.8 Hz, 1H), 9.00-9.00 (m, 1H), 9.02 (d, J=2.8 Hz, 1H), 8.16 (d, J=1.6 Hz, 1H), 8.10-8.00 (m, 2H), 7.85 (s, 1H), 7.39 (d, J=6.4 Hz, 1H), 6.93 (br s, 1H), 4.93-4.62 (m, 2H), 3.92-3.61 (m, 3H), 3.56-3.35 (m, 2H), 3.33-3.20 (m, 1H), 3.14-3.00 (m, 1H), 1.36 (d, J=6.3 Hz, 3H). MS (ES+) m/e 369 (M+H)⁺.

### Example 97

### (S)—N-((7-fluoroquinoxalin-6-yl)methyl)-4-(3-methylpiperazin-1-yl)pyridin-3-amine (Comp. 102)

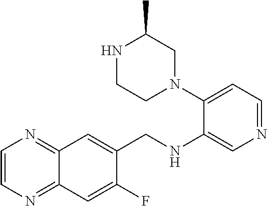 [SMILES: C[C@H]1CN(c2ccncc2NCc2cc3nccnc3cc2F)CCN1]

¹H NMR (400 MHz, DMSO-d₆) 15.1 (s, 1H), 10.0-10.1 (m, 1H), 9.64 (s, 1H), 8.95 (d, J=1.6 Hz, 1H), 8.91 (d, J=1.6 Hz, 1H), 8.12 (d, J=6.4 Hz, 1H), 7.98-7.94 (m, 3H), 7.44 (d, J=6.4 Hz, 1H), 6.75 (t, J=6.4 Hz, 1H), 4.78 (d, J=3.6 Hz, 2H), 3.78-3.75 (m, 2H), 3.66-3.62 (m, 1H), 3.42 (m, 2H), 3.33-3.23 (m, 1H), 3.09-3.04 (m, 1H), 1.34 (d, J=6.4 Hz, 3H). MS (ES+) m/e 353 (M+H)⁺.

### Example 98

### (S)-5-fluoro-4-(3-methylpiperazin-1-yl)-N-(quinoxalin-6-ylmethyl)pyridin-3-amine (Comp. 103)

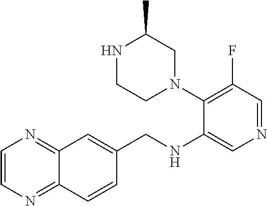 [SMILES: C[C@H]1CN(c2c(F)cncc2NCc2ccc3nccnc3c2)CCN1]

¹H NMR (400 MHz, DMSO-d₆) δ 10.07 (br d, J=9.7 Hz, 1H), 9.41 (br d, J=9.8 Hz, 1H), 9.01-8.86 (m, 2H), 8.27 (d, J=4.6 Hz, 1H), 8.14-8.04 (m, 2H), 7.92 (dd, J=1.8, 8.7 Hz, 1H), 7.82 (s, 1H), 7.26-7.05 (m, 1H), 4.79 (s, 2H), 3.76-3.62 (m, 2H), 3.57-3.32 (m, 6H), 3.31-3.20 (m, 1H), 1.31 (d, J=6.6 Hz, 3H). MS (ES+) m/e 353 (M+H)⁺.

### Example 99

### (S)-5-methyl-4-(3-methylpiperazin-1-yl)-N-(quinoxalin-6-ylmethyl)pyridin-3-amine (Comp. 104)

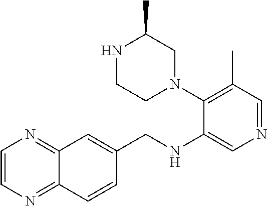 [SMILES: Cc1cncc(NCc2ccc3nccnc3c2)c1N1CCN[C@@H](C)C1]

¹H NMR (400 MHz, DMSO-d₆) δ 15.80-14.91 (m, 1H), 10.10 (br d, J=9.5 Hz, 1H), 9.64-9.32 (m, 1H), 8.92 (s, 2H), 8.18-8.02 (m, 2H), 7.98-7.88 (m, 2H), 7.80 (s, 1H), 7.09 (br s, 1H), 4.78 (br s, 2H), 3.92-3.76 (m, 2H), 3.42 (br t, J=11.6 Hz, 1H), 3.36-3.18 (m, 3H), 2.40 (s, 3H), 1.32 (br d, J=6.4 Hz, 3H). MS (ES+) m/e 349 (M+H)⁺.

### Example 100

### (R)—N-((8-fluoroquinoxalin-6-yl)methyl)-4-(pyrrolidin-3-yloxy)pyridin-3-amine (Comp. 105)

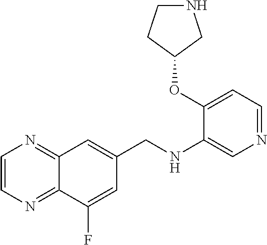 [SMILES: Fc1cc(CNc2cnccc2O[C@@H]2CCNC2)cc2nccnc12]

¹H NMR (400 MHz, DMSO-d₆) δ 15.16 (br s, 1H) 10.47 (br s, 1H) 9.90 (br d, J=1.96 Hz, 1H) 8.88-9.08 (m, 2H) 8.11 (br d, J=6.24 Hz, 1H) 7.99 (s, 1H) 7.75-7.96 (m, 3H) 7.47-7.58 (m, 1H) 5.65 (br s, 1H) 4.71 (br s, 2H) 3.61-3.75 (m, 1H) 3.31-3.55 (m, 3H) 2.19-2.32 (m, 2H). MS (ES+) m/e 340 (M+H)⁺.

### Example 101

### (R)—N-((8-chloroquinoxalin-6-yl)methyl)-4-(pyrrolidin-3-yloxy)pyridin-3-amine (Comp. 106)

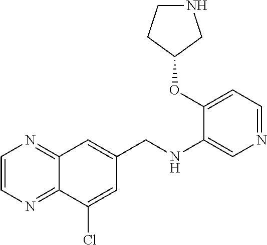 [SMILES: Clc1cc(CNc2cnccc2O[C@@H]2CCNC2)cc2nccnc12]

¹H NMR (400 MHz, DMSO-d₆) δ 15.00 (br d, J=3.18 Hz, 1H) 10.41 (br s, 1H) 9.75 (br d, J=2.45 Hz, 1H) 9.02 (q, J=1.79 Hz, 2H) 8.21 (d, J=1.71 Hz, 1H) 8.06-8.16 (m, 2H) 7.78-7.97 (m, 2H) 7.54 (d, J=6.48 Hz, 1H) 5.64 (br s, 1H) 4.72 (br s, 2H) 3.69 (br dd, J=12.35, 4.89 Hz, 1H) 3.47-3.54 (m, 1H) 3.32-3.45 (m, 2H) 2.20-2.34 (m, 2H). MS (ES+) m/e 356 (M+H)⁺.

### Example 102

### (R)—N-((7-fluoroquinoxalin-6-yl)methyl)-4-(pyrrolidin-3-yloxy)pyridin-3-amine (Comp. 107)

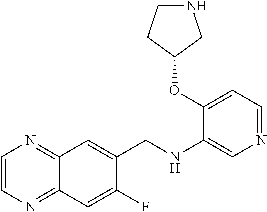 [SMILES: Fc1cc2nccnc2cc1CNc1cnccc1O[C@@H]1CCNC1]

¹H NMR (400 MHz, DMSO-d₆) δ 10.04 (s, 1H), 9.99 (s, 1H), 8.89-9.93 (m, 2H), 8.15 (d, J=6.4 Hz, 1H), 7.91-7.94 (m, 3H), 7.72 (s, 1H), 7.58 (d, J=6.4 Hz, 1H), 5.66 (s, 1H), 4.71-4.80 (m, 2H), 3.65-3.68 (m, 1H), 3.39-3.50 (m, 3H), 2.27-2.32 (m, 2H). MS (ES+) m/e 340 (M+H)⁺.

### Example 103

### (R)—N-((7-chloroquinoxalin-6-yl)methyl)-4-(pyrrolidin-3-yloxy)pyridin-3-amine (Comp. 108)

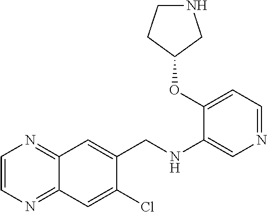 [SMILES: Clc1cc2nccnc2cc1CNc1cnccc1O[C@@H]1CCNC1]

¹H NMR (400 MHz, DMSO-d₆) δ 14.98-15.25 (m, 1H) 10.36 (br s, 1H) 9.85 (br s, 1H) 8.75-9.17 (m, 1H) 8.94 (dd, J=13.02, 1.77 Hz, 1H) 8.28 (s, 1H) 8.17 (d, J=6.36 Hz, 1H) 7.85 (d, J=12.10 Hz, 2H) 7.75 (br s, 1H) 7.68-7.81 (m, 1H) 7.60 (d, J=6.48 Hz, 1H) 5.67 (br s, 1H) 4.64-4.84 (m, 2H) 3.68 (br dd, J=12.53, 5.07 Hz, 1H) 3.49 (td, J=8.07, 3.91 Hz, 1H) 3.33-3.44 (m, 2H) 2.21-2.39 (m, 2H). MS (ES+) m/e 356 (M+H)⁺.

### Example 104

### (R)-5-fluoro-4-(pyrrolidin-3-yloxy)-N-(quinoxalin-6-ylmethyl)pyridin-3-amine (Comp. 109)

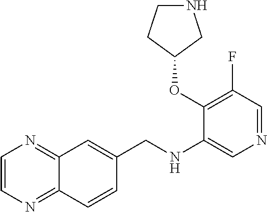 [SMILES: Fc1cncc(NCc2ccc3nccnc3c2)c1O[C@@H]1CCNC1]

¹H NMR (400 MHz, DMSO-d₆) δ 10.50 (br s, 1H), 9.76 (br d, J=1.7 Hz, 1H), 8.92 (q, J=1.8 Hz, 2H), 8.40 (d, J=5.4 Hz, 1H), 8.12 (d, J=1.0 Hz, 1H), 8.09 (d, J=8.7 Hz, 1H), 8.02-7.95 (m, 1H), 7.87 (s, 1H), 5.74 (br d, J=3.1 Hz, 1H), 4.77 (s, 2H), 3.72 (br dd, J=5.9, 13.0 Hz, 2H), 3.52-3.35 (m, 4H), 2.34-2.22 (m, 2H). MS (ES+) m/e 340 (M+H)⁺.

### Example 105

### (R)-5-chloro-4-(pyrrolidin-3-yloxy)-N-(quinoxalin-6-ylmethyl)pyridin-3-amine (Comp. 110)

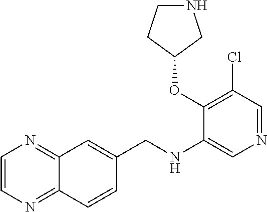 [SMILES: Clc1cncc(NCc2ccc3nccnc3c2)c1O[C@@H]1CCNC1]

¹H NMR (400 MHz, DMSO-d₆) δ 10.60-10.28 (m, 1H), 9.78-9.52 (m, 1H), 8.92 (q, J=1.8 Hz, 2H), 8.22 (s, 1H), 8.15-8.05 (m, 2H), 7.97 (s, 2H), 5.59 (br s, 1H), 4.78 (br s, 2H), 3.66 (br dd, J=5.8, 13.4 Hz, 1H), 3.58-3.32 (m, 3H), 2.31-2.15 (m, 2H). MS (ES+) m/e 356 (M+H)⁺.

### Example 106

### (R)-5-methyl-4-(pyrrolidin-3-yloxy)-N-(quinoxalin-6-ylmethyl)pyridin-3-amine (Comp. 111)

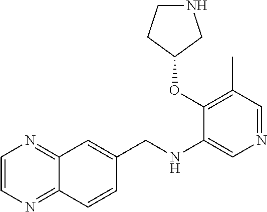 [SMILES: Cc1cncc(NCc2ccc3nccnc3c2)c1O[C@@H]1CCNC1]

¹H NMR (400 MHz, DMSO-d₆) δ 10.61 (br s, 1H) 10.44-10.80 (m, 1H) 9.61-10.04 (m, 1H) 9.59-9.87 (m, 1H) 9.59-10.02 (m, 1H) 8.92 (br s, 2H) 8.06-8.12 (m, 1H) 8.05-8.15 (m, 1H) 7.96-8.05 (m, 1H) 7.95-8.00 (m, 1H) 7.87-7.93 (m, 1H) 7.87-7.92 (m, 1H) 7.80-7.93 (m, 1H) 7.85 (br s, 1H) 5.35-5.48 (m, 1H) 5.42 (br s, 1H) 4.76-4.77 (m, 1H) 4.77 (br s, 1H) 3.32-3.70 (m, 4H) 2.38 (s, 1H) 2.34-2.42 (m, 1H) 2.22 (br s, 2H). MS (ES+) m/e 336 (M+H)⁺.

### Example 107

### 4-(azetidin-3-yloxy)-5-bromo-N-(quinoxalin-6-ylmethyl)pyridin-3-amine (Comp. 112)

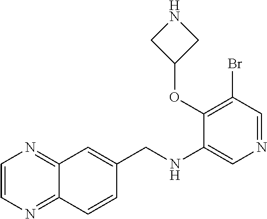 [SMILES: Brc1cncc(NCc2ccc3nccnc3c2)c1OC1CNC1]

¹H NMR (400 MHz, DMSO-d₆) δ 8.87 (s, 2H), 8.17-8.09 (m, 2H), 8.03 (s, 1H), 7.91-7.86 (m, 2H), 5.44-5.30 (m, 1H), 4.79 (s, 2H), 4.70-4.62 (m, 2H), 4.61-4.51 (m, 2H). MS (ES+) m/e 388 (M+H)⁺.

### Example 108

### 4-(azetidin-3-yloxy)-5-methyl-N-(quinoxalin-6-ylmethyl)pyridin-3-amine (Comp. 113)

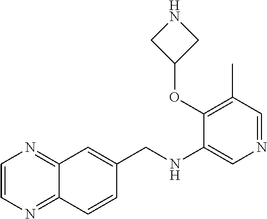 [SMILES: Cc1cncc(NCc2ccc3nccnc3c2)c1OC1CNC1]

¹H NMR (400 MHz, DMSO-d₆) δ 9.39-9.10 (m, 1H), 9.04-8.76 (m, 3H), 8.11 (d, J=8.6 Hz, 1H), 8.06-8.00 (m, 2H), 7.95 (s, 1H), 7.88 (dd, J=1.9, 8.8 Hz, 1H), 7.25 (br s, 1H), 5.20 (quin, J=6.2 Hz, 1H), 4.74 (br d, J=4.9 Hz, 2H), 4.40 (br dd, J=5.4, 11.8 Hz, 5H), 2.27 (s, 3H). MS (ES+) m/e 322 (M+H)⁺.

### Example 109

### 4-(piperazin-1-yl)-N-(quinoxalin-6-ylmethyl)-1H-pyrrolo[2,3-b]pyridin-5-amine (Comp. 114)

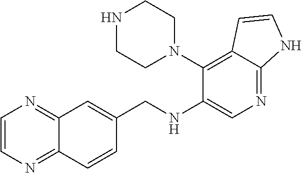 [SMILES: c1cnc2cc(CNc3cnc4[nH]ccc4c3N3CCNCC3)ccc2n1]

¹H NMR (400 MHz, DMSO-d₆) δ 12.1 (s, 1H), 9.53 (s, 2H), 8.92 (s, 2H), 8.11-8.08 (m, 2H), 7.95 (d, J=8.4 Hz, 1H), 7.58 (s, 1H), 7.50 (s, 1H), 6.74 (s, 1H), 4.69 (s, 2H), 3.66 (s, 8H). MS (ES+) m/e 360 (M+H)⁺.

### Example 110

### 4-(piperazin-1-yl)-N-(quinoxalin-6-ylmethyl)quinolin-3-amine (Comp. 115)

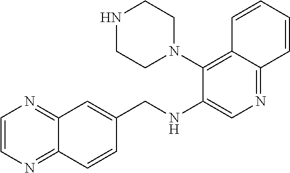 [SMILES: c1ccc2c(N3CCNCC3)c(NCc3ccc4nccnc4c3)cnc2c1]

¹H NMR (400 MHz, DMSO-d₆) δ 9.72 (s, 2H), 8.90 (s, 2H), 8.65 (s, 1H), 8.19 (d, J=8.0 Hz, 1H), 8.14 (d, J=7.2 Hz, 1H), 8.08 (d, J=8.8 Hz, 2H), 7.96 (d, J=8.8 Hz, 1H), 7.71-7.68 (m, 2H), 7.17 (s, 1H), 4.94 (s, 2H), 3.69 (s, 8H). MS (ES+) m/e 371 (M+H)⁺.

### Example 111

### 6-methoxy-4-(piperazin-1-yl)-N-(quinoxalin-6-ylmethyl)pyridin-3-amine (Comp. 116)

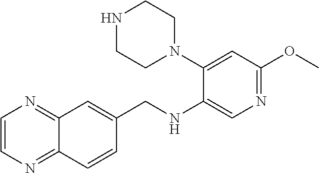 [SMILES: COc1cc(N2CCNCC2)c(NCc2ccc3nccnc3c2)cn1]

¹H NMR (400 MHz, DMSO-d₆) δ 9.87 (s, 2H), 8.92 (s, 2H), 8.08 (d, J=8.4, 1H), 8.04 (s, 1H), 7.91 (d, J=8.8, 1H), 7.15 (s, 1H), 6.78 (s, 1H), 4.63 (s, 3H), 4.00 (s, 3H), 3.59 (s, 4H), 3.39 (s, 4H). MS (ES+) m/e 351 (M+H)⁺.

### Example 112

### 4-(piperazin-1-yl)-N-(quinoxalin-6-ylmethyl)-6-(trifluoromethyl)pyridin-3-amine (Comp. 117)

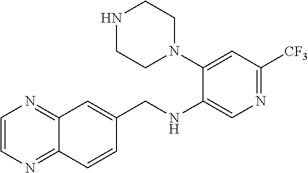 [SMILES: FC(F)(F)c1cc(N2CCNCC2)c(NCc2ccc3nccnc3c2)cn1]

¹H NMR (400 MHz, DMSO-d₆) δ 9.60 (s, 2H), 8.91 (s, 2H), 8.09 (d, J=8.8 Hz, 2H), 8.03 (s, 1H), 7.90 (d, J=1.6 Hz, 1H), 7.86 (s, 1H), 7.26 (m, 1H), 4.78 (s, 2H), 3.37 (s, 4H), 3.24 (s, 4H). MS (ES+) m/e 389 (M+H)⁺.

### Example 113

### 6-methyl-4-(piperazin-1-yl)-N-(quinoxalin-6-ylmethyl)pyridin-3-amine (Comp. 118)

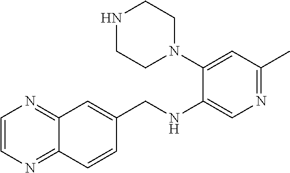 [SMILES: Cc1cc(N2CCNCC2)c(NCc2ccc3nccnc3c2)cn1]

¹H NMR (400 MHz, DMSO-d₆) δ 8.89 (s, 2H), 8.07 (d, J=8.8 Hz, 2H), 8.95 (s, 2H), 7.97 (s, 1H), 7.87 (d, J=8.4 Hz, 1H), 7.53 (s, 1H), 6.71 (s, 1H), 5.46 (s, 1H), 4.64 (d, J=6 Hz, 2H), 2.89-3.24 (m, 8H), 2.23 (s, 3H). MS (ES+) m/e 335 (M+H)⁺.

### Example 114

### (R)-5-chloro-N-((8-fluoroquinoxalin-6-yl)methyl)-4-(pyrrolidin-3-yloxy)pyridin-3-amine (Comp. 119)

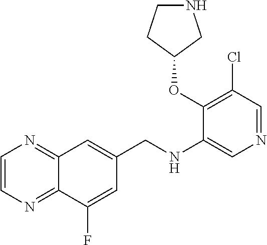 [SMILES: Fc1cc(CNc2cncc(Cl)c2O[C@@H]2CCNC2)cc2nccnc12]

¹H NMR (400 MHz, DMSO-d₆) δ 10.45 (s, 1H), 9.63 (s, 1H), 8.98 (d, J=15.6 Hz, 2H), 8.17 (s, 1H), 7.99 (s, 1H), 7.97 (s, 1H), 7.88 (d, J=9.6 Hz, 1H), 7.76 (s, 1H), 5.55 (s, 1H), 4.79 (s, 3H), 3.64-3.68 (m, 1H), 3.41-3.45 (m, 3H), 2.51 (m, 1H), 2.26-2.29 (m, 2H). MS (ES+) m/e 374 (M+H)⁺.

### Example 115

### (R)-5-chloro-N-((7-fluoroquinoxalin-6-yl)methyl)-4-(pyrrolidin-3-yloxy)pyridin-3-amine (Comp. 120)

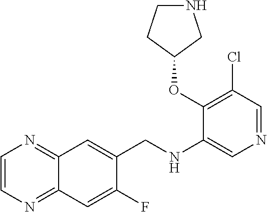 [SMILES: Fc1cc2nccnc2cc1CNc1cncc(Cl)c1O[C@@H]1CCNC1]

¹H NMR (400 MHz, DMSO-d₆) δ 10.24 (s, 1H), 9.55 (s, 1H), 8.92 (d, J=14 Hz, 2H), 8.14 (s, 1H), 7.92-7.98 (m, 3H), 7.41 (s, 1H), 5.49 (s, 1H), 4.75 (s, 2H), 3.50-3.67 (m, 1H), 3.38-3.45 (m, 3H), 2.52 (m, 1H), 2.25-2.28 (m, 2H). MS (ES+) m/e 374 (M+H)⁺.

### Example 116

### (R)-5-chloro-N-((7-chloroquinoxalin-6-yl)methyl)-4-(pyrrolidin-3-yloxy)pyridin-3-amine (Comp. 121)

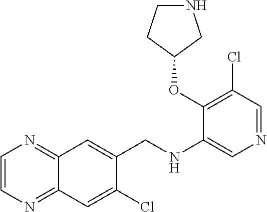 [SMILES: Clc1cc2nccnc2cc1CNc1cncc(Cl)c1O[C@@H]1CCNC1]

¹H NMR (400 MHz, CDCl₃) δ 8.77-8.78 (m, 2H), 8.17 (d, J=0.8 Hz, 1H), 8.00 (s, 1H), 7.82 (s, 1H), 7.79 (s, 1H), 5.86-5.88 (m, 1H), 4.75-4.80 (m, 3H), 3.88 (d, J=13.6 Hz, 1H), 3.58-3.71 (m, 3H), 2.43-2.49 (m, 2H). MS (ES+) m/e 390 (M+H)⁺.

### Example 117

### (R)—N-((8-methylquinoxalin-6-yl)methyl)-4-(pyrrolidin-3-yloxy)pyridin-3-amine (Comp. 122)

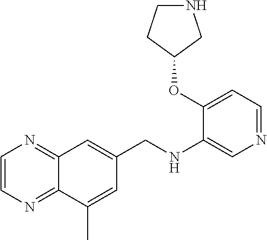 [SMILES: Cc1cc(CNc2cnccc2O[C@@H]2CCNC2)cc2nccnc12]

¹H NMR (400 MHz, DMSO-d₆) δ 10.53-10.28 (m, 1H), 9.91-9.65 (m, 1H), 8.91 (q, J=2.0 Hz, 2H), 8.10 (d, J=6.4 Hz, 1H), 7.89 (s, 1H), 7.81 (m, 3H), 7.53 (d, J=6.4 Hz, 1H), 5.64 (m, 1H), 4.68 (m, 2H), 3.57-3.36 (m, 3H), 2.71 (s, 3H), 2.38-2.18 (m, 2H). MS (ES+) m/e 366 (M+H)⁺.

### Example 118

### (R)—N-((7-methylquinoxalin-6-yl)methyl)-4-(pyrrolidin-3-yloxy)pyridin-3-amine (Comp. 123)

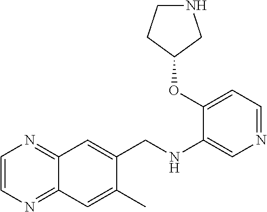 [SMILES: Cc1cc2nccnc2cc1CNc1cnccc1O[C@@H]1CCNC1]

¹H NMR (400 MHz, CD₃OD) δ 8.98 (d, J=2.0 Hz, 1H), 8.94 (d, J=2.2 Hz, 1H), 8.11 (m, 1H), 8.05 (s, 1H), 8.00 (s, 1H), 7.78 (d, J=1.2 Hz, 1H), 7.59 (d, J=6.6 Hz, 1H), 5.74 (m, 1H), 4.81 (s, 2H), 3.92 (d, J=13.2 Hz, 1H), 3.79-3.56 (m, 3H), 2.71 (s, 3H), 2.61-2.50 (m, 2H). MS (ES+) m/e 336 (M+H)⁺.

### Example 119

### (R)-4-(pyrrolidin-3-yloxy)-N-((8-(trifluoromethyl)quinoxalin-6-yl)methyl)pyridin-3-amine (Comp. 124)

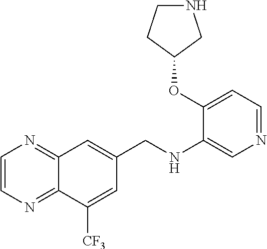 [SMILES: FC(F)(F)c1cc(CNc2cnccc2O[C@@H]2CCNC2)cc2nccnc12]

¹H NMR (400 MHz, DMSO-d₆) δ 10.62-10.21 (m, 1H), 9.77-9.57 (m, 1H), 9.07 (s, 2H), 8.47 (s, 1H), 8.36 (s, 1H), 8.13 (d, J=6.2 Hz, 1H), 7.94 (s, 1H), 7.89 (br s, 1H), 7.54 (d, J=6.5 Hz, 1H), 5.70-5.57 (m, 1H), 4.79 (br s, 2H), 3.68 (br dd, J=4.8, 12.2 Hz, 1H), 3.57-3.33 (m, 3H), 2.33-2.21 (m, 2H). MS (ES+) m/e 389 (M+H)⁺.

### Example 120

### 4-(piperazin-1-yl)-N-(quinoxalin-6-ylmethyl)-5-(trifluoromethyl)pyridin-3-amine (Comp. 125)

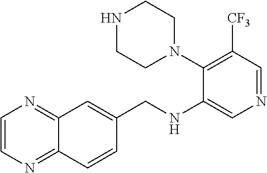 [SMILES: FC(F)(F)c1cncc(NCc2ccc3nccnc3c2)c1N1CCNCC1]

¹H NMR (400 MHz, MeOD) δ 8.94 (d, J=1.8 Hz, 2H), 8.44 (s, 1H), 8.20-8.15 (m, 2H), 8.12 (s, 1H), 8.01 (dd, J=1.6, 8.8 Hz, 1H), 4.97 (s, 2H), 3.64 (s, 8H), MS (ES+) m/z 389.3 (M+H)⁺.

### Example 121

### (S)-4-(3-methylpiperazin-1-yl)-N-(quinoxalin-6-ylmethyl)-5-(trifluoromethyl)pyridin-3-amine (Comp. 126)

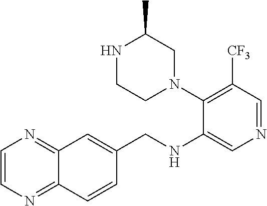 [SMILES: C[C@H]1CN(c2c(NCc3ccc4nccnc4c3)cncc2C(F)(F)F)CCN1]

¹H NMR (400 MHz, MeOD) δ 8.90 (s, 2H), 8.44 (s, 1H), 8.19-8.13 (m, 2H), 8.08 (s, 1H), 8.00-7.94 (m, 1H), 4.95 (s, 2H), 3.89 (dt, 11=3.2 Hz, J2=6.4 Hz, 1H), 3.75-3.66 (m, 1H), 3.64-3.55 (m, 4H), 3.37 (br d, J=12.0 Hz, 1H), 1.42 (d, J=6.40 Hz, 3H). MS (ES+) m/z 403 (M+H)⁺.

### Example 122

### (S)-5-methoxy-4-(3-methylpiperazin-1-yl)-N-(quinoxalin-6-ylmethyl)pyridin-3-amine (Comp. 127)

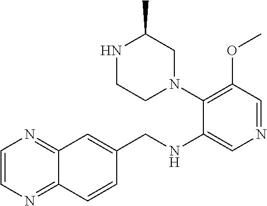 [SMILES: COc1cncc(NCc2ccc3nccnc3c2)c1N1CCN[C@@H](C)C1]

¹H NMR (400 MHz, MeOD) δ 9.04-8.82 (m, 2H), 8.15 (d, J=8.8 Hz, 1H), 8.05 (s, 1H), 7.99-7.91 (m, 2H), 7.69 (s, 1H), 4.86-4.85 (m, 2H), 4.03 (s, 3H), 3.87-3.74 (m, 1H), 3.73-3.57 (m, 2H), 3.55-3.42 (m, 2H), 3.42-3.33 (m, 2H), 1.41 (d, J=6.5 Hz, 3H), MS (ES+) m/z 365.3 (M+H)⁺.

### Example 123

### (S)-5-bromo-4-(3-methylpiperazin-1-yl)-N-(quinoxalin-6-ylmethyl)pyridin-3-amine (Comp. 128)

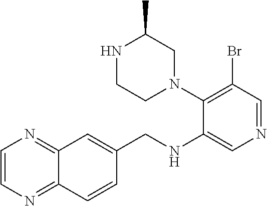 [SMILES: C[C@H]1CN(c2c(Br)cncc2NCc2ccc3nccnc3c2)CCN1]

¹H NMR: (400 MHz, MeOD-d₄) δ 8.94 (q, J=2.0 Hz, 2H), 8.28 (d, J=0.8 Hz, 1H), 8.16 (d, J=8.8 Hz, 1H), 8.09 (s, 1H), 7.99 (dd, J=1.8, 8.7 Hz, 1H), 7.93 (s, 1H), 5.01-4.92 (m, 2H), 4.04-3.90 (m, 2H), 3.84-3.68 (m, 2H), 3.62-3.41 (m, 3H), 1.44 (d, J=6.6 Hz, 3H) MS (ES+) m/z 415 (M+H)⁺

### Example 124

### (S)-5-(difluoromethyl)-4-(3-methylpiperazin-1-yl)-N-(quinoxalin-6-ylmethyl)pyridin-3-amine (Comp. 129)

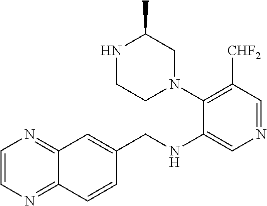 [SMILES: C[C@H]1CN(c2c(NCc3ccc4nccnc4c3)cncc2C(F)F)CCN1]

¹H NMR (400 MHz, MeOD-d₄) δ 8.96-8.85 (m, 2H), 8.29 (s, 1H), 8.16 (d, J=8.6 Hz, 1H), 8.07 (d, J=15.5 Hz, 2H), 7.98 (dd, J=1.8, 8.8 Hz, 1H), 7.33 (t, J=54.2 Hz, 1H), 4.92 (s, 2H), 3.92 (ddd, J=3.4, 6.6, 10.2 Hz, 1H), 3.81-3.70 (m, 1H), 3.68-3.58 (m, 3H), 3.56-3.45 (m, 2H), 1.43 (d, J=6.5 Hz, 3H) MS (ES+) m/z 385 (M+H)⁺

### Example 125

### N-((7-fluoroquinoxalin-6-yl)methyl)-4-(piperazin-1-yl)-5-(trifluoromethyl)pyridin-3-amine (Comp. 130)

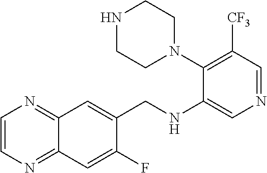 [SMILES: Fc1cc2nccnc2cc1CNc1cncc(C(F)(F)F)c1N1CCNCC1]

¹H NMR (400 MHz, MeOD) δ 8.89 (dd, J=1.8, 17.3 Hz, 2H), 8.48 (s, 1H), 8.30 (s, 1H), 8.08 (d, J=7.8 Hz, 1H), 7.86 (d, J=10.8 Hz, 1H), 5.00 (s, 2H), 3.63 (s, 8H), MS (ES+) m/e 407.3 (M+H)⁺.

### Example 126

### N-((7-fluoroquinoxalin-6-yl)methyl)-5-methoxy-4-(piperazin-1-yl)pyridin-3-amine (Comp. 131)

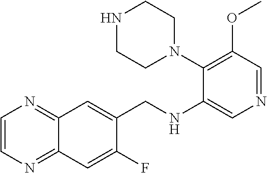 [SMILES: COc1cncc(NCc2cc3nccnc3cc2F)c1N1CCNCC1]

¹H NMR (400 MHz, MeOD) δ 8.87 (br d, J=19.9 Hz, 2H), 8.03-7.96 (m, 2H), 7.87-7.79 (m, 2H), 4.89 (s, 2H), 4.05 (s, 3H), 3.53 (br s, 8H), MS (ES+) m/z 369.3 (M+H)⁺.

### Example 127

### N-((7-chloroquinoxalin-6-yl)methyl)-4-(piperazin-1-yl)-5-(trifluoromethyl)pyridin-3-amine (Comp. 132)

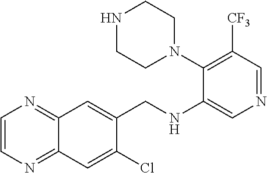 [SMILES: FC(F)(F)c1cncc(NCc2cc3nccnc3cc2Cl)c1N1CCNCC1]

¹H NMR (400 MHz, MeOD) δ 8.91 (d, J=1.6 Hz, 1H), 8.88 (d, J=1.6 Hz, 1H), 8.44 (s, 1H), 8.28 (s, 1H), 8.19 (s, 1H), 8.04 (s, 1H), 4.97 (s, 2H), 3.60 (br d, J=2.8 Hz, 8H). MS (ES+) m/z 423 (M+H)⁺.

### Example 128

### N-((7-chloroquinoxalin-6-yl)methyl)-5-methoxy-4-(piperazin-1-yl)pyridin-3-amine (Comp. 133)

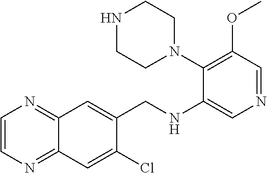 [SMILES: COc1cncc(NCc2cc3nccnc3cc2Cl)c1N1CCNCC1]

¹H NMR (400 MHz, MeOD δ 8.90 (dd, J=1.8, 16.3 Hz, 2H), 8.25 (s, 1H), 7.99 (d, J=5.5 Hz, 2H), 7.73 (s, 1H), 4.88-4.87 (m, 2H), 4.06 (s, 3H), 3.61-3.49 (m, 8H), MS (ES+) m/z 385.1 (M+H)⁺.

### Example 129

### 5-bromo-N-((7-fluoroquinoxalin-6-yl)methyl)-4-(piperazin-1-yl)pyridin-3-amine (Comp. 134)

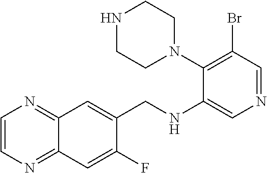 [SMILES: Fc1cc2nccnc2cc1CNc1cncc(Br)c1N1CCNCC1]

¹H NMR: (400 MHz, MeOD-d₄) δ 8.90 (d, J=1.8 Hz, 1H), 8.86 (d, J=1.8 Hz, 1H), 8.32 (d, J=0.6 Hz, 1H), 8.08-7.99 (m, 2H), 7.86 (d, J=10.8 Hz, 1H), 4.93 (s, 2H), 3.74 (br s, 4H), 3.60 (br t, J=4.6 Hz, 4H) MS (ES+) m/z 417 (M+H)⁺

### Example 130

### 5-(difluoromethyl)-N-((7-fluoroquinoxalin-6-yl)methyl)-4-(piperazin-1-yl)pyridin-3-amine (Comp. 135)

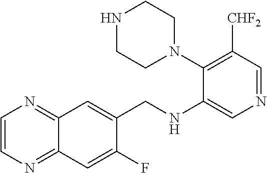 [SMILES: Fc1cc2nccnc2cc1CNc1cncc(C(F)F)c1N1CCNCC1]

¹H NMR (400 MHz, D₂O) δ 8.92 (s, 1H), 8.89 (s, 1H), 8.32 (s, 1H), 8.13-8.03 (m, 2H), 7.88 (d, J=10 Hz, 1H), 7.33 (t, J=54 Hz, 1H), 4.95 (s, 2H), 3.61 (br d, J=4.8 Hz, 8H). MS (ES⁺) m/z 389 (M+H)⁺.

### Example 131

### 5-bromo-N-((7-chloroquinoxalin-6-yl)methyl)-4-(piperazin-1-yl)pyridin-3-amine (Comp. 136)

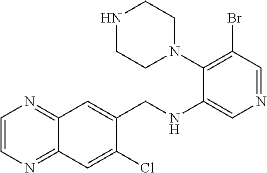 [SMILES: Clc1cc2nccnc2cc1CNc1cncc(Br)c1N1CCNCC1]

¹H NMR (400 MHz, MeOD-d₄) δ 8.92 (d, J=1.8 Hz, 1H), 8.88 (d, J=1.9 Hz, 1H), 8.32 (d, J=0.8 Hz, 1H), 8.27 (s, 1H), 8.00 (s, 1H), 7.97 (s, 1H), 4.91 (s, 2H), 3.74 (br s, 4H), 3.58 (br t, J=4.9 Hz, 4H) MS (ES+) m/z 345 (M+H)⁺

### Example 132

### N-((7-chloroquinoxalin-6-yl)methyl)-5-(difluoromethyl)-4-(piperazin-1-yl)pyridin-3-amine (Comp. 137)

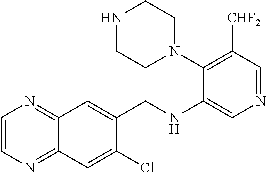 [SMILES: FC(F)c1cncc(NCc2cc3nccnc3cc2Cl)c1N1CCNCC1]

¹H NMR (400 MHz, D₂O) δ 8.93 (d, J=2.0 Hz, 1H), 8.90 (d, J=2.0 Hz, 1H), 8.32 (s, 1H), 8.27 (s, 1H), 8.03 (d, J=9.4 Hz, 2H), 7.35 (t, J=54.0 Hz, 1H), 4.94 (s, 2H), 3.62 (s, 8H). MS (ES⁺) m/z 405 (M+H)⁺.

### Example 133

### (S)-5-fluoro-N-((7-fluoroquinoxalin-6-yl)methyl)-4-(3-methylpiperazin-1-yl)pyridin-3-amine (Comp. 138)

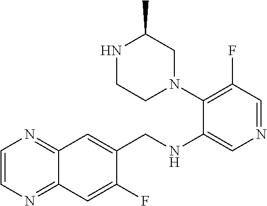 [SMILES: C[C@H]1CN(c2c(F)cncc2NCc2cc3nccnc3cc2F)CCN1]

¹H NMR (400 MHz, DMSO-d₆) δ 10.22-10.06 (m, 1H), 9.49 (br d, J=6.4 Hz, 1H), 8.95 (d, J=2.0 Hz, 1H), 8.91 (d, J=2.0 Hz, 1H), 8.35 (d, J=4.8 Hz, 1H), 8.00 (d, J=8.0 Hz, 1H), 7.98-7.94 (m, 2H), 4.82 (br s, 2H), 3.73-3.63 (m, 1H), 3.52-3.42 (m, 4H), 3.35 (br d, J=2.8 Hz, 1H), 3.31-3.23 (m, 1H), 1.30 (d, J=6.8 Hz, 3H). MS (ES+) m/z 371 (M+H)⁺

### Example 134

### (S)-5-chloro-N-((7-fluoroquinoxalin-6-yl)methyl)-4-(3-methylpiperazin-1-yl)pyridin-3-amine (Comp. 139)

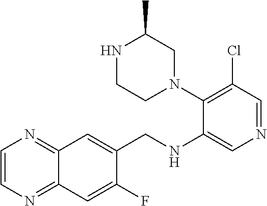 [SMILES: C[C@H]1CN(c2c(Cl)cncc2NCc2cc3nccnc3cc2F)CCN1]

¹H NMR (400 MHz, D₂O) δ=8.93 (d, J=2.0 Hz, 1H), 8.89 (d, J=1.8 Hz, 1H), 8.25 (d, J=0.8 Hz, 1H), 8.08 (d, J=7.6 Hz, 1H), 8.03 (s, 1H), 7.86 (d, J=10.6 Hz, 1H), 4.97-4.94 (m, 2H), 3.99-3.86 (m, 2H), 3.79-3.63 (m, 2H), 3.61-3.47 (m, 3H), 1.43 (d, J=6.6 Hz, 3H). MS (ES⁺) m/z 387 (M+H)⁺.

### Example 135

### (S)-5-bromo-N-((7-fluoroquinoxalin-6-yl)methyl)-4-(3-methylpiperazin-1-yl)pyridin-3-amine (Comp. 140)

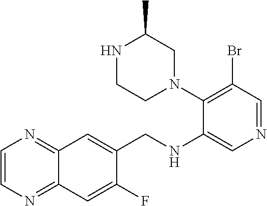 [SMILES: C[C@H]1CN(c2c(Br)cncc2NCc2cc3nccnc3cc2F)CCN1]

¹H NMR (400 MHz, MeOD-d₄) δ 8.90 (d, J=1.6 Hz, 1H), 8.86 (d, J=1.5 Hz, 1H), 8.32 (s, 1H), 8.07-8.01 (m, 2H), 7.86 (d, J=10.8 Hz, 1H), 4.92 (s, 2H), 4.01-3.80 (m, 2H), 3.71 (br dd, J=10.8, 13.1 Hz, 2H), 3.59-3.41 (m, 3H), 1.42 (d, J=6.6 Hz, 3H) MS (ES+) m/z 432 (M+H)+

### Example 136

### (S)-5-(difluoromethyl)-N-((7-fluoroquinoxalin-6-yl)methyl)-4-(3-methylpiperazin-1-yl)pyridin-3-amine (Comp. 141)

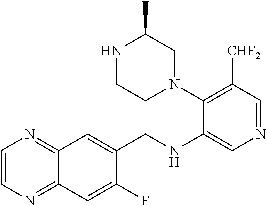 [SMILES: C[C@H]1CN(c2c(NCc3cc4nccnc4cc3F)cncc2C(F)F)CCN1]

¹H NMR (400 MHz, MeOD) δ 8.90 (d, J=1.6 Hz, 1H), 8.86 (d, J=2.0 Hz, 1H), 8.32 (s, 1H), 8.18 (s, 1H), 8.07 (d, J=8.0 Hz, 1H), 7.86 (d, J=10.8 Hz, 1H), 7.51-7.19 (m, 1H), 4.95 (s, 2H), 3.94 (ddd, J1=3.2 Hz, J2=6.4 Hz, J3=10.4 Hz, 1H), 3.81-3.70 (m, 1H), 3.66-3.50 (m, 4H), 3.30-3.24 (m, 1H), 1.42 (d, J=6.4 Hz, 3H). MS (ES+) m/z 403 (M+H)⁺.

### Example 137

### (S)—N-((7-fluoroquinoxalin-6-yl)methyl)-4-(3-methylpiperazin-1-yl)-5-(trifluoromethyl)pyridin-3-amine (Comp. 142)

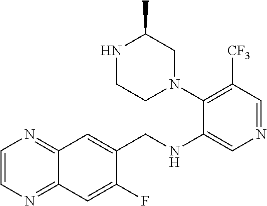 [SMILES: C[C@H]1CN(c2c(NCc3cc4nccnc4cc3F)cncc2C(F)(F)F)CCN1]

### (S)—N-((7-fluoroquinoxalin-6-yl)methyl)-5-methoxy-4-(3-methylpiperazin-1-yl)pyridin-3-amine (Comp. 143)

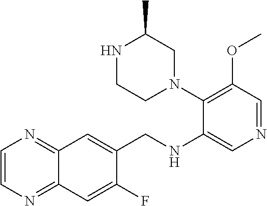 [SMILES: COc1cncc(NCc2cc3nccnc3cc2F)c1N1CCN[C@@H](C)C1]

¹H NMR (400 MHz, MeOD) δ 8.98-8.82 (m, 2H), 8.08-7.96 (m, 2H), 7.89-7.78 (m, 2H), 4.90 (br s, 2H), 4.05 (s, 3H), 3.80 (dt, J=3.6, 6.6 Hz, 1H), 3.74-3.59 (m, 2H), 3.53-3.44 (m, 2H), 3.41-3.32 (m, 2H), 1.40 (d, J=6.6 Hz, 3H), MS (ES+) m/z 383.4 (M+H)⁺.

### Example 139

### (S)—N-((7-chloroquinoxalin-6-yl)methyl)-5-fluoro-4-(3-methylpiperazin-1-yl)pyridin-3-amine (Comp. 144)

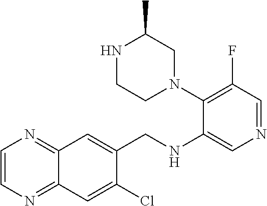 [SMILES: C[C@H]1CN(c2c(F)cncc2NCc2cc3nccnc3cc2Cl)CCN1]

¹H NMR (400 MHz, MeOD-d₄) δ 8.92 (d, J=2.0 Hz, 1H), 8.88 (d, J=2.0 Hz, 1H), 8.27 (s, 1H), 8.25 (d, J=4.8 Hz, 1H), 8.02 (s, 1H), 7.88 (s, 1H), 4.90 (s, 2H), 3.78 (dt, J1=3.2 Hz, J2=6.8 Hz, 1H), 3.75-3.67 (m, 2H), 3.62-3.55 (m, 3H), 3.42-3.34 (m, 1H), 1.42 (d, J=6.8 Hz, 3H). MS (ES+) m/z 387 (M+H)+

### Example 140

### (S)-5-chloro-N-((7-chloroquinoxalin-6-yl)methyl)-4-(3-methylpiperazin-1-yl)pyridin-3-amine (Comp. 145)

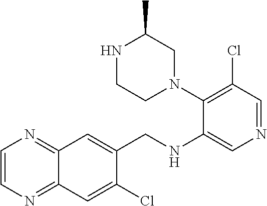 [SMILES: C[C@H]1CN(c2c(Cl)cncc2NCc2cc3nccnc3cc2Cl)CCN1]

¹H NMR (400 MHz, D₂O) δ 8.91 (d, J=1.8 Hz, 1H), 8.88 (d, J=1.8 Hz, 1H), 8.26 (s, 1H), 8.19 (s, 1H), 8.02 (s, 1H), 7.92 (s, 1H), 4.90 (s, 2H), 3.91-3.78 (m, 2H), 3.71-3.55 (m, 3H), 3.55-3.47 (m, 2H), 1.42 (d, J=6.6 Hz, 3H). MS (ES⁺) m/z 403 (M+H)⁺.

### Example 141

### (S)-5-bromo-N-((7-chloroquinoxalin-6-yl)methyl)-4-(3-methylpiperazin-1-yl)pyridin-3-amine (Comp. 146)

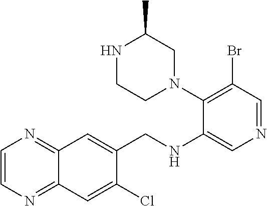 [SMILES: C[C@H]1CN(c2c(Br)cncc2NCc2cc3nccnc3cc2Cl)CCN1]

¹H NMR (400 MHz, MeOD-d₄) δ 8.92 (d, J=1.9 Hz, 1H), 8.88 (d, J=1.9 Hz, 1H), 8.32 (d, J=0.6 Hz, 1H), 8.26 (s, 1H), 8.01 (s, 1H), 7.97 (s, 1H), 4.92 (s, 2H), 4.02-3.92 (m, 1H), 3.86 (br dd, J=3.4, 6.3 Hz, 1H), 3.73 (dd, J=10.6, 13.1 Hz, 2H), 3.60-3.47 (m, 3H), 1.42 (d, J=6.5 Hz, 3H) MS (ES+) m/z 449 (M+H)⁺

### Example 142

### (S)—N-((7-chloroquinoxalin-6-yl)methyl)-5-(difluoromethyl)-4-(3-methylpiperazin-1-yl)pyridin-3-amine (Comp. 147)

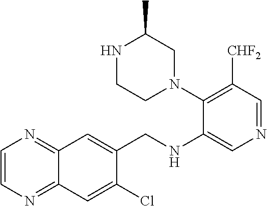 [SMILES: C[C@H]1CN(c2c(NCc3cc4nccnc4cc3Cl)cncc2C(F)F)CCN1]

¹H NMR (400 MHz, MeOD) δ 8.92 (d, J=2.0 Hz, 1H), 8.88 (d, J=2.0 Hz, 1H), 8.33 (s, 1H), 8.27 (s, 1H), 8.11 (s, 1H), 8.05 (s, 1H), 7.52-7.20 (m, 1H), 4.95 (s, 2H), 3.92 (ddd, J1=3.2 Hz, J2=6.6 Hz, J3=10.0 Hz, 1H), 3.80-3.71 (m, 1H), 3.69-3.61 (m, 2H), 3.60-3.51 (m, 2H), 3.26 (s, 1H), 1.42 (d, J=6.4 Hz, 3H). MS (ES+) m/z 419 (M+H)⁺.

### Example 143

### (S)—N-((7-chloroquinoxalin-6-yl)methyl)-4-(3-methylpiperazin-1-yl)-5-(trifluoromethyl)pyridin-3-amine (Comp. 148)

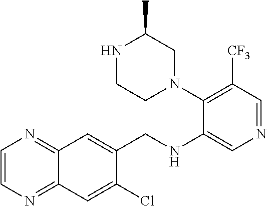 [SMILES: C[C@H]1CN(c2c(NCc3cc4nccnc4cc3Cl)cncc2C(F)(F)F)CCN1]

### Example 144

### (S)—N-((7-chloroquinoxalin-6-yl)methyl)-5-methoxy-4-(3-methylpiperazin-1-yl)pyridin-3-amine (Comp. 149)

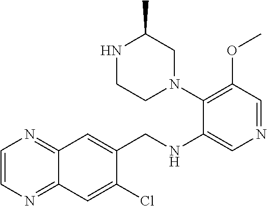 [SMILES: COc1cncc(NCc2cc3nccnc3cc2Cl)c1N1CCN[C@@H](C)C1]

¹H NMR (400 MHz, MeOD) δ 8.91 (dd, J=1.8, 16.5 Hz, 2H), 8.25 (s, 1H), 8.00 (s, 2H), 7.73 (s, 1H), 4.89 (s, 2H), 4.06 (s, 3H), 3.89-3.77 (m, 1H), 3.76-3.58 (m, 2H), 3.55-3.45 (m, 2H), 3.45-3.34 (m, 2H), 1.41 (d, J=6.6 Hz, 3H), MS (ES+) m/z 399.1 (M+H)⁺.

### Example 145

### 5-fluoro-6-methyl-4-(piperazin-1-yl)-N-(quinoxalin-6-ylmethyl)pyridin-3-amine (Comp. 150)

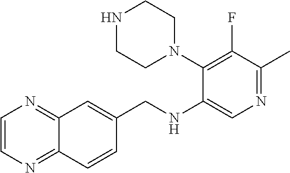 [SMILES: Cc1ncc(NCc2ccc3nccnc3c2)c(N2CCNCC2)c1F]

¹H NMR (400 MHz, MeOD) δ 9.08 (dd, J=2.1, 10.5 Hz, 2H), 8.27-8.16 (m, 2H), 8.13-8.06 (m, 1H), 7.63 (s, 1H), 4.91 (s, 2H), 3.70 (br d, J=3.6 Hz, 4H), 3.66-3.55 (m, 4H), 2.52 (d, J=2.6 Hz, 3H). MS (ES+) m/z 353.2 (M+H)⁺.

### Example 146

### 5-chloro-6-methyl-4-(piperazin-1-yl)-N-(quinoxalin-6-ylmethyl)pyridin-3-amine (Comp. 151)

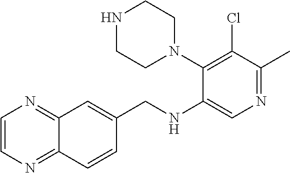 [SMILES: Cc1ncc(NCc2ccc3nccnc3c2)c(N2CCNCC2)c1Cl]

¹H NMR (400 MHz, MeOD) δ 9.05-8.98 (m, 2H), 8.20 (d, J=8.8 Hz, 1H), 8.15 (s, 1H), 8.04 (dd, J=1.8, 8.7 Hz, 1H), 7.71 (s, 1H), 4.90 (br s, 2H), 3.71 (br s, 4H), 3.62 (br s, 4H), 2.60 (s, 3H), MS (ES+) m/z 369.2 (M+H)⁺.

### Example 147

### 5-bromo-6-methyl-4-(piperazin-1-yl)-N-(quinoxalin-6-ylmethyl)pyridin-3-amine (Comp. 152)

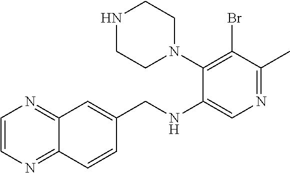 [SMILES: Cc1ncc(NCc2ccc3nccnc3c2)c(N2CCNCC2)c1Br]

¹H NMR (400 MHz, MeOD) δ 9.02-8.91 (m, 2H), 8.17 (d, J=8.8 Hz, 1H), 8.11 (d, J=1.1 Hz, 1H), 8.01 (dd, J=1.9, 8.6 Hz, 1H), 7.73 (s, 1H), 4.90-4.89 (m, 2H), 3.97-3.49 (m, 8H), 2.65 (s, 3H), MS (ES+) m/z 413.3 (M+H)⁺.

### Example 148

### 5,6-dimethyl-4-(piperazin-1-yl)-N-(quinoxalin-6-ylmethyl)pyridin-3-amine (Comp. 153)

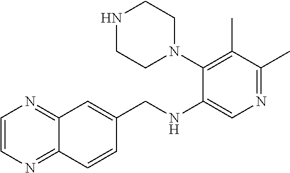 [SMILES: Cc1ncc(NCc2ccc3nccnc3c2)c(N2CCNCC2)c1C]

¹H NMR (400 MHz, MeOD) δ 9.07-8.90 (m, 2H), 8.19 (d, J=8.8 Hz, 1H), 8.12 (s, 1H), 8.03 (dd, J=1.6, 8.8 Hz, 1H), 7.54 (s, 1H), 4.86 (s, 2H), 3.61 (br s, 8H), 2.51 (s, 3H), 2.43 (s, 3H), MS (ES+) m/z 349.2 (M+H)⁺.

### Example 149

### 5-(difluoromethyl)-6-methyl-4-(piperazin-1-yl)-N-(quinoxalin-6-ylmethyl)pyridin-3-amine (Comp. 154)

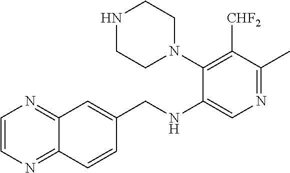 [SMILES: Cc1ncc(NCc2ccc3nccnc3c2)c(N2CCNCC2)c1C(F)F]

¹H NMR (400 MHz, MeOD) δ 8.97-8.88 (m, 2H), 8.21-8.13 (m, 1H), 8.12-8.07 (m, 1H), 8.03-7.93 (m, 1H), 7.82 (s, 1H), 7.48-7.11 (m, 1H), 4.90-4.89 (m, 2H), 3.69 (br d, J=3.6 Hz, 4H), 3.61 (br d, J=3.2 Hz, 4H), 2.68 (s, 3H). MS (ES+) m/z 385 (M+H)⁺.

### Example 150

### 5-fluoro-4-(piperazin-1-yl)-N-(quinoxalin-6-ylmethyl)-6-(trifluoromethyl)pyridin-3-amine (Comp. 155)

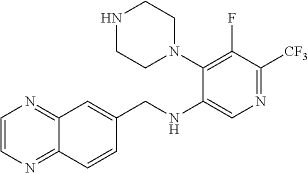 [SMILES: Fc1c(C(F)(F)F)ncc(NCc2ccc3nccnc3c2)c1N1CCNCC1]

¹H NMR (400 MHz, CD₃OD) δ 8.96-8.90 (m, 2H), 8.17 (d, J=8.8 Hz, 1H), 8.05 (s, 1H), 7.99-7.94 (m, 1H), 7.71 (s, 1H), 4.90 (s, 2H), 3.54-3.52 (m, 4H), 3.46 (br s, 4H). MS (ES+) m/z 407 (M+H)⁺.

### Example 151

### 5-chloro-4-(piperazin-1-yl)-N-(quinoxalin-6-ylmethyl)-6-(trifluoromethyl)pyridin-3-amine (Comp. 156)

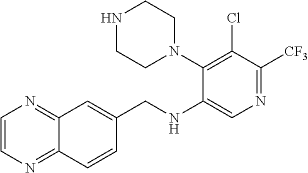 [SMILES: FC(F)(F)c1ncc(NCc2ccc3nccnc3c2)c(N2CCNCC2)c1Cl]

¹H NMR (400 MHz, CD₃OD) δ 9.01 (dd, J1=2.0 Hz, J2=12.6 Hz, 2H), 8.20 (d, J=8.8 Hz, 1H), 8.09 (s, 1H), 8.03 (dd, J1=2.0 Hz, J2=8.8 Hz, 1H), 7.76 (s, 1H), 4.93 (s, 2H), 3.96 (br t, J=12.0 Hz, 2H), 3.73-3.62 (m, 2H), 3.52-3.44 (m, 2H), 3.24 (br d, J=12.6 Hz, 2H). MS (ES+) m/z 423 (M+H)⁺.

### Example 152

### 5-bromo-4-(piperazin-1-yl)-N-(quinoxalin-6-ylmethyl)-6-(trifluoromethyl)pyridin-3-amine (Comp. 157)

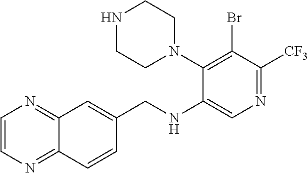 [SMILES: FC(F)(F)c1ncc(NCc2ccc3nccnc3c2)c(N2CCNCC2)c1Br]

¹H NMR (400 MHz, CD₃OD) δ 9.05 (dd, J1=2.0 Hz, J2=15.0 Hz, 2H), 8.23 (d, J=8.8 Hz, 1H), 8.12 (s, 1H), 8.06 (dd, dd, J1=2.0 Hz, J2=8.8 Hz, 1H), 7.77 (s, 1H), 4.95 (s, 2H), 4.08 (br t, J=12.0 Hz, 2H), 3.69 (br t, J=12.0 Hz, 2H), 3.53-3.44 (m, 2H), 3.25-3.21 (m, 2H). MS (ES+) m/z 467 (M+H)⁺.

### Example 153

### 5-methyl-4-(piperazin-1-yl)-N-(quinoxalin-6-ylmethyl)-6-(trifluoromethyl)pyridin-3-amine (Comp. 158)

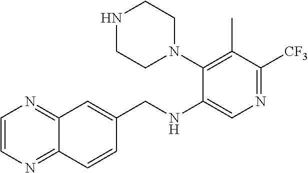 [SMILES: Cc1c(C(F)(F)F)ncc(NCc2ccc3nccnc3c2)c1N1CCNCC1]

¹H NMR (400 MHz, CD₃OD) δ 8.96-8.90 (m, 2H), 8.16 (d, J=8.6 Hz, 1H), 8.06 (s, 1H), 7.97 (dd, 11=2.0 Hz, J2=8.8 Hz, 1H), 7.72 (s, 1H), 4.93 (s, 2H), 3.57 (br s, 8H), 2.50 (d, J=0.8 Hz, 3H). MS (ES+) m/z 403 (M+H)⁺.

### Example 154

### 5-(difluoromethyl)-4-(piperazin-1-yl)-N-(quinoxalin-6-ylmethyl)-6-(trifluoromethyl)pyridin-3-amine (Comp. 159)

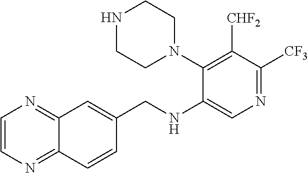 [SMILES: FC(F)c1c(C(F)(F)F)ncc(NCc2ccc3nccnc3c2)c1N1CCNCC1]

### Example 155

### (R)-5-bromo-4-(pyrrolidin-3-yloxy)-N-(quinoxalin-6-ylmethyl)pyridin-3-amine (Comp. 160)

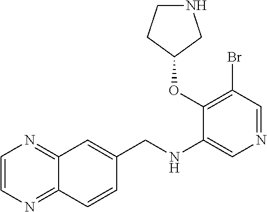 [SMILES: Brc1cncc(NCc2ccc3nccnc3c2)c1O[C@@H]1CCNC1]

¹H NMR: (400 MHz, MeOD-d₄) δ 8.97-8.83 (m, 2H), 8.36 (d, J=1.0 Hz, 1H), 8.14 (d, J=8.6 Hz, 1H), 8.07 (d, J=1.1 Hz, 1H), 7.97-7.94 (m, 2H), 5.83 (t, J=4.6 Hz, 1H), 4.90 (br d, J=0.9 Hz, 2H), 3.93 (d, J=13.6 Hz, 1H), 3.80 (dt, J=7.5, 11.0 Hz, 1H), 3.71 (dd, J=4.4, 13.6 Hz, 1H), 3.60 (ddd, J=3.4, 9.0, 12.0 Hz, 1H), 2.72-2.31 (m, 2H) MS (ES+) m/z 402 (M+H)⁺

### Example 156

### (R)-5-(difluoromethyl)-4-(pyrrolidin-3-yloxy)-N-(quinoxalin-6-ylmethyl)pyridin-3-amine (Comp. 161)

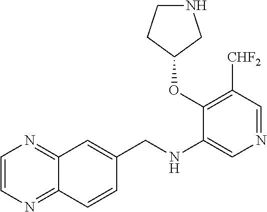 [SMILES: FC(F)c1cncc(NCc2ccc3nccnc3c2)c1O[C@@H]1CCNC1]

¹H NMR (400 MHz, MeOD) δ 8.91 (s, 2H), 8.35 (s, 1H), 8.15 (d, J=8.8 Hz, 1H), 8.10 (s, 2H), 7.99 (dd, J=1.6, 8.8 Hz, 1H), 7.48-7.18 (m, 1H), 5.70 (br s, 1H), 4.90-4.90 (m, 2H), 3.93 (br d, J=13.9 Hz, 1H), 3.78-3.67 (m, 2H), 3.62-3.54 (m, 1H), 2.56-2.36 (m, 2H). MS (ES+) m/z 372.3 (M+H)⁺.

### Example 157

### (R)-4-(pyrrolidin-3-yloxy)-N-(quinoxalin-6-ylmethyl)-5-(trifluoromethyl)pyridin-3-amine (Comp. 162)

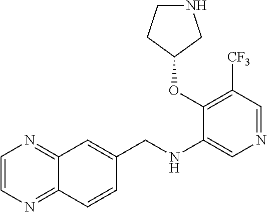 [SMILES: FC(F)(F)c1cncc(NCc2ccc3nccnc3c2)c1O[C@@H]1CCNC1]

¹H NMR (400 MHz, MeOD-d₄) δ 8.88-8.84 (m, 2H), 8.12 (d, J=8.80 Hz, 1H), 8.04 (s, 2H), 8.00 (s, 1H), 7.92 (dd, J1=2.00, J2=8.6 Hz, 1H), 5.24 (br t, J=5.20 Hz, 1H), 4.77 (s, 2H), 3.30-3.19 (m, 2H), 3.02 (dd, J=4.80, 12.9 Hz, 1H), 2.94 (ddd, J=5.40, 8.6, 11.3 Hz, 1H), 2.19-2.01 (m, 2H). MS (ES+) m/z 390 (M+H)⁺

### Example 158

### (R)-5-methoxy-4-(pyrrolidin-3-yloxy)-N-(quinoxalin-6-ylmethyl)pyridin-3-amine (Comp. 163)

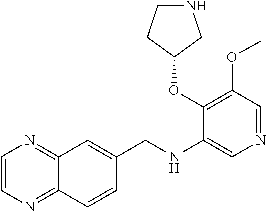 [SMILES: COc1cncc(NCc2ccc3nccnc3c2)c1O[C@@H]1CCNC1]

¹H NMR (400 MHz, MeOD) δ 9.08-9.01 (m, 2H), 8.20 (d, J=8.8 Hz, 1H), 8.16 (s, 1H), 8.08 (dd, J=1.8, 8.8 Hz, 1H), 8.03 (s, 1H), 7.70 (d, J=0.8 Hz, 1H), 5.99-5.91 (m, 1H), 4.88 (s, 2H), 4.05 (s, 3H), 3.88 (br d, J=13.6 Hz, 1H), 3.75-3.62 (m, 2H), 3.60-3.53 (m, 1H), 2.53-2.34 (m, 2H), MS (ES+) m/z 352.2 (M+H)⁺.

### Example 159

### (R)-5-bromo-N-((7-fluoroquinoxalin-6-yl)methyl)-4-(pyrrolidin-3-yloxy)pyridin-3-amine (Comp. 164)

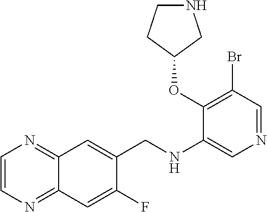 [SMILES: Fc1cc2nccnc2cc1CNc1cncc(Br)c1O[C@@H]1CCNC1]

¹H NMR (400 MHz, MeOD-d₄) δ 8.90 (d, J=1.8 Hz, 1H), 8.86 (d, J=1.8 Hz, 1H), 8.41 (d, J=1.0 Hz, 1H), 8.11-8.01 (m, 2H), 7.86 (d, J=10.8 Hz, 1H), 5.84 (t, J=4.6 Hz, 1H), 4.91 (s, 2H), 3.92 (d, J=13.9 Hz, 1H), 3.84-3.66 (m, 2H), 3.58 (ddd, J=3.5, 8.9, 11.9 Hz, 1H), 2.62-2.53 (m, 1H), 2.52-2.41 (m, 1H) MS (ES+) m/z 419 (M+H)+

### Example 160

### (R)-5-(difluoromethyl)-N-((7-fluoroquinoxalin-6-yl)methyl)-4-(pyrrolidin-3-yloxy)pyridin-3-amine (Comp. 165)

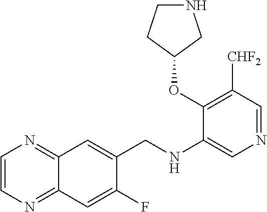 [SMILES: Fc1cc2nccnc2cc1CNc1cncc(C(F)F)c1O[C@@H]1CCNC1]

¹H NMR (400 MHz, MeOD) δ 8.88 (dd, J=1.8, 18.4 Hz, 2H), 8.40 (s, 1H), 8.25 (s, 1H), 8.07 (d, J=7.6 Hz, 1H), 7.87 (d, J=10.8 Hz, 1H), 7.45-7.16 (m, 1H), 5.68 (t, J=4.1 Hz, 1H), 4.91 (s, 2H), 3.90 (d, J=13.6 Hz, 1H), 3.74-3.66 (m, 2H), 3.60-3.55 (m, 1H), 2.51-2.38 (m, 2H). MS (ES+) m/z 390.2 (M+H)⁺.

### Example 161

### (R)—N-((7-fluoroquinoxalin-6-yl)methyl)-4-(pyrrolidin-3-yloxy)-5-(trifluoromethyl)pyridin-3-amine (Comp. 166)

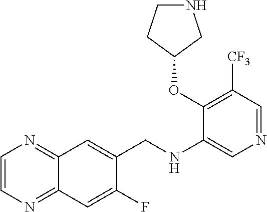 [SMILES: Fc1cc2nccnc2cc1CNc1cncc(C(F)(F)F)c1O[C@@H]1CCNC1]

¹H NMR (400 MHz, MeOD-d₄) δ 8.87 (d, J=2.00 Hz, 1H), 8.83 (d, J=2.00 Hz, 1H), 8.09 (s, 1H), 8.07 (s, 1H), 8.04 (d, J=7.60 Hz, 1H), 7.83 (d, J=10.4 Hz, 1H), 5.23 (br t, J=5.2 Hz, 1H), 4.79 (s, 2H), 3.30-3.15 (m, 2H), 3.00 (dd, J=4.6, 12.9 Hz, 1H), 2.91 (ddd, J1=5.60 Hz, J2=8.60 Hz, J3=11.1 Hz, 1H), 2.20-2.01 (m, 2H). MS (ES+) m/z 408 (M+H)⁺

### Example 162

### (R)—N-((7-fluoroquinoxalin-6-yl)methyl)-5-methoxy-4-(pyrrolidin-3-yloxy)pyridin-3-amine (Comp. 167)

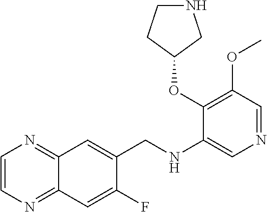 [SMILES: COc1cncc(NCc2cc3nccnc3cc2F)c1O[C@@H]1CCNC1]

¹H NMR (400 MHz, MeOD) δ 8.89 (dd, J=1.8, 19.5 Hz, 2H), 8.07 (s, 1H), 8.05-8.00 (m, 1H), 7.87-7.80 (m, 1H), 7.80-7.76 (m, 1H), 5.95 (t, J=4.2 Hz, 1H), 4.89-4.87 (m, 2H), 4.07 (s, 3H), 3.87 (d, J=13.1 Hz, 1H), 3.74-3.60 (m, 2H), 3.56 (ddd, J=3.3, 9.0, 11.8 Hz, 1H), 2.54-2.34 (m, 2H), MS (ES+) m/z 370.4 (M+H)⁺.

### Example 163

### (R)-5-bromo-N-((7-chloroquinoxalin-6-yl)methyl)-4-(pyrrolidin-3-yloxy)pyridin-3-amine (Comp. 168)

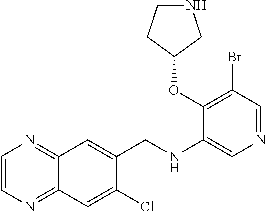 [SMILES: Clc1cc2nccnc2cc1CNc1cncc(Br)c1O[C@@H]1CCNC1]

¹H NMR (400 MHz, MeOD-d₄) δ 8.91 (d, J=1.9 Hz, 1H), 8.87 (d, J=1.9 Hz, 1H), 8.42 (d, J=0.9 Hz, 1H), 8.25 (s, 1H), 8.02-7.97 (m, 2H), 5.88 (t, J=4.6 Hz, 1H), 4.90 (s, 2H), 3.94 (d, J=13.6 Hz, 1H), 3.85-3.66 (m, 2H), 3.59 (ddd, J=3.4, 9.0, 11.9 Hz, 1H), 2.66-2.57 (m, 1H), 2.54-2.42 (m, 1H) MS (ES+) m/z 436 (M+H)⁺

### Example 164

### (R)—N-((7-chloroquinoxalin-6-yl)methyl)-5-(difluoromethyl)-4-(pyrrolidin-3-yloxy)pyridin-3-amine (Comp. 169)

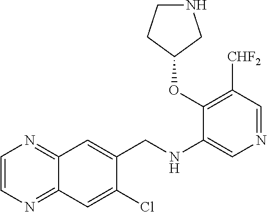 [SMILES: FC(F)c1cncc(NCc2cc3nccnc3cc2Cl)c1O[C@@H]1CCNC1]

¹H NMR (400 MHz, MeOD) δ 8.90 (dd, J=1.9, 15.8 Hz, 2H), 8.41 (s, 1H), 8.28 (s, 1H), 8.17 (s, 1H), 8.03 (s, 1H), 7.50-7.16 (m, 1H), 5.71 (t, J=4.7 Hz, 1H), 4.91 (s, 2H), 3.91 (d, J=13.8 Hz, 1H), 3.75-3.65 (m, 2H), 3.61-3.54 (m, 1H), 2.59-2.37 (m, 2H). MS (ES+) m/z 390.2 (M+H)⁺.

### Example 165

### (R)—N-((7-chloroquinoxalin-6-yl)methyl)-4-(pyrrolidin-3-yloxy)-5-(trifluoromethyl)pyridin-3-amine (Comp. 170)

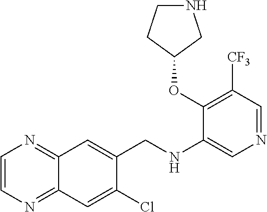 [SMILES: FC(F)(F)c1cncc(NCc2cc3nccnc3cc2Cl)c1O[C@@H]1CCNC1]

¹H NMR (400 MHz, MeOD-d₄) δ 8.89 (d, J=2.00 Hz, 1H), 8.85 (d, J=2.00 Hz, 1H), 8.25 (s, 1H), 8.10 (s, 1H), 8.03 (s, 1H), 7.97 (s, 1H), 5.26 (br t, J=5.20 Hz, 1H), 4.80 (s, 2H), 3.33 (br s, 1H), 3.22 (td, J1=7.60 Hz, J2=11.2 Hz, 1H), 3.03 (dd, 11=4.80 Hz, J2=12.8 Hz, 1H), 2.94 (ddd, J1=5.60 Hz, J2=8.80 Hz, J3=11.2 Hz, 1H), 2.21-2.04 (m, 2H). MS (ES+) m/z 424 (M+H)⁺.

### Example 166

### (R)—N-((7-chloroquinoxalin-6-yl)methyl)-5-methoxy-4-(pyrrolidin-3-yloxy)pyridin-3-amine (Comp. 171)

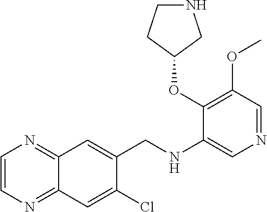 [SMILES: COc1cncc(NCc2cc3nccnc3cc2Cl)c1O[C@@H]1CCNC1]

¹H NMR (400 MHz, MeOD) δ 8.90 (dd, J=1.8, 16.9 Hz, 2H), 8.22 (s, 1H), 8.07 (s, 1H), 7.97 (s, 1H), 7.68 (s, 1H), 5.97 (t, J=3.9 Hz, 1H), 4.86 (s, 2H), 4.08 (s, 3H), 3.88 (br d, J=13.3 Hz, 1H), 3.76-3.62 (m, 2H), 3.61-3.51 (m, 1H), 2.67-2.30 (m, 2H), MS (ES+) m/e 386.2 (M+H)⁺.

### Example 167

### 4-(((3R,5S)-5-methylpyrrolidin-3-yl)oxy)-N-(quinoxalin-6-ylmethyl)pyridin-3-amine (Comp. 172)

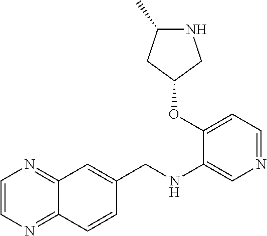 [SMILES: C[C@H]1C[C@@H](Oc2ccncc2NCc2ccc3nccnc3c2)CN1]

¹H NMR (400 MHz, MeOD-d₄) δ 9.03 (s, 2H), 8.22-8.15 (m, 2H), 8.11-8.04 (m, 2H), 7.80 (s, 1H), 7.51 (d, J=6.4 Hz, 1H), 5.67 (m, 1H), 4.99-4.93 (s, 2H), 4.87 (s, 2H), 4.08-3.90 (m, 2H), 3.79 (dd, J=5.2, 13.2 Hz, 1H), 2.97 (ddd, J=6.4, 8.5, 14.8 Hz, 1H), 2.11 (m, 1H), 1.59 (d, J=6.8 Hz, 3H). MS (ES+) m/z 336.2 (M+H)⁺.

### Example 168

### 4-(((3R,5R)-5-methylpyrrolidin-3-yl)oxy)-N-(quinoxalin-6-ylmethyl)pyridin-3-amine (Comp. 173)

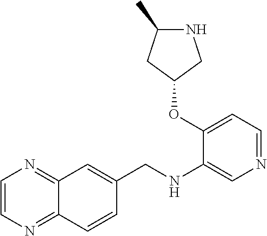 [SMILES: C[C@@H]1C[C@@H](Oc2ccncc2NCc2ccc3nccnc3c2)CN1]

¹H NMR (400 MHz, DMSO-d₆) δ 15.18 (s, 1H), 10.74-10.34 (m, 1H), 9.60 (s, 1H), 8.91 (s, 2H), 8.14-8.03 (m, 3H), 7.95 (d, J=8.8 Hz, 1H), 7.82 (s, 1H), 7.51 (d, J=6.4 Hz, 1H), 5.59 (m, 1H), 4.74 (s, 2H), 4.04-3.87 (m, 1H), 3.82-3.66 (m, 1H), 3.64-3.53 (m, 1H), 2.42 (dd, J=5.9, 14.3 Hz, 1H), 2.05-1.90 (m, 1H), 1.42 (d, J=6.5 Hz, 3H). MS (ES+) m/z 336.2 (M+H)⁺.

### Example 169

### 5-fluoro-4-(((3R,5S)-5-methylpyrrolidin-3-yl)oxy)-N-(quinoxalin-6-ylmethyl)pyridin-3-amine (Comp. 174)

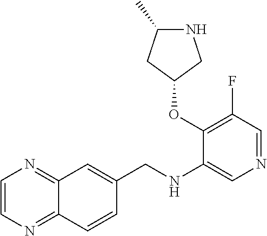 [SMILES: C[C@H]1C[C@@H](Oc2c(F)cncc2NCc2ccc3nccnc3c2)CN1]

¹H NMR (400 MHz, MeOD) δ 8.95 (s, 2H), 8.30 (d, J=6.0 Hz, 1H), 8.17-8.11 (m, 2H), 8.03 (d, J=8.9 Hz, 1H), 7.82 (s, 1H), 5.93 (s, 1H), 4.90 (s, 4H), 4.09-3.92 (m, 2H), 3.75 (dd, J=5.1, 13.4 Hz, 1H), 2.95 (td, J=7.5, 15.0 Hz, 1H), 2.28 (dd, J=7.5, 14.9 Hz, 1H), 1.63 (d, J=6.6 Hz, 3H). MS (ES+) m/z 354.2 (M+H)⁺.

### Example 170

### 5-fluoro-4-(((3R,5R)-5-methylpyrrolidin-3-yl)oxy)-N-(quinoxalin-6-ylmethyl)pyridin-3-amine Comp. 175)

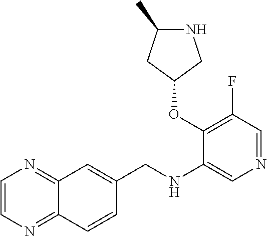 [SMILES: C[C@@H]1C[C@@H](Oc2c(F)cncc2NCc2ccc3nccnc3c2)CN1]

¹H NMR (400 MHz, MeOD) δ 9.01 (s, 2H), 8.30 (d, J=6.0 Hz, 1H), 8.22-8.14 (m, 2H), 8.06 (dd, J=1.7, 8.7 Hz, 1H), 7.81 (s, 1H), 5.93 (q, J=3.8 Hz, 1H), 4.91 (s, 4H), 4.34-4.19 (m, 1H), 4.02-3.85 (m, 2H), 2.78 (dd, J=6.1, 14.8 Hz, 1H), 2.20 (ddd, J=4.4, 11.2, 15.1 Hz, 1H), 1.57 (d, J=6.5 Hz, 3H). MS (ES+) m/z 354.2 (M+H)⁺.

### Example 171

### 5-methyl-4-(((3R,5S)-5-methylpyrrolidin-3-yl)oxy)-N-(quinoxalin-6-ylmethyl)pyridin-3-amine (Comp. 176)

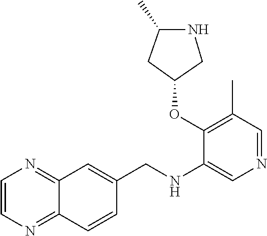 [SMILES: Cc1cncc(NCc2ccc3nccnc3c2)c1O[C@H]1CN[C@@H](C)C1]

¹H NMR (400 MHz, MeOD-d₄) δ 8.90 (s, 2H), 8.14 (d, J=8.8 Hz, 1H), 8.08 (s, 1H), 7.99-7.94 (m, 2H), 7.83 (s, 1H), 5.52-5.45 (m, 1H), 4.89-4.86 (m, 2H), 3.91-3.76 (m, 2H), 3.73-3.61 (m, 1H), 2.88 (td, J=7.5, 14.5 Hz, 1H), 2.50 (s, 3H), 2.23-2.11 (m, 1H), 1.61 (d, J=6.6 Hz, 3H) MS (ES+) m/z 350 (M+H)+

### Example 172

### 5-methyl-4-(((3R,5R)-5-methylpyrrolidin-3-yl)oxy)-N-(quinoxalin-6-ylmethyl)pyridin-3-amine (Comp. 177)

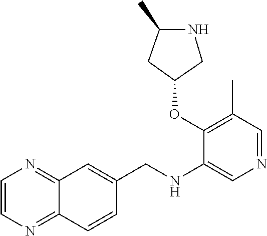 [SMILES: Cc1cncc(NCc2ccc3nccnc3c2)c1O[C@H]1CN[C@H](C)C1]

¹H NMR (400 MHz, MeOD-d₄) δ 8.94 (s, 2H), 8.16 (d, J=8.6 Hz, 1H), 8.09 (s, 1H), 8.01-7.97 (m, 2H), 7.82 (s, 1H), 5.56 (br s, 1H), 4.85 (s, 2H), 4.36-4.23 (m, 1H), 3.85 (s, 2H), 2.60 (dd, J=6.1, 14.8 Hz, 1H), 2.49 (s, 3H), 2.08 (ddd, J=4.6, 11.0, 15.1 Hz, 1H), 1.53 (d, J=6.6 Hz, 3H) MS (ES+) m/z 350 (M+H)⁺

### Example 173

### 5-chloro-4-(((3R,5S)-5-methylpyrrolidin-3-yl)oxy)-N-(quinoxalin-6-ylmethyl)pyridin-3-amine (Comp. 178)

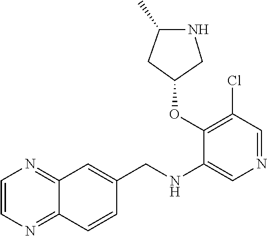 [SMILES: C[C@H]1C[C@@H](Oc2c(Cl)cncc2NCc2ccc3nccnc3c2)CN1]

¹H NMR (400 MHz, MeOD) δ 9.98-8.53 (m, 2H), 8.45-8.06 (m, 3H), 8.05-7.77 (m, 2H), 5.86 (br s, 1H), 3.97-3.80 (m, 2H), 3.71 (br dd, J=5.4, 13.2 Hz, 1H), 2.95 (td, J=7.5, 14.6 Hz, 1H), 2.36-2.21 (m, 1H), 1.62 (d, J=6.6 Hz, 3H), MS (ES+) m/z 370.0 (M+H)⁺.

### Example 174

### 5-chloro-4-(((3R,5R)-5-methylpyrrolidin-3-yl)oxy)-N-(quinoxalin-6-ylmethyl)pyridin-3-amine (Comp. 179)

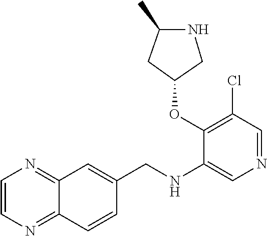 [SMILES: C[C@@H]1C[C@@H](Oc2c(Cl)cncc2NCc2ccc3nccnc3c2)CN1]

¹H NMR (400 MHz, MeOD) δ 8.97-8.91 (m, 2H), 8.27 (d, J=1.1 Hz, 1H), 8.16 (d, J=8.8 Hz, 1H), 8.11 (d, J=1.1 Hz, 1H), 8.00 (dd, J=1.9, 8.6 Hz, 1H), 7.92 (d, J=1.1 Hz, 1H), 5.90 (br s, 1H), 4.89 (br s, 2H), 4.30 (td, J=6.2, 11.9 Hz, 1H), 3.97-3.82 (m, 2H), 2.71 (dd, J=6.0, 14.9 Hz, 1H), 2.23-2.08 (m, 1H), 1.54 (d, J=6.6 Hz, 3H), MS (ES+) m/z 370.2 (M+H)⁺.

### Example 175

### 5-bromo-4-(((3R,5S)-5-methylpyrrolidin-3-yl)oxy)-N-(quinoxalin-6-ylmethyl)pyridin-3-amine (Comp. 180)

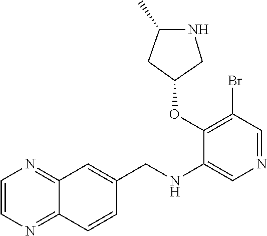 [SMILES: C[C@H]1C[C@@H](Oc2c(Br)cncc2NCc2ccc3nccnc3c2)CN1]

¹H NMR (400 MHz, MeOD-d₄) δ 8.90 (s, 2H), 8.34 (d, J=1.0 Hz, 1H), 8.14-8.09 (m, 2H), 8.01-7.96 (m, 2H), 5.81 (dddd, J=1.8, 3.9, 5.6, 7.3 Hz, 1H), 4.89 (br s, 2H), 3.97-3.80 (m, 2H), 3.71 (dd, J=5.9, 13.4 Hz, 1H), 2.97 (td, J=7.6, 14.8 Hz, 1H), 2.40-2.24 (m, 1H), 1.64 (d, J=6.6 Hz, 3H) MS (ES+) m/z 414 (M+H)+

### Example 176

### 5-bromo-4-(((3R,5R)-5-methylpyrrolidin-3-yl)oxy)-N-(quinoxalin-6-ylmethyl)pyridin-3-amine (Comp. 181)

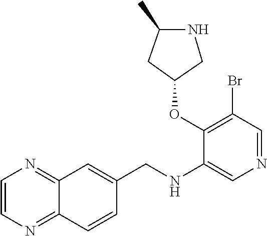 [SMILES: C[C@@H]1C[C@@H](Oc2c(Br)cncc2NCc2ccc3nccnc3c2)CN1]

¹H NMR (400 MHz, MeOD-d₄) δ 8.89 (s, 2H), 8.36 (d, J=0.9 Hz, 1H), 8.14 (d, J=8.6 Hz, 1H), 8.07 (d, J=1.1 Hz, 1H), 7.97-7.93 (m, 2H), 5.81 (br d, J=3.9 Hz, 1H), 4.89 (br s, 2H), 4.33 (td, J=6.1, 11.9 Hz, 1H), 3.89 (d, J=3.0 Hz, 2H), 2.71 (dd, J=6.1, 14.9 Hz, 1H), 2.12 (ddd, J=4.8, 11.3, 15.0 Hz, 1H), 1.54 (d, J=6.6 Hz, 3H) MS (ES+) m/z 416 (M+H)⁺

### Example 177

### 5-(difluoromethyl)-4-(((3R,5S)-5-methylpyrrolidin-3-yl)oxy)-N-(quinoxalin-6-ylmethyl)pyridin-3-amine (Comp. 182)

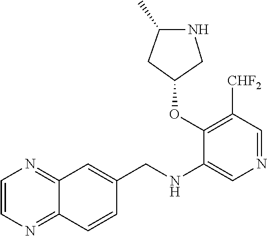 [SMILES: C[C@H]1C[C@@H](Oc2c(NCc3ccc4nccnc4c3)cncc2C(F)F)CN1]

### Example 178

### 5-(difluoromethyl)-4-(((3R,5R)-5-methylpyrrolidin-3-yl)oxy)-N-(quinoxalin-6-ylmethyl)pyridin-3-amine (Comp. 183)

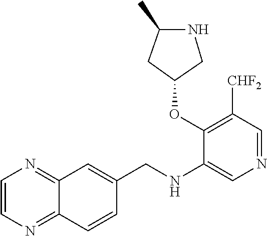 [SMILES: C[C@@H]1C[C@@H](Oc2c(NCc3ccc4nccnc4c3)cncc2C(F)F)CN1]

### Example 179

### 4-(((3R,5S)-5-methylpyrrolidin-3-yl)oxy)-N-(quinoxalin-6-ylmethyl)-5-(trifluoromethyl)pyridin-3-amine (Comp. 184)

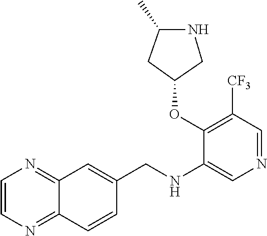 [SMILES: C[C@H]1C[C@@H](Oc2c(NCc3ccc4nccnc4c3)cncc2C(F)(F)F)CN1]

¹H NMR (400 MHz, MeOD-d₄) δ 8.89 (s, 2H), 8.14 (d, J=8.40 Hz, 1H), 8.08 (d, J=1.20 Hz, 1H), 8.05 (s, 1H), 8.03 (s, 1H), 5.21-5.06 (m, 1H), 4.79 (s, 2H), 3.39 (br d, J=12.8 Hz, 2H), 3.21-3.10 (m, 1H), 2.96 (dd, J1=5.20 Hz, J2=12.8 Hz, 1H), 2.48 (td, J1=7.3, J2=14.3 Hz, 1H), 1.77-1.66 (m, 1H), 1.33 (d, J=6.4 Hz, 3H). MS (ES+) m/z 404 (M+H)⁺.

### Example 180

### 4-(((3R,5R)-5-methylpyrrolidin-3-yl)oxy)-N-(quinoxalin-6-ylmethyl)-5-(trifluoromethyl)pyridin-3-amine (Comp. 185)

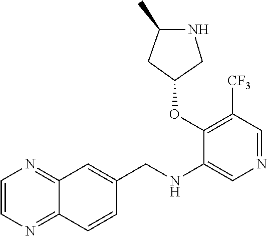 [SMILES: C[C@@H]1C[C@@H](Oc2c(NCc3ccc4nccnc4c3)cncc2C(F)(F)F)CN1]

### Sequence Information:

TABLE 1
Aptamer sequences identified from library N1
in which nucleotides at positions X₇-X₁₂
where randomized as provided in the
sequence CTGGGGAGTCCTTCATGCGGGGCTGAGAGGA
TGGAAGCAATCGACCATCGACCCX₇X₈X₉X₁₀X₁₁X₁₂
CCTGATCCGGATCATGCCGGCGCAGGGAG (SEQ ID NO: 4).
The sequences provided in Table 1, below, for
the J2-4 (X₇X₈X₉X₁₀X₁₁X₁₂) are in the context
provided in SEQ ID NO: 4.

| SEQ ID | Aptamer | Sequence | |
|---|---|---|---|
| NO: | Name | J2-4 | Fold |
| 4 | | X₇X₈X₉X₁₀X₁₁X₁₂ | |
| 1 | 12C6-1 | ATTGCA | |
| 7 | N1_1F1_2 | ACACCA | 356.65 |
| 8 | N1_2H3 | ATCGA | 338.25 |
| 9 | N1_1A10_5 | ACTGCA | 289.89 |
| 10 | N1_1D6_5 | ATAAAA | 277.63 |
| 11 | N1_1H8_1 | ATTGTA | 253.58 |
| 12 | N1_2F6 | AAAATA | 185.92 |
| 13 | N1_2F11 | ATAATA | 150.18 |
| 14 | N1_1H4_1 | ATATGA | 133.71 |
| 15 | N1_2C7 | TTCAAA | 109.60 |
| 16 | N1_2D2 | AACGTA | 102.23 |
| 17 | N1_2G1 | ATCCTA | 100.11 |
| 18 | N1_1G9 | AGACGT | 57.55 |
| 19 | N1_2A10 | AGACTT | 43.14 |
| 20 | N1_2F7 | AACTGA | 41.88 |
| 21 | N1_2B8 | TTTTTC | 41.84 |
| 22 | N1_2G3 | GTACGA | 36.58 |
| 23 | N1_2D5 | TTTCAC | 35.71 |
| 24 | N1_2E5 | TTCAAC | 35.05 |
| 25 | N1_2G9 | TTTCGC | 30.93 |
| 26 | N1_2E6 | TTCACC | 30.39 |
| 27 | N1_1H4_4 | GTTTGT | 27.89 |
| 28 | N1_1A10_2 | TATATA | 25.13 |
| 29 | N1_2B6 | ACGAGT | 24.49 |
| 30 | N1_2A3 | ACTTAT | 22.20 |
| 31 | N1_1D6_2 | TTAAGT | 20.22 |
| 32 | N1_2E7 | TCCTAA | 19.91 |
| 33 | N1_2C9 | TCGACA | 19.52 |
| 34 | N1_2A7 | ATGGTC | 17.13 |
| 35 | N1_2C6 | ATTTTG | 17.04 |
| 36 | N1_2H10 | TTATGT | 16.82 |
| 37 | N1_2F3 | TCTGTA | 15.56 |
| 38 | N1_2D10 | AATTAG | 15.53 |
| 39 | N1_2E11 | ATCACG | 15.45 |
| 40 | N1_2D6 | GTATTG | 13.57 |
| 41 | N1_2B11 | TTTGTG | 12.85 |
| 42 | N1_2B3 | TACCCC | 12.35 |
| 43 | N1_2A11 | TTCGTG | 11.48 |
| 44 | N1_2E9 | TCTGAT | 11.27 |
| 45 | N1_2G6 | AGAGGC | 11.00 |
| 46 | N1_2F10 | TTACTG | 10.84 |
| 47 | N1_2E4 | TCAATG | 10.44 |
| 48 | N1_2B2 | TTTTAG | 10.02 |
| 49 | N1_2D9 | AGTAAA | 9.74 |
| 50 | N1_2A12 | GGACTA | 9.72 |
| 51 | N1_2B7 | AATCGT | 9.58 |
| 52 | N1_2H1 | GTGTAG | 9.41 |
| 53 | N1_2G4 | GGTGAA | 9.06 |
| 54 | N1_2C3 | GTTGAT | 8.87 |
| 55 | N1_2C10 | GAGTGT | 8.74 |
| 56 | N1_2G8 | AGTTAC | 8.46 |
| 57 | N1_2F1 | AAATCT | 8.34 |
| 58 | N1_2E2 | ATGCGT | 7.86 |
| 59 | N1_2E8 | AATGCT | 5.81 |
| 60 | N1_2E3 | TTCCCG | 5.79 |
| 61 | N1_2D3 | GCGAGA | 5.40 |
| 62 | N1_2F4 | TCTTAG | 4.74 |
| 63 | N1_2G10 | GAAGGG | 4.40 |
| 64 | N1_2D1 | AGGGAA | 4.38 |
| 65 | N1_1D6_1 | AGTTTC | 4.13 |
| 66 | N1_2C4 | GCGCAT | 3.80 |
| 67 | N1_2B12 | ATGGGG | 3.79 |
| 68 | N1_2A9 | TTCCTC | 3.41 |
| 69 | N1_2D12 | TGGCAG | 3.26 |
| 70 | N1_2E10 | TCTTGG | 2.86 |
| 71 | N1_2H2 | CCGTTC | 2.85 |
| 72 | N1_2H11 | CTTTCC | 2.83 |
| 73 | N1_2C8 | GACAAG | 2.67 |
| 74 | N1_2B5 | GGAGGT | 2.51 |
| 75 | N1_2A8 | GCCTGG | 2.24 |
| 76 | N1_2D7 | CCACGG | 2.16 |
| 77 | N1_2G7 | AGGTTG | 2.06 |
| 78 | N1_2D11 | GGCGGG | 1.96 |
| 79 | N1_1A10_3 | TTTCGG | 1.93 |
| 80 | N1_2H9 | CTTTTA | 1.91 |
| 81 | N1_2F12 | CGCACA | 1.83 |
| 82 | N1_2F9 | CTCCGG | 1.71 |
| 83 | N1_2A5 | CTCTCA | 1.65 |
| 84 | N1_2H5 | CCTGCG | 1.62 |
| 85 | N1_2H8 | CCGCCC | 1.45 |
| 86 | N1_1F1_1 | CCCCCA | 1.22 |
| 87 | N1_1F7 | CGGACC | 1.22 |
| 88 | N1_2H7 | GGTCGG | 1.16 |

TABLE 2
Aptamer sequences identified from library
N3 in which nucleotides at positions
X₁X₂AX₃X₄X₅X₆ were randomized as provided
in the sequence CTGGGGAGTCCTTCATG
CGGGGCTGAGAGGATGGAAGX₁X₂AX₃X₄X₅X₆
CCATCGACCCATTGCACCTGATCCGGATCA
TGCCGGCGCAGGGAG (SEQ ID NO: 3).
The sequences provided in Table 2, below,
for the J2-4 (X₁X₂AX₃X₄X₅X₆) are in the
context provided in SEQ ID NO: 3.

| SEQ ID | Aptamer | Sequence | |
|---|---|---|---|
| NO: | Name | L3a | Fold |
| 3 | | X₁X₂AX₃X₄X₅X₆ | |
| 1 | 12C6-1 | CAATCGA | |
| 89 | N3_G6 | CGATGAG | 858.1 |
| 90 | N3_B2 | CTATTGA | 435.1 |
| 91 | N3_C1 | CTATTGA | 418.7 |
| 92 | N3_B8 | CCAAGAG | 301.6 |
| 93 | N3_B11 | TAACGTG | 299.6 |
| 94 | N3_F2 | CCACGGA | 207.3 |
| 95 | N3_F8 | TAAGTGG | 155.6 |
| 96 | N3_G11 | CGAAGGA | 128.2 |
| 97 | N3_B12 | TAAAGGC | 70.0 |
| 98 | N3_B1 | TAACAAT | 67.1 |
| 99 | N3_A1 | TTATCAT | 28.2 |
| 100 | N3_B5 | TTAAAGC | 20.5 |
| 101 | N3_E1 | TTACTGT | 18.9 |
| 102 | N3_C7 | TTATTCT | 15.2 |
| 103 | N3_B9 | GTAAAAT | 13.2 |
| 104 | N3_A12 | CTATCAT | 13.2 |
| 105 | N3_F9 | TTACCGT | 12.7 |
| 106 | N3_B7 | TTATAGC | 10.5 |
| 107 | N3_C2 | AAACCGA | 9.9 |
| 108 | N3_G2 | CAAGGTT | 9.8 |
| 109 | N3_C8 | GTATTGT | 8.6 |
| 110 | N3_G12 | TTAGTTA | 7.6 |
| 111 | N3_F12 | CTACTAG | 7.5 |
| 112 | N3_H3 | CAACGTC | 7.2 |
| 113 | N3_A3 | GTACAGT | 7.1 |
| 114 | N3_E7 | TCATTCT | 6.6 |
| 115 | N3_F5 | TGATGGC | 6.5 |
| 116 | N3_E8 | TCATGTG | 6.3 |
| 117 | N3_H9 | ATAATGT | 5.9 |
| 118 | N3_H5 | GTATTGT | 5.9 |
| 119 | N3_F6 | GCATAGA | 5.9 |
| 120 | N3_H8 | GTATGTT | 5.6 |
| 121 | N3_G5 | AGACGCC | 5.1 |
| 122 | N3_D1 | CGAACCC | 4.4 |
| 123 | N3_D4 | TCAACTG | 4.3 |
| 124 | N3_D5 | TCATGGC | 4.3 |
| 125 | N3_F10 | GTATACT | 4.1 |
| 126 | N3_C4 | TCAGCGG | 3.9 |
| 127 | N3_E2 | CAAATTG | 3.7 |
| 128 | N3_B6 | GCAGTGT | 3.7 |
| 129 | N3_E9 | ATATGTG | 3.5 |
| 130 | N3_G8 | GAAAGTT | 3.5 |
| 131 | N3_D10 | GAATAGT | 3.5 |
| 132 | N3_F3 | ACAGGCC | 3.3 |
| 133 | N3_G10 | GTATTCG | 3.2 |
| 134 | N3_H6 | GCAGGAA | 3.1 |
| 135 | N3_G4 | GTACAGC | 3.0 |
| 136 | N3_E12 | TGATTTG | 3.0 |
| 137 | N3_C9 | TCATACC | 2.9 |
| 138 | N3_B4 | GTAGTTC | 2.8 |
| 139 | N3_C5 | TGAAGCT | 2.7 |
| 140 | N3_H7 | GAAAGTT | 2.7 |
| 141 | N3_D11 | GGAATAT | 2.7 |
| 142 | N3_E6 | ACAACTT | 2.5 |
| 143 | N3_C10 | AAACTTG | 2.5 |
| 144 | N3_D7 | TGAGACT | 2.4 |
| 145 | N3_C3 | CCAGTTG | 2.3 |
| 146 | N3_A11 | ACAAATG | 2.2 |
| 147 | N3_E4 | AGAATGG | 2.1 |
| 148 | N3_H4 | AGAAAAC | 2.1 |
| 149 | N3_E10 | ATAGTGG | 2.1 |
| 150 | N3_H2 | CCAAGGG | 2.1 |
| 151 | N3_C6 | AGAACAG | 2.1 |
| 152 | N3_H12 | ATAGGCG | 2.1 |
| 153 | N3_H11 | TGACTTG | 2.1 |
| 154 | N3_D6 | TGACTTA | 2.1 |
| 155 | N3_D8 | TGAGCAG | 2.0 |
| 156 | N3_A4 | TTATCTT | 2.0 |
| 157 | N3_G9 | GAAGCAA | 2.0 |
| 158 | N3_D9 | AGAAGTT | 1.9 |
| 159 | N3_G1 | GAAGGAG | 1.9 |
| 160 | N3_A8 | GAATATC | 1.9 |
| 161 | N3_F7 | TCAGGTA | 1.9 |
| 162 | N3_E3 | TCAGCTT | 1.8 |
| 163 | N3_H10 | TCATGGG | 1.7 |
| 164 | N3_E5 | AGAGTAA | 1.6 |
| 165 | N3_F11 | GGAGCGT | 1.5 |
| 166 | N3_H1 | CGAATTC | 1.4 |
| 167 | N3_A10 | CAATGGG | 1.4 |
| 168 | N3_B3 | GGATAGT | 1.4 |
| 169 | N3_B10 | GGACGCT | 1.3 |
| 170 | N3_G7 | GGATCAT | 1.3 |
| 171 | N3_A2 | GGACCAA | 1.2 |
| 172 | N3_A7 | GAAGGGA | 1.2 |
| 173 | N3_F1 | TGATGCG | 1.1 |

TABLE 3
Aptamer sequences identified from library N4
in which nucleotides at positions X₁₃X₁₄X₁₅
and X₂₂X₂₃ were randomized as provided in
the sequence CTGGGGAGTCCTTCATGCGGGGCTGA
GAGGATGGAAGCAATCGACCATCGACCCATTGCACCTX₁₃
X₁₄X₁₅CCGGATCATGCCGGX₂₂X₂₃CAGGGAG
(SEQ ID NO: 5). The sequences provided
in Table 3, below, for P4/J4-5 to J5-4
(X₁₃X₁₄X₁₅CCGGATCATGCCGGX₂₂X₂₃) are in
the context provided in SEQ ID NO: 5.

| SEQ ID | Aptamer | Sequence | |
|---|---|---|---|
| NO: | Name | P4/J4-5 to J5-4 | Fold |
| 5 | | X₁₃X₁₄X₁₅CCGGATCATGCC | |
| | | GGX₂₂X₂₃ | |
| 1 | 12C6-1 | GATCCGGATCATGCCGGCG | |
| 174 | N4_1C11 | CGTCCGGATCATGCCGGTA | 2096.5 |
| 175 | N4_1E10 | GATCCGGATCATGCCGGTG | 1405.5 |
| 176 | N4_1B04 | ACGCCGGATCATGCCGGTG | 1347.4 |
| 177 | N4_6D7 | GACCCGGATCATGCCGGTA | 1227.9 |
| 178 | N4_3A3 | GGTCCGGATCATGCCGGTA | 1147.0 |
| 179 | N4_2C6 | GTGCCGGATCATGCCGGTT | 1115.8 |
| 180 | N4_4F10 | CGTCCGGATCATGCCGGAA | 1092.2 |
| 181 | N4_3E4 | GGTCCGGATCATGCCGGCT | 1086.5 |
| 182 | N4_7A9 | CGGCCGGATCATGCCGGTA | 1075.9 |
| 183 | N4_7A10 | CGACCGGATCATGCCGGGT | 1049.9 |
| 184 | N4_1H9 | AGCCCGGATCATGCCGGGT | 1044.8 |
| 185 | N4_7B3 | TTCCCGGATCATGCCGGGA | 1042.5 |
| 186 | N4_2B6 | GATCCGGATCATGCCGGTA | 1030.5 |
| 187 | N4_9B7 | GAGCCGGATCATGCCGGTT | 1021.6 |
| 188 | N4_9B9 | AGGCCGGATCATGCCGGTC | 1009.4 |
| 189 | N4_5F12 | GGCCCGGATCATGCCGGAG | 1009.3 |
| 190 | N4_7B8 | GCGCCGGATCATGCCGGTA | 1009.2 |
| 191 | N4_1B10 | GACCCGGATCATGCCGGAG | 1002.5 |
| 192 | N4_7B12 | GGACCGGATCATGCCGGCT | 1000.4 |
| 193 | N4_9B7 | TGCCCGGATCATGCCGGCG | 989.9 |
| 194 | N4_6B3 | GTCCCGGATCATGCCGGTA | 988.7 |
| 195 | N4_2E5 | GCCCCGGATCATGCCGGTG | 980.6 |
| 196 | N4_2A10 | GCACCGGATCATGCCGGGC | 979.3 |
| 197 | N4_9E4 | AGCCCGGATCATGCCGGTG | 972.3 |
| 198 | N4_9C7 | GGACCGGATCATGCCGGTT | 969.3 |
| 199 | N4_9A3 | GTCCCGGATCATGCCGGCT | 960.2 |
| 200 | N4_5B12 | GCTCCGGATCATGCCGGAT | 956.4 |
| 201 | N4_1E8 | GCACCGGATCATGCCGGGT | 954.3 |
| 202 | N4_1F12 | TCCCCGGATCATGCCGGGG | 947.8 |
| 203 | N4_1C03 | CAGCCGGATCATGCCGGTT | 941.1 |
| 204 | N4_A62 | GTACCGGATCATGCCGGAT | 933.7 |
| 205 | N4_1G02 | GTGCCGGATCATGCCGGTG | 929.0 |
| 206 | N4_1C02 | GCACCGGATCATGCCGGAA | 927.7 |
| 207 | N4_1G5 | AGTCCGGATCATGCCGGGA | 927.6 |
| 208 | N4_1D6 | CGGCCGGATCATGCCGGGC | 927.0 |
| 209 | N4_8G4 | GTGCCGGATCATGCCGGCG | 926.3 |
| 210 | N4_1G7 | GAGCCGGATCATGCCGGTA | 924.9 |
| 211 | N4_8E2 | GACCCGGATCATGCCGGTG | 917.8 |
| 212 | N4_2C5 | GGACCGGATCATGCCGGAG | 915.5 |
| 213 | N4_2G2 | GGTCCGGATCATGCCGGTT | 908.3 |
| 214 | N4_7F3 | CGACCGGATCATGCCGGAT | 905.6 |
| 215 | N4_2D3 | CGTCCGGATCATGCCGGGA | 897.4 |
| 216 | N4_2G5 | GGGCCGGATCATGCCGGCG | 896.8 |
| 217 | N4_1C10 | GATCCGGATCATGCCGGAA | 886.8 |
| 218 | N4_1F7 | GGTCCGGATCATGCCGGAG | 884.7 |
| 219 | N4_5E4 | GGGCCGGATCATGCCGGTT | 884.6 |
| 220 | N4_1E6 | GTACCGGATCATGCCGGGT | 878.6 |
| 221 | N4_1B12 | GATCCGGATCATGCCGGGA | 878.0 |
| 222 | N4_7B2 | GTACCGGATCATGCCGGGG | 876.1 |
| 223 | N4_8B3 | CTACCGGATCATGCCGGTA | 875.0 |
| 224 | N4_1F6 | GCTCCGGATCATGCCGGCA | 868.3 |
| 225 | N4_7H9 | GCACCGGATCATGCCGGTC | 865.6 |
| 226 | N4_1F10 | TACCCGGATCATGCCGGGG | 865.5 |
| 227 | N4_1C5 | GGTCCGGATCATGCCGGCC | 863.5 |
| 228 | N4_8F1 | GACCCGGATCATGCCGGAC | 858.6 |
| 229 | N4_3B2 | GGCCCGGATCATGCCGGAT | 850.6 |
| 230 | N4_1A11 | CACCCGGATCATGCCGGGG | 849.3 |
| 231 | N4_1H6 | GGCCCGGATCATGCCGGCG | 838.4 |
| 232 | N4_6B3 | GGACCGGATCATGCCGGTA | 828.1 |
| 233 | N4_1E3 | GCACCGGATCATGCCGGCC | 819.8 |
| 234 | N4_1H1 | TAGCCGGATCATGCCGGCT | 818.7 |
| 235 | N4_1G11 | TGGCCGGATCATGCCGGCT | 817.0 |
| 236 | N4_1D10 | GAGCCGGATCATGCCGGCT | 813.1 |
| 237 | N4_1C7 | GCCCCGGATCATGCCGGGC | 809.0 |
| 238 | N4_2G9 | CATCCGGATCATGCCGGTA | 798.5 |
| 239 | N4_1E10 | GGTCCGGATCATGCCGGGC | 797.0 |
| 240 | N4_6F6 | GGGCCGGATCATGCCGGTC | 796.6 |
| 241 | N4_3D3 | AATCCGGATCATGCCGGTA | 792.4 |
| 242 | N4_1D1 | GTTCCGGATCATGCCGGGG | 790.8 |
| 243 | N4_4D4 | ATGCCGGATCATGCCGGTA | 777.1 |
| 244 | N4_7D10 | GTTCCGGATCATGCCGGTC | 765.6 |
| 245 | N4_3F3 | GGGCCGGATCATGCCGGTA | 759.8 |
| 246 | N4_1A12 | AGGCCGGATCATGCCGGCT | 755.5 |
| 247 | N4_3A4 | TGCCCGGATCATGCCGGGC | 754.4 |
| 248 | N4_7D1 | GTGCCGGATCATGCCGGAT | 751.6 |
| 249 | N4_4F5 | GCCCCGGATCATGCCGGTA | 751.2 |
| 250 | N4_3G11 | GAGCCGGATCATGCCGGAG | 743.9 |
| 251 | N4_7H7 | TTGCCGGATCATGCCGGTA | 742.8 |
| 252 | N4_7E12 | TCTCCGGATCATGCCGGAG | 740.1 |
| 253 | N4_7G7 | GAACCGGATCATGCCGGCG | 736.8 |
| 254 | N4_1H7 | TGACCGGATCATGCCGGTA | 728.2 |
| 255 | N4_7G5 | CGGCCGGATCATGCCGGCT | 727.2 |
| 256 | N4_1E2 | GAACCGGATCATGCCGGCC | 724.9 |
| 257 | N4_3F8 | GGACCGGATCATGCCGGGT | 723.6 |
| 258 | N4_4H1 | GTACCGGATCATGCCGGGA | 719.6 |
| 259 | N4_4H4 | GCACCGGATCATGCCGGGA | 719.3 |
| 260 | N4_3F4 | GTTCCGGATCATGCCGGTT | 713.7 |
| 261 | N4_4B5 | GGTCCGGATCATGCCGGCG | 703.4 |
| 262 | N4_5A7 | GAACCGGATCATGCCGGGT | 701.3 |
| 263 | N4_7C11 | GTGCCGGATCATGCCGGCT | 692.4 |
| 264 | N4_5H7 | GAGCCGGATCATGCCGGAA | 687.0 |
| 265 | N4_7H1 | TGGCCGGATCATGCCGGCC | 681.4 |
| 266 | N4_1A4 | AAGCCGGATCATGCCGGCG | 679.6 |
| 267 | N4_7D7 | GGACCGGATCATGCCGGCG | 674.8 |
| 268 | N4_4E3 | GTGCCGGATCATGCCGGGA | 660.7 |
| 269 | N4_7G9 | AGACCGGATCATGCCGGTT | 658.4 |
| 270 | N4_1A7 | GGGCCGGATCATGCCGGGT | 648.6 |
| 271 | N4_7H10 | AGTCCGGATCATGCCGGAA | 648.2 |
| 272 | N4_1B1 | CTGCCGGATCATGCCGGCG | 645.9 |
| 273 | N4_3C2 | GGGCCGGATCATGCCGGTG | 633.8 |
| 274 | N4_7D5 | GACCCGGATCATGCCGGGC | 633.1 |
| 275 | N4_7E11 | GCCCCGGATCATGCCGGGA | 629.2 |
| 276 | N4_5G7 | GTTCCGGATCATGCCGGGT | 621.4 |
| 277 | N4_1D11 | TGCCCGGATCATGCCGGCA | 620.4 |
| 278 | N4_7A11 | GGTCCGGATCATGCCGGGT | 617.9 |
| 279 | N4_1B2 | GTACCGGATCATGCCGGTT | 617.8 |
| 280 | N4_5B9 | GAACCGGATCATGCCGGGA | 612.3 |
| 281 | N4_1E4 | GGTCCGGATCATGCCGGAC | 609.9 |
| 282 | N4_7A3 | GGACCGGATCATGCCGGAT | 604.3 |
| 283 | N4_3B11 | ACACCGGATCATGCCGGTG | 597.1 |
| 284 | N4_7B10 | CGTCCGGATCATGCCGGAG | 593.9 |
| 285 | N4_7D9 | CTGCCGGATCATGCCGGCA | 592.8 |
| 286 | N4_1B5 | TAACCGGATCATGCCGGCA | 590.8 |
| 287 | N4_3C7 | AGTCCGGATCATGCCGGTC | 582.5 |
| 289 | N4_7B11 | GAACCGGATCATGCCGGTT | 555.1 |
| 290 | N4_7B7 | GACCCGGATCATGCCGGGA | 552.6 |
| 291 | N4_7C7 | CCACCGGATCATGCCGGTG | 534.4 |
| 292 | N4_1H2 | CATCCGGATCATGCCGGTG | 528.5 |
| 293 | N4_7B1 | TAGCCGGATCATGCCGGTT | 518.2 |
| 294 | N4_7A2 | TACCCGGATCATGCCGGTA | 518.2 |
| 295 | N4_1A2 | AAGCCGGATCATGCCGGCC | 512.7 |
| 296 | N4_7G4 | GTACCGGATCATGCCGGAA | 500.9 |
| 297 | N4_7E6 | GGGCCGGATCATGCCGGAG | 493.4 |
| 298 | N4_1D12 | TTACCGGATCATGCCGGTG | 489.6 |
| 299 | N4_1D5 | ACGCCGGATCATGCCGGTT | 488.4 |
| 300 | N4_1C12 | TGGCCGGATCATGCCGGAA | 471.2 |
| 301 | N4_1C8 | CTACCGGATCATGCCGGCA | 467.1 |
| 302 | N4_3F10 | AGCCCGGATCATGCCGGGA | 464.5 |
| 303 | N4_1H11 | TTCCCGGATCATGCCGGAA | 452.0 |
| 304 | N4_1B7 | TCGCCGGATCATGCCGGTA | 445.0 |
| 305 | N4_7F5 | AGCCCGGATCATGCCGGGG | 436.3 |
| 306 | N4_7G11 | CGGCCGGATCATGCCGGCG | 430.4 |
| 307 | N4_7A6 | TCACCGGATCATGCCGGTA | 424.0 |
| 308 | N4_7E3 | ATGCCGGATCATGCCGGCG | 414.2 |
| 309 | N4_7A5 | AGACCGGATCATGCCGGCA | 402.4 |
| 310 | N4_7E8 | ACTCCGGATCATGCCGGAT | 393.6 |
| 311 | N4_7D2 | AAGCCGGATCATGCCGGTG | 388.9 |
| 312 | N4_2E10 | TGACCGGATCATGCCGGCG | 361.5 |
| 313 | N4_7E4 | TCACCGGATCATGCCGGCG | 348.8 |
| 314 | N4_1H12 | ATACCGGATCATGCCGGGA | 344.8 |
| 315 | N4_1C1 | AAGCCGGATCATGCCGGAC | 342.9 |
| 316 | N4_7H5 | ATCCCGGATCATGCCGGGG | 341.6 |
| 317 | N4_1A10 | ATACCGGATCATGCCGGCG | 337.4 |
| 318 | N4_1F3 | CCCCCGGATCATGCCGGGA | 333.5 |
| 319 | N4_7F10 | GGTCCGGATCATGCCGGTG | 319.6 |
| 320 | N4_1A3 | CGGCCGGATCATGCCGGAG | 317.2 |
| 321 | N4_7F2 | TATCCGGATCATGCCGGAA | 302.5 |
| 322 | N4_1G1 | AATCCGGATCATGCCGGTC | 282.3 |
| 323 | N4_1H1C | TATCCGGATCATGCCGGTG | 279.0 |
| 324 | N4_7H8 | TGCCCGGATCATGCCGGCC | 276.6 |
| 325 | N4_1H8 | TAGCCGGATCATGCCGGAA | 246.1 |
| 326 | N4_1F4 | CAACCGGATCATGCCGGGT | 245.3 |
| 327 | N4_1C9 | TGGCCGGATCATGCCGGAG | 241.2 |
| 328 | N4_7E10 | AGTCCGGATCATGCCGGTT | 238.9 |
| 329 | N4_7C1 | TTGCCGGATCATGCCGGGA | 238.8 |
| 330 | N4_1E5 | TGACCGGATCATGCCGGAT | 235.5 |
| 331 | N4_1E7 | TATCCGGATCATGCCGGGC | 232.9 |
| 332 | N4_7G6 | CGCCCGGATCATGCCGGCA | 218.0 |
| 333 | N4_7F12 | TCACCGGATCATGCCGGGA | 215.9 |
| 334 | N4_1H4 | GTCCCGGATCATGCCGGGG | 214.7 |
| 335 | N4_7H11 | ATGCCGGATCATGCCGGAG | 207.8 |
| 336 | N4_7E7 | GTTCCGGATCATGCCGGAA | 205.9 |
| 337 | N4_1F1 | TGTCCGGATCATGCCGGTT | 199.3 |
| 338 | N4_1G9 | AAACCGGATCATGCCGGCC | 174.6 |
| 339 | N4_7E1 | TCACCGGATCATGCCGGAG | 170.7 |
| 340 | N4_7C10 | ACTCCGGATCATGCCGGGA | 153.8 |
| 341 | N4_1B3 | TCGCCGGATCATGCCGGGA | 151.6 |
| 342 | N4_7D3 | TGTCCGGATCATGCCGGGG | 150.9 |
| 343 | N4_1D4 | TTTCCGGATCATGCCGGGT | 144.5 |
| 344 | N4_7D6 | TCTCCGGATCATGCCGGTG | 144.4 |
| 345 | N4_1A8 | TTTCCGGATCATGCCGGTC | 135.7 |
| 346 | N4_1B11 | TAGCCGGATCATGCCGGGA | 135.0 |
| 347 | N4_7A7 | AAGCCGGATCATGCCGGGG | 120.1 |
| 348 | N4_1G4 | TAGCCGGATCATGCCGGGT | 115.8 |
| 349 | N4_7B5 | GTCCCGGATCATGCCGGTG | 108.9 |
| 350 | N4_7F6 | AGGCCGGATCATGCCGGTG | 84.5 |
| 351 | N4_1F9 | CCACCGGATCATGCCGGCT | 82.6 |
| 352 | N4_1D8 | ATTCCGGATCATGCCGGTG | 80.7 |
| 353 | N4_1A5 | CAGCCGGATCATGCCGGGT | 73.5 |
| 354 | N4_7A4 | ATTCCGGATCATGCCGGGT | 58.9 |
| 355 | N4_1G10 | CTCCCGGATCATGCCGGTG | 54.5 |
| 356 | N4_1B9 | ATTCCGGATCATGCCGGAC | 21.1 |
| 357 | N4_1D3 | AGGCCGGATCATGCCAGAT | 6.5 |

TABLE 4
Aptamer sequences identified from library
N5 in which nucleotides at positions
X₁₆X₁₇X₁₈X₁₉X₂₀X₂₁ were randomized as provided
in the sequence CTGGGGAGTCCTTCATGCGGGGCTG
AGAGGATGGAAGCAATCGACCATCGACCCATTGCACCTGAT
CCGGX₁₆X₁₇X₁₈X₁₉X₂₀X₂₁CCGGCGCAGGGAG (SEQ ID
NO: 6). The sequences provided in Table 4,
below, for L5 (X₁₆X₁₇X₁₈X₁₉X₂₀X₂₁) are in
the context provided in SEQ ID NO: 6.

| SEQ ID | Aptamer | Sequence | |
|---|---|---|---|
| NO: | Name | L5 | Fold |
| 6 | | X₁₆X₁₇X₁₈X₁₉X₂₀X₂₁ | |
| 1 | 12C6-1 | ATCATG | |
| 358 | N5_12G6 | TATGTC | 2750.8 |
| 359 | N5_6D12 | TAGGAC | 2318.1 |
| 360 | N5_13E8 | TACGGT | 2096.6 |
| 361 | N5_12F4 | GTGAGG | 2096.2 |
| 362 | N5_7B1 | GTAAGG | 2062.3 |
| 363 | N5_12E5 | TAGGCT | 1898.6 |
| 364 | N5_14G11 | TCGGTC | 1775.3 |
| 365 | N5_7F1 | ATGTTC | 1744.2 |
| 366 | N5_7E1 | AGAGTG | 1733.0 |
| 367 | N5_11D10 | AAATAG | 1719.3 |
| 368 | N5_6F5 | GTGATG | 1665.4 |
| 369 | N5_3F11 | TATGGT | 1628.3 |
| 370 | N5_13G10 | TGGCGG | 1604.0 |
| 371 | N5_11B9 | TCAGTC | 1595.4 |
| 372 | N5_1E12 | CAGCCG | 1588.2 |
| 373 | N5_7G11 | GTTGAT | 1533.8 |
| 374 | N5_9H1 | GTACGT | 1494.7 |
| 375 | N5_6H10 | GTAAGA | 1466.1 |
| 376 | N5_9E12 | TGGACG | 1458.4 |
| 377 | N5_6B4 | ACGTTG | 1435.7 |
| 378 | N5_1B6 | TATGTG | 1381.8 |
| 379 | N5_7C4 | TTGGTA | 1363.2 |
| 380 | N5_7B10 | TCAGTT | 1361.5 |
| 381 | N5_5B7 | GTTGGG | 1317.2 |
| 382 | N5_7C8 | ATCGAG | 1313.8 |
| 383 | N5_1G6 | TATGGC | 1311.6 |
| 384 | N5_13H3 | GTGAGT | 1288.5 |
| 385 | N5_8F11 | AGTTAG | 1282.8 |
| 386 | N5_7F8 | TTGGGA | 1279.7 |
| 387 | N5_6G10 | GTTGCA | 1262.0 |
| 388 | N5_7C3 | ACTTCA | 1258.4 |
| 389 | N5_6A8 | CAAGTA | 1252.2 |
| 390 | N5_2G10 | GCTGGT | 1249.7 |
| 391 | N5_7F4 | ATGTAC | 1239.0 |
| 392 | N5_5C4 | GATTAG | 1235.2 |
| 393 | N5_7E2 | GTACAG | 1234.5 |
| 394 | N5_1G9 | GTTTAG | 1230.3 |
| 395 | N5_11F4 | AACAGT | 1230.1 |
| 396 | N5_6F4 | AGAGAT | 1226.6 |
| 397 | N5_7A7 | GCTTGT | 1226.2 |
| 398 | N5_3D3 | TATAGA | 1222.7 |
| 399 | N5_8H7 | GTGAGC | 1218.5 |
| 400 | N5_7D6 | TCGTTG | 1210.5 |
| 401 | N5_13G12 | GCTTCG | 1199.4 |
| 402 | N5_13G5 | CGGTTA | 1198.5 |
| 403 | N5_7H12 | CATGCG | 1194.5 |
| 404 | N5_8H1 | TAGGTT | 1191.9 |
| 405 | N5_11G11 | GGTAGT | 1186.4 |
| 406 | N5_7D5 | TGGTCG | 1182.3 |
| 407 | N5_12F12 | CGCATA | 1169.8 |
| 408 | N5_7E6 | TAACCG | 1153.2 |
| 409 | N5_7D4 | GCTGTC | 1144.9 |
| 410 | N5_13C10 | AACGTA | 1144.6 |
| 411 | N5_8D1 | ACTGTG | 1139.6 |
| 412 | N5_7F3 | TAGAGC | 1139.0 |
| 413 | N5_14B10 | AATGCA | 1133.0 |
| 414 | N5_5E4 | AGAGTT | 1116.0 |
| 415 | N5_7F5 | GTACTA | 1115.4 |
| 416 | N5_7D2 | GTTCCG | 1114.3 |
| 417 | N5_7A10 | TAGTCG | 1108.5 |
| 418 | N5_2B5 | GCATAA | 1107.3 |
| 419 | N5_7H11 | TAACAC | 1105.2 |
| 420 | N5_2F12 | GGCAGC | 1100.0 |
| 421 | N5_1E11 | TGTGAA | 1095.6 |
| 422 | N5_9C12 | ATTGGA | 1094.3 |
| 423 | N5_7C10 | CTGTTT | 1090.7 |
| 424 | N5_8A9 | CGATAT | 1090.4 |
| 425 | N5_7D3 | ATGGTC | 1075.7 |
| 426 | N5_12G2 | AATGTT | 1074.1 |
| 427 | N5_1F10 | TCTACG | 1071.3 |
| 428 | N5_5B6 | TAAAGC | 1068.3 |
| 429 | N5_8G9 | GCGTTG | 1066.5 |
| 430 | N5_3A3 | TAACAG | 1066.3 |
| 431 | N5_1F12 | TAAATT | 1057.8 |
| 432 | N5_2C5 | AAAGAG | 1055.1 |
| 433 | N5_2D8 | TAGCGA | 1047.3 |
| 434 | N5_7B7 | TGAATG | 1046.3 |
| 435 | N5_12G11 | TGGTAG | 1043.8 |
| 436 | N5_2E11 | ATGCTA | 1038.1 |
| 437 | N5_6E6 | CAGTCA | 1032.8 |
| 438 | N5_1H7 | TGATGG | 1032.1 |
| 439 | N5_9C2 | GTTGAG | 1027.6 |
| 440 | N5_1E10 | GTTGTA | 1026.1 |
| 441 | N5_6A6 | CCTGAA | 1024.4 |
| 442 | N5_9E6 | GTTTGG | 1020.7 |
| 443 | N5_2F10 | AGTAGT | 1014.6 |
| 444 | N5_1B10 | AGTTTG | 1013.6 |
| 445 | N5_2B11 | GGTTCA | 1009.4 |
| 446 | N5_6G6 | ACAGTG | 1004.4 |
| 447 | N5_1G10 | CGCATG | 1000.2 |
| 448 | N5_7A4 | GGTATC | 988.3 |
| 449 | N5_1C5 | ACACTA | 966.1 |
| 450 | N5_1F4 | AAATGT | 958.5 |
| 451 | N5_7E9 | GCAGGT | 955.3 |
| 452 | N5_6F6 | CGGTTG | 953.0 |
| 453 | N5_7G7 | AATTCC | 952.9 |
| 454 | N5_8F1 | ACAGTA | 951.8 |
| 455 | N5_14D10 | GTCAAT | 950.5 |
| 456 | N5_1B9 | ATCAGG | 941.5 |
| 457 | N5_7A11 | GACCTA | 941.2 |
| 458 | N5_9G7 | TTGCTT | 937.7 |
| 459 | N5_7F2 | GCAGTT | 937.4 |
| 460 | N5_7G1 | TCAAGA | 935.4 |
| 461 | N5_7E12 | TTGAAG | 933.3 |
| 462 | N5_11G4 | CGGGGG | 931.9 |
| 463 | N5_12F11 | GAATGG | 926.7 |
| 464 | N5_14H11 | CACACA | 918.4 |
| 465 | N5_1C9 | GTGAAT | 915.3 |
| 466 | N5_3F9 | AAAGGT | 909.7 |
| 467 | N5_7D1 | AGTTTT | 909.3 |
| 468 | N5_7B12 | GTGTCA | 908.4 |
| 469 | N5_7G6 | TAAACT | 902.8 |
| 470 | N5_7F6 | GAATCT | 898.5 |
| 471 | N5_14B9 | ATATGG | 897.5 |
| 472 | N5_7A1 | CACTCT | 897.3 |
| 473 | N5_2C2 | GTTAGG | 894.7 |
| 474 | N5_7G12 | GTTCCA | 894.6 |
| 475 | N5_3F10 | ATAGGG | 894.3 |
| 476 | N5_1G8 | AATGTG | 888.7 |
| 477 | N5_2D10 | CTCAGG | 884.5 |
| 478 | N5_7E8 | AAGTGG | 879.9 |
| 479 | N5_9F1 | TTGTGT | 875.6 |
| 480 | N5_7B6 | CATGTG | 875.4 |
| 481 | N5_7E7 | TTGCTG | 870.2 |
| 482 | N5_7E5 | CCGGTT | 867.4 |
| 483 | N5_8F3 | GGTAAT | 862.0 |
| 484 | N5_7H9 | CGTGAA | 860.0 |
| 485 | N5_7H5 | CGCTTG | 848.4 |
| 486 | N5_1D5 | ATTAGG | 841.7 |
| 487 | N5_7G9 | AGCGGT | 841.7 |
| 488 | N5_3C8 | GACAGA | 835.1 |
| 489 | N5_1E4 | CAACGA | 834.9 |
| 490 | N5_3D8 | CATGGA | 824.4 |
| 491 | N5_8C7 | GTCTCC | 817.6 |
| 492 | N5_1A9 | AGTCCT | 804.7 |
| 493 | N5_1G5 | GGGATC | 804.6 |
| 494 | N5_7G3 | GAGTTG | 799.3 |
| 495 | N5_1A11 | GAGACC | 793.5 |
| 496 | N5_9B8 | GAACGG | 792.8 |
| 497 | N5_1B3 | CCGTTG | 792.0 |
| 498 | N5_1G11 | TGGCAG | 789.0 |
| 499 | N5_7A5 | TCATTA | 785.5 |
| 500 | N5_1H10 | TGGAAT | 781.1 |
| 501 | N5_1B5 | AACCGC | 780.8 |
| 502 | N5_7B5 | TGCGTG | 779.9 |
| 503 | N5_7G8 | TTGCCC | 775.6 |
| 504 | N5_5D7 | TTCGGG | 769.4 |
| 505 | N5_1H3 | GATGCA | 764.7 |
| 506 | N5_1D10 | TTCCAA | 762.7 |
| 507 | N5_7D11 | TATCTA | 759.2 |
| 508 | N5_3G12 | AAGTTA | 756.5 |
| 509 | N5_7B3 | TTCCGT | 756.3 |
| 510 | N5_3C10 | TAGTCA | 751.6 |
| 511 | N5_13D5 | CACCAT | 749.4 |
| 512 | N5_1B2 | GATTAC | 747.7 |
| 513 | N5_2D9 | GTTCTA | 735.9 |
| 514 | N5_7E11 | CAAATT | 732.1 |
| 515 | N5_1E2 | AGTATC | 728.6 |
| 516 | N5_9G5 | TGTAAA | 725.3 |
| 517 | N5_6E1 | TTTGGT | 725.2 |
| 518 | N5_13D1 | AGGCAG | 722.9 |
| 519 | N5_3A2 | ACAGGT | 718.0 |
| 520 | N5_2G5 | TTAATT | 715.9 |
| 521 | N5_4H7 | ACGTAG | 714.3 |
| 522 | N5_1H9 | TCTTGG | 714.2 |
| 523 | N5_7H10 | TTAAGA | 713.9 |
| 524 | N5_5D9 | GCCAAG | 712.5 |
| 525 | N5_1E9 | TCTAAG | 699.7 |
| 526 | N5_1D3 | CGAATA | 697.1 |
| 527 | N5_6A3 | ACTTAG | 696.0 |
| 528 | N5_1C10 | GCCATC | 693.8 |
| 529 | N5_7B8 | TGCCAT | 693.7 |
| 530 | N5_7A9 | TGTATT | 692.6 |
| 531 | N5_1E8 | TTGTAA | 691.8 |
| 532 | N5_1C2 | GTTTCT | 687.2 |
| 533 | N5_2E6 | TCAAAC | 685.6 |
| 534 | N5_1F9 | TTTATT | 685.1 |
| 535 | N5_1D6 | ATATAA | 684.9 |
| 536 | N5_1E5 | GCGTCA | 681.3 |
| 537 | N5_1A10 | GCGCTC | 674.4 |
| 538 | N5_1A2 | CCAGCG | 672.2 |
| 539 | N5_1A1 | ACTGCA | 670.2 |
| 540 | N5_1C4 | GTGGCA | 665.0 |
| 541 | N5_1C3 | TCAATG | 664.1 |
| 542 | N5_1B7 | CGGTGC | 662.1 |
| 543 | N5_1A7 | GTTGCG | 659.9 |
| 544 | N5_6B6 | CACCGA | 659.7 |
| 545 | N5_1H6 | TTGCAC | 654.9 |
| 546 | N5_1B8 | GGATGA | 645.1 |
| 547 | N5_7F9 | ACTTGT | 642.4 |
| 548 | N5_1A6 | TCGAGA | 638.8 |
| 549 | N5_7C12 | GGCTCC | 628.9 |
| 550 | N5_7C2 | CCCCTT | 627.0 |
| 551 | N5_1H8 | GTCCAG | 622.6 |
| 552 | N5_1A8 | GATAAG | 589.4 |
| 553 | N5_4D7 | GCTACA | 588.1 |
| 554 | N5_7F10 | TTCTCA | 572.7 |
| 555 | N5_1E1 | GTTATG | 566.4 |
| 556 | N5_4D8 | ATGTAA | 565.6 |
| 557 | N5_1D2 | TCGTGG | 561.3 |
| 558 | N5_7H4 | CTAATG | 560.0 |
| 559 | N5_1F1 | TTTGTC | 557.2 |
| 560 | N5_7G10 | CTGCGT | 530.8 |
| 561 | N5_1D4 | TCGGAG | 522.9 |
| 562 | N5_1G2 | GCCATG | 517.7 |
| 563 | N5_7C7 | AGGCGA | 489.1 |
| 564 | N5_1C7 | TAGTCT | 485.7 |
| 565 | N5_1C11 | AGTGGG | 484.2 |
| 566 | N5_1H4 | CTAGAA | 482.5 |
| 567 | N5_1B11 | TGCACA | 477.1 |
| 568 | N5_7C11 | GGCGGT | 459.9 |
| 569 | N5_1F2 | TCTAAA | 415.1 |
| 570 | N5_1H11 | ACCCGC | 413.6 |
| 571 | N5_1H2 | GGGACA | 386.6 |
| 572 | N5_1D8 | ATATTT | 383.5 |
| 573 | N5_1E6 | CTTGTT | 365.1 |
| 574 | N5_7H3 | TGGGGG | 360.5 |
| 575 | N5_7F11 | GTCGGG | 339.7 |
| 576 | N5_1D1 | AAGACG | 321.6 |
| 577 | N5_7A6 | TGCATT | 314.5 |
| 578 | N5_1A5 | GGGAGT | 291.5 |
| 579 | N5_1F6 | TTATTG | 247.6 |
| 580 | N5_1A3 | ACGGGA | 228.1 |
| 581 | N5_1F3 | ATAATG | 212.8 |
| 582 | N5_7H1 | GGGTCG | 158.5 |
| 583 | N5_7D7 | TGCGAC | 105.5 |
| 584 | N5_7C1 | AGGGGG | 87.5 |
| 585 | N5_1G4 | CGCCCA | 86.0 |
| 586 | N5_1H1 | AAGGAT | 84.1 |
| 587 | N5_1D11 | GGACGC | 4.8 |
| 588 | N5_1D12 | CGGTAA | 3.6 |

TABLE 5
Aptamer sequences identified from library
N2 in which nucleotides at positions denoted
by N were randomized as provided in the
sequence CTGGGGAGTCCTTCATGCGGGGCTGAGAGGATGG
AAGCAATCGACCATCGACCCNNNNNNCCTGATCCGGATCATG
CCGGCGCAGGGAG (SEQ ID NO: 589). The
sequences provided in Table 5, below, for
J2-3, P3-J3-3a, and J3a-2 are in the context
provided in SEQ ID NO: 589.

| SEQ ID | Aptamer | Sequence | | | |
|---|---|---|---|---|---|
| NO: | Name | J2-3 | P3-J3-3a | J3a-2 | Fold |
| 589 | | NN | NGAGAN | NN | |
| 1 | 12C6-1 | GA | TGAGAG | GA | |
| 590 | N2_2A1 | AG | CGAGAA | CT | 1.0 |
| 591 | N2_2B1 | GA | TGAGAA | CT | 1.0 |
| 592 | N2_2C1 | TA | TGAGAA | AG | 1.1 |
| 593 | N2_2D1 | TT | AGAGAG | GT | 1.1 |
| 594 | N2_2F1 | AG | CGAGAT | CA | 1.1 |
| 595 | N2_2G1 | GA | AGAGAT | TA | 1.7 |
| 596 | N2_2H1 | GT | GGAGAC | CC | 1.1 |
| 597 | N2_2A2 | TC | GGAGAT | TA | 0.7 |
| 598 | N2_2C2 | GG | GAGATG | GC | 0.8 |
| 599 | N2_2D2 | TG | GAGAGG | GC | 1.1 |
| 600 | N2_2E2 | GA | TGAGAA | GG | 1.1 |
| 601 | N2_2F2 | GA | GGAGAA | GG | 1.0 |
| 602 | N2_2G2 | AA | TGAGAA | AC | 1.3 |
| 603 | N2_2H2 | TA | TGAGAA | GG | 0.8 |
| 604 | N2_2A3 | CG | CGAGAT | TA | 0.8 |
| 605 | N2_2B3 | CG | AGAGAA | TT | 1.2 |
| 606 | N2_2C3 | TG | TGAGAC | TT | 1.1 |
| 607 | N2_2D3 | AT | TGAGAA | CA | 0.9 |
| 608 | N2_2E3 | GA | CGAGAA | TA | 1.3 |
| 609 | N2_2F3 | AC | AGAGAA | AG | 0.9 |
| 610 | N2_2G3 | AC | TGAGAA | TG | 1.1 |
| 611 | N2_2H3 | AG | GGAGAC | AG | 1.0 |
| 612 | N2_2B4 | TA | GGAGAG | GA | 0.8 |
| 613 | N2_2C4 | GT | TGAGAC | TA | 2.0 |
| 614 | N2_2D4 | AG | AGAGAG | GG | 0.8 |
| 615 | N2_2E4 | TC | TGAGAC | GA | 0.7 |
| 616 | N2_2F4 | GG | GAGAGG | GC | 1.2 |
| 617 | N2_2G4 | CT | GGAGAG | TG | 1.1 |
| 618 | N2_2H4 | TA | CGAGAT | GC | 1.0 |
| 619 | N2_2A5 | TC | CGAGAA | TG | 1.0 |
| 620 | N2_2B5 | TT | GGAGAG | AC | 1.0 |
| 621 | N2_2C5 | TC | AGAGAA | CT | 1.0 |
| 622 | N2_2D5 | TG | AGAGAT | GG | 0.6 |
| 623 | N2_2E5 | AG | TGAGAC | AG | 1.2 |
| 624 | N2_2G5 | TG | TGAGAT | GA | 1.0 |
| 625 | N2_2A6 | GA | GGAGAT | AC | 1.2 |
| 626 | N2_2B6 | AT | TGAGAC | TG | 0.8 |
| 627 | N2_2C6 | GA | AGAGAT | GT | 0.9 |
| 628 | N2_2D6 | TC | AGAGAA | GG | 1.1 |
| 629 | N2_2E6 | TG | GGAGAA | TG | 0.9 |
| 630 | N2_2F6 | TT | GGAGAA | GT | 1.0 |
| 631 | N2_2G6 | TT | TGAGAA | AT | 1.1 |
| 632 | N2_2H6 | CC | CGAGAG | GA | 1.0 |
| 633 | N2_2A7 | AT | AGAGAT | TT | 0.9 |
| 634 | N2_2B7 | GT | TGAGAC | GG | 1.2 |
| 635 | N2_2C7 | AT | TGAGAG | GG | 0.8 |
| 636 | N2_2D7 | CG | AGAGAT | AG | 1.0 |
| 637 | N2_2E7 | GC | CGAGAG | TC | 1.1 |
| 638 | N2_2F7 | TA | TGAGAT | AG | 1.0 |
| 639 | N2_2G7 | TC | TGAGAT | AT | 1.0 |
| 640 | N2_2H7 | AA | GGAGAA | CT | 0.8 |
| 641 | N2_2A8 | TG | GGAGAT | GA | 1.0 |
| 642 | N2_2B8 | TG | GGAGAG | TT | 1.1 |
| 643 | N2_2C8 | GC | AGAGAG | CA | 0.9 |
| 644 | N2_2D8 | GT | CGGAGA | CT | 1.1 |
| 645 | N2_2E8 | GT | CGAGAG | CC | 0.9 |
| 646 | N2_2F8 | AC | AGAGAA | CT | 1.0 |
| 647 | N2_2G8 | AT | GGAGAT | TA | 1.1 |
| 648 | N2_2H8 | GC | AGAGAG | TT | 1.4 |
| 649 | N2_2A9 | TA | TGAGAG | GC | 1.1 |
| 650 | N2_2B9 | AG | GGAGAT | GG | 1.1 |
| 651 | N2_2C9 | GA | CAGAGA | CA | 1.2 |
| 652 | N2_2D9 | GG | CAGAGA | CG | 1.0 |
| 653 | N2_2G9 | TT | GGAGAT | TA | 1.3 |
| 654 | N2_2H9 | GT | TGAGAG | AT | 0.9 |
| 655 | N2_2A10 | TT | AGAGAT | AA | 1.1 |
| 656 | N2_2B10 | AC | AGAGAA | GC | 1.1 |
| 657 | N2_2C10 | AA | AGAGAC | TG | 1.2 |
| 658 | N2_2D10 | GT | AGAGAA | TA | 1.2 |
| 659 | N2_2E10 | GA | AGAGAT | TC | 1.2 |
| 660 | N2_2F10 | TA | GGAGAG | GG | 1.1 |
| 661 | N2_2G10 | TT | CGAGAC | GG | 1.0 |
| 662 | N2_2H10 | TT | GGAGAG | TT | 1.0 |
| 663 | N2_2A11 | AA | CGAGAG | AT | 1.1 |
| 664 | N2_2B11 | AT | AGAGAG | TC | 1.2 |
| 665 | N2_2D11 | GG | AGAGAA | GA | 1.3 |
| 666 | N2_2F11 | GT | CGAGAG | AT | 1.1 |
| 667 | N2_2G11 | GT | AGAGAT | GT | 0.9 |
| 668 | N2_2H11 | TT | GGAGAG | GT | 1.2 |
| 669 | N2_2A12 | TT | GGAGAA | GA | 1.1 |
| 670 | N2_2B12 | GG | CGAGAT | AG | 1.0 |
| 671 | N2_2C12 | TC | AGAGAC | CT | 1.1 |
| 672 | N2_2D12 | TC | CGAGAT | AG | 1.1 |
| 673 | N2_2E12 | AA | TGAGAA | GG | 1.3 |
| 674 | N2_2F12 | TA | TGAGAG | CC | 1.0 |

TABLE 6
Additional sequences.

| SEQ ID | | |
|---|---|---|
| NO: | Description | Sequence |
| 1 | 12C6-1 aptamer | CTGGGGAGTCCTTCATGCGGGGCTGAGAGG |
| | | ATGGAAGCAATCGACCATCGACCCATTGCA |
| | | CCTGATCCGGATCATGCCGGCGCAGGGAG |
| 2 | 12C6-1 aptamer with | CTGGGGAGTCCTTCATGCGGGGCTGAGAGG |
| | randomized nucleotides | ATGGAAGX₁X₂AX₃X₄X₅X₆CCATCGACCCX₇X₈ |
| | (combined N1, N3-N5 | X₉X₁₀X₁₁X₁₂CCTX₁₃X₁₄X₁₅CCGGX₁₆X₁₇X₁₈X₁₉ |
| | libraries) | X₂₀X₂₁CCGGX₂₂X₂₃CAGGGAG |
| 3 | 12C6-1 aptamer with | CTGGGGAGTCCTTCATGCGGGGCTGAGAGG |
| | randomized nucleotides | ATGGAAGX₁X₂AX₃X₄X₅X₆CCATCGACCCATT |
| | (N3_library) | GCACCTGATCCGGATCATGCCGGCGCAGGG |
| | | AG |
| 4 | 12C6-1 aptamer with | CTGGGGAGTCCTTCATGCGGGGCTGAGAGG |
| | randomized nucleotides | ATGGAAGCAATCGACCATCGACCCX₇X₈X₉ |
| | (N1_library) | X₁₀X₁₁X₁₂CCTGATCCGGATCATGCCGGCGC |
| | | AGGGAG |
| 5 | 12C6-1 aptamer with | CTGGGGAGTCCTTCATGCGGGGCTGAGAGG |
| | randomized nucleotides | ATGGAAGCAATCGACCATCGACCCATTGCA |
| | (N4_library) | CCTX₁₃X₁₄X₁₅CCGGATCATGCCGGX₂₂X₂₃CA |
| | | GGGAG |
| 6 | 12C6-1 aptamer with | CTGGGGAGTCCTTCATGCGGGGCTGAGAGG |
| | randomized nucleotides | ATGGAAGCAATCGACCATCGACCCATTGCA |
| | (N5_library) | CCTGATCCGGX₁₆X₁₇X₁₈X₁₉X₂₀X₂₁CCGGCG |
| | | CAGGGAG |
| 675 | 12C6-1 alternate | gtgagtctatgggacccttgatgttttctt |
| | splicing gene | tccccttcttttctatggttaagttcatgt |
| | regulation cassette | cataggaaggggagaagtaacagggtacac |
| | Caps: 12C6 aptamer; | atattgaccaaatcagggtaattttgcatt |
| | Grey: alt exon; | tgtaattttaaaaaatgctttcttctttta |
| | Underline: riboswitch | atatacttttttgtttatcttatttctaat |
| | stem forming sequence; | actttccctaatctctttctttcagggcaa |
| | Ital.: 5′ intron and | taatgatacaatgtatcatgccgagtaacg |
| | 3′ intron | ctgtttctctaacttgtaggaatgaattca |
| | | gatatttccagagaatgaaaaaaaaatctt |
| | | cagtagaaggtaatgtCTGGGGAGTCCTTC |
| | | ATGCGGGGCTGAGAGGATGGAAGCAATCGA |
| | | CCATCGACCCATTGCACCTGATCCGGATCA |
| | | TGCCGGCGCAGGGAGacattacgcaccatt |
| | | ctaaagaataacagtgataatttctgggtt |
| | | aaggcaatagcaatatttctgcatataaat |
| | | atttctgcatataaattgtaactgatgtaa |
| | | gaggtttcatattgctaatagcagctacaa |
| | | tccagctaccattctgcttttattttatgg |
| | | ttgggataaggctggattattctgagtcca |
| | | agctaggcccttttgctaatcatgttcata |
| | | cctcttatcttcctcccacag |
| 676 | Alternative splicing | GTGAGTCTATGGGACCCTTGATGTTTTCTT |
| | gene regulation | TCCCCTTCTTTTCTATGGTTAAGTTCATGT |
| | cassette | CATAGGAAGGGGAGAAGTAACAGGGTACAC |
| | -X- represents an | ATATTGACCAAATCAGGGTAATTTTGCATT |
| | aptamer encoding | TGTAATTTTAAAAAATGCTTTCTTCTTTTA |
| | sequence disclosed | ATATACTTTTTTGTTTATCTTATTTCTAAT |
| | herein; | ACTTTCCCTAATCTCTTTCTTTCAGGGCAA |
| | alternative exon is | TAATGATACAATGTATCATGCCGAGTAACG |
| | underlined | CTGTTTCTCTAACTTGTAGGAATGAATTCA |
| | | GATATTTCCAGAGAATGAAAAAAAAATCTT |
| | | CAGTAGAAGgtaatgt-X-acattacGCAC |
| | | CATTCTAAAGAATAACAGTGATAATTTCTG |
| | | GGTTAAGGCAATAGCAATATTTCTGCATAT |
| | | AAATATTTCTGCATATAAATTGTAACTGAT |
| | | GTAAGAGGTTTCATATTGCTAATAGCAGCT |
| | | ACAATCCAGCTACCATTCTGCTTTTATTTT |
| | | ATGGTTGGGATAAGGCTGGATTATTCTGAG |
| | | TCCAAGCTAGGCCCTTTTGCTAATCATGTT |
| | | CATACCTCTTATCTTCCTCCCACAG. |
| 677 | Modified DHFR exon 2 | GAATGAATTCAGATATTTCCAGAGAATGAA |
| | | AAAAAAATCTTCAGTAGAAG |
| 678 | Modified DHFR exon 2 | GAATGAATTCAGATATTTCCAGAGAATGAA |
| | | AAAAAATCTTCAGTAGAAG |
| 679 | thiC | GUAAUGUGUCGGAGUGCCUUAGGGAUUAU |
| | | UCCCCUAAAGCUGAGACCGCAUUGCGGGA |
| | | UCCGUUGAACCUGAUCAGGCUAAUACCUG |
| | | CGAAGGGAACACAUUAC |
| 680 | thiM | GUAAUGUCUCGGGGUGCCCUUCUGCGUGA |
| | | AGGCUGAGAAAUACCCGUAUCACCUGAUC |
| | | UGGAUAAUGCCAGCGUAGGGAAGACAUUA |
| | | C |

We claim:

1. A polynucleotide cassette for regulating the expression of a target gene, wherein the polynucleotide cassette comprises a sequence encoding an aptamer that binds to a small molecule, wherein the aptamer encoding sequence comprises:
CTGGGGAGTCCTTCATGCGGGGCTGAGAGGATGGAAGX₁X₂AX₃X₄X₅X₆CCAT CGACCCX₇X₈X₉X₁₀X₁₁X₁₂CCTX₁₃X₁₄X₁₅CCGGX₁₆X₁₇X₁₈X₁₉X₂₀X₂₁CCGGX₂₂X₂₃C AGGGAG (SEQ ID NO:2); wherein:
X₁ is C or T;
X₂ is any nucleotide;
X₃ is any nucleotide;
X₄ is G or T;
X₅ is A, G, or T;
X₆ is A or G;
X₇ is A or T;
X₈ is A, C, or T;
X₉ is A, C, or T;
X₁₀ is any nucleotide;
X₁₁ is any nucleotide or no nucleotide;
X₁₂ is A;
X₁₃ is A, C, or G;
X₁₄ is any nucleotide;
X₁₅ is C, G, or T;
X₁₆ is G or T;
X₁₇ is A or T;
X₁₈ is any nucleotide;
X₁₉ is A or G;
X₂₀ is A, G, T;
X₂₁ is C, G, T;
X₂₂ is T; and
X₂₃ is A, G, or T.

2. A polynucleotide cassette for regulating the expression of a target gene, wherein the polynucleotide cassette comprises a sequence encoding an aptamer that binds to a small molecule, wherein the aptamer encoding sequence comprises:
CTGGGGAGTCCTTCATGCGGGGCTGAGAGGATGGAAGCAATCGACCATCGA CCCX₇X₈X₉X₁₀X₁₁X₁₂CCTGATCCGGATCATGCCGGCGCAGGGAG (SEQ ID NO:4); wherein:
X₇ is A, G, or T;
X₈ is any nucleotide;
X₉ is any nucleotide;
X₁₀ is any nucleotide;
X₁₁ is any nucleotide or no nucleotide;
X₁₂ is A, C, or T.

3. The polynucleotide cassette of claim 2, wherein the polynucleotide cassette comprises a sequence encoding an aptamer that binds to a small molecule, wherein the aptamer encoding sequence comprises:
CTGGGGAGTCCTTCATGCGGGGCTGAGAGGATGGAAGCAATCGACCATCGA CCCX₇X₈X₉X₁₀X₁₁X₁₂CCTGATCCGGATCATGCCGGCGCAGGGAG (SEQ ID NO:4); wherein:
X₇ is A or T;
X₈ is A, C, or T;
X₉ is A, C, or T;
X₁₀ is any nucleotide;
X₁₁ is any nucleotide or no nucleotide; and
X₁₂ is A.

4. The polynucleotide cassette of claim 2, wherein the aptamer encoding sequence comprises
CTGGGGAGTCCTTCATGCGGGGCTGAGAGGATGGAAGCAATCGACCATCGA CCCX₇X₈X₉X₁₀X₁₁X₁₂CCTGATCCGGATCATGCCGGCGCAGGGAG (SEQ ID NO:4); wherein:
X₇ is A;
X₈ is A, C, or T;
X₉ is A, C, or T;
X₁₀ is any nucleotide;
X₁₁ is any nucleotide or no nucleotide; and
X₁₂ is A.

5. A polynucleotide cassette for regulating the expression of a target gene, wherein the polynucleotide cassette comprises a sequence encoding an aptamer that binds to a small molecule, wherein the aptamer encoding sequence comprises:
(SEQ ID NO: 3)
CTGGGGAGTCCTTCATGCGGGGCTGAGAGGATGGAAGX₁X₂AX₃X₄X₅
X₆CCATCGACCCATTGCACCTGATCCGGATCATGCCGGCGCAGGGAG;
wherein:
X₁ is C G, or T;
X₂ is any nucleotide;
X₃ is any nucleotide;
X₄ is any nucleotide;
X₅ is any nucleotide; and
X₆ is any nucleotide.

6. The polynucleotide cassette of claim 5, wherein the aptamer encoding sequence comprises
(SEQ ID NO: 3)
CTGGGGAGTCCTTCATGCGGGGCTGAGAGGATGGAAGX₁X₂AX₃X₄X₅
X₆CCATCGACCCATTGCACCTGATCCGGATCATGCCGGCGCAGGGAG;
wherein:
X₁ is C or T;
X₂ is any nucleotide;
X₃ is any nucleotide;
X₄ is any nucleotide;
X₅ is A, G, or T; and
X₆ is any nucleotide.

7. The polynucleotide cassette of claim 5, wherein the aptamer encoding sequence comprises:
(SEQ ID NO: 3)
CTGGGGAGTCCTTCATGCGGGGCTGAGAGGATGGAAGX₁X₂AX₃X₄X₅
X₆CCATCGACCCATTGCACCTGATCCGGATCATGCCGGCGCAGGGAG;
wherein:
X₁ is C or T;
X₂ is any nucleotide;
X₃ is any nucleotide;
X₄ is G or T;
X₅ is A, G, or T; and
X₆ is A or G.

8. A polynucleotide cassette for regulating the expression of a target gene, wherein the polynucleotide cassette comprises a sequence encoding an aptamer that binds to a small molecule, wherein the aptamer encoding sequence comprises:
CTGGGGAGTCCTTCATGCGGGGCTGAGAGGATGGAAGCAATCGACCATCGA CCCATTGCACCTX₁₃X₁₄X₁₅CCGGATCATGCCGGX₂₂X₂₃CAGGGAG (SEQ ID NO:5); wherein:
X₁₃, X₁₄, X₁₅, X₂₂, and X₂₃ is any nucleotide.

9. The polynucleotide cassette of claim 8, wherein the aptamer encoding sequence comprises:
CTGGGGAGTCCTTCATGCGGGGCTGAGAGGATGGAAGCAATCGACCATCGA CCCATTGCACCTX₁₃X₁₄X₁₅CCGGATCATGCCGGX₂₂X₂₃CAGGGAG (SEQ ID NO:5); wherein:
X₁₃ is A, C, or G;
X₁₄ is any nucleotide;
X₁₅ is C, G, or T;
X₂₂ is T; and
X₂₃ is A, G, or T.

10. A polynucleotide cassette for regulating the expression of a target gene, wherein the polynucleotide cassette comprises a sequence encoding an aptamer that binds to a small molecule, wherein the aptamer encoding sequence comprises:
CTGGGGAGTCCTTCATGCGGGGCTGAGAGGATGGAAGCAATCGACCATCGA CCCATTGCACCTGATCCGGX₁₆X₁₇X₁₈X₁₉X₂₀X₂₁CCGGCGCAGGGAG (SEQ ID NO:6); wherein:
X₁₆ is any nucleotide;
X₁₇ is any nucleotide;
X₁₈ is any nucleotide;
X₁₉ is any nucleotide;
X₂₀ is any nucleotide; and
X₂₁ is C, G, T.

11. The polynucleotide cassette of claim 10, wherein the aptamer encoding sequence comprises:
CTGGGGAGTCCTTCATGCGGGGCTGAGAGGATGGAAGCAATCGACCATCGA CCCATTGCACCTGATCCGGX₁₆X₁₇X₁₈X₁₉X₂₀X₂₁CCGGCGCAGGGAG (SEQ ID NO:6); wherein:
X₁₆ is G or T;
X₁₇ is A or T;
X₁₈ is any nucleotide;
X₁₉ is A or G;
X₂₀ is A, G, T; and
X₂₁ is C, G, T.

12. A polynucleotide cassette for regulating the expression of a target gene, wherein the polynucleotide cassette comprises a sequence encoding an aptamer that binds to a small molecule, wherein the aptamer encoding sequence comprises a sequence that is at least 95% identical, or at least 99% identical to a sequence selected from the group consisting of SEQ ID NOs: 1 and 7-558.

13. A polynucleotide cassette for regulating the expression of a target gene, wherein the polynucleotide cassette comprises a sequence encoding an aptamer that binds to a small molecule, wherein the aptamer encoding sequence is selected from the group consisting of SEQ ID NOs: 1 and 7-558.

14. A polynucleotide cassette for regulating the expression of a target gene, wherein the polynucleotide cassette comprises a sequence encoding an aptamer that binds to a small molecule, wherein the aptamer encoding sequence comprises a sequence that is at least 95% identical, or at least 99% identical to a sequence selected from the group consisting of SEQ ID NOs: 7-17, 89-96, 174-349, and 358-583.

15. A polynucleotide cassette for regulating the expression of a target gene, wherein the polynucleotide cassette comprises a sequence encoding an aptamer that binds to a small molecule, wherein the aptamer encoding is sequence selected from the group consisting of SEQ ID NOs: 7-17, 89-96, 174-349, and 358-583.

16. A polynucleotide cassette for regulating the expression of a target gene, wherein the polynucleotide cassette comprises a sequence encoding an aptamer that binds to a small molecule, wherein the aptamer encoding sequence comprises a sequence that is at least 95% identical, or at least 99% identical to a sequence selected from the group consisting of SEQ ID NOs: 7-11, 89-94, 174-349, and 358-447.

17. A polynucleotide cassette for regulating the expression of a target gene, wherein the polynucleotide cassette comprises a sequence encoding an aptamer that binds to a small molecule, wherein the aptamer encoding sequence is selected from the group consisting of SEQ ID NOs: 7-11, 89-94, 174-349, and 358-447.

18. A polynucleotide cassette for regulating the expression of a target gene, wherein the polynucleotide cassette comprises a sequence encoding an aptamer that binds to a small molecule, wherein the aptamer encoding sequence comprises a sequence that is at least 95% identical, or at least 99% identical to a sequence selected from the group consisting of SEQ ID NOs: 174, 358, 363, and 378.

19. A polynucleotide cassette for regulating the expression of a target gene, wherein the polynucleotide cassette comprises a sequence encoding an aptamer that binds to a small molecule, wherein the aptamer encoding sequence is selected from the group consisting of SEQ ID NOs: 174, 358, 363, and 378.

20. A nucleic acid sequence encoding an aptamer, wherein the aptamer encoding sequence comprises:
CTGGGGAGTCCTTCATGCGGGGCTGAGAGGATGGAAGX₁X₂AX₃X₄X₅X₆CCAT CGACCCX₇X₈X₉X₁₀X₁₁X₂CCTX₁₃X₁₄X₁₅CCGGX₁₆X₁₇X₁₈X₁₉X₂₀X₂₁CCGGX₂₂X₂₃C AGGGAG (SEQ ID NO:2); wherein:
X₁ is C or T;
X₂ is any nucleotide;
X₃ is any nucleotide;
X₄ is G or T;
X₅ is A, G, or T;
X₆ is A or G;
X₇ is A;
X₈ is A, C, or T;
X₉ is A, C, or T;
X₁₀ is any nucleotide;
X₁₁ is any nucleotide or no nucleotide;
X₁₂ is A;
X₁₃ is A, C, or G;
X₁₄ is any nucleotide;
X₁₅ is C, G, or T;
X₁₆ is G or T;
X₁₇ is A or T;
X₁₈ is any nucleotide;
X₁₉ is A or G;
X₂₀ is A, G, T;
X₂₁ is C, G, T;
X₂₂ is T; and
X₂₃ is A, G, or T.

21. A nucleic acid sequence encoding an aptamer, wherein the aptamer encoding sequence comprises:
CTGGGGAGTCCTTCATGCGGGGCTGAGAGGATGGAAGCAATCGACCATCGA CCCX₇X₈X₉X₁₀X₁₁X₁₂CCTGATCCGGATCATGCCGGCGCAGGGAG (SEQ ID NO:4); wherein:
X₇ is A, G, or T;
X₈ is any nucleotide;
X₉ is any nucleotide;
X₁₀ is any nucleotide;
X₁₁ is any nucleotide or no nucleotide;
X₁₂ is A, C, or T,
wherein X₇—X₂ are not simultaneously A, T, T, G, C, and A, respectively.

22. The nucleic acid sequence of claim 21, wherein the aptamer encoding sequence comprises:
CTGGGGAGTCCTTCATGCGGGGCTGAGAGGATGGAAGCAATCGACCATCGA CCCX₇X₈X₉X₁₀X₁₁X₁₂CCTGATCCGGATCATGCCGGCGCAGGGAG (SEQ ID NO:4); wherein:
X₇ is A or T;
X₈ is A, C, or T;
X₉ is A, C, or T;
X₁₀ is any nucleotide;
X₁₁ is any nucleotide or no nucleotide; and
X₁₂ is A;
wherein X₇—X₂ are not simultaneously A, T, T, G, C, and A, respectively.

23. The nucleic acid sequence of claim 21, wherein the aptamer encoding sequence comprises:
CTGGGGAGTCCTTCATGCGGGGCTGAGAGGATGGAAGCAATCGACCATCGA CCCX₇X₈X₉X₁₀X₁₁X₁₂CCTGATCCGGATCATGCCGGCGCAGGGAG (SEQ ID NO:4); wherein:
X₇ is A;
X₈ is A, C, or T;
X₉ is A, C, or T;
X₁₀ is any nucleotide;
X₁₁ is any nucleotide or no nucleotide; and
X₁ is A;
wherein X₇—X₂ are not simultaneously A, T, T, G, C, and A, respectively.

24. A nucleic acid sequence encoding an aptamer that binds to a small molecule, wherein the aptamer encoding sequence comprises:
(SEQ ID NO: 3)
CTGGGGAGTCCTTCATGCGGGGCTGAGAGGATGGAAGX₁X₂AX₃X₄X₅
X₆CCATCGACCCATTGCACCTGATCCGGATCATGCCGGCGCAGGGAG;
wherein:
X₁ is C G, or T;
X₂ is any nucleotide;
X₃ is any nucleotide;
X₄ is any nucleotide;
X₅ is any nucleotide; and
X₆ is any nucleotide,
wherein X₁—X₆ are not simultaneously C, A, T, C, G, and A, respectively.

25. The nucleic acid sequence of claim 15, wherein the aptamer encoding sequence comprises:
(SEQ ID NO: 3)
CTGGGGAGTCCTTCATGCGGGGCTGAGAGGATGGAAGX₁X₂AX₃X₄X₅
X₆CCATCGACCCATTGCACCTGATCCGGATCATGCCGGCGCAGGGAG;
wherein:
X₁ is C or T;
X₂ is any nucleotide;
X₃ is any nucleotide;
X₄ is any nucleotide;
X₅ is A, G, or T; and
X₆ is any nucleotide;
wherein X₁—X₆ are not simultaneously C, A, T, C, G, and A, respectively.

26. The nucleic acid sequence of claim 15, wherein the aptamer encoding sequence comprises:
(SEQ ID NO: 3)
CTGGGGAGTCCTTCATGCGGGGCTGAGAGGATGGAAGX₁X₂AX₃X₄X₅
X₆CCATCGACCCATTGCACCTGATCCGGATCATGCCGGCGCAGGGAG;
wherein:
X₁ is C or T;
X₂ is any nucleotide;
X₃ is any nucleotide;
X₄ is G or T;
X₅ is A, G, or T;
X₆ is A or G.

27. A nucleic acid sequence encoding an aptamer that binds to a small molecule, wherein the aptamer encoding sequence comprises:
CTGGGGAGTCCTTCATGCGGGGCTGAGAGGATGGAAGCAATCGACCATCGA CCCATTGCACCTX₁₃X₁₄X₁₅CCGGATCATGCCGGX₂₂X₂₃CAGGGAG (SEQ ID NO:5); wherein:
X₁₃, X₁₄, X₁₅, X₂₂, and X₂₃ is any nucleotide,
wherein X₁₃, X₁₄, X₁₅, X₂₂, and X₂₃ are not simultaneously G, A, T, C, and G, respectively.

28. The nucleic acid sequence of claim 21, wherein the aptamer encoding sequence comprises:
CTGGGGAGTCCTTCATGCGGGGCTGAGAGGATGGAAGCAATCGACCATCGA CCCATTGCACCTX₁₃X₁₄X₁₅CCGGATCATGCCGGX₂₂X₂₃CAGGGAG (SEQ ID NO:5); wherein:
X₁₃ is A, C, or G;
X₁₄ is any nucleotide;
X₁₅ is C, G, or T;
X₂₂ is T; and
X₂₃ is A, G, or T.

29. A nucleic acid sequence encoding an aptamer that binds to a small molecule, wherein the aptamer encoding sequence comprises:
CTGGGGAGTCCTTCATGCGGGGCTGAGAGGATGGAAGCAATCGACCATCGA CCCATTGCACCTGATCCGGX₁₆X₁₇X₁₈X₁₉X₂₀X₂₁CCGGCGCAGGGAG (SEQ ID NO:6); wherein:
X₁₆ is any nucleotide;
X₁₇ is any nucleotide;
X₁₈ is any nucleotide;
X₁₉ is any nucleotide;
X₂₀ is any nucleotide; and
X₂₁ is C, G, T;
wherein X₁₆—X₂₁, are not simultaneously A, T, C, A, T, and G, respectively.

30. The nucleic acid sequence of claim 23, wherein the aptamer encoding sequence comprises:
CTGGGGAGTCCTTCATGCGGGGCTGAGAGGATGGAAGCAATCGACCATCGA CCCATTGCACCTGATCCGGX₁₆X₁₇X₁₈X₁₉X₂₀X₂₁CCGGCGCAGGGAG (SEQ ID NO:6); wherein:
X₁₆ is G or T;
X₁₇ is A or T;
X₁₈ is any nucleotide;
X₁₉ is A or G;
X₂₀ is A, G, T; and
X₂₁ is C, G, T.

31. A nucleic acid sequence encoding an aptamer that binds to a small molecule, wherein the aptamer encoding sequence comprises a sequence that is at least 95% identical, or at least 99% identical to a sequence selected from the group consisting of SEQ ID NOs: 1 and 7-558.

32. A nucleic acid sequence encoding an aptamer that binds to a small molecule, wherein the aptamer encoding sequence comprises a sequence encoding an aptamer that binds to a small molecule, wherein the aptamer encoding sequence is selected from the group consisting of SEQ ID NOs: 1 and 7-558.

33. A nucleic acid sequence encoding an aptamer that binds to a small molecule, wherein the aptamer encoding sequence comprises a sequence that is at least 95% identical, or at least 99% identical to a sequence selected from the group consisting of SEQ ID NOs: 7-17, 89-96, 174-349, and 358-583.

34. A nucleic acid sequence encoding an aptamer that binds to a small molecule, wherein the aptamer encoding sequence comprises a sequence encoding an aptamer that binds to a small molecule, wherein the aptamer encoding is sequence selected from the group consisting of SEQ ID NOs: 7-17, 89-96, 174-349, and 358-583.

35. A nucleic acid sequence encoding an aptamer that binds to a small molecule, wherein the aptamer encoding sequence comprises a sequence that is at least 95% identical, or at least 99% identical to a sequence selected from the group consisting of SEQ ID NOs: 7-11,89-94, 174-349, and 358-447.

36. A nucleic acid sequence encoding an aptamer that binds to a small molecule, wherein the aptamer encoding sequence comprises a sequence encoding an aptamer that binds to a small molecule, wherein the aptamer encoding sequence is selected from the group consisting of SEQ ID NOs: 7-11, 89-94, 174-349, and 358-447.

37. A nucleic acid sequence encoding an aptamer that binds to a small molecule, wherein the aptamer encoding sequence comprises a sequence that is at least 95% identical, or at least 99% identical to a sequence selected from the group consisting of SEQ ID NOs: 174, 358, 363, and 378.

38. A nucleic acid sequence encoding an aptamer that binds to a small molecule, wherein the aptamer encoding sequence comprises a sequence encoding an aptamer that binds to a small molecule, wherein the aptamer encoding sequence is selected from the group consisting of SEQ ID NOs: 174, 358, 363, and 378.

39. A nucleic acid sequence encoding a recombinant riboswitch for the regulation of target gene expression in response to a small molecule, wherein the riboswitch comprises an aptamer encoded by SEQ ID NOs: 1 and 7-558 or a sequence that is at least 95% or at least 99% identical to SEQ ID NOs: 1 and 7-558.

40. A polynucleotide cassette for the regulation of the expression of a target gene in response to a small molecule, the polynucleotide cassette comprising:
(c) a riboswitch; and
(d) an alternatively-spliced exon, flanked by a 5′ intron and a 3′ intron,
wherein the riboswitch comprises (i) an effector region comprising a stem that includes the 5′ splice site sequence of the 3′ intron, and (ii) the aptamer comprises a sequence of SEQ ID NOs: 1 and 7-558 or a sequence that is at least 95% or at least 99% identical to SEQ ID NOs: 1 and 7-558; and
wherein the alternatively-spliced exon comprises a stop codon that is in-frame with the target gene when the alternatively-spliced exon is spliced into the target gene mRNA.

41. The polynucleotide cassette of claim 40, wherein the polynucleotide cassette is located in the protein coding sequence of the target gene.

42. The polynucleotide cassette of claim 40, wherein the polynucleotide cassette is located in an untranslated region of the target gene or in an intron of the target gene.

43. The polynucleotide cassette of any one of claims 1-19 and 40-42 or the nucleic acid sequence of any one of claims 20-39, wherein the aptamer binds to, or otherwise responds to the presence of, a small molecule having the structure according to Formula I: 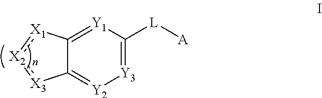
wherein
X₁, X₂, and X₃ are, in each instance, independently selected from CR₁, CHR₁, N, NH, O and S, wherein adjacent X₁, X₂, and X₃ are not simultaneously selected to be O or S;
the dashed lines represent optional double bonds;
Y₁, Y₂, and Y₃ are, in each instance, independently selected from CR₂ and N;
n is 1 or 2, wherein when n is 1, only one of the dashed lines is a double bond;
L-A is 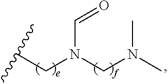
or
L is selected from 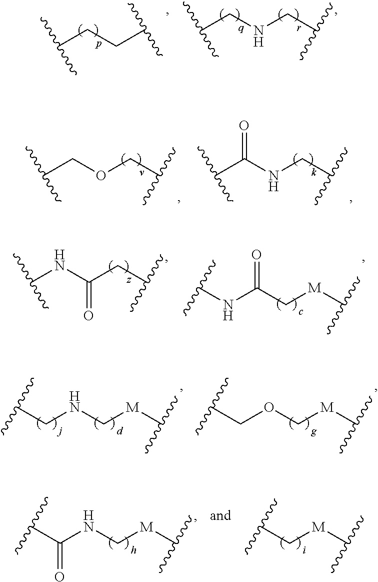
wherein k, p, q, r, and v are independently selected from integers 0, 1, 2, 3, 4, 5, 6, 7, 8, 9, and 10, z is selected from integers 1, 2, 3, 4, and 5;
c, d, e, f, g, h and i are independently selected from integers 1, 2, 3, 4, 5, 6, 7, 8, 9, and 10; j is selected from integers 0, 1, 2, 3, 4, 5, 6, 7, 8, 9, and 10;
M is selected from —NH—, —O—, —NHC(═O)—, —C(═O)NH—, —S—, and —C(═O)—; and
A is selected from 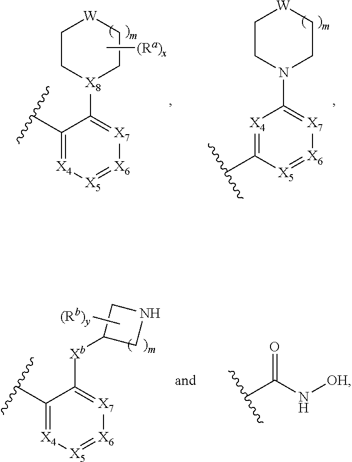
wherein X₄, X₅, X₆, and X₇, are independently selected from CR₃ and N;
X₈ is N or CH;
X^{b} is selected from O, NH, and NCH₃;
wherein each of R₁, R₂, and R₃ are independently selected from —H, —Cl, —Br, —I, —F, —CF₃, —CH₂F, —CHF₂, —OH, —CN, —NO₂, —NH₂, —NH(C₁-C₆ alkyl), —N(C₁-C₆ alkyl)₂, —COOH, —COO(C₁-C₆ alkyl), —CO(C₁-C₆ alkyl), —O(C₁-C₆ alkyl), —OCO(C₁-C₆ alkyl), —NCO(C₁-C₆ alkyl), —CONH(C₁-C₆ alkyl), and substituted or unsubstituted C₁-C₆ alkyl;
additionally or alternatively, two R₃ on adjacent ring positions may be taken together to form a 5- or 6-membered aromatic ring having from 0 to 2 heteroatoms selected from O, S, N and NH—;
m is 1 or 2;
each R^{a} is independently selected from C₁ to C₃ alkyl, —OCH₃, —CF₃, —CH₂F, —CHF₂, halo, hydroxyl and amino; or additionally or alternatively, two R^{a} attached to the same carbon atom form an oxo group, or two R^{a} attached to different carbon atoms form a 4- to 6-membered carbocyclic ring or a 4- to 6-membered heterocyclic ring having 1 or 2 heteroatoms selected from O and NH;
x is 0, 1, 2 or 3;
each R^{b} is independently selected from C₁ to C₃ alkyl, —OCH₃, —CF₃, —CH₂F, —CHF₂, halo, hydroxyl and amino; or additionally or alternatively, two R^{b} attached to the same carbon atom form an oxo group; or two R^{b} attached to different carbon atoms form a 4- to 6-membered carbocyclic ring or a 4- to 6-membered heterocyclic ring having 1 or 2 heteroatoms selected from O and NH;
y is 0, 1, 2 or 3; and
W is O or NR₄, wherein R₄ is selected from selected from —H, —CO(C₁-C₆ alkyl), substituted or unsubstituted C₁-C₆ alkyl, substituted or unsubstituted aryl, substituted or unsubstituted heteroaryl, substituted or unsubstituted cycloalkyl, —CO(aryl), —CO(heteroaryl), and —CO(cycloalkyl);
provided that at least two of X₁, X₂, X₃, X₄, X₅, X₆, and X₇ are N;
or a pharmaceutically acceptable salt thereof.

44. The polynucleotide cassette of claim 43, wherein the small molecule has a structure according to formula XIII 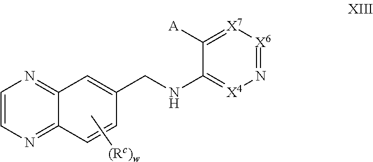
or a pharmaceutically acceptable salt thereof, wherein
X⁴ is selected from CH, CR^{d} and N;
X⁶ is selected from CH, CR^{d} and N;
X⁷ is selected from CH, CR^{d} and N;
wherein 0 or 1 of X⁴, X⁶ or X⁷ is N;
A is selected from the group consisting of: 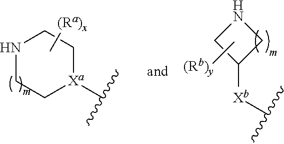
X^{a} is selected from N and CH;
X^{b} is selected from O, NH, and NCH₃;
each R^{a} is independently selected from C₁ to C₃ alkyl, —OCH₃, —CF₃, —CH₂F, —CHF₂, halo, hydroxyl and amino; or additionally or alternatively, two R^{a} attached to the same carbon atom form an oxo group; or two R^{a} attached to different carbon atoms form a 4- to 6-membered carbocyclic ring or a 4- to 6-membered heterocyclic ring having 1 or 2 heteroatoms selected from O and NH;
each R^{b} is independently selected from C₁ to C₃ alkyl, —OCH₃, —CF₃, —CH₂F, —CHF₂, halo, hydroxyl and amino; or additionally or alternatively, two R^{b} attached to the same carbon atom form an oxo group; or two R^{b} attached to different carbon atoms form a 4- to 6-membered carbocyclic ring or a 4- to 6-membered heterocyclic ring having 1 or 2 heteroatoms selected from O and NH;
m is 1 or 2;
x is 0, 1, 2 or 3;
y is 0, 1, 2 or 3;
each R^{c} is independently selected from halo, C₁ to C₃ alkyl, —OCH₃, —CF₃, —CH₂F, —CHF₂, —CN, hydroxyl and amino;
each R^{d} is independently selected from halo, C₁ to C₃ alkyl, —OCH₃, —CF₃, —CH₂F, —CHF₂, —CN, hydroxyl and amino;
alternatively, two R^{d} on adjacent ring positions may be taken together to form a 5- or 6-membered aromatic ring having from 0 to 2 heteroatoms selected from O, S, N and NH; and
w is 0, 1 or 2.

45. The polynucleotide cassette of claim 44, wherein the small molecule has a structure according to formula XIV 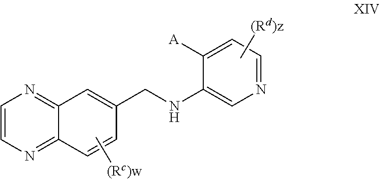
or a pharmaceutically acceptable salt thereof, wherein
or a pharmaceutically acceptable salt thereof, wherein
A is selected from the group consisting of: 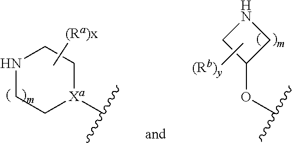
X^{a} is selected from N and CH;
each R^{a} is independently selected from C₁ to C₃ alkyl, —OCH₃, —CF₃, —CH₂F, —CHF₂, halo, hydroxyl and amino; or additionally or alternatively, two R^{a} attached to the same carbon atom form an oxo group; or two R^{a} attached to different carbon atoms form a 4- to 6-membered carbocyclic ring or a 4- to 6-membered heterocyclic ring having 1 or 2 heteroatoms selected from O and NH;
each R^{b} is independently selected from C₁ to C₃ alkyl, —OCH₃, —CF₃, —CH₂F, —CHF₂, halo, hydroxyl and amino; or additionally or alternatively, two R^{b} attached to the same carbon atom form an oxo group; or two R^{b} attached to different carbon atoms form a 4- to 6-membered carbocyclic ring or a 4- to 6-membered heterocyclic ring having 1 or 2 heteroatoms selected from O and NH;
m is 1 or 2;
x is 0, 1, 2 or 3;
y is 0, 1, 2 or 3;
each R^{c} is independently selected from halo, C₁ to C₃ alkyl, —OCH₃, —CF₃, —CH₂F, —CHF₂, —CN, hydroxyl and amino;
each R^{d} is independently selected from halo, C₁ to C₃ alkyl, —OCH₃, —CF₃, —CH₂F, —CHF₂, —CN, hydroxyl and amino; alternatively, two R^{d} on adjacent ring positions may be taken together to form a 5- or 6-membered aromatic ring having from 0 to 2 heteroatoms selected from O, S, N and NH;
w is 0, 1 or 2; and
z is 0, 1 or 2.

46. The polynucleotide cassette of claim 44, wherein the small molecule has a structure according to formula XVI 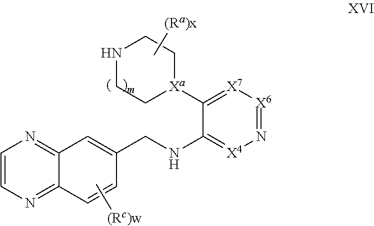
or a pharmaceutically acceptable salt thereof, wherein
X⁴ is selected from CH, CR^{d} and N;
X⁶ is selected from CH, CR^{d} and N;
X⁷ is selected from CH, CR^{d} and N;
wherein 0 or 1 of X⁴, X⁶ or X⁷ is N;
X^{a} is selected from N and CH;
each R^{a} is independently selected from C₁ to C₃ alkyl, —OCH₃, —CF₃, —CH₂F, —CHF₂, halo, hydroxyl and amino; or additionally or alternatively, two R^{a} attached to the same carbon atom form an oxo group; or two R^{a} attached to different carbon atoms form a 4- to 6-membered carbocyclic ring or a 4- to 6-membered heterocyclic ring having 1 or 2 heteroatoms selected from O and NH;
m is 1 or 2;
x is 0, 1, 2 or 3;
each R^{c} is independently selected from halo, C₁ to C₃ alkyl, —OCH₃, —CF₃, —CH₂F, —CHF₂, —CN, hydroxyl and amino;
each R^{d} is independently selected from halo, C₁ to C₃ alkyl, —OCH₃, —CF₃, —CH₂F, —CHF₂, —CN, hydroxyl and amino;
alternatively, two R^{d} on adjacent ring positions may be taken together to form a 5- or 6-membered aromatic ring having from 0 to 2 heteroatoms selected from O, S, N and NH; and
w is 0, 1 or 2.

47. The polynucleotide cassette of claim 44, wherein the small molecule has a structure according to formula XVII 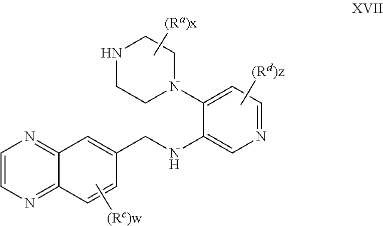
or a pharmaceutically acceptable salt thereof, wherein
each R^{a} is independently selected from C₁ to C₃ alkyl, —OCH₃, —CF₃, —CH₂F, —CHF₂, halo, hydroxyl and amino; or additionally or alternatively, two R^{a} attached to the same carbon atom form an oxo group; or two R^{a} attached to different carbon atoms form a 4- to 6-membered carbocyclic ring or a 4- to 6-membered heterocyclic ring having 1 or 2 heteroatoms selected from O and NH;
each R^{c} is independently selected from halo, Cᵣ to C₃ alkyl, —OCH₃, —CF₃, —CH₂F, —CHF₂, —CN, hydroxyl and amino;
each R^{d} is independently selected from halo, C₁ to C₃ alkyl, —OCH₃, —CF₃, —CH₂F, —CHF₂, —CN, hydroxyl and amino;
alternatively, two R^{d} on adjacent ring positions may be taken together to form a 5- or 6-membered aromatic ring having from 0 to 2 heteroatoms selected from O, S, N and NH;
w is 0, 1 or 2;
x is 0, 1, 2 or 3; and
z is 0, 1 or 2.

48. The polynucleotide cassette of claim 44, wherein the small molecule has a structure according to formula XX 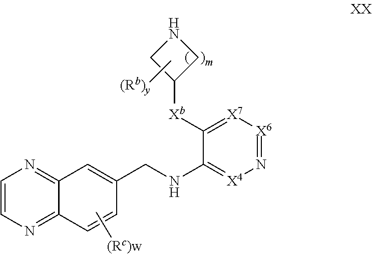
or a pharmaceutically acceptable salt thereof, wherein
X⁴ is selected from CH, CR^{d} and N;
X⁶ is selected from CH, CR^{d} and N;
X⁷ is selected from CH, CR^{d} and N;
wherein 0 or 1 of X⁴, X⁶ or X⁷ is N;
X^{b} is selected from O, NH, and NCH₃;
each R^{b} is independently selected from C₁ to C₃ alkyl, —OCH₃, —CF₃, —CH₂F, —CHF₂, halo, hydroxyl and amino; or additionally or alternatively, two R^{b} attached to the same carbon atom form an oxo group; or two R^{b} attached to different carbon atoms form a 4- to 6-membered carbocyclic ring or a 4- to 6-membered heterocyclic ring having 1 or 2 heteroatoms selected from O and NH;
m is 1 or 2;
y is 0, 1, 2 or 3;
each R^{c} is independently selected from halo, C₁ to C₃ alkyl, —OCH₃, —CF₃, —CH₂F, —CHF₂, —CN, hydroxyl and amino;
each R^{d} is independently selected from halo, C₁ to C₃ alkyl, —OCH₃, —CF₃, —CH₂F, —CHF₂, —CN, hydroxyl and amino;
alternatively, two R^{d} on adjacent ring positions may be taken together to form a 5- or 6-membered aromatic ring having from 0 to 2 heteroatoms selected from O, S, N and NH; and
w is 0, 1 or 2.

49. The polynucleotide cassette of claim 44, wherein the small molecule has a structure according to formula XXI 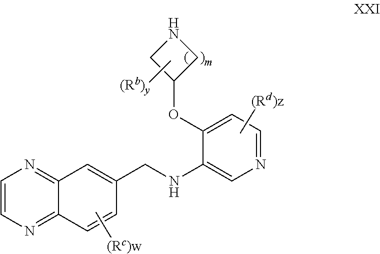
or a pharmaceutically acceptable salt thereof, wherein
each R^{b} is independently selected from C₁ to C₃ alkyl, —OCH₃, —CF₃, —CH₂F, —CHF₂, halo, hydroxyl and amino; or additionally or alternatively, two R^{b} attached to the same carbon atom form an oxo group; or two R^{b} attached to different carbon atoms form a 4- to 6-membered carbocyclic ring or a 4- to 6-membered heterocyclic ring having 1 or 2 heteroatoms selected from O and NH;
each R^{c} is independently selected from halo, C₁ to C₃ alkyl, —OCH₃, —CF₃, —CH₂F, —CHF₂, —CN, hydroxyl and amino;
each R^{d} is independently selected from halo, C₁ to C₃ alkyl, —OCH₃, —CF₃, —CH₂F, —CHF₂, —CN, hydroxyl and amino;
alternatively, two R^{d} on adjacent ring positions may be taken together to form a 5- or 6-membered aromatic ring having from 0 to 2 heteroatoms selected from O, S, N and NH;
m is 1 or 2;
w is 0, 1 or 2
y is 0, 1 or 2; and
z is 0, 1 or 2.

50. A vector comprising the polynucleotide cassette of any one of claims 1-19 and 40-49, the nucleic acid sequence of any one of claims 20-39, or the polynucleotide cassette of any one of claims x-x.

51. The vector of claim 44, wherein the vector is a viral vector.

52. The vector of claim 45, wherein the viral vector is selected from the group consisting of an adenoviral vector, an adeno-associated virus vector, and a lentiviral vector.

53. A cell comprising the vector of any one of claims 44-46, the polynucleotide cassette of any one of claims 1-19 and 40-42, or the nucleic acid sequence of any one of claims 20-39.

54. A compound having the structure according to formula XIII 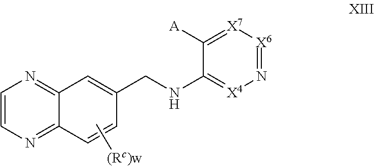
or a pharmaceutically acceptable salt thereof, wherein
X⁴ is selected from CH, CR^{d} and N;
X⁶ is selected from CH, CR^{d} and N;
X⁷ is selected from CH, CR^{d} and N;
wherein 0 or 1 of X⁴, X⁶ or X⁷ is N;
A is selected from the group consisting of: 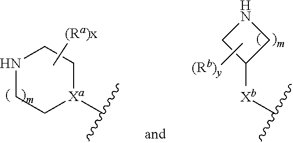
X^{a} is selected from N and CH;
X^{b} is selected from O, NH, and NCH₃;
each R^{a} is independently selected from C₁ to C₃ alkyl, —OCH₃, —CF₃, —CH₂F, —CHF₂, halo, hydroxyl and amino; or additionally or alternatively, two R^{a} attached to the same carbon atom form an oxo group; or two R^{a} attached to different carbon atoms form a 4- to 6-membered carbocyclic ring or a 4- to 6-membered heterocyclic ring having 1 or 2 heteroatoms selected from O and NH;
each R^{b} is independently selected from C₁ to C₃ alkyl, —OCH₃, —CF₃, —CH₂F, —CHF₂, halo, hydroxyl and amino; or additionally or alternatively, two R^{b} attached to the same carbon atom form an oxo group; or two R^{b} attached to different carbon atoms form a 4- to 6-membered carbocyclic ring or a 4- to 6-membered heterocyclic ring having 1 or 2 heteroatoms selected from O and NH;
m is 1 or 2;
x is 0, 1, 2 or 3;
y is 0, 1, 2 or 3;
each R^{c} is independently selected from halo, C₁ to C₃ alkyl, —OCH₃, —CF₃, —CH₂F, —CHF₂, —CN, hydroxyl and amino;
each R^{d} is independently selected from halo, C₁ to C₃ alkyl, —OCH₃, —CF₃, —CH₂F, —CHF₂, —CN, hydroxyl and amino;
alternatively, two R^{d} on adjacent ring positions may be taken together to form a 5- or 6-membered aromatic ring having from 0 to 2 heteroatoms selected from O, S, N and NH; and
w is 0, 1 or 2.

55. The compound of claim 54, having the structure according to formula XIV 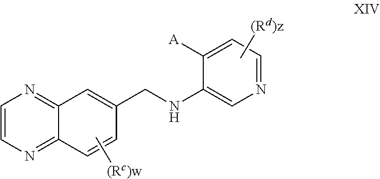
or a pharmaceutically acceptable salt thereof, wherein
or a pharmaceutically acceptable salt thereof, wherein
A is selected from the group consisting of: 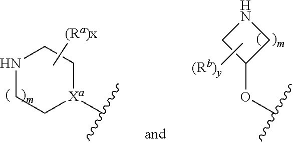
X^{a} is selected from N and CH;
each R^{a} is independently selected from C₁ to C₃ alkyl, —OCH₃, —CF₃, —CH₂F, —CHF₂, halo, hydroxyl and amino; or additionally or alternatively, two R^{a} attached to the same carbon atom form an oxo group; or two R^{a} attached to different carbon atoms form a 4- to 6-membered carbocyclic ring or a 4- to 6-membered heterocyclic ring having 1 or 2 heteroatoms selected from O and NH;
each R^{b} is independently selected from C₁ to C₃ alkyl, —OCH₃, —CF₃, —CH₂F, —CHF₂, halo, hydroxyl and amino; or additionally or alternatively, two R^{b} attached to the same carbon atom form an oxo group; or two R^{b} attached to different carbon atoms form a 4- to 6-membered carbocyclic ring or a 4- to 6-membered heterocyclic ring having 1 or 2 heteroatoms selected from O and NH;
m is 1 or 2;
x is 0, 1, 2 or 3;
y is 0, 1, 2 or 3;
each R^{c} is independently selected from halo, C₁ to C₃ alkyl, —OCH₃, —CF₃, —CH₂F, —CHF₂, —CN, hydroxyl and amino;
each R^{d} is independently selected from halo, C₁ to C₃ alkyl, —OCH₃, —CF₃, —CH₂F, —CHF₂, —CN, hydroxyl and amino; alternatively, two R^{d} on adjacent ring positions may be taken together to form a 5- or 6-membered aromatic ring having from 0 to 2 heteroatoms selected from O, S, N and NH;
w is 0, 1 or 2; and
z is 0, 1 or 2;
wherein when A is selected to be 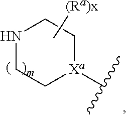
x is 1, 2 or 3; and/or
two R^{d} on adjacent ring positions are taken together to form a 5- or 6-membered aromatic ring having from 0 to 2 heteroatoms selected from O, S, N and NH.

56. The compound of claim 54, having the structure according to formula XVI 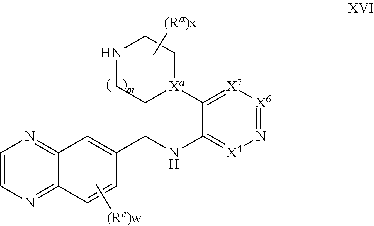
or a pharmaceutically acceptable salt thereof, wherein
X⁴ is selected from CH, CR^{d} and N;
X⁶ is selected from CH, CR^{d} and N;
X⁷ is selected from CH, CR^{d} and N;
wherein 0 or 1 of X⁴, X⁶ or X⁷ is N;
X^{a} is selected from N and CH;
each R^{a} is independently selected from C₁ to C₃ alkyl, —OCH₃, —CF₃, —CH₂F, —CHF₂, halo, hydroxyl and amino; or additionally or alternatively, two R^{a} attached to the same carbon atom form an oxo group; or two R^{a} attached to different carbon atoms form a 4- to 6-membered carbocyclic ring or a 4- to 6-membered heterocyclic ring having 1 or 2 heteroatoms selected from O and NH;
m is 1 or 2;
x is 1, 2 or 3;
each R^{c} is independently selected from halo, C₁ to C₃ alkyl, —OCH₃, —CF₃, —CH₂F, —CHF₂, —CN, hydroxyl and amino;
each R^{d} is independently selected from halo, C₁ to C₃ alkyl, —OCH₃, —CF₃, —CH₂F, —CHF₂, —CN, hydroxyl and amino;
alternatively, two R^{d} on adjacent ring positions may be taken together to form a 5- or 6-membered aromatic ring having from 0 to 2 heteroatoms selected from O, S, N and NH; and
w is 0, 1 or 2.

57. The compound of claim 54, having the structure according to formula XVII 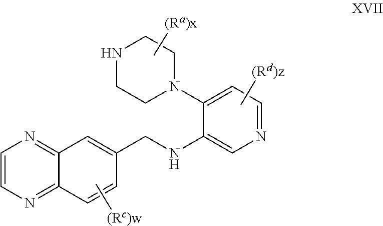
or a pharmaceutically acceptable salt thereof, wherein
each R^{a} is independently selected from C₁ to C₃ alkyl, —OCH₃, —CF₃, —CH₂F, —CHF₂, halo, hydroxyl and amino; or additionally or alternatively, two R^{a} attached to the same carbon atom form an oxo group; or two R^{a} attached to different carbon atoms form a 4- to 6-membered carbocyclic ring or a 4- to 6-membered heterocyclic ring having 1 or 2 heteroatoms selected from O and NH;
each R^{c} is independently selected from halo, C₁ to C₃ alkyl, —OCH₃, —CF₃, —CH₂F, —CHF₂, —CN, hydroxyl and amino;
each R^{d} is independently selected from halo, C₁ to C₃ alkyl, —OCH₃, —CF₃, —CH₂F, —CHF₂, —CN, hydroxyl and amino;
alternatively, two R^{d} on adjacent ring positions may be taken together to form a 5- or 6-membered aromatic ring having from 0 to 2 heteroatoms selected from O, S, N and NH;
w is 0, 1 or 2;
x is 1, 2 or 3; and
z is 0, 1 or 2.

58. The compound of claim 54, having the structure according to formula XX 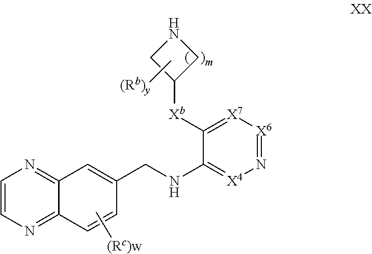
or a pharmaceutically acceptable salt thereof, wherein
X⁴ is selected from CH, CR^{d} and N;
X⁶ is selected from CH, CR^{d} and N;
X⁷ is selected from CH, CR^{d} and N;
wherein 0 or 1 of X⁴, X⁶ or X⁷ is N;
X is selected from O, NH, and NCH₃;
each R^{b} is independently selected from C₁ to C₃ alkyl, —OCH₃, —CF₃, —CH₂F, —CHF₂, halo, hydroxyl and amino; or additionally or alternatively, two R^{b} attached to the same carbon atom form an oxo group; or two R^{b} attached to different carbon atoms form a 4- to 6-membered carbocyclic ring or a 4- to 6-membered heterocyclic ring having 1 or 2 heteroatoms selected from O and NH;
m is 1 or 2;
y is 0, 1, 2 or 3;
each R^{c} is independently selected from halo, C₁ to C₃ alkyl, —OCH₃, —CF₃, —CH₂F, —CHF₂, —CN, hydroxyl and amino;
each R^{d} is independently selected from halo, C₁ to C₃ alkyl, —OCH₃, —CF₃, —CH₂F, —CHF₂, —CN, hydroxyl and amino;
alternatively, two R^{d} on adjacent ring positions may be taken together to form a 5- or 6-membered aromatic ring having from 0 to 2 heteroatoms selected from O, S, N and NH; and
w is 0, 1 or 2.

59. The compound of claim 54, having the structure according to formula XXI 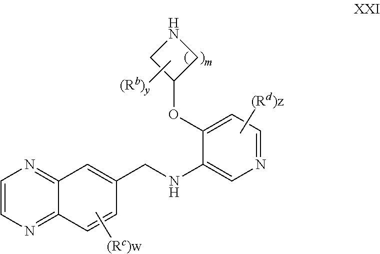
or a pharmaceutically acceptable salt thereof, wherein
each R^{b} is independently selected from C₁ to C₃ alkyl, —OCH₃, —CF₃, —CH₂F, —CHF₂, halo, hydroxyl and amino; or additionally or alternatively, two R^{b} attached to the same carbon atom form an oxo group; or two R^{b} attached to different carbon atoms form a 4- to 6-membered carbocyclic ring or a 4- to 6-membered heterocyclic ring having 1 or 2 heteroatoms selected from O and NH;
each R^{c} is independently selected from halo, C₁ to C₃ alkyl, —OCH₃, —CF₃, —CH₂F, —CHF₂, —CN, hydroxyl and amino;
each R^{d} is independently selected from halo, C₁ to C₃ alkyl, —OCH₃, —CF₃, —CH₂F, —CHF₂, —CN, hydroxyl and amino;
alternatively, two R^{d} on adjacent ring positions may be taken together to form a 5- or 6-membered aromatic ring having from 0 to 2 heteroatoms selected from O, S, N and NH;
m is 1 or 2;
w is 0, 1 or 2
y is 0, 1 or 2; and
z is 0, 1 or 2.

60. The compound of claim 54, having the structure according to one of:

| Ref. | |
|---|---|
| No. | Structure |
| 001 | 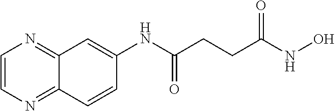 [SMILES: O=C(CCC(=O)Nc1ccc2nccnc2c1)NO] |
| 002 | 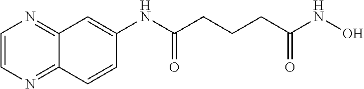 [SMILES: O=C(CCCC(=O)Nc1ccc2nccnc2c1)NO] |
| 003 | 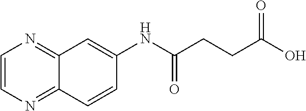 [SMILES: O=C(O)CCC(=O)Nc1ccc2nccnc2c1] |
| 004 | 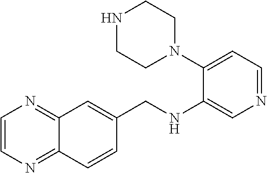 [SMILES: c1cc(N2CCNCC2)c(NCc2ccc3nccnc3c2)cn1] |
| 005 | 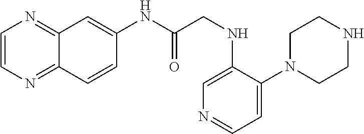 [SMILES: O=C(CNc1cnccc1N1CCNCC1)Nc1ccc2nccnc2c1] |
| 006 | 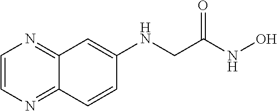 [SMILES: O=C(CNc1ccc2nccnc2c1)NO] |
| 007 | 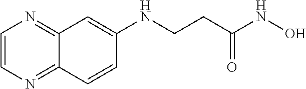 [SMILES: O=C(CCNc1ccc2nccnc2c1)NO] |
| 008 | 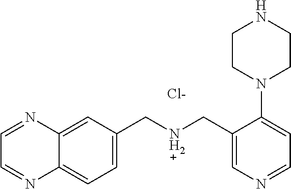 [SMILES: c1cc(N2CCNCC2)c(C[NH2+]Cc2ccc3nccnc3c2)cn1 ; [Cl-]] |
| 009 | 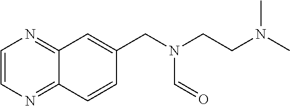 [SMILES: CN(C)CCN(C=O)Cc1ccc2nccnc2c1] |
| 010 | 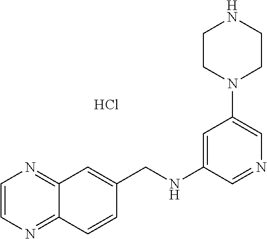 [SMILES: c1cnc2cc(CNc3cncc(N4CCNCC4)c3)ccc2n1 ; Cl] |
| 011 | 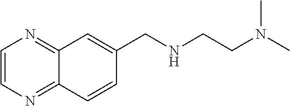 [SMILES: CN(C)CCNCc1ccc2nccnc2c1] |
| 012 | 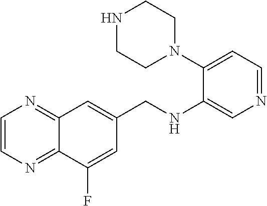 [SMILES: Fc1cc(CNc2cnccc2N2CCNCC2)cc2nccnc12] |
| 013 | 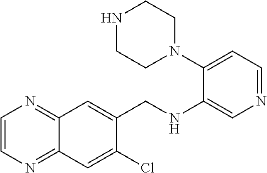 [SMILES: Clc1cc2nccnc2cc1CNc1cnccc1N1CCNCC1] |
| 014 | 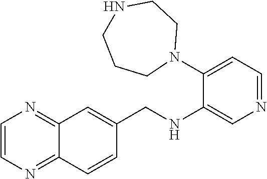 [SMILES: c1cc(N2CCCNCC2)c(NCc2ccc3nccnc3c2)cn1] |
| 015 | 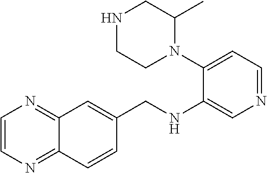 [SMILES: CC1CNCCN1c1ccncc1NCc1ccc2nccnc2c1] |
| 016 | 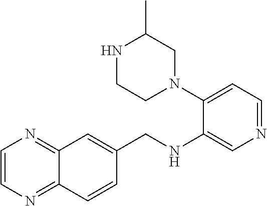 [SMILES: CC1CN(c2ccncc2NCc2ccc3nccnc3c2)CCN1] |
| 017 | 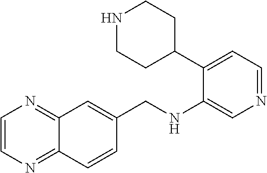 [SMILES: c1cc(C2CCNCC2)c(NCc2ccc3nccnc3c2)cn1] |
| 018 | 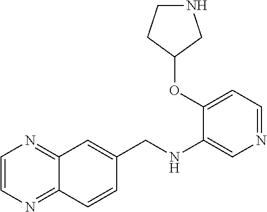 [SMILES: c1cc(OC2CCNC2)c(NCc2ccc3nccnc3c2)cn1] |
| 019 | 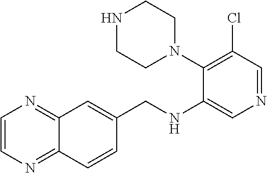 [SMILES: Clc1cncc(NCc2ccc3nccnc3c2)c1N1CCNCC1] |
| 020 | 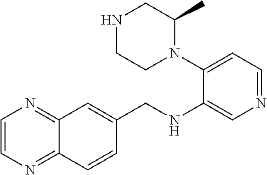 [SMILES: C[C@@H]1CNCCN1c1ccncc1NCc1ccc2nccnc2c1] |
| 021 | 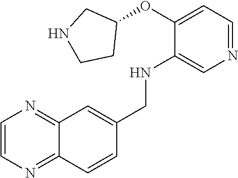 [SMILES: c1cc(O[C@@H]2CCNC2)c(NCc2ccc3nccnc3c2)cn1] |
| 022 | 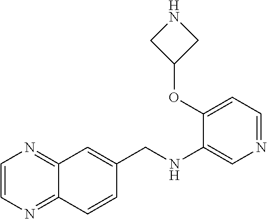 [SMILES: c1cc(OC2CNC2)c(NCc2ccc3nccnc3c2)cn1] |
| 023 | 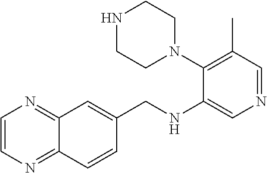 [SMILES: Cc1cncc(NCc2ccc3nccnc3c2)c1N1CCNCC1] |
| 024 | 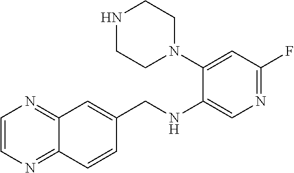 [SMILES: Fc1cc(N2CCNCC2)c(NCc2ccc3nccnc3c2)cn1] |
| 025 | 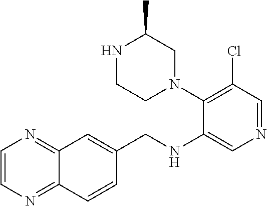 [SMILES: C[C@H]1CN(c2c(Cl)cncc2NCc2ccc3nccnc3c2)CCN1] |
| 026 | 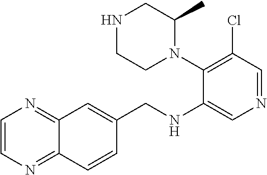 [SMILES: C[C@@H]1CNCCN1c1c(Cl)cncc1NCc1ccc2nccnc2c1] |
| 027 | 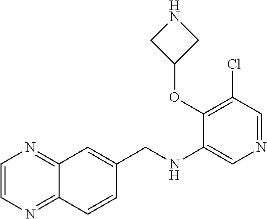 [SMILES: Clc1cncc(NCc2ccc3nccnc3c2)c1OC1CNC1] |
| 028 | 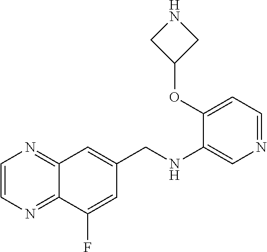 [SMILES: Fc1cc(CNc2cnccc2OC2CNC2)cc2nccnc12] |
| 029 | 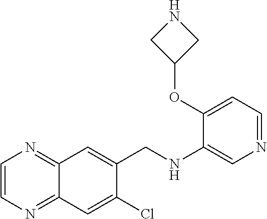 [SMILES: Clc1cc2nccnc2cc1CNc1cnccc1OC1CNC1] |
| 030 | 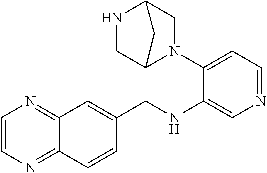 [SMILES: c1cc(N2CC3CC2CN3)c(NCc2ccc3nccnc3c2)cn1] |
| 031 | 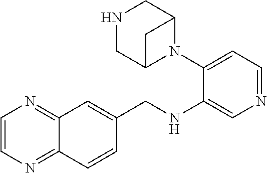 [SMILES: c1cc(N2C3CNCC2C3)c(NCc2ccc3nccnc3c2)cn1] |
| 032 | 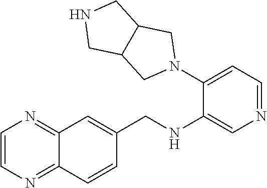 [SMILES: c1cc(N2CC3CNCC3C2)c(NCc2ccc3nccnc3c2)cn1] |
| 033 | 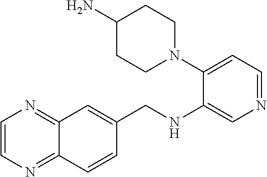 [SMILES: NC1CCN(c2ccncc2NCc2ccc3nccnc3c2)CC1] |
| 034 | 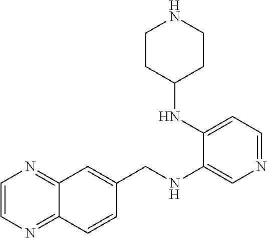 [SMILES: c1cc(NC2CCNCC2)c(NCc2ccc3nccnc3c2)cn1] |
| 035 | 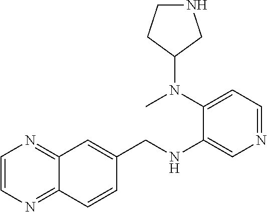 [SMILES: CN(c1ccncc1NCc1ccc2nccnc2c1)C1CCNC1] |
| 036 | 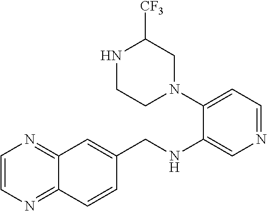 [SMILES: FC(F)(F)C1CN(c2ccncc2NCc2ccc3nccnc3c2)CCN1] |
| 037 | 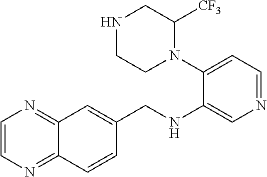 [SMILES: FC(F)(F)C1CNCCN1c1ccncc1NCc1ccc2nccnc2c1] |
| 038 | 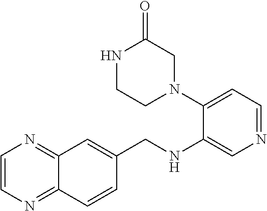 [SMILES: O=C1CN(c2ccncc2NCc2ccc3nccnc3c2)CCN1] |
| 039 | 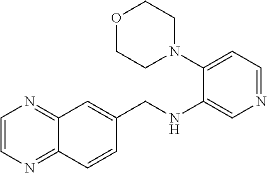 [SMILES: c1cc(N2CCOCC2)c(NCc2ccc3nccnc3c2)cn1] |
| 040 | 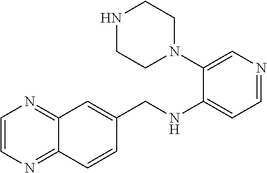 [SMILES: c1cc(NCc2ccc3nccnc3c2)c(N2CCNCC2)cn1] |
| 041 | 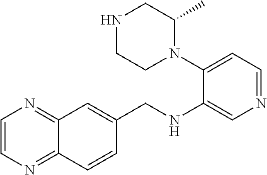 [SMILES: C[C@H]1CNCCN1c1ccncc1NCc1ccc2nccnc2c1] |
| 042 | 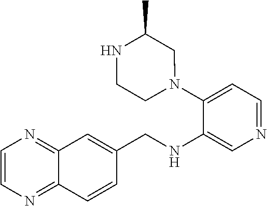 [SMILES: C[C@H]1CN(c2ccncc2NCc2ccc3nccnc3c2)CCN1] |
| 043 | 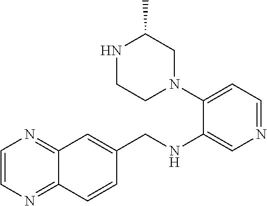 [SMILES: C[C@@H]1CN(c2ccncc2NCc2ccc3nccnc3c2)CCN1] |
| 044 | 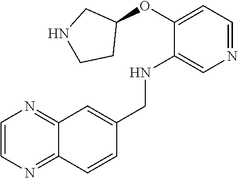 [SMILES: c1cc(O[C@H]2CCNC2)c(NCc2ccc3nccnc3c2)cn1] |
| 045 | 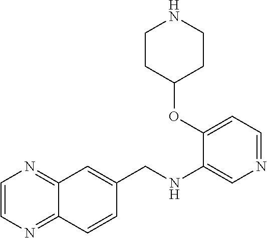 [SMILES: c1cc(OC2CCNCC2)c(NCc2ccc3nccnc3c2)cn1] |
| 046 | 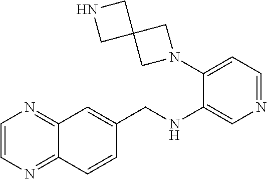 [SMILES: c1cc(N2CC3(CNC3)C2)c(NCc2ccc3nccnc3c2)cn1] |
| 047 | 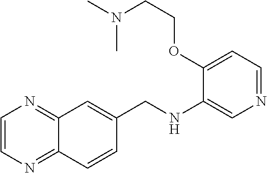 [SMILES: CN(C)CCOc1ccncc1NCc1ccc2nccnc2c1] |
| 048 | 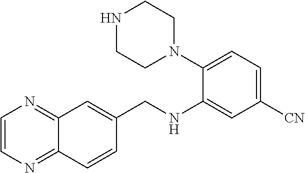 [SMILES: N#Cc1ccc(N2CCNCC2)c(NCc2ccc3nccnc3c2)c1] |
| 049 | 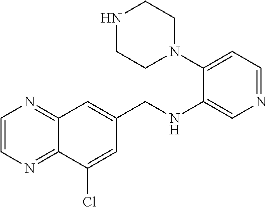 [SMILES: Clc1cc(CNc2cnccc2N2CCNCC2)cc2nccnc12] |
| 050 | 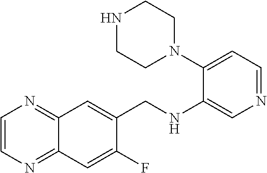 [SMILES: Fc1cc2nccnc2cc1CNc1cnccc1N1CCNCC1] |
| 051 | 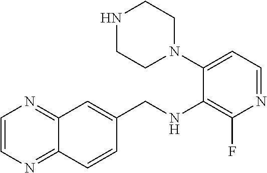 [SMILES: Fc1nccc(N2CCNCC2)c1NCc1ccc2nccnc2c1] |
| 052 | 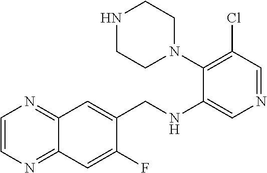 [SMILES: Fc1cc2nccnc2cc1CNc1cncc(Cl)c1N1CCNCC1] |
| 053 | 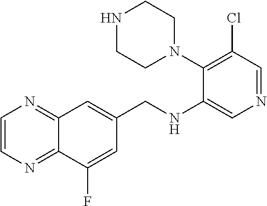 [SMILES: Fc1cc(CNc2cncc(Cl)c2N2CCNCC2)cc2nccnc12] |
| 054 | 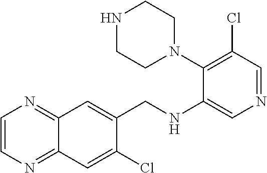 [SMILES: Clc1cc2nccnc2cc1CNc1cncc(Cl)c1N1CCNCC1] |
| 055 | 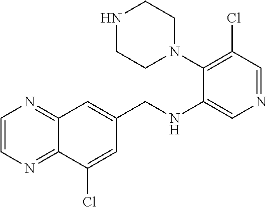 [SMILES: Clc1cncc(NCc2cc(Cl)c3nccnc3c2)c1N1CCNCC1] |
| 056 | 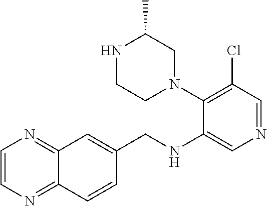 [SMILES: C[C@@H]1CN(c2c(Cl)cncc2NCc2ccc3nccnc3c2)CCN1] |
| 057 | 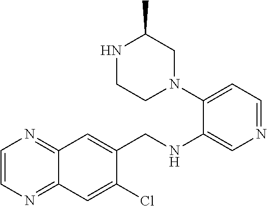 [SMILES: C[C@H]1CN(c2ccncc2NCc2cc3nccnc3cc2Cl)CCN1] |
| 058 | 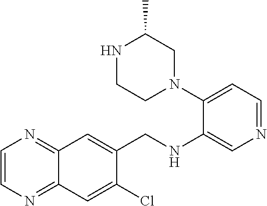 [SMILES: C[C@@H]1CN(c2ccncc2NCc2cc3nccnc3cc2Cl)CCN1] |
| 059 | 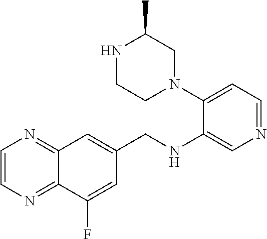 [SMILES: C[C@H]1CN(c2ccncc2NCc2cc(F)c3nccnc3c2)CCN1] |
| 060 | 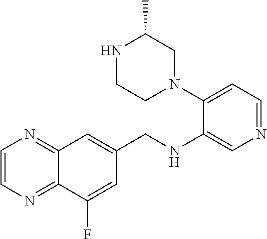 [SMILES: C[C@@H]1CN(c2ccncc2NCc2cc(F)c3nccnc3c2)CCN1] |
| 061 | 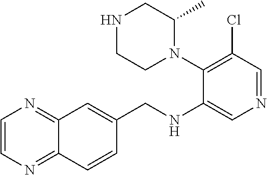 [SMILES: C[C@H]1CNCCN1c1c(Cl)cncc1NCc1ccc2nccnc2c1] |
| 062 | 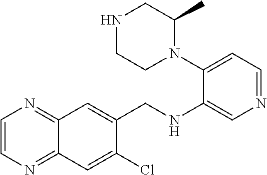 [SMILES: C[C@@H]1CNCCN1c1ccncc1NCc1cc2nccnc2cc1Cl] |
| 063 | 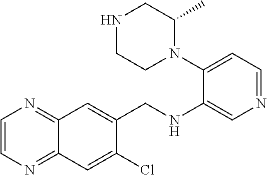 [SMILES: C[C@H]1CNCCN1c1ccncc1NCc1cc2nccnc2cc1Cl] |
| 064 | 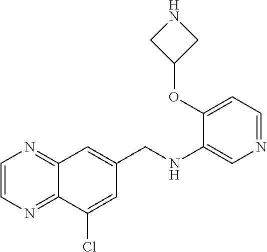 [SMILES: Clc1cc(CNc2cnccc2OC2CNC2)cc2nccnc12] |
| 065 | 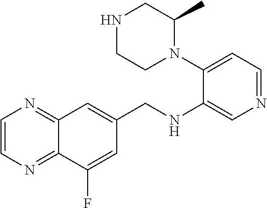 [SMILES: C[C@@H]1CNCCN1c1ccncc1NCc1cc(F)c2nccnc2c1] |
| 066 | 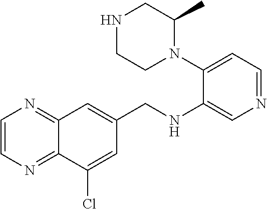 [SMILES: C[C@@H]1CNCCN1c1ccncc1NCc1cc(Cl)c2nccnc2c1] |
| 067 | 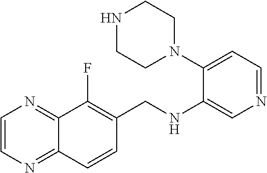 [SMILES: Fc1c(CNc2cnccc2N2CCNCC2)ccc2nccnc12] |
| 068 | 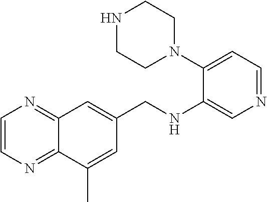 [SMILES: Cc1cc(CNc2cnccc2N2CCNCC2)cc2nccnc12] |
| 069 | 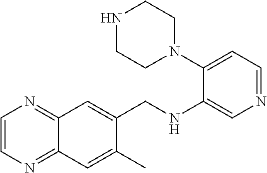 [SMILES: Cc1cc2nccnc2cc1CNc1cnccc1N1CCNCC1] |
| 070 | 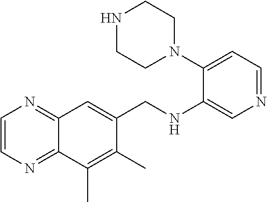 [SMILES: Cc1c(CNc2cnccc2N2CCNCC2)cc2nccnc2c1C] |
| 071 | 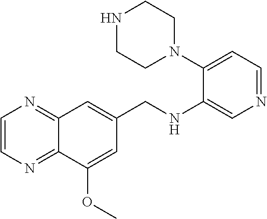 [SMILES: COc1cc(CNc2cnccc2N2CCNCC2)cc2nccnc12] |
| 072 | 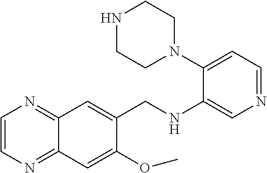 [SMILES: COc1cc2nccnc2cc1CNc1cnccc1N1CCNCC1] |
| 073 | 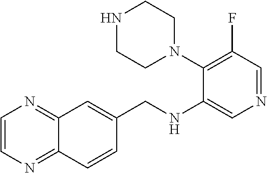 [SMILES: Fc1cncc(NCc2ccc3nccnc3c2)c1N1CCNCC1] |
| 074 | 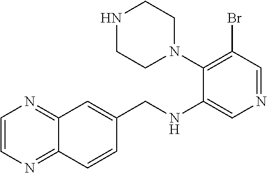 [SMILES: Brc1cncc(NCc2ccc3nccnc3c2)c1N1CCNCC1] |
| 075 | 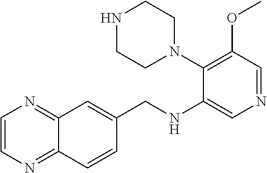 [SMILES: COc1cncc(NCc2ccc3nccnc3c2)c1N1CCNCC1] |
| 076 | 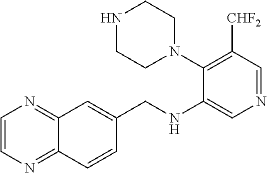 [SMILES: FC(F)c1cncc(NCc2ccc3nccnc3c2)c1N1CCNCC1] |
| 077 | 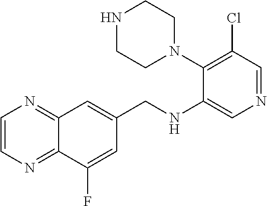 [SMILES: Fc1cc(CNc2cncc(Cl)c2N2CCNCC2)cc2nccnc12] |
| 078 | 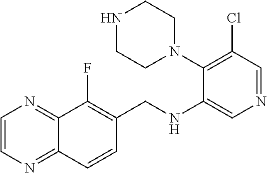 [SMILES: Fc1c(CNc2cncc(Cl)c2N2CCNCC2)ccc2nccnc12] |
| 079 | 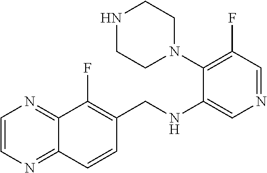 [SMILES: Fc1cncc(NCc2ccc3nccnc3c2F)c1N1CCNCC1] |
| 080 | 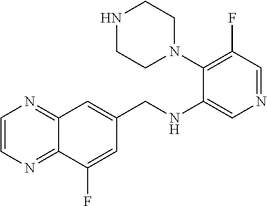 [SMILES: Fc1cncc(NCc2cc(F)c3nccnc3c2)c1N1CCNCC1] |
| 081 | 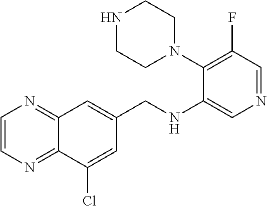 [SMILES: Fc1cncc(NCc2cc(Cl)c3nccnc3c2)c1N1CCNCC1] |
| 082 | 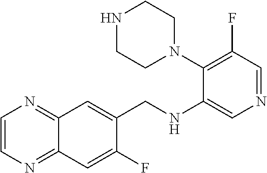 [SMILES: Fc1cc2nccnc2cc1CNc1cncc(F)c1N1CCNCC1] |
| 083 | 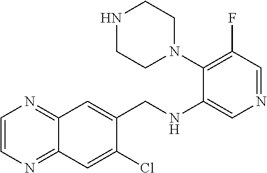 [SMILES: Fc1cncc(NCc2cc3nccnc3cc2Cl)c1N1CCNCC1] |
| 084 | 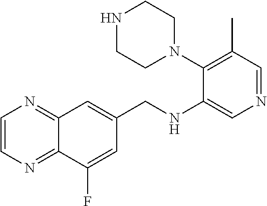 [SMILES: Cc1cncc(NCc2cc(F)c3nccnc3c2)c1N1CCNCC1] |
| 085 | 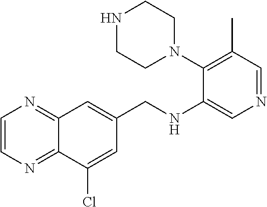 [SMILES: Cc1cncc(NCc2cc(Cl)c3nccnc3c2)c1N1CCNCC1] |
| 086 | 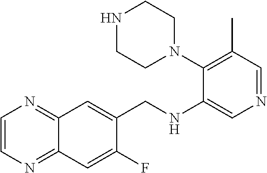 [SMILES: Cc1cncc(NCc2cc3nccnc3cc2F)c1N1CCNCC1] |
| 087 | 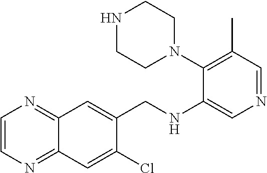 [SMILES: Cc1cncc(NCc2cc3nccnc3cc2Cl)c1N1CCNCC1] |
| 088 | 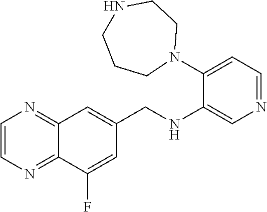 [SMILES: Fc1cc(CNc2cnccc2N2CCCNCC2)cc2nccnc12] |
| 089 | 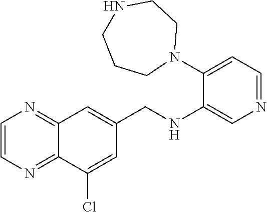 [SMILES: Clc1cc(CNc2cnccc2N2CCCNCC2)cc2nccnc12] |
| 090 | 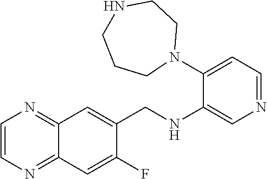 [SMILES: Fc1cc2nccnc2cc1CNc1cnccc1N1CCCNCC1] |
| 091 | 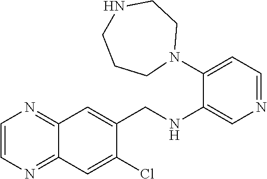 [SMILES: Clc1cc2nccnc2cc1CNc1cnccc1N1CCCNCC1] |
| 092 | 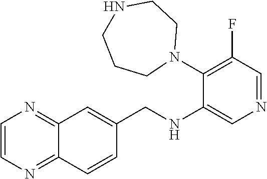 [SMILES: Fc1cncc(NCc2ccc3nccnc3c2)c1N1CCCNCC1] |
| 093 | 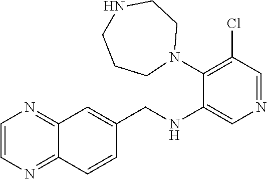 [SMILES: Clc1cncc(NCc2ccc3nccnc3c2)c1N1CCCNCC1] |
| 094 | 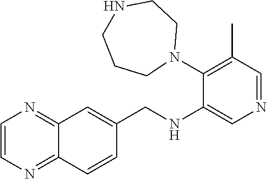 [SMILES: Cc1cncc(NCc2ccc3nccnc3c2)c1N1CCCNCC1] |
| 095 | 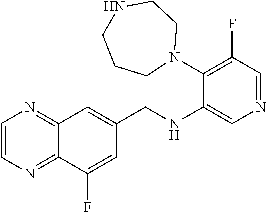 [SMILES: Fc1cncc(NCc2cc(F)c3nccnc3c2)c1N1CCCNCC1] |
| 096 | 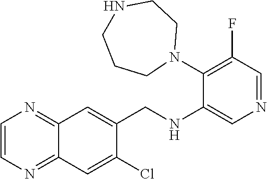 [SMILES: Fc1cncc(NCc2cc3nccnc3cc2Cl)c1N1CCCNCC1] |
| 097 | 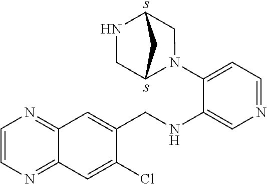 |
| 098 | 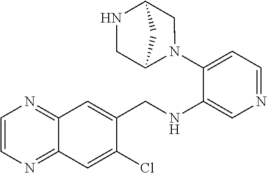 [SMILES: Clc1cc2nccnc2cc1CNc1cnccc1N1C[C@H]2C[C@@H]1CN2] |
| 099 | 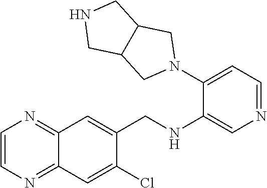 [SMILES: Clc1cc2nccnc2cc1CNc1cnccc1N1CC2CNCC2C1] |
| 100 | 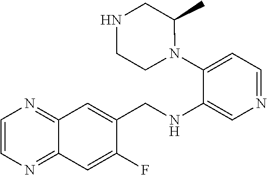 [SMILES: C[C@@H]1CNCCN1c1ccncc1NCc1cc2nccnc2cc1F] |
| 101 | 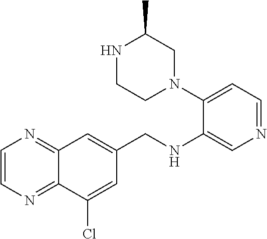 [SMILES: C[C@H]1CN(c2ccncc2NCc2cc(Cl)c3nccnc3c2)CCN1] |
| 102 | 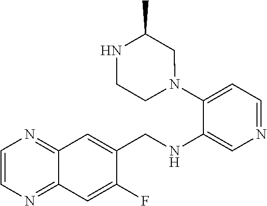 [SMILES: C[C@H]1CN(c2ccncc2NCc2cc3nccnc3cc2F)CCN1] |
| 103 | 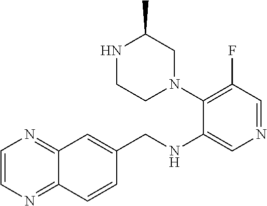 [SMILES: C[C@H]1CN(c2c(F)cncc2NCc2ccc3nccnc3c2)CCN1] |
| 104 | 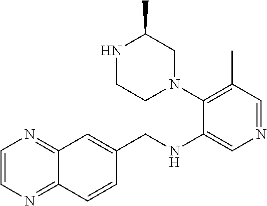 [SMILES: Cc1cncc(NCc2ccc3nccnc3c2)c1N1CCN[C@@H](C)C1] |
| 105 | 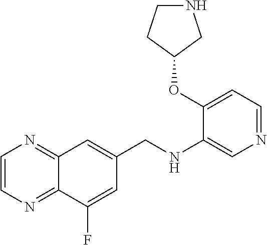 [SMILES: Fc1cc(CNc2cnccc2O[C@@H]2CCNC2)cc2nccnc12] |
| 106 | 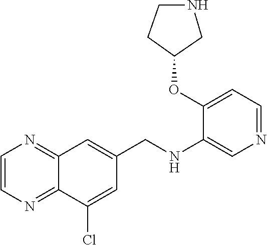 [SMILES: Clc1cc(CNc2cnccc2O[C@@H]2CCNC2)cc2nccnc12] |
| 107 | 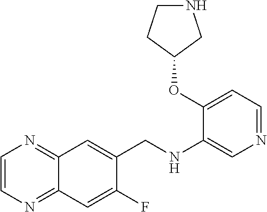 [SMILES: Fc1cc2nccnc2cc1CNc1cnccc1O[C@@H]1CCNC1] |
| 108 | 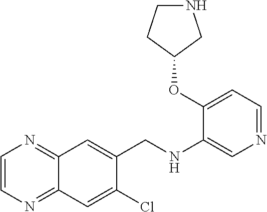 [SMILES: Clc1cc2nccnc2cc1CNc1cnccc1O[C@@H]1CCNC1] |
| 109 | 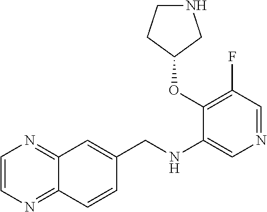 [SMILES: Fc1cncc(NCc2ccc3nccnc3c2)c1O[C@@H]1CCNC1] |
| 110 | 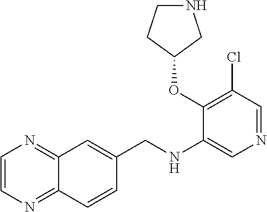 [SMILES: Clc1cncc(NCc2ccc3nccnc3c2)c1O[C@@H]1CCNC1] |
| 111 | 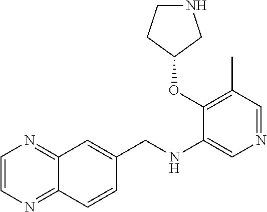 [SMILES: Cc1cncc(NCc2ccc3nccnc3c2)c1O[C@@H]1CCNC1] |
| 112 | 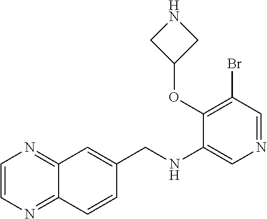 [SMILES: Brc1cncc(NCc2ccc3nccnc3c2)c1OC1CNC1] |
| 113 | 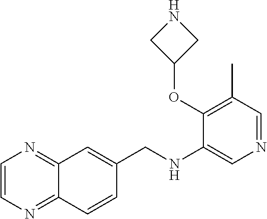 [SMILES: Cc1cncc(NCc2ccc3nccnc3c2)c1OC1CNC1] |
| 114 | 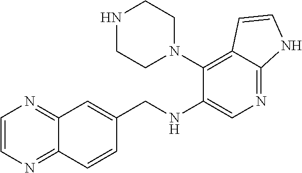 [SMILES: c1cnc2cc(CNc3cnc4[nH]ccc4c3N3CCNCC3)ccc2n1] |
| 115 | 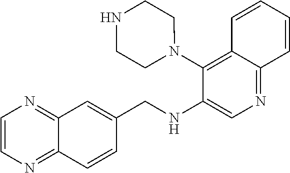 [SMILES: c1ccc2c(N3CCNCC3)c(NCc3ccc4nccnc4c3)cnc2c1] |
| 116 | 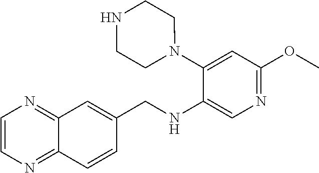 [SMILES: COc1cc(N2CCNCC2)c(NCc2ccc3nccnc3c2)cn1] |
| 117 | 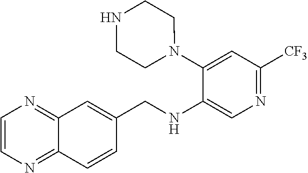 [SMILES: FC(F)(F)c1cc(N2CCNCC2)c(NCc2ccc3nccnc3c2)cn1] |
| 118 | 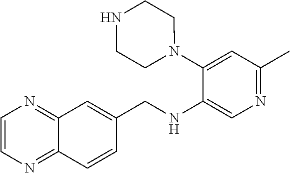 [SMILES: Cc1cc(N2CCNCC2)c(NCc2ccc3nccnc3c2)cn1] |
| 119 | 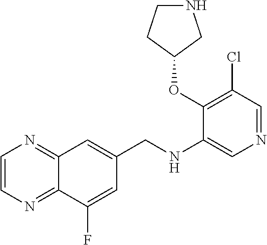 [SMILES: Fc1cc(CNc2cncc(Cl)c2O[C@@H]2CCNC2)cc2nccnc12] |
| 120 | 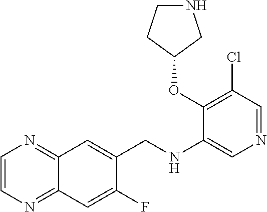 [SMILES: Fc1cc2nccnc2cc1CNc1cncc(Cl)c1O[C@@H]1CCNC1] |
| 121 | 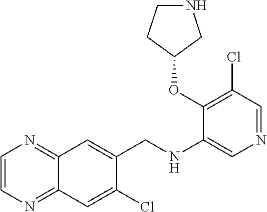 [SMILES: Clc1cc2nccnc2cc1CNc1cncc(Cl)c1O[C@@H]1CCNC1] |
| 122 | 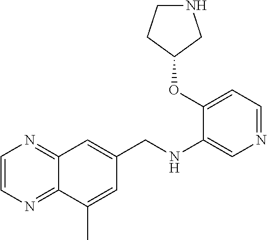 [SMILES: Cc1cc(CNc2cnccc2O[C@@H]2CCNC2)cc2nccnc12] |
| 123 | 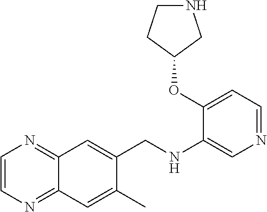 [SMILES: Cc1cc2nccnc2cc1CNc1cnccc1O[C@@H]1CCNC1] |
| 124 | 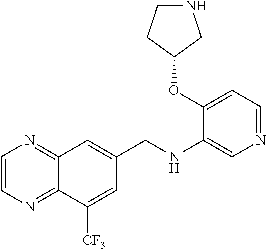 [SMILES: FC(F)(F)c1cc(CNc2cnccc2O[C@@H]2CCNC2)cc2nccnc12] |
| 125 | 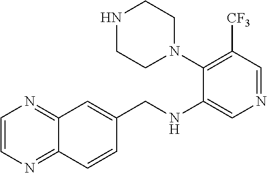 [SMILES: FC(F)(F)c1cncc(NCc2ccc3nccnc3c2)c1N1CCNCC1] |
| 126 | 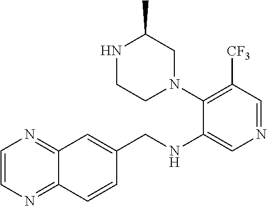 [SMILES: C[C@H]1CN(c2c(NCc3ccc4nccnc4c3)cncc2C(F)(F)F)CCN1] |
| 127 | 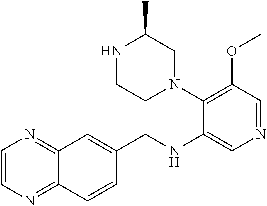 [SMILES: COc1cncc(NCc2ccc3nccnc3c2)c1N1CCN[C@@H](C)C1] |
| 128 | 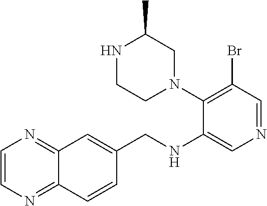 [SMILES: C[C@H]1CN(c2c(Br)cncc2NCc2ccc3nccnc3c2)CCN1] |
| 129 | 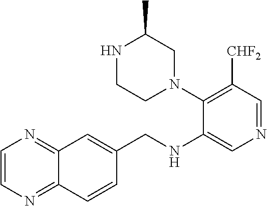 [SMILES: C[C@H]1CN(c2c(NCc3ccc4nccnc4c3)cncc2C(F)F)CCN1] |
| 130 | 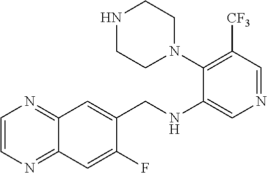 [SMILES: Fc1cc2nccnc2cc1CNc1cncc(C(F)(F)F)c1N1CCNCC1] |
| 131 | 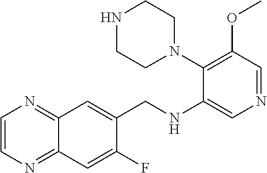 [SMILES: COc1cncc(NCc2cc3nccnc3cc2F)c1N1CCNCC1] |
| 132 | 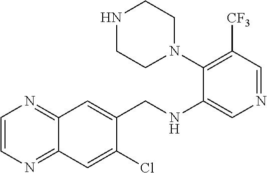 [SMILES: FC(F)(F)c1cncc(NCc2cc3nccnc3cc2Cl)c1N1CCNCC1] |
| 133 | 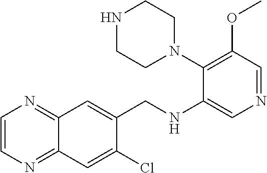 [SMILES: COc1cncc(NCc2cc3nccnc3cc2Cl)c1N1CCNCC1] |
| 134 | 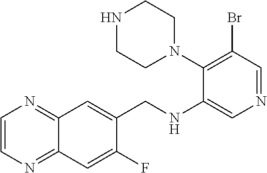 [SMILES: Fc1cc2nccnc2cc1CNc1cncc(Br)c1N1CCNCC1] |
| 135 | 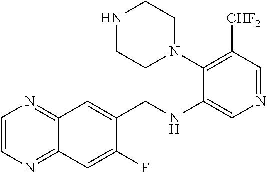 [SMILES: Fc1cc2nccnc2cc1CNc1cncc(C(F)F)c1N1CCNCC1] |
| 136 | 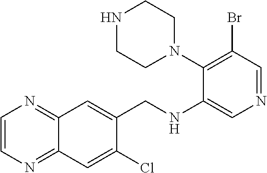 [SMILES: Clc1cc2nccnc2cc1CNc1cncc(Br)c1N1CCNCC1] |
| 137 | 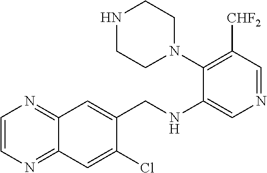 [SMILES: FC(F)c1cncc(NCc2cc3nccnc3cc2Cl)c1N1CCNCC1] |
| 138 | 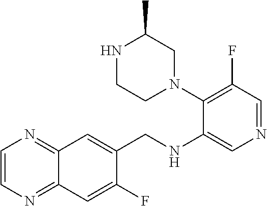 [SMILES: C[C@H]1CN(c2c(F)cncc2NCc2cc3nccnc3cc2F)CCN1] |
| 139 | 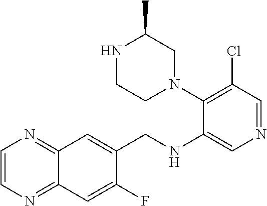 [SMILES: C[C@H]1CN(c2c(Cl)cncc2NCc2cc3nccnc3cc2F)CCN1] |
| 140 | 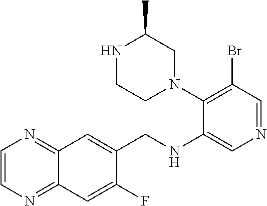 [SMILES: C[C@H]1CN(c2c(Br)cncc2NCc2cc3nccnc3cc2F)CCN1] |
| 141 | 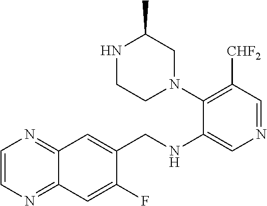 [SMILES: C[C@H]1CN(c2c(NCc3cc4nccnc4cc3F)cncc2C(F)F)CCN1] |
| 142 | 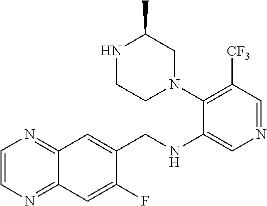 [SMILES: C[C@H]1CN(c2c(NCc3cc4nccnc4cc3F)cncc2C(F)(F)F)CCN1] |
| 143 | 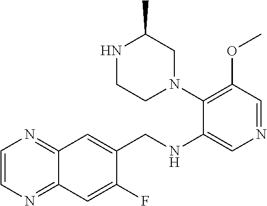 [SMILES: COc1cncc(NCc2cc3nccnc3cc2F)c1N1CCN[C@@H](C)C1] |
| 144 | 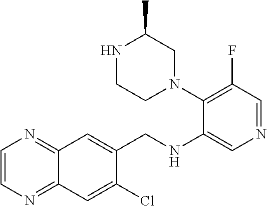 [SMILES: C[C@H]1CN(c2c(F)cncc2NCc2cc3nccnc3cc2Cl)CCN1] |
| 145 | 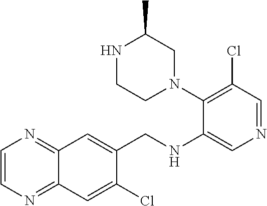 [SMILES: C[C@H]1CN(c2c(Cl)cncc2NCc2cc3nccnc3cc2Cl)CCN1] |
| 146 | 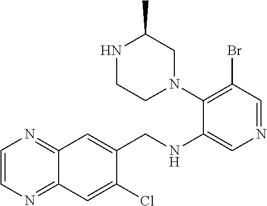 [SMILES: C[C@H]1CN(c2c(Br)cncc2NCc2cc3nccnc3cc2Cl)CCN1] |
| 147 | 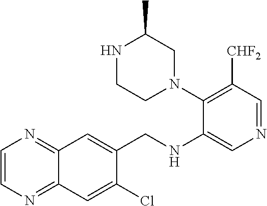 [SMILES: C[C@H]1CN(c2c(NCc3cc4nccnc4cc3Cl)cncc2C(F)F)CCN1] |
| 148 | 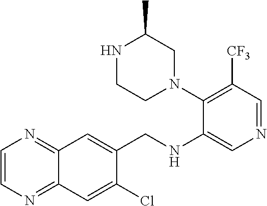 [SMILES: C[C@H]1CN(c2c(NCc3cc4nccnc4cc3Cl)cncc2C(F)(F)F)CCN1] |
| 149 | 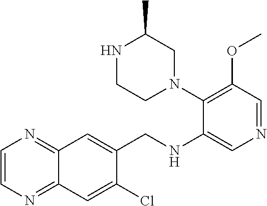 [SMILES: COc1cncc(NCc2cc3nccnc3cc2Cl)c1N1CCN[C@@H](C)C1] |
| 150 | 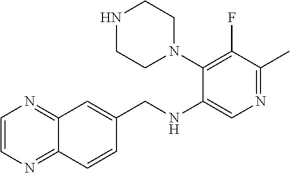 [SMILES: Cc1ncc(NCc2ccc3nccnc3c2)c(N2CCNCC2)c1F] |
| 151 | 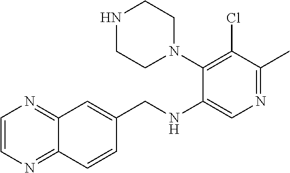 [SMILES: Cc1ncc(NCc2ccc3nccnc3c2)c(N2CCNCC2)c1Cl] |
| 152 | 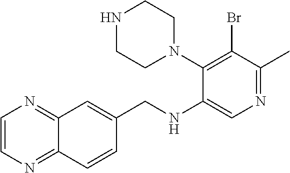 [SMILES: Cc1ncc(NCc2ccc3nccnc3c2)c(N2CCNCC2)c1Br] |
| 153 | 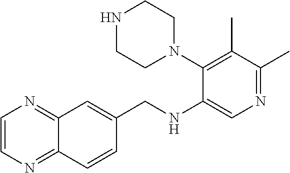 [SMILES: Cc1ncc(NCc2ccc3nccnc3c2)c(N2CCNCC2)c1C] |
| 154 | 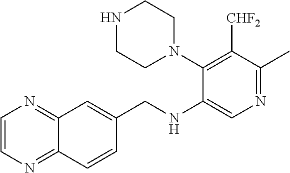 [SMILES: Cc1ncc(NCc2ccc3nccnc3c2)c(N2CCNCC2)c1C(F)F] |
| 155 | 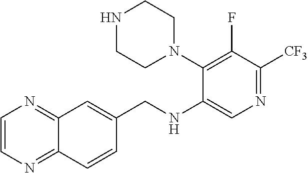 [SMILES: Fc1c(C(F)(F)F)ncc(NCc2ccc3nccnc3c2)c1N1CCNCC1] |
| 156 | 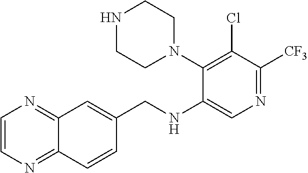 [SMILES: FC(F)(F)c1ncc(NCc2ccc3nccnc3c2)c(N2CCNCC2)c1Cl] |
| 157 | 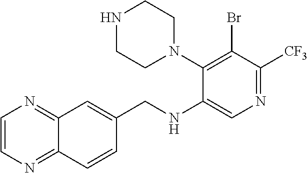 [SMILES: FC(F)(F)c1ncc(NCc2ccc3nccnc3c2)c(N2CCNCC2)c1Br] |
| 158 | 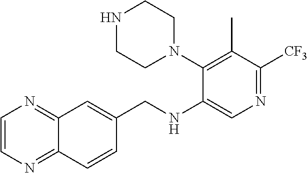 [SMILES: Cc1c(C(F)(F)F)ncc(NCc2ccc3nccnc3c2)c1N1CCNCC1] |
| 159 | 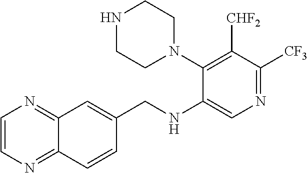 [SMILES: FC(F)c1c(C(F)(F)F)ncc(NCc2ccc3nccnc3c2)c1N1CCNCC1] |
| 160 | 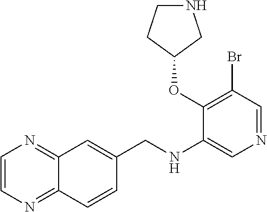 [SMILES: Brc1cncc(NCc2ccc3nccnc3c2)c1O[C@@H]1CCNC1] |
| 161 | 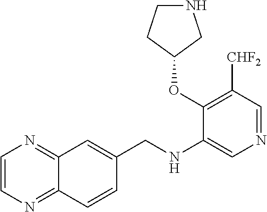 [SMILES: FC(F)c1cncc(NCc2ccc3nccnc3c2)c1O[C@@H]1CCNC1] |
| 162 | 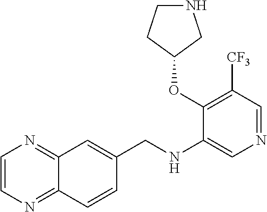 [SMILES: FC(F)(F)c1cncc(NCc2ccc3nccnc3c2)c1O[C@@H]1CCNC1] |
| 163 | 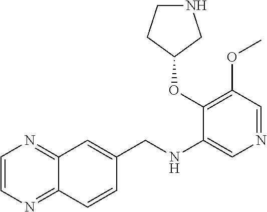 [SMILES: COc1cncc(NCc2ccc3nccnc3c2)c1O[C@@H]1CCNC1] |
| 164 | 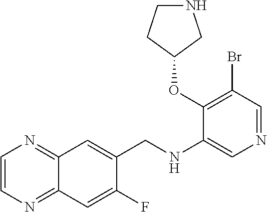 [SMILES: Fc1cc2nccnc2cc1CNc1cncc(Br)c1O[C@@H]1CCNC1] |
| 165 | 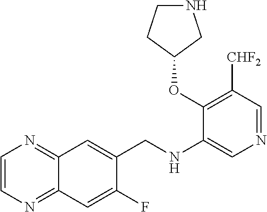 [SMILES: Fc1cc2nccnc2cc1CNc1cncc(C(F)F)c1O[C@@H]1CCNC1] |
| 166 | 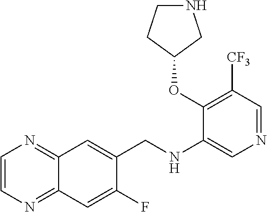 [SMILES: Fc1cc2nccnc2cc1CNc1cncc(C(F)(F)F)c1O[C@@H]1CCNC1] |
| 167 | 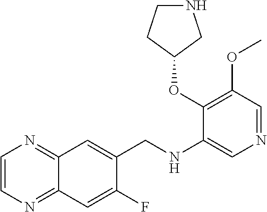 [SMILES: COc1cncc(NCc2cc3nccnc3cc2F)c1O[C@@H]1CCNC1] |
| 168 | 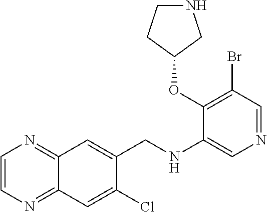 [SMILES: Clc1cc2nccnc2cc1CNc1cncc(Br)c1O[C@@H]1CCNC1] |
| 169 | 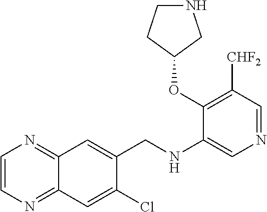 [SMILES: FC(F)c1cncc(NCc2cc3nccnc3cc2Cl)c1O[C@@H]1CCNC1] |
| 170 | 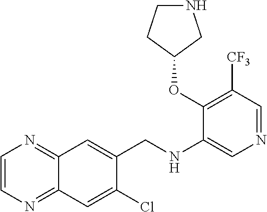 [SMILES: FC(F)(F)c1cncc(NCc2cc3nccnc3cc2Cl)c1O[C@@H]1CCNC1] |
| 171 | 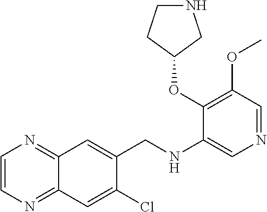 [SMILES: COc1cncc(NCc2cc3nccnc3cc2Cl)c1O[C@@H]1CCNC1] |
| 172 | 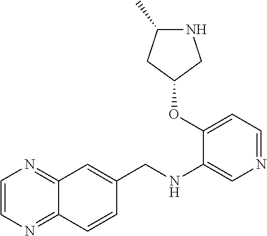 [SMILES: C[C@H]1C[C@@H](Oc2ccncc2NCc2ccc3nccnc3c2)CN1] |
| 173 | 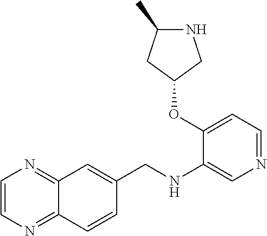 [SMILES: C[C@@H]1C[C@@H](Oc2ccncc2NCc2ccc3nccnc3c2)CN1] |
| 174 | 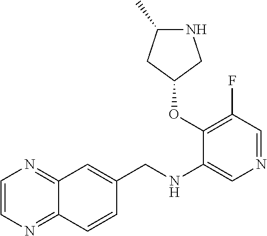 [SMILES: C[C@H]1C[C@@H](Oc2c(F)cncc2NCc2ccc3nccnc3c2)CN1] |
| 175 | 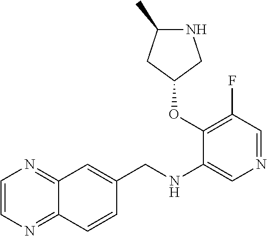 [SMILES: C[C@@H]1C[C@@H](Oc2c(F)cncc2NCc2ccc3nccnc3c2)CN1] |
| 176 | 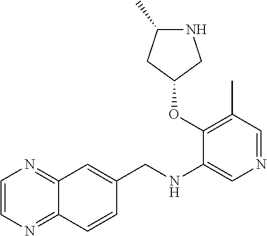 [SMILES: Cc1cncc(NCc2ccc3nccnc3c2)c1O[C@H]1CN[C@@H](C)C1] |
| 177 | 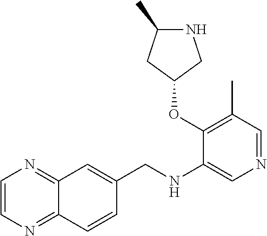 [SMILES: Cc1cncc(NCc2ccc3nccnc3c2)c1O[C@H]1CN[C@H](C)C1] |
| 178 | 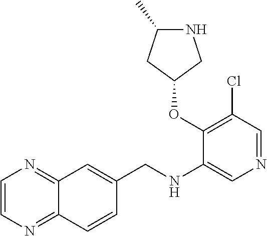 [SMILES: C[C@H]1C[C@@H](Oc2c(Cl)cncc2NCc2ccc3nccnc3c2)CN1] |
| 179 | 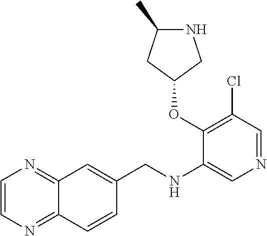 [SMILES: C[C@@H]1C[C@@H](Oc2c(Cl)cncc2NCc2ccc3nccnc3c2)CN1] |
| 180 | 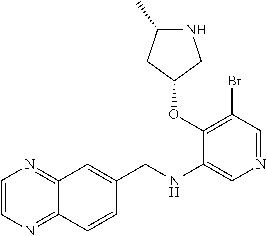 [SMILES: C[C@H]1C[C@@H](Oc2c(Br)cncc2NCc2ccc3nccnc3c2)CN1] |
| 181 | 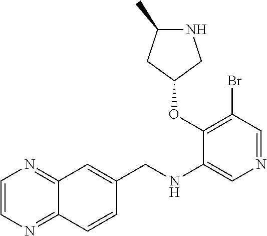 [SMILES: C[C@@H]1C[C@@H](Oc2c(Br)cncc2NCc2ccc3nccnc3c2)CN1] |
| 182 | 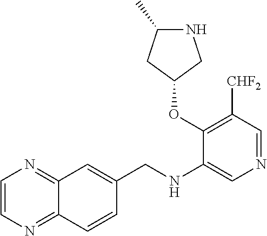 [SMILES: C[C@H]1C[C@@H](Oc2c(NCc3ccc4nccnc4c3)cncc2C(F)F)CN1] |
| 183 | 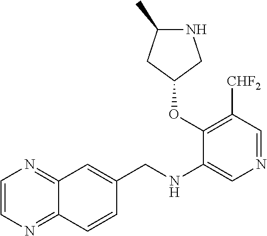 [SMILES: C[C@@H]1C[C@@H](Oc2c(NCc3ccc4nccnc4c3)cncc2C(F)F)CN1] |
| 184 | 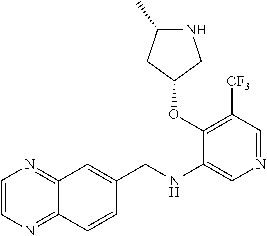 [SMILES: C[C@H]1C[C@@H](Oc2c(NCc3ccc4nccnc4c3)cncc2C(F)(F)F)CN1] |
| 185 | 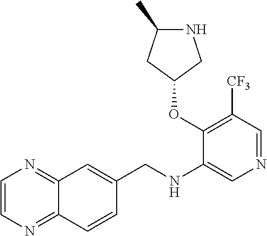 [SMILES: C[C@@H]1C[C@@H](Oc2c(NCc3ccc4nccnc4c3)cncc2C(F)(F)F)CN1] |

61. The compound of claim 54, having the structure according to one of: 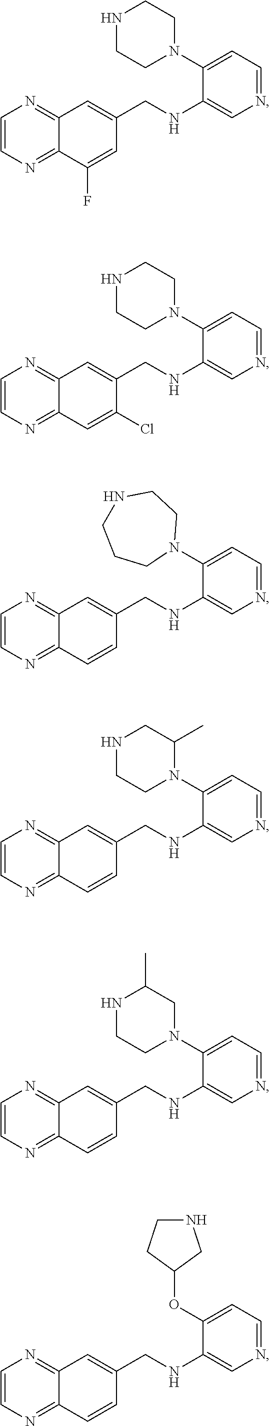 [SMILES: Fc1cc(CNc2cnccc2N2CCNCC2)cc2nccnc12 ; Clc1cc2nccnc2cc1CNc1cnccc1N1CCNCC1 ; c1cc(N2CCCNCC2)c(NCc2ccc3nccnc3c2)cn1 ; CC1CNCCN1c1ccncc1NCc1ccc2nccnc2c1 ; CC1CN(c2ccncc2NCc2ccc3nccnc3c2)CCN1 ; c1cc(OC2CCNC2)c(NCc2ccc3nccnc3c2)cn1] 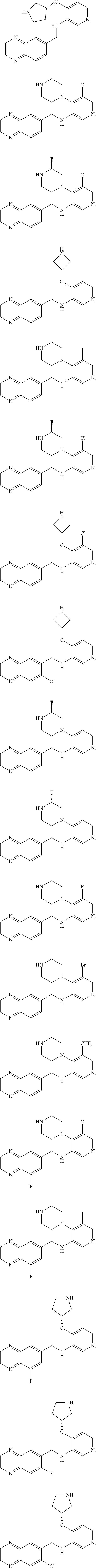 [chemical structure]
